(19) United States
(12) Patent Application Publication
(10) Pub. No.: US 2007/0238694 A1
(43) Pub. Date: Oct. 11, 2007

(54) Purine compounds and methods of use thereof

(75) Inventors: Andrew L. Salzman, Belmont, MA (US); Prakash Jagtap, North Andover, MA (US)

Correspondence Address: WILMER CUTLER PICKERING HALE AND DORR LLP, 399 PARK AVENUE, NEW YORK, NY, 10022, US

(73) Assignee: Inotek Pharmaceuticals Corporation, Beverly, MA (US)

(21) Appl. No.: 11/726,572

(22) Filed: Mar. 22, 2007

Related U.S. Application Data
(60) Provisional application No. 60/785,093, filed on Mar. 23, 2006.
(60) Provisional application No. 60/785,092, filed on Mar. 23, 2006.
(60) Provisional application No. 60/785,014, filed on Mar. 23, 2006.
(60) Provisional application No. 60/785,094, filed on Mar. 23, 2006.

(51) Int. Cl. A61K 31/7076; C07H 19/20; C07H 19/19

(52) U.S. Cl. 514/47; 536/27.4; 536/26.71

(57) ABSTRACT

The invention relates to Purine Compounds; compositions comprising an effective amount of a Purine Compound; and methods for reducing a subject's rate of metabolism or protecting a subject's heart against myocardial damage during cardioplegia; or for treating or preventing a cardiovascular disease, a neurological disorder, an ischemic condition, a reperfusion injury, obesity, a wasting disease, diabetes, a cellular proliferative disorder, a skin disorder, a radiation-induced injury, a wound or an inflammatory disease comprising administering an effective amount of a Purine Compound to a subject in need thereof.

This application claims the benefit of U.S. Provisional Application No. 60/785,093, filed Mar. 23, 2006, U.S. Provisional Application No. 60/785,092, filed Mar. 23, 2006, U.S. Provisional Application No. 60/785,014, filed Mar. 23, 2006, and U.S. Provisional Application No. 60/785,094, filed Mar. 23, 2006, the entire disclosure of each of which is incorporated by reference herein in its entirety.

### 1. FIELD OF THE INVENTION

The invention relates to Purine Compounds; compositions comprising an effective amount of a Purine Compound; and methods for reducing a subject's rate of metabolism or protecting a subject's heart against myocardial damage during cardioplegia; or for treating or preventing a cardiovascular disease, a neurological disorder, an ischemic condition, a reperfusion injury, obesity, a wasting disease, diabetes, a cellular proliferative disorder, a skin disorder, a radiation-induced injury, a wound or an inflammatory disease comprising administering an effective amount of a Purine Compound to a subject in need thereof.

### 2. BACKGROUND OF THE INVENTION

Adenosine is a naturally occurring purine nucleoside that is ubiquitous in mammalian cell types. Adenosine exerts its biological effects by interacting with A₁, A₂ (further subclassified as A_{2A} and A_{2B}) and A₃ cell surface receptors, which modulate important physiological processes.

The A₁ and A_{2A} receptor subtypes are believed to play complementary roles in adenosine's regulation of a cell's energy supply. Adenosine, which is a metabolic product of ATP, diffuses from the cell and locally activates the A₁ receptor to decrease the oxygen demand or activates the A_{2A} receptor to increase the oxygen supply, thereby reinstating the balance of energy supply and demand within the tissue. The combined action of A₁ and A₂ subtypes increases the amount of available oxygen to tissue and protects cells against damage caused by a short-term imbalance of oxygen. One of the important functions of endogenous adenosine is to prevent tissue damage during traumas such as hypoxia, an ischemic condition, hypotension and seizure activity.

Modulation of A₁ receptors slows conduction velocity in the heart's atrioventricular node, resulting in the normalization of supraventricular tachycardias and control of ventricular rate during atrial fibrillation and flutter. Modulation of A_{2A} receptors also regulates coronary vasodilation.

In addition, modulation of A_{2A} receptors also regulates coronary vasodilation and A_{2A} agonists are known to down-regulate the production of multiple inflammatory mediators and are beneficial in various animal models of inflammation.

Adenosine is also a neuromodulator, which modulates molecular mechanisms underlying many aspects of physiological brain function by mediating central inhibitory effects. An increase in neurotransmitter release follows traumas such as hypoxia, ischemia and seizures. Neurotransmitters are ultimately responsible for neural degeneration and neural death, which can cause brain damage or death. Adenosine is thought to be an endogenous anticonvulsant agent that inhibits glutamate release from excitory neurons and neuronal firing. Adenosine agonists, therefore, are useful as antiepileptic agents.

Adenosine plays an important role as a cardioprotective agent. Levels of endogenous adenosine increase in response to ischemia and hypoxia and protect cardiac tissue during and after trauma (preconditioning). Adenosine agonists thus are useful as cardioprotective agents.

The preparation and use of a number of adenosine A₁ receptor agonists have been described (Moos et al., J. Med. Chem. 28:1383-1384 (1985); Thompson et al., J. Med. Chem. 34:3388-3390 (1991); Vittori et al., J. Med. Chem. 43:250-260 (2000); Roelen et al., J Med. Chem, 39:1463-1471 (1996); van der Wenden et al., J Med. Chem. 41102-108 (1998); Dalpiaz et al., Pharm. Res. 18:531-536 (2001), Beakers et al., J. Med. Chem. 46,1492-1503 (2003); U.S. Pat. No. 5,589,467 to Lau et al.; U.S. Pat. No. 5,789,416, to Lum et al.; and C. E. Muller, Current Medicinal Chemistry 2000, 7, 1269-1288).

Nucleoside 5′-nitrate esters are reported in Lichtenthaler et al., Synthesis, 199-201 (1974), and U.S. Pat. No. 3,832,341 to Duchinsky et al.

Adenosine A_{2B} receptors are ubiquitous and regulate multiple biological activities. A_{2B} receptors have been implicated in mast-cell activation, asthma, vasodilation, regulation of cell growth, intestinal function, and modulation of neurosecretion. For example, adenosine binds to A_{2B} receptors on endothelial cells and stimulates angiogenesis. Adenosine also regulates the growth of smooth muscle cell populations in blood vessels and stimulates A_{2B} receptors on mast cells, thus modulating Type I hypersensitivity reactions. In addition, Adenosine stimulates gastrosecretory activity by ligation with A_{2B} in the intestine.

In vitro studies have shown that adenosine receptor agonists promote the migration of endothelial cells and fibroblasts, and adenosine receptor agonists have proven to be useful to treat wounds and promote wound healing.

Adenosine A₃ receptors modulate cell proliferation processes. See Bradley et al., J. Pharmacl. Exptl. Ther. 2001, 299:748-52.

International Publication No. WO 95/02604 discloses A₃ adenosine receptor agonists and their use as locomotor depressants, hypotensive agents, anxiolytic agents, cerebroprotectants and antiseizure agents. U.S. Pat. No. 5,443,836 to Downey et al., discloses the use of adenosine A₃ agonists for preventing ischemic heart damage. International Publication Nos. WO 98/50047 and WO 99/20284 also relate to ischemic protection.

International Publication No. WO 01/19360 discloses the use of A₃ agonists to induce G-CSF secretion, induce proliferation or differentiation of bone marrow or white blood cells, treat or prevent leukopenia, treat or prevent toxic side effects of certain drugs, inhibit abnormal cell growth, and treat cancer.

International Publication No. WO 01/083152 discloses the use of adenosine A₃ receptor agonists to activate natural killer (NK) cells.

International Publication No. WO 02/055085 discloses the use of adenosine A₃ agonists to inhibit viral replication.

For a review of recent developments in the field of adenosine receptor agonists, see C. E. Muller, “Adenosine Receptor Ligands-Recent Developments Part I. Agonists,” in Current Medicinal Chemistry 2000, 7:1269-1288.

2-(N′-Alkylidenehydrazino)adenosines and their 5′-S-alkyl-5′-thio derivatives are reported in U.S. Pat. No. 5,278,150 to Olsson et al.; International Publication No. WO 9602553 to Di Ayres; Niiya et al. J. Med. Chem. 35:4557-4561 (1992); Niiya et al., J. Med. Chem. 35:4562-4566 (1992); Maget et al., Eur. J. Med. Chem. 30:15-25 (1995); Viziano et al., J. Med. Chem. 38:3581-3585 (1995); and Tilburg et al., J. Med. Chem. 45:420-429 (2002).

2-Cyanoadenosine derivatives are reported in Nair et al., J. Am. Chem. Soc. 111:8502-8504 (1989) and Ohno et al., Bioorg. Med. Chem., 12:2995-3007 (2004).

2-Cyano-6-substituted purines are disclosed in U.S. Pat. No. 5,219,840 to Gadient et al.; U.S. Pat. No. 6,448,236 to Monaghan; U.S. Pat. No. 6,638,914 to Fishman et al.; U.S. Pat. No. 6,921,753 to Mandell et al.; U.S. Patent Publication No. US 2002/0032168 to Mantell et al.; and U.S. Patent Publication No. US 2002/0058641 to Mantell et al.

2-Aminosubstituted adenosines and their 5′-amide derivatives are reported in Francis et al., J. Med. Chem. 34:2570-2579 (1991); Hutchison et al., J. Med. Chem. 33:1919-1924 (1990); U.S. Pat. No. 4,968,697 to Hutchison et al.; U.S. Pat. No. 5,280,015 to Jacobsen et al.; and U.S. Pat. No. 6,368,573 to Leung et al.

2-Alkylideneadenosines, 2-Alkyleneadenosines and 5′-carboxamides thereof are reported in Cristalli et al., J. Med. Chem. 38:1462-1472 (1995); Cristalli et al., J. Med. Chem. 37:1720-1726 (1994); Homma et al., J. Med. Chem. 35:2881-2890 (1992); Matsuda et al., J. Med. Chem. 35:241-252 (1992); Rieger et al., J. Med. Chem. 44:531-539 (2001); Beraldi et al., J. Med. Chem. 41:3174-3185 (1998); Vittori et al., J. Med. Chem. 39:4211-4217; U.S. Pat. No. 6,531,457 to Linden et al.; and U.S. Pat. No. 6,180,615 to Zablocki et al.

2-Chloro and 5′-substituted adenosines are disclosed in U.S. Pat. No. 5,589,467 to Lau et al.

2-Pyrazole and thiophene derivatives are disclosed in U.S. Pat. No. 6,403,567 to Zablocki et al.; U.S. Pat. No. 6,214,807 to Zablocki et al.; and U.S. Pat. No. 6,440,948 to Zablocki et al.

2-Carboxamides and aminomethyleneadenosine derivatives are disclosed in U.S. Pat. No. 6,525,032 to Mantell et al.; U.S. Patent Publication No. US 2002/0032168 to Mantell et al.; and U.S. Patent Publication No. US 2002/0058641 to Mantell et al.

2-Alkyl and aminoalkyl adenosine are disclosed in U.S. Pat. No. 6,326,359 to Monaghan et al.; U.S. Pat. No. 6,448,236 to Monaghan et al.; and U.S. Patent Publication No. US 2003/0013675 to Yeadon et al.

2-Thioether nucleosides are reported in U.S. Patent Publication No. US 2001/0051612 to Cristalli.

2-Aminoalkyl and 5′-heterocyclic nucleosides are disclosed in U.S. Pat. No. 6,426,337 to Cox et al.; U.S. Pat. No. 6,534,486 to Allen et al.; and U.S. Pat. No. 6,528,494 to Cox et al.

2-Alkoxyadenosines are reported in U.S. Pat. No. 5,140,015 to Olsson et al.

3′-Aminoadenosine derivatives are reported as highly selective A₃ agonists in DiNinno et al., J. Med. Chem., 46:353-355, (2003); and U.S. Patent Publication No. 2003/0055021 to DeNinno et al.

2′,3′-cyclic phosphate-substituted inosine derivatives are disclosed as being useful for the treatment or prevention of a reperfusion disease or an inflammation disease in U.S. Patent Publication No. 2003/0040502 to Salzman et al.

The 2′ and 3′-monophosphate derivatives of adenosine, as well as the 2′,3′-cyclic phosphate derivative of adenosine are disclosed in Brown et al., J. Chem. Soc., 52-58 (1952).

Monophosphate esters of adenosine are disclosed in Sakakura et al., Org. Letters 7:1999-2002 (2005).

Non-adenosine adenosine A_{2B} receptor agonists are reported in Beukers et al., J. Med. Chem., 47:3707-3709 (2004).

PCT/US2006/045845 filed on Nov. 30, 2006 discloses purine compounds.

The citation of any reference in Section 2 of this application is not an admission that the reference is prior art to this application.

### 3. SUMMARY OF THE INVENTION

In a first aspect, the invention provides a compound having the Formula (I): 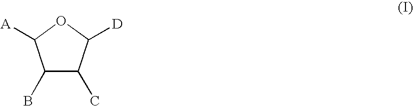
and pharmaceutically acceptable salts thereof, wherein

A is —C(O)NHR³, —CH₂NHR¹¹, —CH₂OSO₂NH₂, —CH₂ONO₂, —CH₂ONO, —CH₂OSO₃H, —CH₂OSO₂NH(C₁-C₁₀ alkyl), —CH₂OSO₂N(C₁-C₁₀ alkyl)₂, —CH₂OH or —CH₂OSO₂NH-aryl, where each C₁-C₁₀ alkyl is independent;

B is —OR⁹;

C is —OR¹⁰;

R⁹ and R¹⁰ are independently the residue of a naturally occurring amino acid that is attached via its C-terminus, or R⁹ and R¹⁰ join to form a —P(O)(OH)— group;

D is:

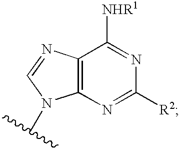

A and B are trans with respect to each other;

B and C are cis with respect to each other;

C and D are cis or trans with respect to each other;

when A is —C(O)NHR³, —CH₂OSO₂NH(C₁-C₁₀ alkyl), —CH₂OSO₂N(C₁-C₁₀ alkyl)₂, or —CH₂OSO₂NH-aryl, where each C₁-C₁₀ alkyl is independent, then R¹ is H, —C₁-C₁₀ alkyl, -aryl, —(C₁-C₆alkylene)-aryl, —(C₁-C₆alkylene)-(arylene)-halo, -3 to 7-membered monocyclic heterocycle, -8 to 12-membered bicyclic heterocycle, —(CH₂)ₙ—C₃-C₈ monocyclic cycloalkyl, —(CH₂)ₙ—C₃-C₈ monocyclic cycloalkenyl, —(C₃-C₈ monocyclic cycloalkene)-OH, —(CH₂)ₙ—C₈-C₁₂ bicyclic cycloalkyl, —(CH₂)ₙ—C₈-C₁₂ bicyclic cycloalkenyl, or —(CH₂)ₙ-aryl;

when A is —CH₂OSO₂NH₂, then R¹ is —C₃-C₈ monocyclic cycloalkyl, —(C₃-C₈ monocyclic cycloalkylene)-OH, —C₃-C₈ monocyclic cycloalkenyl, —(CH₂)ₙ—(C₃-C₈ monocyclic cycloalkyl), —(CH₂)ₙ—(C₃-C₈ monocyclic cycloalkenyl), —C₈-C₁₂ bicyclic cycloalkyl, or —C₈-C₁₂ bicyclic cycloalkenyl;

when A is —CH₂NHR¹¹, —CH₂ONO₂, —CH₂ONO, —CH₂OH, or —CH₂OSO₃H, then R¹ is —H, —C₁-C₁₀ alkyl, -aryl, -3 to 7-membered monocyclic heterocycle, -8 to 12 -membered bicyclic heterocycle, —C₃-C₈ monocyclic cycloalkyl, —C₃-C₈ monocyclic cycloalkenyl, —(C₃-C₈ monocyclic cycloalkyl)-OH, —(C₃-C₈ monocyclic cycloalkylene)-OH, —C₈-C₁₂ bicyclic cycloalkyl, —C₈-C₁₂ bicyclic cycloalkenyl, —(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkyl), —(CH₂)ₙ—(C₈-C₁₂ bicyclic cycloalkenyl), or —(CH₂)ₙ-aryl;

R² is —H, halo, —CN, —NHR⁴, —OR⁴, —SR⁴, —NHC(O)OR⁴, —NHC(O)R⁴—NHC(O)NHR⁴, —NHNHC(O)R₄, —NHNHC(O)NHR₄, —NHNHC(O)OR⁴, —NH—N═C(R⁵)R⁶, —NR⁵—N═C(R⁵)R⁶ or —NR⁵—N(R⁷)R⁸;

R³ is —C₁-C₁₀ alkyl, -aryl, -3 to 7-membered monocyclic heterocycle, -8 to 12-membered bicyclic heterocycle, —C₃-C₈ monocyclic cycloalkyl, —(CH₂)—(C₃-C₈ monocyclic cycloalkyl), —C₃-C₈ monocyclic cycloalkenyl, —C₈-C₁₂ bicyclic cycloalkyl or —C₈-C₁₂ bicyclic cycloalkenyl;

R⁴ is —H, —C₁-C₁₅ alkyl, -aryl, —(CH₂)ₙ-aryl, —(CH₂)ₙ—(C₃-C₈ monocyclic cycloalkyl), —O—(CH₂)ₙ—(C₃-C₈ monocyclic cycloalkyl), —(CH₂)ₙ—(C₃-C₈ monocyclic cycloalkenyl), —O—(CH₂)ₙ—(C₃-C₈ monocyclic cycloalkenyl), —(CH₂)ₙ—(C₈-C₁₂ bicyclic cycloalkyl), —O—(CH₂)ₙ—(C₈-C₁₂ bicyclic cycloalkyl), —(CH₂)ₙ—(C₈-C₁₂ bicyclic cycloalkenyl), —(CH₂)ₙ-(3 to 7-membered monocyclic heterocycle) or —(CH₂)ₙ-(8 to 12-membered bicyclic heterocycle) —C≡C—(C₁-C₁₀ alkyl) or —C≡C-aryl;

each occurrence of R⁵ is independently —H, —C₁-C₁₀ alkyl, -aryl, —(CH₂)ₙ-aryl, —(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkyl), —(CH₂)ₙ—(C₈-C₁₂ bicyclic cycloalkenyl), —(CH₂)ₙ-(3 to 7-membered monocyclic heterocycle), —(CH₂)ₙ-(8 to 12-membered bicyclic heterocycle), —(CH₂)ₘ-phenylene-(C₂-C₁₀ alkynyl), —(CH₂)ₘ-phenylene-(CH₂)ₘCOOH, —(CH₂)ₘ-phenylene-(CH₂)ₘCOO—(C₁-C₁₀ alkyl), —(CH₂)ₘ-phenylene-(CH₂)ₘ—(C₃-C₇-membered monocyclic heterocycle), or —(CH₂)ₘ—C(O)—(C₁-C₁₀ alkyl);

or R⁵ and R⁶, together with the carbon atom to which they are attached, join to form a cyclopentyl, 2-cyclopentenyl, 3-cyclopentenyl, cyclohexyl, 2-cyclohexenyl, 3-cyclohexenyl ring or 1,2,3,4-tetrahydronaphthalene group;

or when A is —CH₂OSO₂NH₂, —CH₂ONO, —CH₂OH or —CH₂OSO₃H, then R⁵ and R⁶, together with the carbon atom to which they are attached, join to form —C₃-C₈ monocyclic cycloalkyl, a —C₈-C₁₂ bicyclic cycloalkyl, a —C₃-C₈ monocyclic cycloalkenyl or a —C₈-C₁₂ bicyclic cycloalkenyl;

R⁶ is —H, —C₁-C₁₀ alkyl, -aryl, —(CH₂)ₙ-aryl, —(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkyl), —(CH₂)ₙ—(C₈-C₁₂ bicyclic cycloalkenyl), —(CH₂)ₙ-(3 to 7-membered monocyclic heterocycle), —(CH₂)ₙ-(8 to 12-membered bicyclic heterocycle), —(CH₂)ₘ-phenylene-(C₂-C₁₀ alkynyl), —(CH₂)ₘ-phenylene-(CH₂)ₘ-(-3 to 7-membered monocyclic heterocycle), —(CH₂)ₘ-phenylene-(CH₂)ₘCOOH or —(CH₂)ₘ-phenylene-(CH₂)ₘCOO—(C₁-C₁₀ alkyl);

R⁷ is —C₁-C₁₀ alkyl, -aryl, —(CH₂)ₙ-aryl, —(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkyl), —(CH₂)ₙ—(C₈-C₁₂ bicyclic cycloalkenyl), —(CH₂)ₙ-(-3 to 7-membered monocyclic heterocycle), —(CH₂)ₙ-(-8 to 12-membered bicyclic heterocycle), —(CH₂)ₘ-phenylene-(C₂-C₁₀ alkynyl), —(CH₂)ₘ-phenylene-(CH₂)ₘ—(C₃-C₇membered monocyclic heterocycle), —(CH₂)ₘ-phenylene-(CH₂)ₘCOOH, —(CH₂)ₘ-phenylene-(CH₂)ₘCOO—(C₁-C₁₀ alkyl), —(CH₂)ₘ—C(O)—(C₁-C₁₀ alkyl), or R⁷ and R⁸, together with the nitrogen atom to which they are attached, join to form a -3- to 7-membered nitrogen-containing monocyclic heterocycle or a -8- to 12-membered nitrogen-containing bicyclic heterocycle;

R⁸ is —C₁-C₁₀ alkyl, -aryl, —(CH₂)ₙ-aryl, —(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkyl), —(CH₂)ₙ—(C₈-C₁₂ bicyclic cycloalkenyl), —(CH₂)ₙ-(-3- to 7-membered monocyclic heterocycle), —(CH₂)ₙ-(-8- to 12-membered bicyclic heterocycle), —(CH₂)ₘ-phenylene-(C₂-C₁₀ alkynyl), —(CH₂)ₘ-phenylene-(CH₂)ₘCOOH, —(CH₂)ₘ-phenylene-(CH₂)ₘCOO—(C₁-C₁₀ alkyl), or —(CH₂)ₘ—C(O)—(C₁-C₁₀ alkyl);

R¹¹ is —C(O)O(C₁-C₁₀ alkyl), —C(O)NH(C₁-C₁₀ alkyl), —C(O)N(C₁-C₁₀alkyl)₂, —C(O)NH-aryl, —CH(NH₂)NH₂ or —CH(NH₂)NH(C₁-C₁₀ alkyl);

each m independently is an integer ranging from 0 to 6; and

each n is independently an integer ranging from 0 to 5.

In a further aspect, the present invention also provides compounds having the Formula (II): 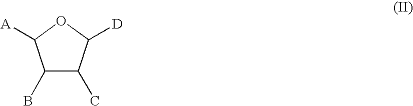
and pharmaceutically acceptable salts thereof, wherein

A is —CH₂OH,

B is —OR³;

C is —OR⁴;

wherein R³ and R⁴ are independently the residue of a naturally occurring amino acid that is attached via its C-terminus, or R³ and R⁴ join to form a —P(O)(OH)—group;

D is:

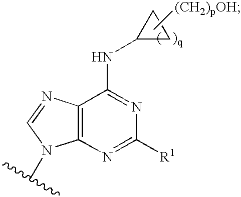

A and B are trans with respect to each other;

B and C are cis with respect to each other;

C and D are cis or trans with respect to each other;

R¹ is —H, -halo, —CN, —N(R²)₂, —OR², —SR², —NHC(O)R², —NHC(O)N(R²), —NHC(O)OR², —C(O)OR₂, —C(O)R², —C(O)N(R²)₂, —OC(O)N(R²)₂, —C(halo)₃, or —NO₂;

each R² is independently —H, —C₁-C₁₀ alkyl, —C₂-C₆ alkenyl, —C₂-C₆ alkynyl, —(CH₂)ₙ-aryl, —(CH₂)ₙ-(3- to 7-membered monocyclic heterocycle), —(CH₂)ₙ-(8- to 12-membered bicyclic heterocycle), —(CH₂)ₙ-(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkyl), or —(CH₂)ₙ—(C₈-C₁₂ bicyclic cycloalkenyl);

each n is an integer ranging from 0 to 6;

each p is an integer ranging from 1 to 6; and

each q is an integer ranging from 1 to 6.

In a still further aspect the present invention provides a compound of formula (III) 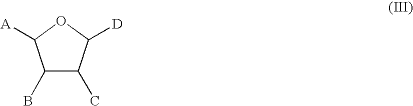
and pharmaceutically acceptable salts thereof, wherein

A is —C(O)NHR³; —CH₂OH, —CH₂ONO₂ or —CH₂OSO₃H;

B is —OR⁵;

C is —OR⁶;

wherein R⁵ and R⁶ are independently the residue of a naturally occurring amino acid that is attached via its C-terminus, or join to form a —P(O)(OH)— group;

D is:

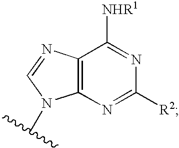

A and B are trans with respect to each other;

B and C are cis with respect to each other;

C and D are cis or trans with respect to each other;

when A is —C(O)NHR³, then R¹ is —H, —C₁-C₆ alkyl, —(C₁-C₆ alkylene)-aryl, or —(C₁-C₆ alkylene)-(arylene)-halo;

when A is —CH₂OH, —CH₂ONO₂ or —CH₂OSO₃H, then R¹ is —H, —C₁-C₆ alkyl, —aryl, -(arylene)-C₁-C₆ alkyl, -3- to 7-membered monocyclic heterocycle, -8- to 12-membered bicyclic heterocycle, —C₃-C₈ monocyclic cycloalkyl, —(C₃-C₈ monocyclic cycloalkylene)-OH, —(CH₂)ₙOH—(C₃-C₈ monocyclic cycloalkylene)-OH, —C₈-C₁₂ bicyclic cycloalkyl, -(3- to 7-membered monocyclic heterocyclene)-S-aryl, —(C₁-C₆ alkylene)-S-(8- to 12-membered bicyclic heterocycle) or —(C₁-C₆ alkylene)-aryl;

R² is —H, -halo, —C₁-C₆ alkyl, -aryl, —CN, —OR⁴, —C(O)NH(CH₂)ₙR⁴, —C≡C—R⁴, —CH═CHR⁴, —NH—N═CHR⁴, —NH(C₁-C₆ alkyl), 3- to 7-membered monocyclic heterocycle, -8- to 12-membered bicyclic heterocycle, —NH((C₁-C₆ alkylene)-C₃-C₈ monocyclic cycloalkyl), —NH—((C₁-C₆ alkylene)-C₈-C₁₂ bicyclic cycloalkyl), —NH((C₁-C₆ alkylene)-aryl), —NH((C₁-C₆ alkylene)-(arylene)-(CH₂)ₙ—COOH),—NH((C₁-C₆ alkylene)-3- to 7-membered monocyclic heterocycle), —CH₂—O—(C₁-C₆ alkyl), —CH₂—NH(C₁-C₆ alkyl) or —CH₂—NH-aryl;

R³ is —C₁-C₆ alkyl;

R⁴ is —H, —C₁-C₆ alkyl, -aryl, -3- to 7-membered monocyclic heterocycle, -8- to 12-membered bicyclic heterocycle, —C₃-C₈ monocyclic cycloalkyl, —CH₂—(C₃-C₈ monocyclic cycloalkyl), —C₈-C₁₂ bicyclic cycloalkyl, —(C₁-C₆ alkylene)-(C₃-C₈ monocyclic cycloalkylene)-CH₂OH; and

n is an integer ranging from 0 to 6.

A compound of Formula (I), Formula (II) or Formula (III), or a pharmaceutically acceptable salt thereof (a “Purine Compound”) is useful for (i) treating or preventing a cardiovascular disease, a neurological disorder, an ischemic condition, a reperfusion injury, obesity, a wasting disease, diabetes, a cellular proliferative disorder, a skin disorder, a radiation-induced injury, a wound or an inflammatory disease (each being a “Condition”); (ii) reducing a subject's rate of metabolism; or (iii) protecting a subject's heart against myocardial damage during cardioplegia.

The invention also provides of compositions comprising an effective amount of a Purine Compound and a physiologically acceptable carrier or vehicle. The compositions are useful for: (i) treating or preventing a Condition; (ii) reducing a subject's rate of metabolism, or (iii) protecting a subject's heart against myocardial damage during cardioplegia.

The invention further provides methods for (i) treating or preventing a Condition, (ii) reducing a subject's rate of metabolism, or (iii) protecting a subject's heart against myocardial damage during cardioplegia, comprising administering an effective amount of a Purine Compound to a subject in need thereof.

The details of the invention are set forth in the accompanying description below. Other features, objects, and advantages of the invention will be apparent from the description and from the claims. All patents, patent applications and publications cited in this specification are incorporated herein by reference for all purposes.

### 4. BRIEF DESCRIPTION OF THE DRAWING

FIG. 1 shows the plasma levels of Compound 54 and the mean peak area of Compound X after intra-tracheal administration of Compound 54 to rats. Blood plasma levels of each compound were measured at 10 minutes, 30 minutes, 60 minutes, and 120 minutes after the administration of Compound 54. The line denoted —▪— represents Compound 54 and the line denoted —♦— represents Compound X. The x-axis represents time after administration in minutes, the left-hand y-axis represents plasma concentration of Compound 54 in ng/mL, and the right-hand y-axis represents the mean peak area of Compound X. The mean peak area of Compound X correlates with the blood plasma levels of Compound X.

### 5. DETAILED DESCRIPTION OF THE INVENTION

### 5.1 Definitions

The term “C₁-C₁₅ alkyl” as used herein refers to a straight or branched chain, saturated hydrocarbon having from 1 to 15 carbon atoms. Representative C₁-C₁₅ alkyl groups include, but are not limited to methyl, ethyl, propyl, isopropyl, butyl, sec-butyl, tert-butyl, pentyl, isopentyl, neopentyl, hexyl, isohexyl, neohexyl heptyl, isoheptyl, neoheptyl, octyl, isooctyl, neooctyl, nonyl, isononyl, neononyl, decyl, isodecyl, neodecyl, undecyl, dodecyl, tridecyl, tetradecyl and pentadecyl. In one embodiment, the C₁-C₁₅ alkyl group is substituted with one or more of the following groups: -halo, —O—(C₁-C₆ alkyl), —OH, —CN, —COOR′, —OC(O)R′, —N(R′)₂, —NHC(O)R′ or —C(O)NHR′ groups wherein each R′ is independently —H or unsubstituted —C₁-C₆ alkyl. Unless indicated, the C₁-C₁₅ alkyl is unsubstituted.

The term “C₁-C₁₀ alkyl” as used herein refers to a straight or branched chain, saturated hydrocarbon having from 1 to 10 carbon atoms. Representative C₁-C₁₀ alkyl groups include, but are not limited to methyl, ethyl, propyl, isopropyl, butyl, sec-butyl, tert-butyl, pentyl, isopentyl, neopentyl, hexyl, isohexyl, neohexyl, heptyl, isoheptyl, neoheptyl, octyl, isooctyl, neooctyl, nonyl, isononyl, neononyl, decyl, isodecyl and neodecyl. In one embodiment, the C₁-C₁₀ alkyl group is substituted with one or more of the following groups: -halo, —O—(C₁-C₆ alkyl), —OH, —CN, —COOR′, —OC(O)R′, —N(R′)₂, —NHC(O)R′ or —C(O)NHR′ groups wherein each R′ is independently —H or unsubstituted —C₁-C₆ alkyl. Unless indicated, the C₁-C₁₀ alkyl is unsubstituted.

The term “C₁-C₆ alkyl” as used herein refers to a straight or branched chain, saturated hydrocarbon having from 1 to 6 carbon atoms. Representative C₁-C₆ alkyl groups include, but are not limited to methyl, ethyl, propyl, isopropyl, butyl, sec-butyl, tert-butyl, pentyl, isopentyl, neopentyl, hexyl, isohexyl, and neohexyl. Unless indicated, the C₁-C₆ alkyl is unsubstituted.

The term “C₁-C₆ alkylene” as used herein refers to a C₁-C₆ alkyl group, wherein one of the C₁-C₆ alkyl group's hydrogen atoms is replaced with a bond. A C₁-C₆ alkylene group can be linear or branched. Representative C₁-C₆ alkylene groups include, but are not limited to —(CH₂)—, —(CH₂CH₂)—, —(CH₂CH₂CH₂)—, —(CH(CH₃)CH₂)—, —(CH₂CH₂CH₂CH₂)—, —(CH₂CH(CH₃)CH₂)—, —(CH₂CH₂CH₂CH₂CH₂)— and —(CH₂CH₂CH₂CH₂CH₂CH₂)—. Unless indicated, the C₁-C₆ alkylene is unsubstituted.

The term “C₁-C₁₀ alkyl” as used herein refers to a straight or branched chain, saturated hydrocarbon having from 1 to 10 carbon atoms. Representative C₁-C₁₀ alkyl groups include, but are not limited to methyl, ethyl, propyl, isopropyl, butyl, sec-butyl, tert-butyl, pentyl, isopentyl, neopentyl, hexyl, isohexyl, neohexyl, heptyl, isoheptyl, neoheptyl, octyl, isooctyl, neooctyl, nonyl, isononyl, neononyl, decyl, isodecyl and neodecyl. In one embodiment, the C₁-C₁₀ alkyl group is substituted with one or more of the following groups: -halo, —O—(C₁-C₆ alkyl), —OH, —CN, —COOR′, —OC(O)R′, —N(R′)₂, —NHC(O)R′ or —C(O)NHR′ groups wherein each R′ is independently —H or unsubstituted —C₁-C₆ alkyl. Unless indicated, the C₁-C₁₀ alkyl group is unsubstituted.

The term “C₂-C₆ alkenyl” refers to a straight or branched chain hydrocarbon containing 2-6 carbon atoms and at least one double bond. Representative C₂-C₆ alkenyl groups include, but are not limited to, ethylene, propylene, 1-butylene, 2-butylene, isobutylene, sec-butylene, 1-pentene, 2-pentene, isopentene, 1-hexene, 2-hexene, 3-hexene and isohexene. In one embodiment, the C₂-C₆ alkenyl group is substituted with one or more of the following groups: -halo, —O—(C₁-C₆ alkyl), —OH, —CN, —COOR′, —OC(O)R′, —N(R′)₂, —NHC(O)R′ or —C(O)NHR′ groups wherein each R′ is independently —H or unsubstituted —C₁-C₆ alkyl. Unless indicated, the C₂-C₆ alkenyl group is unsubstituted.

The term “C₂-C₆ alkynyl” refers to a straight or branched chain unsaturated hydrocarbon containing 2-6 carbon atoms and at least one triple bond. Examples of a C₂-C₆ alkynyl group include, but are not limited to, acetylene, propyne, 1-butyne, 2-butyne, isobutyne, sec-butyne, 1-pentyne, 2-pentyne, isopentyne, 1-hexyne, 2-hexyne, 3-hexyne and isohexyne.

The term “C₂-C₁₀ alkynyl” refers to a straight or branched chain unsaturated hydrocarbon containing 2-10 carbon atoms and at least one triple bond. Examples of a C₂-C₁₀ alkynyl group include, but are not limited to, acetylene, propyne, 1-butyne, 2-butyne, isobutyne, sec-butyne, 1-pentyne, 2-pentyne, isopentyne, 1-hexyne, 2-hexyne, 3-hexyne, isohexyne, 1-heptyne, 2-heptyne, 3-heptyne, isoheptyne, 1-octyne, 2-octyne, 3-octyne, 4-octyne, isooctyne, 1-nonyne, 2-nonyne, 3-nonyne, 4-nonyne, isononyne, 1-decyne, 2-decyne, 3-decyne, 4-decyne, 5-decyne, and isodecyne.

The term “aryl” as used herein refers to a phenyl group or a naphthyl group. In one embodiment, the aryl group is substituted with one or more of the following groups: -halo, —O—(C₁-C₆ alkyl), —OH, —CN, —COOR′, —OC(O)R′, —N(R′)₂, —NHC(O)R′ or —C(O)NHR′ groups wherein each R′ is independently —H or unsubstituted —C₁-C₆ alkyl. Unless indicated, the aryl group is unsubstituted.

The term “C₃-C₈ monocyclic cycloalkyl” as used herein is a 3-, 4-, 5-, 6-, 7- or 8-membered saturated non-aromatic monocyclic cycloalkyl ring.

Representative C₃-C₈ monocyclic cycloalkyl groups include, but are not limited to, cyclopropyl, cyclobutyl, cyclopentyl, cyclohexyl, cycloheptyl and cyclooctyl. In one embodiment, the C₃-C₈ monocyclic cycloalkyl group is substituted with one or more of the following groups: -halo, —O—(C₁-C₆ alkyl), —OH, —CN, —COOR′, —OC(O)R′, —N(R′)₂, —NHC(O)R′ or —C(O)NHR′ groups wherein each R′ is independently —H or unsubstituted —C₁-C₆ alkyl. Unless indicated, the C₃-C₈ monocyclic cycloalkyl group is unsubstituted.

The term “C₃-C₈ monocyclic cycloalkenyl” as used herein is a 3-, 4-, 5-, 6-, 7- or 8-membered non-aromatic monocyclic carbocyclic ring having at least one endocyclic double bond, but which is not aromatic. It is to be understood that when any two groups, together with the carbon atom to which they are attached form a C₃-C₈ monocyclic cycloalkenyl group, the carbon atom to which the two groups are attached remains tetravalent. Representative C₃-C₈ monocyclic cycloalkenyl groups include, but are not limited to, cyclopropenyl, cyclobutenyl, 1,3-cyclobutadienyl, cyclopentenyl, 1,3-cyclopentadienyl, cyclohexenyl, 1,3-cyclohexadienyl, cycloheptenyl, 1,3-cycloheptadienyl, 1,4-cycloheptadienyl, -1,3,5-cycloheptatrienyl, cyclooctenyl, 1,3-cyclooctadienyl, 1,4-cyclooctadienyl, -1,3,5-cyclooctatrienyl. In one embodiment, the C₃-C₈ monocyclic cycloalkenyl group is substituted with one or more of the following groups: -halo, —O—(C₁-C₆ alkyl), —OH, —CN, —COOR′, —OC(O)R′, —N(R′)₂, —NHC(O)R′ or —C(O)NHR′ groups wherein each R′ is independently —H or unsubstituted —C₁-C₆ alkyl. Unless indicated, the C₃-C₈ monocyclic cycloalkenyl group is unsubstituted.

The term “C₃-C₈ monocyclic cycloalkylene” as used herein is a C₃-C₈ monocyclic cycloalkyl group, wherein one of the C₃-C₈ monocyclic cycloalkyl group's hydrogen atoms is replaced with a bond forming enantiomers, diastereomers or mixtures of diastereomers. Representative C₃-C₈ monocyclic cycloalkylene groups include, but are not limited to:

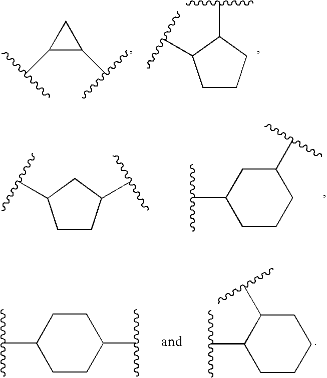

The term “C₈-C₁₂ bicyclic cycloalkyl” as used herein is a 8-, 9-, 10-, 11- or 12-membered saturated, non-aromatic bicyclic cycloalkyl ring system. Representative C₈-C₁₂ bicyclic cycloalkyl groups include, but are not limited to, decahydronaphthalene, octahydroindene, decahydrobenzocycloheptene, and dodecahydroheptalene. In one embodiment, the C₈-C₁₂ bicyclic cycloalkyl group is substituted with one or more of the following groups: -halo, —O—(C₁-C₆ alkyl), —OH, —CN, —COOR′, —OC(O)R′, —N(R′)₂, —NHC(O)R′ or —C(O)NHR′ groups wherein each R′ is independently —H or unsubstituted —C₁-C₆ alkyl. Unless indicated, the C₈-C₁₂ bicyclic cycloalkyl group is unsubstituted.

The term “C₈-C₁₂ bicyclic cycloalkenyl” as used herein is a 8-, 9-, 10-, 11- or 12-membered, aromatic or non-aromatic bicyclic cycloalkyl ring system, having at least one endocyclic double bond. It is to be understood that when any two groups, together with the carbon atom to which they are attached form a C₈-C₁₂ bicyclic cycloalkenyl group, the carbon atom to which the two groups are attached remains tetravalent. Representative C₈-C₁₂ bicyclic cycloalkenyl groups include, but are not limited to, tetrahydronaphthalene, octahydronaphthalene, hexahydronaphthalene, hexahydroindene, tetrahydroindene, octahydrobenzocycloheptene, hexahydrobenzocycloheptene, tetrahydrobenzocyclopheptene, decahydroheptalene, octahydroheptalene, hexahydroheptalene, and tetrahydroheptalene. In one embodiment, the C₈-C₁₂ bicyclic cycloalkyl group is substituted with one or more of the following groups: -halo, —O—(C₁-C₆ alkyl), —OH, —CN, —COOR′, —OC(O)R′, —N(R′)₂, —NHC(O)R′ or —C(O)NHR′ groups wherein each R′ is independently —H or unsubstituted —C₁-C₆ alkyl. Unless indicated, the C₈-C₁₂ bicyclic cycloalkenyl group is unsubstituted.

The term “2-cyclopentenyl” as used herein, refers to the following chemical group:

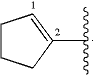

The term “3-cyclopentenyl” as used herein, refers to the following chemical group:

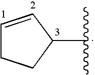

The term “4-cyclopentenyl” as used herein, refers to the following chemical group:

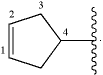

The term “2-cyclohexenyl” as used herein, refers to the following chemical group:

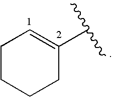

The term “3-cyclohexenyl” as used herein, refers to the following chemical group:

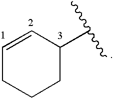

The term “4-cyclohexenyl” as used herein, refers to the following chemical group:

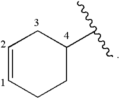

The term “effective amount” as used herein, refers to an amount of a Purine Compound that is effective for treating or preventing a Condition.

The term “halo” as used herein refers to —F, —Cl, —Br or —I.

The term “3- to 7-membered monocyclic heterocycle” refers to: (i) a 3- or 4-membered non-aromatic monocyclic cycloalkyl in which 1 of the ring carbon atoms has been replaced with an N, O or S atom; or (ii) a 5-, 6-, or 7-membered aromatic or non-aromatic monocyclic cycloalkyl in which 1-4 of the ring carbon atoms have been independently replaced with a N, O or S atom. The non-aromatic 3- to 7-membered monocyclic heterocycles can be attached via a ring nitrogen, sulfur, or carbon atom. The aromatic 3- to 7-membered monocyclic heterocycles are attached via a ring carbon atom. Representative examples of a 3- to 7-membered monocyclic heterocycle group include, but are not limited to furanyl, furazanyl, imidazolidinyl, imidazolinyl, imidazolyl, isothiazolyl, isoxazolyl, morpholinyl, oxadiazolyl, oxazolidinyl, oxazolyl, oxazolidinyl, pyrimidinyl, phenanthridinyl, phenanthrolinyl, piperazinyl, piperidinyl, pyranyl, pyrazinyl, pyrazolidinyl, pyrazolinyl, pyrazolyl, pyridazinyl, pyridooxazole, pyridoimidazole, pyridothiazole, pyridinyl, pyrimidinyl, pyrrolidinyl, pyrrolinyl, quinuclidinyl, tetrahydrofuranyl, thiadiazinyl, thiadiazolyl, thienyl, thienothiazolyl, thienooxazolyl, thienoimidazolyl, thiomorpholinyl, thiophenyl, triazinyl, triazolyl, In one embodiment, the 3- to 7-membered monocyclic heterocycle group is substituted with one or more of the following groups: -halo, —C₁-C₆ alkyl, —(C₁-C₆ alkylene)-OR′, —(C₁-C₆ alkylene)-C(O)OR′, —O—(C₁-C₆ alkyl), —OH, —CN, —COOR′, —OC(O)R′, —N(R′)₂, —NHC(O)R′ or —C(O)NHR′ groups wherein each R′ is independently —H or unsubstituted —C₁-C₆ alkyl. Unless indicated, the 3- to 7-membered monocyclic heterocycle group is unsubstituted.

In one embodiment, the 3- to 7-membered monocyclic heterocycle is tetrazolyl.

In another embodiment, the 3- to 7-membered monocyclic heterocycle is oxazolyl.

In still another embodiment, the 3- to 7-membered monocyclic heterocycle is imidazolyl.

In yet another embodiment, the 3- to 7-membered monocyclic heterocycle is triazolyl.

The term “3- to 7-membered monocyclic heterocyclene” refers to a 3- to 7-membered monocyclic heterocycle, wherein one of the the 3- to 7-membered monocyclic heterocycle's hydrogen atoms is replaced with a bond. Representative examples of a 3- to 7-membered monocyclic heterocyclene group include, but are not limited to 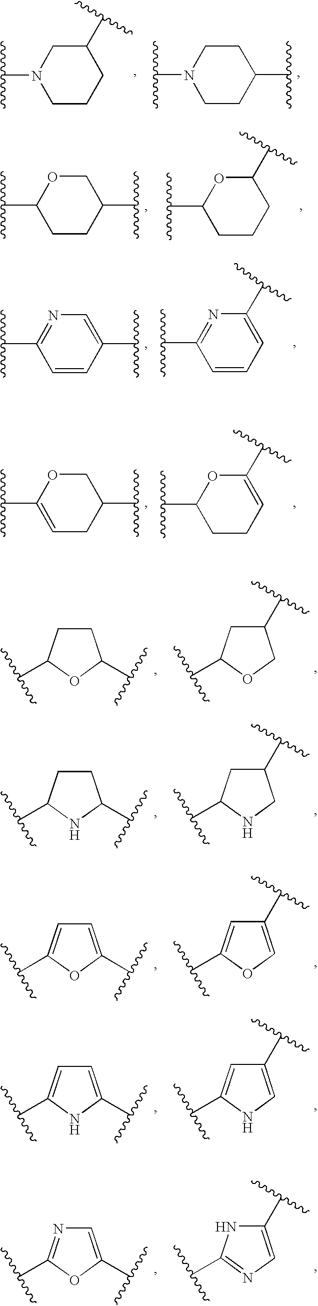 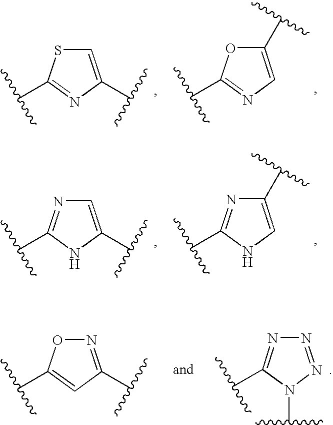
In one embodiment, the 3- to 7-membered monocyclic heterocyclene group is substituted with one or more of the following groups: -halo, —O—(C₁-C₆ alkyl), —OH, —CN, —COOR′, —OC(O)R′, —N(R′)₂, —NHC(O)R′ or —C(O)NHR′ groups wherein each R′ is independently —H or unsubstituted —C₁-C₆ alkyl. Unless indicated, the 3- to 7-membered monocyclic heterocyclene is unsubstituted.

The term “8- to 12-membered bicyclic heterocycle” refers to a bicyclic 8- to 12-membered aromatic or non-aromatic bicyclic cycloalkyl in which one or both of the of the rings of the bicyclic ring system have 1-4 of its ring carbon atoms independently replaced with a N, O or S atom. Included in this class are 3- to 7-membered monocyclic heterocycles that are fused to a benzene ring. A non-aromatic ring of an 8- to 12-membered monocyclic heterocycle is attached via a ring nitrogen, sulfur, or carbon atom. An aromatic 8- to 12-membered monocyclic heterocycles are attached via a ring carbon atom. Examples of 8- to 12-membered bicyclic heterocycles include, but are not limited to, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazolinyl, cinnolinyl, decahydroquinolinyl, 1H-indazolyl, indolenyl, indolinyl, indolizinyl, indolyl, isobenzofuranyl, isoindazolyl, isoindolyl, isoindolinyl, isoquinolinyl, naphthyridinyl, octahydroisoquinolinyl, phthalazinyl, pteridinyl, purinyl, quinoxalinyl, tetrahydroisoquinolinyl, tetrahydroquinolinyl, and xanthenyl. In one embodiment, each ring of the -8- to 12-membered bicyclic heterocycle group can substituted with one or more of the following groups: -halo, —O—(C₁-C₆ alkyl), —OH, —CN, —COOR′, —OC(O)R′, —N(R′)₂, —NHC(O)R′ or —C(O)NHR′ groups wherein each R′ is independently —H or unsubstituted —C₁-C₆ alkyl. Unless indicated, the 8- to 12-membered bicyclic heterocycle group is unsubstituted.

The term “3- to 7-membered nitrogen-containing monocyclic heterocycle” refers to: (i) a 3- or 4-membered non-aromatic monocyclic cycloalkyl in which 1 of the ring carbon atoms has been replaced with a N atom; or (ii) a 5-, 6-, or 7-membered aromatic or non-aromatic monocyclic cycloalkyl in which 1 of the ring carbon atoms has been replaced with a N atom and 0-3 of the remaining ring carbon atoms have been independently replaced with a N, O or S atom. The non-aromatic 3- to 7-membered nitrogen-containing monocyclic heterocycles can be attached via a ring nitrogen, sulfur, or carbon atom. The aromatic 3- to 7-membered nitrogen-containing monocyclic heterocycles are attached via a ring carbon atom. Representative examples of a 3- to 7-membered nitrogen-containing monocyclic heterocycle group include, but are not limited to furanyl, furazanyl, imidazolidinyl, imidazolinyl, imidazolyl, isothiazolyl, isoxazolyl, morpholinyl, oxadiazolyl, oxazolidinyl, oxazolyl, oxazolidinyl, pyrimidinyl, phenanthridinyl, phenanthrolinyl, piperazinyl, piperidinyl, pyranyl, pyrazinyl, pyrazolidinyl, pyrazolinyl, pyrazolyl, pyridazinyl, pyridooxazole, pyridoimidazole, pyridothiazole, pyridinyl, pyrimidinyl, pyrrolidinyl, pyrrolinyl, quinuclidinyl, tetrahydrofuranyl, tetrazolyl, thiadiazinyl, thiadiazolyl, thienyl, thienothiazolyl, thienooxazolyl, thienoimidazolyl, thiomorpholinyl, triazinyl or triazolyl. In one embodiment, the 3- to 7-membered nitrogen-containing monocyclic heterocycle group is substituted with one or more of the following groups: -halo, —O—(C₁-C₆ alkyl), —OH, —CN, —COOR′, —OC(O)R′, —N(R′)₂, —NHC(O)R′ or —C(O)NHR′ groups wherein each R′ is independently —H or unsubstituted —C₁-C₆ alkyl. Unless indicated, the 3- to 7-membered nitrogen-containing monocyclic heterocycle group is unsubstituted.

The term “8- to 12-membered nitrogen-containing bicyclic heterocycle” refers to an 8- to 12-membered aromatic or non-aromatic bicyclic cycloalkyl in which 1 of the ring carbon atoms has been replaced with a N atom and 0-3 of the remaining ring carbon atoms have been independently replaced with a N, O or S atom. Included in this class are 3- to 7-membered nitrogen-containing monocyclic heterocycles that are fused to a benzene ring. A non-aromatic ring of an 8- to 12-membered nitrogen-containing monocyclic heterocycle is attached via a ring nitrogen, sulfur, or carbon atom. The aromatic 8- to 12-membered nitrogen-containing monocyclic heterocycles are attached via a ring carbon atom. Examples of 8- to 12-membered nitrogen-containing bicyclic heterocycles include, but are not limited to, benzimidazolyl, benzoxazolyl, benztriazolyl, benztetrazolyl, benzisoxazolyl, benzisothiazolyl, benzimidazolinyl, cinnolinyl, decahydroquinolinyl, 1H-indazolyl, indolenyl, indolinyl, indolizinyl, indolyl, isoindazolyl, isoindolyl, isoindolinyl, isoquinolinyl, naphthyridinyl, octahydroisoquinolinyl, phthalazinyl, pteridinyl, purinyl, quinoxalinyl, tetrahydroisoquinolinyl, tetrahydroquinolinyl, and xanthenyl. In one embodiment, each ring of the -8- to 12-membered nitrogen-containing bicyclic heterocycle group can substituted with one or more of the following groups: -halo, —O—(C₁-C₆ alkyl), —OH, —CN, —COOR′, —OC(O)R′, —N(R′)₂, —NHC(O)R′ or —C(O)NHR′ groups wherein each R′ is independently —H or unsubstituted —C₁-C₆ alkyl. Unless indicated, the -8- to 12-membered nitrogen-containing bicyclic heterocycle group is unsubstituted.

The term “arylene” as used herein refers to an aryl group, wherein one of the aryl group's hydrogen atoms is replaced with a bond. Representative arylene groups include, but are not limited to: 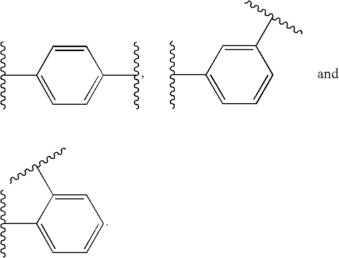
In one embodiment, the arylene group is substituted with one or more of the following groups: -halo, —O—(C₁-C₆ alkyl), —OH, —CN, —COOR′, —OC(O)R′, —N(R′)₂, —NHC(O)R′ or —C(O)NHR′ groups wherein each R′ is independently —H or unsubstituted —C₁-C₆ alkyl. Unless indicated, the arylene is unsubstituted.

The term “phenylene” as used herein, refers to a benzene ring in which two of the benzene ring's hydrogen atoms have been replaced with single bonds. Representative examples of a “phenylene group” are depicted below:

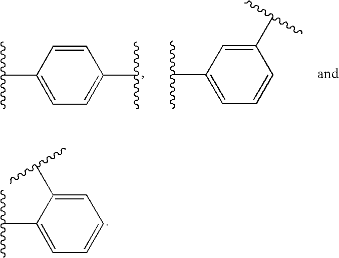

The phrase “pharmaceutically acceptable salt,” as used herein, is a salt of an acid and a basic nitrogen atom of a Purine Compound. Illustrative salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts. The term “pharmaceutically acceptable salt” also refers to a salt of a Purine Compound having an acidic functional group, such as a carboxylic acid functional group, and a base. Suitable bases include, but are not limited to, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or tri-alkylamines, dicyclohexylamine; tributyl amine; pyridine; N-methyl, N-ethylamine; diethylamine; triethylamine; mono-, bis-, or tris-(2-OH-lower alkylamines), such as mono-; bis-, or tris-(2-hydroxyethyl)amine, 2-hydroxy-tert-butylamine, or tris-(hydroxymethyl)methylamine, N,N-di-lower alkyl-N-(hydroxyl-lower alkyl)-amines, such as N,N-dimethyl-N-(2-hydroxyethyl)amine or tri-(2-hydroxyethyl)amine; N-methyl-D-glucamine; and amino acids such as arginine, lysine, and the like. The term “pharmaceutically acceptable salt” also includes a hydrate of a Purine Compound.

A “subject” is a mammal, e.g., a human, mouse, rat, guinea pig, dog, cat, horse, cow, pig, or non-human primate, such as a monkey, chimpanzee or baboon. In one embodiment, the monkey is a rhesus. In one embodiment, the subject is a human.

The term “isolated and purified” as used herein means separate from other components of a reaction mixture or natural source. In certain embodiments, the isolate contains at least 30%, at least 35%, at least 40%, at least 45%, at least 50%, at least 55%, at least 60%, at least 65%, at least 70%, at least 75%, at least 80%, at least 85%, at least 90%, at least 95% or at least 98% of a Purine Compound by weight of the isolate. In one embodiment, the isolate contains at least 95% of a Purine Compound by weight of the isolate.

The term “substantially free of its corresponding opposite enantiomer” as used herein, means that a Purine Compound contains no more than about 10% by weight of its corresponding opposite enantiomer. In one embodiment the Purine Compound that is substantially free of its corresponding opposite enantiomer contains no more than about 5% by weight of its corresponding opposite enantiomer. In a further embodiment a Purine Compound that is substantially free of its corresponding opposite enantiomer contains no more than about 1% by weight of its corresponding opposite enantiomer. In another embodiment a Purine Compound that is substantially free of its corresponding opposite enantiomer contains no more than about 0.5% by weight of its corresponding opposite enantiomer. In still another embodiment a Purine Compound that is substantially free of its corresponding opposite enantiomer contains no more than about 0.1% by weight of its corresponding opposite enantiomer.

The term “substantially free of its corresponding other anomer” as used herein, means that a Purine Compound contains no more than about 10% by weight of its corresponding other anomer. In one embodiment the Purine Compound that is substantially free of its corresponding other anomer contains no more than about 5% by weight of its corresponding other anomer. In a further embodiment a Purine Compound that is substantially free of its corresponding other anomer contains no more than about 1% by weight of its corresponding other anomer. In another embodiment a Purine Compound that is substantially free of its corresponding other anomer contains no more than about 0.5% by weight of its corresponding other anomer. In still another embodiment a Purine Compound that is substantially free of its corresponding other anomer contains no more than about 0.1% by weight of its corresponding other anomer.

Some chemical structures herein are depicted using bold and dashed lines to represent chemical bonds. These bold and dashed lines depict absolute stereochemistry. A bold line indicates that a substituent is above the plane of the carbon atom to which it is attached and a dashed line indicates that a substituent is below the plane of the carbon atom to which it is attached. For example, in the illustration below:

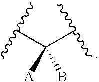

group A is above the plane of the carbon atom to which it is attached and group B is below the plane of the carbon atom to which it is attached.

A “naturally occurring amino acid” is: L-glycine, L-alanine, L-valine, L-leucine, L-isoleucine, L-serine, L-threonine, L-asparagine, L-glutamine, L-phenylalanine, L-tyrosine, L-tryptophan, L-cysteine, L-methionine, L-proline, L-aspartate, L-glutamate, L-lysine, L-arginine or L-histidine.

The following abbreviations are used herein: Ac₂O is acetic anhydride; ATP is adenosine triphosphate; BAIB is iodobenzene diacetate; Bu₃N is n-butylamine; CBZCl is carbobenzyloxy chloride; CCPA is 2-chloro-N⁶-cyclopentyladenosine; CDI is 4,5-dicyanoimidazole; CHO is chinese hamster ovary; CSA is camphorsulfonic acid; DCC is N,N-dicyclohexylcarbodiimide; DMF is N,N-dimethylformamide; EDAC is N-(3-dimethylaminopropyl)-N′-ethylcarbodiimide hydrochloride; EGTA is ethylene glycol bis(3-aminoethyl ether)-N,N,N′,N′-tetraacetic acid; EtNH₂ is ethylamine; EtNO₂ is nitroethane; EtOAc is ethyl acetate; EtOH is ethanol; Et₃SiCl is triethylsilyl chloride; LiHMDS is lithium hexamethyldisilazide; MeOH is methanol; MS is mass spectrometry; NECA is adenosine-5′-(N-ethyl)carboxamido; NMR is nuclear magnetic resonance; R-PIA is N⁶-(2-phenyl-isopropyl)adenosine, R-isomer; TEMPO is 2,2,6,6-tetramethyl-1-piperidinyloxy, free radical; TFA is trifluoroacetic acid; THF is tetrahydrofuran; TMS is trimethylsilyl; TMSOTf is trimethylsilyl triflate.

### 5.1.1 The Purine Compounds of Formula (I)

As stated above, the present invention encompasses Purine Compounds having the Formula (I): 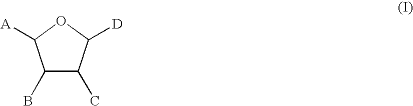
wherein A, B, C and D are defined above for the Purine Compounds of Formula (I), and A and B are trans with respect to each other; B and C are cis with respect to each other; and C and D are cis or trans with respect to each other.

In one embodiment, R¹ is —C₁-C₁₀ alkyl or -3- to 7-membered monocyclic heterocycle.

In another embodiment, R¹ is —C₁-C₁₀ alkyl or -8- to 12-membered bicyclic heterocycle.

In one embodiment, R² is —CN.

In another embodiment, R² is —NHC(O)OR⁴ or —NHC(O)NHR⁴.

In another embodiment, R² is —NHNHC(O)R⁴, —NHNHC(O)OR⁴ or —NHNHC(O)NHR⁴.

In yet another embodiment, R² is —NH—N═C(R⁵)R⁶.

In one embodiment, R³ is —C₁-C₁₀ alkyl.

In another embodiment, R³ is -aryl.

In another embodiment, R³ is -3- to 7-membered monocyclic heterocycle or -8- to 12-membered bicyclic heterocycle.

In still another embodiment, R³ is —C₃-C₈ monocyclic cycloalkyl, —C₃-C₈ monocyclic cycloalkenyl, —C₈-C₁₂ bicyclic cycloalkyl or —C₈-C₁₂ bicyclic cycloalkenyl.

In another embodiment, C and D are trans with respect to each other.

In one embodiment, R⁹ and R¹⁰ are independently the residue of a naturally occurring amino acid.

In a specific embodiment, R⁹ and R¹⁰ are each:

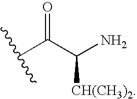

In another embodiment R⁹ and R¹⁰ join to form a —P(O)(OH)— group.

In one embodiment, A is —CH₂SO₃Na.

In one embodiment, R¹ is —H.

In another embodiment, R¹ is —C₁-C₁₀ alkyl.

In a specific embodiment, R¹ is methyl or ethyl.

In another embodiment, R¹ is -aryl or —(CH₂)ₙ-aryl.

In another embodiment, R¹ is —C₃-C₈ monocyclic cycloalkyl.

In a specific embodiment R¹ is cyclopentyl.

In another embodiment, R¹ is —C₃-C₈ monocyclic cycloalkenyl.

In another embodiment, R¹ is —C₈-C₁₂ bicyclic cycloalkyl or —C₈-C₁₂ bicyclic cycloalkenyl.

In another embodiment, R² is —NH—N═C(R⁹)R¹⁰.

In still another embodiment, R² is —NH—N═CH—(C₃-C₈ monocyclic cycloalkenyl).

In another embodiment, R²is —NH—N═CH-phenylene-(CH₂)ₘCOOH.

In a further embodiment, R² is —NH—N═CH-phenylene-(CH₂)ₘ—COO—(C₁-C₁₀ alkyl).

In another embodiment, R³ is -3- to 7-membered monocyclic heterocycle.

In a specific embodiment, R³ is methyl.

In another specific embodiment, R³ is ethyl.

In one embodiment, R¹ is —H and R³ is —C₁-C₁₀ alkyl.

In a specific embodiment, R¹ is —H and R³ is ethyl.

In another embodiment, R¹ is —C₁-C₁₀ alkyl and R³ is —C₁-C₁₀ alkyl.

In a specific embodiment, R¹ and R³ are each ethyl.

In one embodiment, R¹ is —H, R² is —NH—N═C(R⁹)R¹⁰, and R³is —C₁-C₁₀ alkyl.

In a specific embodiment, R¹ is —H, R² is —NH—N═C(R⁹)R¹⁰, and R³ is ethyl.

In another specific embodiment, R² is —H and is R³ is ethyl.

In one embodiment, R³ is —C₃-C₈ monocyclic cycloalkenyl.

In another embodiment, R³ is —C₈-C₁₂ bicyclic cycloalkyl or —C₈-C₁₂ bicyclic cycloalkenyl.

In still another embodiment, R³ is -8- to 12-membered bicyclic heterocycle.

In one embodiment, R¹ is —H, R² is —CN, and R³ is —C₁-C₁₀ alkyl.

In another embodiment, R¹ is —C₁-C₁₀ alkyl, R² is —CN, and R³ is —C₁-C₁₀ alkyl.

In still another embodiment, R¹ is —C₁-C₁₀ alkyl, R² is —CN and R³ is -methyl.

In a further embodiment, R¹ is -methyl, R² is —CN and R³ is —C₁-C₁₀ alkyl.

In still another embodiment, R¹ is —(CH₂)ₙ—(C₃-C₈ monocyclic cycloalkyl) or —(CH₂)ₙ—(C₃-C₈ monocyclic cycloalkenyl).

In one embodiment, R² is -halo.

In a specific embodiment, R² is —Cl.

In another embodiment, R² is —NHR⁴, —OR⁴ or —SR⁴.

In another embodiment, R² is —NH—N═C(R⁵)R⁶ and R⁵ and R together with the carbon atom to which they are attached form a —C₃-C₈ monocyclic cycloalkyl, a —C₈-C₁₂ bicyclic cycloalkyl, a —C₃-C₈ monocyclic cycloalkenyl or a —C₈-C₁₂ bicyclic cycloalkenyl.

In one embodiment, R⁶ is —(CH₂)ₙ—(C₈-C₁₂ bicyclic cycloalkenyl).

In still another embodiment, R¹ is —(CH₂)ₙ—(C₃-C₈ monocyclic cycloalkyl) or —(CH₂)ₙ—(C₃-C₈ monocyclic cycloalkenyl).

In another embodiment, R¹ is -3- to 7-membered monocyclic heterocycle or -8- to 12-membered bicyclic heterocycle.

In one embodiment, R¹¹ is —C(O)O(C₁-C₁₀ alkyl).

In another embodiment, R¹¹ is —C(O)NH(C₁-C₁₀ alkyl), —C(O)N(C₁-C₁₀ alkyl)₂ or —C(O)NH-aryl.

In another embodiment, R¹¹ is —CH(NH₂)NH₂ or —CH(NH₂)NH(C₁-C₁₀ alkyl).

In one embodiment, R¹ is —(CH₂)ₙ-aryl.

In one embodiment, R¹ is —C₅-C₆ monocyclic cycloalkyl.

In another embodiment A is —CH₂ONO, —CH₂OH, —CH₂OSO₃H or —CH₂OSO3Na and R¹ is —H, —C₁-C₁₀ alkyl or C—C₃-C₈ monocyclic cycloalkyl.

In a further embodiment A is —C(O)NHR³, R¹ is —H or —C₁-C₁₀ alkyl, and R² is —CN or NH—N═C(R⁵)R⁶.

The present invention also provides compositions comprising an effective amount of a Purine Compound of Formula (I) and a physiologically acceptable vehicle.

The invention further provides Purine Compounds of Formula (I) that are in isolated and purified form.

The invention still further provides methods for treating or preventing a Condition, comprising administering an effective amount of a Purine Compound of Formula (I) to a subject in need thereof.

The invention further provides methods for reducing a subject's rate of metabolism, comprising administering an effective amount of a Purine Compound of Formula (I) to a subject in need thereof.

The invention further provides methods protecting a subject's heart against myocardial damage during cardioplegia, comprising administering an effective amount of a Purine Compound of Formula (I) to a subject in need thereof.

In one embodiment the invention provides compounds of Formula (167-Ia): 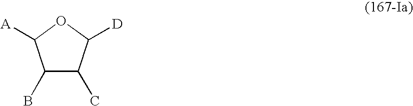
and pharmaceutically acceptable salts thereof, wherein

A is —C(O)NHR³;

B is —OR⁹;

C is —OR¹⁰;

wherein R⁹ and R¹⁰ are independently the residue of a naturally occurring amino acid that is attached via its C-terminus, or R⁹ and R¹⁰ join to form a —P(O)(OH)— group;

D is:

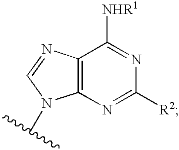

A and B are trans with respect to each other;

B and C are cis with respect to each other;

C and D are cis or trans with respect to each other;

R¹ is -3- to 7-membered monocyclic heterocycle or -8- to 12-membered bicyclic heterocycle;

R² is —CN, —NHCOOR⁴, —NHCONHR⁴, —NHNHCOR⁴, —NHNHCONHR⁴, —NHNHCOOR⁴, —NH—N═C(R⁵)R⁶, —NR⁵—N═C(R⁵)R⁶ or —NR⁵—N(R⁷)R⁸;

R³ is —C₁-C₁₀ alkyl, -aryl, -3- to 7-membered monocyclic heterocycle, -8- to 12-membered bicyclic heterocycle, —C₃-C₈ monocyclic cycloalkyl, —C₃-C₈ monocyclic cycloalkenyl, —C₈-C₁₂ bicyclic cycloalkyl or —C₈-C₁₂ bicyclic cycloalkenyl;

R⁴ is —C₁-C₁₀ alkyl, -aryl, —(CH₂)ₙ-aryl, —(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkyl), —(CH₂)ₙ—(C₈-C₁₂ bicyclic cycloalkenyl), —(CH₂)ₙ-(3- to 7-membered monocyclic heterocycle) or —(CH₂)ₙ-(8- to 12-membered bicyclic heterocycle);

each occurrence of R⁵ is independently —C₁-C₁₀ alkyl, -aryl, —(CH₂),-aryl, —(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkyl), —(CH₂)ₙ—(C₈-C₁₂ bicyclic cycloalkenyl), —(CH₂)ₙ-(-3- to 7-membered monocyclic heterocycle), —(CH₂)ₙ-(-8- to 12-membered bicyclic heterocycle), -phenylene-(C₂-C₁₀ alkynyl), —(CH₂)ₘ-phenylene-(CH₂)ₘCOOH, —(CH₂)ₘ-phenylene-(CH₂)ₘCOO—(C₁-C₁₀ alkyl), —(CH₂)ₘ-phenylene-(CH₂)ₘ-(-3- to 7-membered monocyclic heterocycle), or —(CH₂)ₘ—C(O)—(C₁-C₁₀ alkyl), or R⁵ and R⁶, together with the carbon atom to which they are attached, join to form a cyclopentyl, 2-cyclopentenyl, 3-cyclopentenyl, cyclohexyl, 2-cyclohexenyl or 3-cyclohexenyl ring;

R⁶ is —H, —C₁-C₁₀ alkyl, -aryl, —(CH₂)ₙ-aryl, —(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkyl), —(CH₂)ₙ—(C₈-C₁₂ bicyclic cycloalkenyl), —(CH₂)ₙ-(-3- to 7-membered monocyclic heterocycle), —(CH₂)ₙ-(-8- to 12-membered bicyclic heterocycle), —(CH₂)ₘ-phenylene-(CH₂)ₘ-(-3- to 7-membered monocyclic heterocycle), —(CH₂)ₘ-phenylene-(CH₂)ₘCOOH or —(CH₂)ₘ-phenylene-(CH₂)ₘCOO—(C₁-C₁₀ alkyl);

R⁷ is —C₁-C₁₀ alkyl, -aryl, —(CH₂)ₘ-aryl, —(CH₂)ₘ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkyl), —(CH₂)ₙ—(C₈-C₁₂ bicyclic cycloalkenyl), —(CH₂)ₙ-(-3- to 7-membered monocyclic heterocycle), —(CH₂)ₙ-(-8- to 12-membered bicyclic heterocycle), —(CH₂)ₘ-phenylene-(C₂-C₁₀ alkynyl), —(CH₂)ₘ-phenylene-(CH₂)ₘ-(-3- to 7-membered monocyclic heterocycle), —(CH₂)ₘ-phenylene-(CH₂)ₘCOOH, —(CH₂)ₘ-phenylene-(CH₂)ₘCOO—(C₁-C₁₀ alkyl), —(CH₂)ₘ—C(O)—(C₁-C₁₀ alkyl), or R⁷ and R⁸, together with the nitrogen atom to which they are attached, join to form a -3- to 7-membered nitrogen-containing monocyclic heterocycle or a -8- to 12-membered nitrogen-containing bicyclic heterocycle;

R⁸ is —C₁-C₁₀ alkyl, -aryl, —(CH₂)ₙ-aryl, —(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkyl), —(CH₂)ₙ—(C₈-C₁₂ bicyclic cycloalkenyl), —(CH₂)ₙ-(-3- to 7-membered monocyclic heterocycle), —(CH₂)ₙ-(-8- to 12-membered bicyclic heterocycle), —(CH₂)ₘ-phenylene-(C₂-C₁₀ alkynyl), —(CH₂)ₘ-phenylene-(CH₂)ₘCOOH, —(CH₂)ₘ-phenylene-(CH₂)ₘCOO—(C₁-C₁₀ alkyl), or —(CH₂)ₘ—C(O)—(C₁-C₁₀ alkyl);

each m independently is an integer ranging from 0-4; and

each n is independently an integer ranging from 1 to 5.

In one embodiment, R¹ is -3- to 7-membered monocyclic heterocycle.

In another embodiment, R¹ is -8- to 12-membered bicyclic heterocycle.

In one embodiment, R² is —CN.

In another embodiment, R² is —NHC(O)OR⁴ or —NHC(O)NHR⁴.

In another embodiment, R² is —NHNHC(O)R⁴, —NHNHC(O)OR⁴ or —NHNHC(O)NHR⁴.

In yet another embodiment, R² is —NH—N═C(R⁵)R⁶.

In one embodiment, R³ is —C₁-C₁₀ alkyl.

In another embodiment, R³ is -aryl.

In another embodiment, R³ is -3- to 7-membered monocyclic heterocycle or -8- to 12-membered bicyclic heterocycle.

In still another embodiment, R³ is —C₃-C₈ monocyclic cycloalkyl, —C₃-C₈ monocyclic cycloalkenyl, —C₈-C₁₂ bicyclic cycloalkyl or —C₈-C₁₂ bicyclic cycloalkenyl.

In one embodiment, C and D are cis with respect to each other.

In another embodiment, C and D are trans with respect to each other.

In one embodiment, R⁹ and R¹⁰ are independently the residue of a naturally occurring amino acid.

In a specific embodiment, R⁹ and R¹⁰ are each:

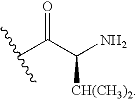

In another embodiment R⁹ and R¹⁰ join to form a —P(O)(OH)— group.

The present invention also provides compositions comprising an effective amount of a Purine Compound of Formula (167-Ia) and a physiologically acceptable vehicle.

The invention further provides Purine Compounds of Formula (167-Ia) that are in isolated and purified form.

The invention still further provides methods for treating or preventing a Condition, comprising administering an effective amount of a Purine Compound of Formula (167-Ia) to a subject in need thereof.

In one embodiment the invention provides compounds having the Formula (167-Ib): 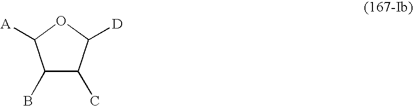
and pharmaceutically acceptable salts thereof, wherein

A is —C(O)NHR³;

B is —OR¹¹;

C is —OR¹²;

wherein R¹¹ and R¹² are independently the residue of a naturally occurring amino acid that is attached via its C-terminus, or R¹¹ and R¹² join to form a —P(O)(OH)— group;

D is:

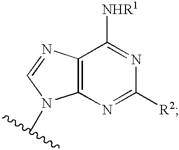

A and B are trans with respect to each other;

B and C are cis with respect to each other;

C and D are cis or trans with respect to each other;

R¹ is —H, —C₁-C₁₀ alkyl, -aryl, —C₃-C₈ monocyclic cycloalkyl, —C₃-C₈ monocyclic cycloalkenyl, —C₈-C₁₂ bicyclic cycloalkyl, —C₈-C₁₂ bicyclic cycloalkenyl, or —(CH₂)ₙ-aryl;

R² is —NHCOOR⁴, —NHCONHR⁴, —NHNHCOR⁴, —NHNHCONHR⁴, —NHNHOOR⁴, —NH—N═C(R⁹)R¹⁰, —NR⁵—N═C(R⁵)R⁶ or —NR⁵—N(R⁷)R⁸;

R³ is —C₁-C₁₀ alkyl, -aryl or -3- to 7-membered monocyclic heterocycle;

R⁴ is —C₁-C₁₀ alkyl, -aryl, —(CH₂)ₙ-aryl, —(CH₂)ₙ-(C₃-C₈ monocyclic cycloalkyl), —(CH₂)ₙ—(C₃-C₈ monocyclic cycloalkenyl), —(CH₂))—(C₈-C₁₂ bicyclic cycloalkyl), —(CH₂)ₙ—(C₈-C₁₂ bicyclic cycloalkenyl), —(CH₂)ₙ-(-3- to 7-membered monocyclic heterocycle) or —(CH₂)ₙ-(-8- to 12-membered bicyclic heterocycle);

each occurrence of R⁵ is independently —C₁-C₁₀ alkyl, —(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkyl), —(CH₂)ₙ—(C₈-C₁₂ bicyclic cycloalkenyl), —(CH₂)ₙ-(-3- to 7-membered monocyclic heterocycle), —(CH₂)ₙ-(-8- to 12-membered bicyclic heterocycle), -phenylene-(C₂-C₁₀ alkynyl), -phenylene-(CH₂)ₘCOOH, -phenylene-(CH₂)ₘ-(-3- to 7-membered monocyclic heterocycle), -phenylene-(CH₂)ₘCOO—(C₁-C₁₀ alkyl) or —C(O)—(C₁-C₁₀ alkyl), or R⁵ and R⁶, together with the carbon atom to which they are attached, join to form a cyclopentyl, 2-cyclopentenyl, 3-cyclopentenyl, cyclohexyl, 2-cyclohexenyl or 3-cyclohexenyl ring;

R⁶ is —H, —C₁-C₁₀ alkyl, —(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkyl), —(CH₂)ₙ—(C₈-C₁₂ bicyclic cycloalkenyl), —(CH₂)ₙ-(-3- to 7-membered monocyclic heterocycle), —(CH₂)ₙ-(-8- to 12-membered bicyclic heterocycle), -phenylene-(C₂-C₁₀ alkynyl), phenylene-(CH₂)ₘCOOH, -phenylene-(CH₂)ₘ-(-3- to 7-membered monocyclic heterocycle), or -phenylene-(CH₂)ₘCOO—(C₁-C₁₀ alkyl);

R⁷ is —C₁-C₁₀ alkyl, -aryl, —(CH₂)ₙ-aryl, —(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkyl), —(CH₂)ₙ—(C₈-C₁₂ bicyclic cycloalkenyl), —(CH₂)ₙ-(-3- to 7-membered monocyclic heterocycle), —(CH₂)ₙ-(-8- to 12-membered bicyclic heterocycle), -phenylene-(C₂-C₁₀ alkynyl), -phenylene-(CH₂)ₘCOOH, -phenylene-(CH₂)ₘ-(-3- to 7-membered monocyclic heterocycle), -phenylene-(CH₂)ₘCOO—(C₁-C₁₀ alkyl) or —C(O)—(C₁-C₁₀ alkyl), or R⁷ and R⁸, together with the nitrogen atom to which they are attached, join to form a -3- to 7-membered nitrogen-containing monocyclic heterocycle or a -8- to 12-membered nitrogen-containing bicyclic heterocycle;

R⁸ is —C₁-C₁₀ alkyl, -aryl, —(CH₂)ₙ-aryl, —(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkyl), —(CH₂)ₙ—(C₈-C₁₂ bicyclic cycloalkenyl), —(CH₂)ₙ-(-3- to 7-membered monocyclic heterocycle), —(CH₂)ₙ-(-8- to 12-membered bicyclic heterocycle), -phenylene-(C₂-C₁₀ alkynyl), -phenylene-(CH₂)ₘCOOH, -phenylene-(CH₂)ₘ-(-3- to 7-membered monocyclic heterocycle), -phenylene-(CH₂)ₘCOO—(C₁-C₁₀ alkyl) or —C(O)—(C₁-C₁₀ alkyl);

R⁹ is —C₁-C₁₀ alkyl, —(CH₂)ₚ—(C₃-C₈ monocyclic cycloalkenyl), —(CH₂)ₚ—(C₈-C₁₂ bicyclic cycloalkyl), —(CH₂)ₚ—(C₈-C₁₂ bicyclic cycloalkenyl), —(CH₂)ₚ-(-3- to 7-membered monocyclic heterocycle), —(CH₂)ₚ-(substituted -3- to 7-membered monocyclic heterocycle), —(CH₂)ₚ-(-8- to 12-membered bicyclic heterocycle), -phenylene-(C₂-C₁₀ alkynyl), -phenylene-(CH₂)ₘCOOH, -phenylene-(CH₂)ₘ-(-3- to 7-membered monocyclic heterocycle), -phenylene-(CH₂)ₘCOO—(C₁-C₁₀ alkyl), —C(O)-phenyl or —C(O)—(C₁-C₁₀ alkyl), or R⁹ and R₁₀, together with the carbon atom to which they are attached, join to form a cyclopentyl, 2-cyclopentenyl, 3-cyclopentenyl, cyclohexyl, 2-cyclohexenyl, 3-cyclohexenyl or 1,2,3,4-tetrahydronaphthalene group;

R¹⁰ is —H, —C₁-C₁₀ alkyl, —(CH₂)ₚ—(C₃-C₈ monocyclic cycloalkenyl), —(CH₂)ₚ—(C₈-C₁₂ bicyclic cycloalkyl), —(CH₂)ₚ—(C₈-C₁₂ bicyclic cycloalkenyl), —(CH₂)ₚ-(-3- to 7-membered monocyclic heterocycle), —(CH₂)ₚ-(-8- to 12-membered bicyclic heterocycle), -phenylene-(C₂-C₁₀ alkynyl), —(CH₂)ₘ-phenylene-(CH₂)ₘCOOH, -phenylene-(CH₂)ₘ-(-3- to 7-membered monocyclic heterocycle) or —(CH₂)ₘ-phenylene-(CH₂)ₘCOO—(C₁-C₁₀ alkyl);

each m is independently an integer ranging from 1 to 4;

each n is independently an integer ranging from 1 to 5; and

each p is independently an integer ranging from 0 to 5.

In one embodiment, R¹ is —H.

In another embodiment, R¹ is —C₁-C₁₀ alkyl.

In a specific embodiment, R¹ is ethyl.

In another embodiment, R¹ is -aryl or —(CH₂)ₙ-aryl.

In another embodiment, R¹ is —C₃-C₈ monocyclic cycloalkyl.

In another embodiment, R¹ is —C₃-C₈ monocyclic cycloalkenyl.

In another embodiment, R¹ is —C₈-C₁₂ bicyclic cycloalkyl or —C₈-C₁₂ bicyclic cycloalkenyl.

In one embodiment, R² is —NHC(O)OR⁴ or —NHC(O)NHR⁴.

In another embodiment, R² is —NHNHC(O)R⁴, —NHNHC(O)OR⁴ or —NHNHC(O)NHR⁴.

In another embodiment, R² is —NH—N═C(R⁹)R¹⁰.

In still another embodiment, R² is —NH—N═CH—(C₃-C₈ monocyclic cycloalkenyl).

In another embodiment, R² is —NH—N═CH-phenylene-(CH₂))ₘCOOH.

In a further embodiment, R² is —NH—N═CH-phenylene-(CH₂)ₘ—COO—(C₁-C₁₀ alkyl).

In one embodiment, R³ is —C₁-C₁₀ alkyl.

In another embodiment, R³ is -aryl.

In another embodiment, R³ is -3- to 7-membered monocyclic heterocycle.

In a specific embodiment, R³ is methyl.

In another specific embodiment, R³ is ethyl.

In one embodiment, R¹ is —H and R³ is —C₁-C₁₀ alkyl.

In a specific embodiment, R¹ is —H and R³ is ethyl.

In another embodiment, R¹ is —C₁-C₁₀ alkyl and R³ is —C₁-C₁₀ alkyl.

In a specific embodiment, R¹ and R³ are each ethyl.

In one embodiment, R¹ is —H, R² is —NH—N═C(R⁹)R¹⁰, and R³ is —C₁-C₁₀ alkyl.

In a specific embodiment, R¹ is —H, R² is —NH—N═C(R⁹)R¹⁰, and R³ is ethyl.

In another specific embodiment, R² is —H and is R³ is ethyl.

In one embodiment, C and D are cis with respect to each other.

In another embodiment, C and D are trans with respect to each other.

In one embodiment, R¹¹ and R¹² are independently the residue of a naturally occurring amino acid.

In a specific embodiment, R¹¹ and R¹² are each:

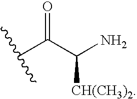

In another embodiment R¹¹ and R¹² join to form a —P(O)(OH)— group.

The present invention also provides compositions comprising an effective amount of a Purine Compound of Formula (167-Ib) and a physiologically acceptable vehicle.

The invention further provides Purine Compounds of Formula (167-Ib) that are in isolated and purified form.

The invention still further provides methods for treating or preventing a Condition, comprising administering an effective amount of a Purine Compound of Formula (167-Ib) to a subject in need thereof.

In still another embodiment the invention provides compounds having the Formula (167-Ic): 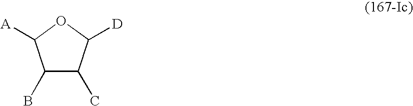
and pharmaceutically acceptable salts thereof,. wherein

A is —C(O)NHR³;

B is —OR⁹;

C is —OR¹⁰;

wherein R⁹ and R¹⁰ are independently the residue of a naturally occurring amino acid that is attached via its C-terminus, or R⁹ and R¹⁰ join to form a —P(O)(OH)— group;

D is:

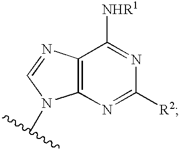

A and B are trans with respect to each other;

B and C are cis with respect to each other;

C and D are cis or trans with respect to each other;

R¹ is —H, —C₁-C₁₀ alkyl, -aryl, —C₃-C₈ monocyclic cycloalkyl, —C₃-C₈ monocyclic cycloalkenyl, —C₈-C₁₂ bicyclic cycloalkyl, —C₈-C₁₂ bicyclic cycloalkenyl, or —(CH₂)ₙ-aryl;

R² is —CN, —NHCOOR⁴, —NHCONHR⁴, —NHNHCOR⁴, —NHNHCONHR⁴, —NHNHCOOR⁴, —NR⁵—N═C(R⁵)R⁶ or —NR⁵—N(R⁷)R⁸;

R³ is —C₃-C₈ monocyclic cycloalkenyl, —C₈-C₁₂ bicyclic cycloalkyl, —C₈-C₁₂ bicyclic cycloalkenyl, -3- to 7-membered monocyclic heterocycle or -8- to 12-membered bicyclic heterocycle;

R⁴ is —C₁-C₁₀ alkyl, -aryl, —(CH₂)ₙ-aryl, —(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkyl), —(CH₂)ₙ—(C₈-C₁₂ bicyclic cycloalkenyl), —(CH₂)ₙ-(-3- to 7-membered monocyclic heterocycle) or —(CH₂)ₙ-(-8- to 12-membered bicyclic heterocycle);

each occurrence of R⁵ is independently —C₁-C₁₀ alkyl, -aryl, —(CH₂)ₙ-aryl, —(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkyl), —(CH₂)ₙ—(C₈-C₁₂ bicyclic cycloalkenyl), —(CH₂)ₙ-(-3- to 7-membered monocyclic heterocycle), —(CH₂)ₙ-(-8- to 12-membered bicyclic heterocycle), —(CH₂)ₘ-phenylene-(C₂-C₁₀ alkynyl), —(CH₂)ₘ-phenylene-(CH₂)ₘCOOH, —(CH₂)ₘ-phenylene-(CH₂)ₘ-(-3- to 7-membered monocyclic heterocycle), —(CH₂)ₘ-phenylene-(CH₂)ₘCOO—(C₁-C₁₀ alkyl) or —(CH₂)ₘ—C(O)—(C₁-C₁₀ alkyl), or R⁵ and R⁶, together with the carbon atom to which they are attached, join to form a cyclopentyl, 2-cyclopentenyl, 3-cyclopentenyl, cyclohexyl, 2-cyclohexenyl or 3-cyclohexenyl ring;

R⁶ is —H, —C₁-C₁₀ alkyl, -aryl, —(CH₂)ₙ-aryl, —(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkyl), —(CH₂)ₙ—(C₈-C₁₂ bicyclic cycloalkenyl), —(CH₂)ₙ-(-3- to 7-membered monocyclic heterocycle), —(CH₂)ₙ-(-8- to 12-membered bicyclic heterocycle), -phenylene-(C₂-C₁₀ alkynyl), -phenylene-(CH₂)ₘCOOH, -phenylene-(CH₂)ₘ-(-3- to 7-membered monocyclic heterocycle) or -phenylene-(CH₂)ₘCOO—(C₁-C₁₀ alkyl);

R⁷ is —C₁-C₁₀ alkyl, -aryl, —(CH₂)ₙ-aryl, —(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkyl), —(CH₂)ₙ—(C₈-C₁₂ bicyclic cycloalkenyl), —(CH₂)ₙ-(-3- to 7-membered monocyclic heterocycle), —(CH₂)ₙ-(-8- to 12-membered bicyclic heterocycle-(CH₂)ₘ-phenylene-(C₂-C₁₀ alkynyl), —(CH₂)ₘ-phenylene-(CH₂)ₘCOOH, —(CH₂)ₘ-phenylene-(CH₂)ₘ-(-3- to 7-membered monocyclic heterocycle), —(CH₂)ₘ-phenylene-(CH₂)ₘCOO—(C₁-C₁₀ alkyl) or —(CH₂)ₘ—C(O)—(C₁-C₁₀ alkyl), or R⁷ and R⁸, together with the nitrogen atom to which they are attached, join to form a a -3- to 7-membered nitrogen-containing monocyclic heterocycle or a -8- to 12-membered nitrogen-containing bicyclic heterocycle;

R⁸ is —C₁-C₁₀ alkyl, -aryl, —(CH₂)ₙ-aryl, —(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkyl), —(CH₂)ₙ—(C₈-C₁₂ bicyclic cycloalkenyl), —(CH₂)ₙ-(-3- to 7-membered monocyclic heterocycle), —(CH₂)ₙ-(-8- to 12-membered bicyclic heterocycle), -phenylene-(C₂-C₁₀ alkynyl), -phenylene-(CH₂)ₘCOOH, -phenylene-(CH₂)ₘ-(-3- to 7-membered monocyclic heterocycle), -phenylene-(CH₂)ₘCOO—(C₁-C₁₀ alkyl) or —C(O)—(C₁-C₁₀ alkyl);

each m is independently an integer ranging from 0 to 4; and

each n is independently an integer ranging from 1 to 5.

In one embodiment, R¹ is —H.

In another embodiment, R¹ is —C₁-C₁₀ alkyl.

In a specific embodiment, R¹ is methyl.

In a specific embodiment, R¹ is ethyl.

In one embodiment, R¹ is -aryl or —(CH₂)ₙ-aryl.

In another embodiment, R¹ is —C₃-C₈ monocyclic cycloalkyl.

In a specific embodiment, R¹ is cyclopentyl.

In another embodiment, R¹ is —C₃-C₈ monocyclic cycloalkenyl.

In another embodiment, R¹ is —C₈-C₁₂ bicyclic cycloalkyl or —C₈-C₁₂ bicyclic cycloalkenyl.

In one embodiment, R² is —CN.

In another embodiment, R² is —NHC(O)OR⁴ or —NHC(O)NHR⁴.

In still another embodiment, R² is —NHNHC(O)R⁴, —NHNHC(O)OR⁴ or —NHNHC(O)NHR⁴.

In one embodiment, R³ is —C₃-C₈ monocyclic cycloalkenyl.

In another embodiment, R³ is —C₈-C₁₂ bicyclic cycloalkyl or —C₈-C₁₂ bicyclic cycloalkenyl.

In yet another embodiment, R³ is -3- to 7-membered monocyclic heterocycle or -8- to 12-membered bicyclic heterocycle;

In one embodiment, C and D are cis with respect to each other.

In another embodiment, C and D are trans with respect to each other.

In one embodiment, R⁹ and R¹⁰ are independently the residue of a naturally occurring amino acid.

In a specific embodiment, R⁹ and R¹⁰ are each:

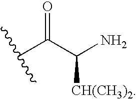

In another embodiment R⁹ and R¹⁰ join to form a —P(O)(OH)— group.

The present invention also provides compositions comprising an effective amount of a Purine Compound of Formula (167-Ic) and a physiologically acceptable vehicle.

The invention further provides Purine Compounds of Formula (167-Ic) that are in isolated and purified form.

The invention still further provides methods for treating or preventing a Condition, comprising administering an effective amount of a Purine Compound of Formula (167-Ic) to a subject in need thereof.

In one further embodiment the invention provides compounds having the Formula (167-Id): 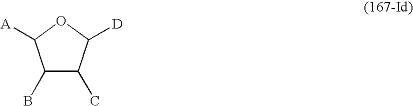
and pharmaceutically acceptable salts thereof, wherein

A is —C(O)NHR³;

B is —OR⁹;

C is —OR¹⁰;

wherein R⁹ and R¹⁰ are independently the residue of a naturally occurring amino acid that is attached via its C-terminus, or R9 and R₁₀ join to form a —P(O)(OH)— group;

D is:

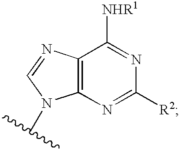

A and B are trans with respect to each other;

B and C are cis with respect to each other;

C and D are cis or trans with respect to each other;

R¹ is —H, —C₁-C₁₀ alkyl, -aryl, -3- to 7-membered monocyclic heterocycle, -8- to 12-membered bicyclic heterocycle, —C₃-C₈ monocyclic cycloalkyl, —C₃-C₈ monocyclic cycloalkenyl, —C₈-C₁₂ bicyclic cycloalkyl, —C₈-C₁₂ bicyclic cycloalkenyl, or —(CH₂)ₙ-aryl;

R² is —CN, —NHCOOR⁴, —NHCONHR⁴, —NHNHCOR⁴, —NHNHCONHR⁴, —NHNHCOOR⁴, —NH—N═C(R⁵)R⁶, —NR⁵—N═C(R⁵)R⁶ or —NR⁵—N(R⁷)R⁸;

R³ is —C₁-C₁₀ alkyl, -aryl, -3- to 7-membered monocyclic heterocycle, -8- to 12-membered bicyclic heterocycle, —C₃-C₈ monocyclic cycloalkyl, —C₃-C₈ monocyclic cycloalkenyl, —C₈-C₁₂ bicyclic cycloalkyl or —C₈-C₁₂ bicyclic cycloalkenyl;

R⁴ is —C₁-C₁₀ alkyl, -aryl, —(CH₂)ₙ-aryl, —(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkyl), —(CH₂)ₙ—(C₈-C₁₂ bicyclic cycloalkenyl), —(CH₂)ₙ-(-3- to 7-membered monocyclic heterocycle) or —(CH₂)ₙ-(-8- to 12-membered bicyclic heterocycle);

each occurrence of R⁵ is independently —C₁-C₁₀ alkyl, -aryl, —(CH₂)ₙ-aryl, —(CH₂)ₙ—(C₃-C₈ monocyclic cycloalkyl), —(CH₂)ₘ—(C₃-C₈ monocyclic cycloalkenyl), —(CH₂)ₘ—(C₈-C₁₂ bicyclic cycloalkyl), —(CH₂)ₘ—(C₈-C₁₂ bicyclic cycloalkenyl), —(CH₂)ₘ-(-3- to 7-membered monocyclic heterocycle), —(CH₂)ₘ-(-8- to 12-membered bicyclic heterocycle), -phenylene-(C₂-C₁₀ alkynyl), -phenylene-(CH₂)ₘCOOH, -phenylene-(CH₂)ₘ-(-3- to 7-membered monocyclic heterocycle), -phenylene-(CH₂)ₘCOO—(C₁-C₁₀ alkyl) or —C(O)—(C₁-C₁₀ alkyl), or R⁵ and R⁶, together with the carbon atom to which they are attached, join to form a cyclopentyl, 2-cyclopentenyl, 3-cyclopentenyl, cyclohexyl, 2-cyclohexenyl or 3-cyclohexenyl ring;

R⁶ is —H, —C₁-C₁₀ alkyl, -aryl, —(CH₂)ₙ-aryl, —(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkyl), —(CH₂)ₙ—(C₈-C₁₂ bicyclic cycloalkenyl), —(CH₂)ₙ-(-3- to 7-membered monocyclic heterocycle), —(CH₂)ₙ-(-8- to 12-membered bicyclic heterocycle), -phenylene-(C₂-C₁₀ alkynyl), -phenylene-(CH₂)ₘCOOH, -phenylene-(CH₂)ₘ-(-3- to 7-membered monocyclic heterocycle) or -phenylene-(CH₂)ₘCOO—(C₁-C₁₀ alkyl);

R⁷ is —C₁-C₁₀ alkyl, -aryl, —(CH₂)ₙ-aryl, —(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkyl), —(CH₂)ₙ—(C₈-C₁₂ bicyclic cycloalkenyl), —(CH₂)ₙ-(-3- to 7-membered monocyclic heterocycle), —(CH₂)ₙ-(-8- to 12-membered bicyclic heterocycle), -phenylene-(C₂-C₁₀ alkynyl), —(CH₂)ₘ-phenylene-(CH₂)ₘCOOH, -phenylene-(CH₂)ₘ-(-3- to 7-membered monocyclic heterocycle), -phenylene-(CH₂)ₘCOO—(C₁-C₁₀ alkyl) or —C(O)—(C₁-C₁₀ alkyl), or R⁷ and R⁸, together with the nitrogen atom to which they are attached, join to form a -3- to 7-membered nitrogen-containing monocyclic heterocycle or a -8- to 12-membered nitrogen-containing bicyclic heterocycle;

R⁸ is —C₁-C₁₀ alkyl, -aryl, —(CH₂)ₙ-aryl, —(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkyl), —(CH₂)ₙ—(C₈-C₁₂ bicyclic cycloalkenyl), —(CH₂)ₙ-(-3- to 7-membered monocyclic heterocycle), —(CH₂)ₙ-(-8- to 12-membered bicyclic heterocycle), -phenylene-(C₂-C₁₀ alkynyl), -phenylene-(CH₂)ₘCOOH, -phenylene-(CH₂)ₘ-(-3- to 7-membered monocyclic heterocycle), -phenylene-(CH₂)ₘCOO—(C₁-C₁₀ alkyl) or —C(O)—(C₁-C₁₀ alkyl);

each m is independently an integer ranging from 0 to 4; and

each n is independently an integer ranging from 1 to 5.

In one embodiment, R¹ is —H.

In another embodiment, R¹ is —C₁-C₁₀ alkyl.

In a specific embodiment, R¹ is ethyl.

In another embodiment, R¹ is -aryl or —(CH₂)ₙ-aryl.

In another embodiment, R¹ is —C₃-C₈ monocyclic cycloalkyl.

In another embodiment, R¹ is —C₃-C₈ monocyclic cycloalkenyl.

In another embodiment, R¹ is —C₈-C₁₂ bicyclic cycloalkyl or —C₈-C₁₂ bicyclic cycloalkenyl.

In one embodiment, R² is —CN.

In another embodiment, R² is —NHC(O)OR⁴ or —NHC(O)NHR⁴.

In another embodiment, R² is —NHNHC(O)R⁴, —NHNHC(O)OR⁴ or —NHNHC(O)NHR⁴.

In another embodiment, R² is —NH—N═C(R⁵)R⁶.

In still another embodiment, R² is —NH—N═CH—(C₃-C₈ monocyclic cycloalkenyl).

In another embodiment, R² is —NH—N═CH-phenylene-(CH₂)ₘCOOH.

In a further embodiment, R² is —NH—N═CH-phenylene-(CH₂)ₘ—COO—(C₁-C₁₀ alkyl).

In one embodiment, R³ is —C₁-C₁₀ alkyl.

In another embodiment, R³ is -aryl.

In another embodiment, R³ is -3- to 7-membered monocyclic heterocycle.

In still another embodiment, R³ is -8- to 12-membered bicyclic heterocycle. In yet another embodiment, R³ is —C₃-C₈ monocyclic cycloalkyl.

In a further embodiment, R³ is —C₈-C₁₂ bicyclic cycloalkyl or —C₈-C₁₂ bicyclic cycloalkenyl.

In a specific embodiment, R³ is methyl.

In another specific embodiment, R³ is ethyl.

In one embodiment, R¹ is —H and R³ is —C₁-C₁₀ alkyl.

In a specific embodiment, R¹ is —H and R³ is ethyl.

In another embodiment, R¹ is —C₁-C₁₀ alkyl and R³ is —C₁-C₁₀ alkyl.

In a specific embodiment, R¹ and R³ are each ethyl.

In one embodiment, R¹ is —H, R² is —NH—N═C(R⁵)R⁶, and R³ is —C₁-C₁₀ alkyl.

In a specific embodiment, R¹ is —H, R² is —NH—N═C(R⁵)R⁶, and R³ is ethyl.

In another specific embodiment, R² is —H and is R³ is ethyl.

In one embodiment, R¹ is —H, R² is —CN, and R³ is —C₁-C₁₀ alkyl.

In another embodiment, R¹ is —C₁-C₁₀ alkyl, R² is —CN, and R³ is —C₁-C₁₀ alkyl.

In still another embodiment, R¹ is —C₁-C₁₀ alkyl, R² is —CN and R³ is -methyl.

In a further embodiment, R¹ is -methyl, R² is —CN and R³ is —C₁-C₁₀ alkyl.

In one embodiment, C and D are cis with respect to each other.

In another embodiment, C and D are trans with respect to each other.

In one embodiment, R⁹ and R¹⁰ are independently the residue of a naturally occurring amino acid.

In a specific embodiment, R⁹ and R¹⁰ are each:

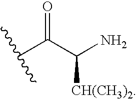

In another embodiment R⁹ and R¹⁰ join to form a —P(O)(OH)— group.

The present invention also provides compositions comprising an effective amount of a Purine Compound of Formula (167-Id) and a physiologically acceptable vehicle.

The invention further provides Purine Compounds of Formula (167-Id) that are in isolated and purified form.

The invention still further provides methods for treating or preventing a Condition, comprising administering an effective amount of a Purine Compound of Formula (167-Id) to a subject in need thereof.

In one embodiment, the invention provides compounds of the Formula (168-Ia): 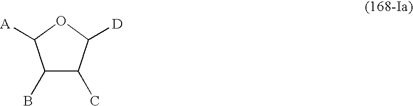
and pharmaceutically acceptable salts thereof, wherein

A is —CH₂OSO₂NH₂;

B is —OR⁹;

C is —OR¹⁰;

wherein R⁹ and R¹⁰ are independently the residue of a naturally occurring amino acid that is attached via its C-terminus, or R⁹ and R¹⁰ join to form a —P(O)(OH)— group;

D is:

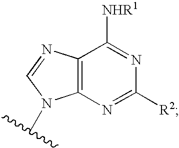

A and B are trans with respect to each other;

B and C are cis with respect to each other;

C and D are cis or trans with respect to each other;

R¹ is —C₃-C₈ monocyclic cycloalkyl, —(C₃-C₈ monocyclic cycloalkylene)-OH, —C₃-C₈ monocyclic cycloalkenyl, —(CH₂)ₙ—(C₃-C₈ monocyclic cycloalkyl), —(CH₂)ₙ—(C₃-C₈ monocyclic cycloalkenyl), —C₈-C₁₂ bicyclic cycloalkyl, or —C₈-C₁₂ bicyclic cycloalkenyl;

R² is -halo, —CN, —NHR⁸, —OR⁸, —SR⁸, —NHC(O)OR⁸, —NHC(O)R⁴, —NHC(O)NHR⁸, —NHNHC(O)R⁴, —NHNHC(O)OR⁸, —NHNHC(O)NHR⁸, or —NH—N═C(R⁶)R⁷;

R⁴ is —H, —C₁-C₁₅ alkyl, -aryl, —(CH₂)ₙ-aryl, —(CH₂)ₙ-(3- to 7-membered monocyclic heterocycle), —(CH₂)ₙ-(8- to 12-membered bicyclic heterocycle), —(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkyl), —(CH₂)ₙ—(C₈-C₁₂ bicyclic cycloalkenyl), —C≡C—(C₁-C₁₀ alkyl) or —C≡C-aryl;

R⁶ is —C₁-C₁₀ alkyl, -aryl, —(CH₂)ₙ-aryl, —(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkyl), —(CH₂)ₙ—(C₈-C₁₂ bicyclic cycloalkenyl), —(CH₂)ₙ-(3- to 7-membered monocyclic heterocycle), —(CH₂)ₙ-(8- to 12-membered bicyclic heterocycle), -phenylene-(CH₂)ₙCOOH, or -phenylene-(CH₂)ₙCOO—(C₁-C₁₀ alkyl);

R⁷ is —H, —C₁-C₁₀ alkyl, -aryl, —(CH₂)ₙ-aryl, —(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkyl), —(CH₂)ₙ—(C₈-C₁₂ bicyclic cycloalkenyl), —(CH₂)ₙ-(3- to 7-membered monocyclic heterocycle), or —(CH₂)ₙ-(8- to 12-membered bicyclic heterocycle), or R⁶ and R⁷ together with the carbon atom to which they are attached form a —C₃-C₈ monocyclic cycloalkyl, a —C₈-C₁₂ bicyclic cycloalkyl, a —C₃-C₈ monocyclic cycloalkenyl or a —C₈-C₁₂ bicyclic cycloalkenyl;

R⁸ is —C₁-C₁₅ alkyl, -aryl, —(CH₂)ₙ-aryl, —(CH₂)ₙ-(3- to 7-membered monocyclic heterocycle), —(CH₂)ₙ-(8- to 12-membered bicyclic heterocycle), —(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkyl), —(CH₂)ₙ—(C₈-C₁₂ bicyclic cycloalkenyl), —C≡C—(C₁-C₁₀ alkyl) or —C≡C-aryl; and

each n is independently an integer ranging from 1 to 5.

In one embodiment, R¹ is —C₃-C₈ monocyclic cycloalkyl.

In a specific embodiment, R¹ is cyclopentyl.

In another embodiment, R¹ is —C₃-C₈ monocyclic cycloalkenyl.

In another embodiment, R¹ is —C₈-C₁₂ bicyclic cycloalkyl or —C₈-C₁₂ bicyclic cycloalkenyl.

In still another embodiment, R¹ is —(CH₂)ₙ—(C₃-C₈ monocyclic cycloalkyl) or —(CH₂)ₙ—(C₃-C₈ monocyclic cycloalkenyl).

In one embodiment, R² is -halo.

In a specific embodiment, R² is —Cl.

In another embodiment, R² is —CN.

In another embodiment, R² is —NHR⁸, —OR⁸ or —SR⁸.

In a further embodiment, R² is —NHC(O)R⁴, —NHC(O)OR⁸ or —NHC(O)NHR⁸.

In another embodiment, R² is —NHNHC(O)R⁴, —NHNHC(O)OR⁸ or —NHNHC(O)NHR⁸.

In yet another embodiment, R² is —NH—N═C(R⁶)R⁷.

In another embodiment, R² is —NH—N═C(R⁶)R⁷ and R⁶ and R⁷ together with the carbon atom to which they are attached form a —C₃-C₈ monocyclic cycloalkyl, a —C₈-C₁₂ bicyclic cycloalkyl, a —C₃-C₈ monocyclic cycloalkenyl or a —C₈-C₁₂ bicyclic cycloalkenyl.

In one embodiment, R⁷ is —(CH₂)ₙ—(C₈-C₁₂ bicyclic cycloalkenyl).

In one embodiment, C and D are cis with respect to each other.

In another embodiment, C and D are trans with respect to each other.

In one embodiment, R⁹ and R¹⁰ are independently the residue of a naturally occurring amino acid.

In a specific embodiment, R⁹ and R¹⁰ are each:

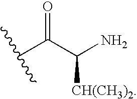

In another embodiment R⁹ and R¹⁰ join to form a —P(O)(OH)— group.

The present invention also provides compositions comprising an effective amount of a Purine Compound of Formula (Ia) and a physiologically acceptable carrier or vehicle.

The invention further provides Purine Compounds of Formula (Ia) that are in isolated and purified form.

The invention still further provides methods for treating or preventing a Condition, comprising administering an effective amount of a Purine Compound of Formula (168-Ia) to a subject in need thereof.

The invention further provides methods for reducing a subject's rate of metabolism, comprising administering an effective amount of a Purine Compound of Formula (168-Ia) to a subject in need thereof.

The invention further provides methods for protecting a subject's heart against myocardial damage during cardioplegia, comprising administering an effective amount of a Purine Compound of Formula (168-Ia) to a subject in need thereof.

In another embodiment, the invention provides compounds of Formula (168-Ib): 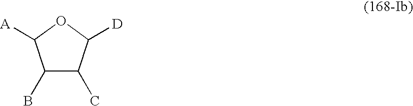
and pharmaceutically acceptable salts thereof, wherein

A is —CH₂ONO₂;

B is —OR⁸;

C is —OR⁹;

wherein R⁸ and R⁹ are independently the residue of a naturally occurring amino acid that is attached via its C-terminus, or R⁸ and R⁹ join to form a —P(O)(OH)— group;

D is:

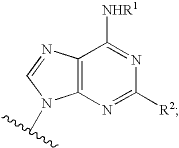

A and B are trans with respect to each other;

B and C are cis with respect to each other;

C and D are cis or trans with respect to each other;

R¹ is —H, —C₁-C₁₀ alkyl, -aryl, -3- to 7-membered monocyclic heterocycle, -8- to 12-membered bicyclic heterocycle, —C₃-C₈ monocyclic cycloalkyl, —C₃-C₈ monocyclic cycloalkenyl, —(C₃-C₈ monocyclic cycloalkylene)-OH, —C₈-C₁₂ bicyclic cycloalkyl, —C₈-C₁₂ bicyclic cycloalkenyl —(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkyl), —(CH₂)ₙ—(C₈-C₁₂ bicyclic cycloalkenyl), or —(CH₂)ₙ-aryl;

R² is —CN, —NHR⁴, —NHC(O)R⁴, —NHC(O)OR⁴, —NHC(O)NHR⁴, —NHNHC(O)R⁴, —NHNHC(O)OR⁴, —NHNHC(O)NHR⁴, or —NH—N═C(R⁶)R⁷;

R⁴ is —C₁-C₁₅ alkyl, -aryl, —(CH₂)ₙ-aryl, —(CH₂)ₙ-(3- to 7-membered monocyclic heterocycle), —(CH₂)ₙ-(8- to 12-membered bicyclic heterocycle), —(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkyl), —(CH₂)ₙ—(C₈-C₁₂ bicyclic cycloalkenyl), —C≡C—(C₁-C₁₀ alkyl) or —C≡C-aryl;

R⁶ is —C₁-C₁₀ alkyl, -aryl, —(CH₂)ₙ-aryl, —(CH₂)ₙ-(3- to 7-membered monocyclic heterocycle), —(CH₂)ₙ-(8- to 12-membered bicyclic heterocycle), —(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkyl), —(CH₂)ₙ—(C₈-C₁₂ bicyclic cycloalkenyl), -phenylene-(CH₂)ₙCOOH, or -phenylene-(CH₂)ₙCOO—(C₁-C₁₀ alkyl);

R⁷ is —H, —C₁-C₁₀ alkyl, -aryl, —(CH₂)ₙ-aryl, —(CH₂)ₙ-(3- to 7-membered monocyclic heterocycle), —(CH₂)ₙ-(8- to 12-membered bicyclic heterocycle), —(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkenyl) or —(CH₂)ₙ—(C₈-C₁₂ bicyclic cycloalkyl), or R⁶ and R⁷ together with the carbon atom to which they are attached form a —C₃-C₈ monocyclic cycloalkyl, a —C₈-C₁₂ bicyclic cycloalkyl, a —C₃-C₈ monocyclic cycloalkenyl or a —C₈-C₁₂ bicyclic cycloalkenyl; and

each n is independently an integer ranging from 1 to 5.

In one embodiment, R¹ is —H.

In another embodiment, R¹ is —C₃-C₈ monocyclic cycloalkyl.

In a specific embodiment, R¹ is cyclopentyl.

In another embodiment, R¹ is —C₃-C₈ monocyclic cycloalkenyl.

In another embodiment, R¹ is —C₈-C₁₂ bicyclic cycloalkyl or —C₈-C₁₂ bicyclic cycloalkenyl.

In still another embodiment, R¹ is —(CH₂)ₙ—(C₃-C₈ monocyclic cycloalkyl) or —(CH₂)ₙ—(C₃-C₈ monocyclic cycloalkenyl).

In another embodiment, R² is —CN.

In another embodiment, R² is —NHR⁴.

In a further embodiment, R² is —NHC(O)R⁴, —NHC(O)OR⁴ or —NHC(O)NHR⁴.

In another embodiment, R² is —NHNHC(O)R⁴, —NHNHC(O)OR⁴ or —NHNHC(O)NHR⁴.

In yet another embodiment, R² is —NH—N═C(R⁶)R⁷.

In another embodiment, R² is —NH—N═C(R⁶)R⁷ and R⁶ and R⁷ together with the carbon atom to which they are attached form a —C₃-C₈ monocyclic cycloalkyl, a —C₈-C₁₂ bicyclic cycloalkyl, a —C₃-C₈ monocyclic cycloalkenyl or a —C₈-C₁₂ bicyclic cycloalkenyl.

In one embodiment, C and D are cis with respect to each other.

In another embodiment, C and D are trans with respect to each other.

In one embodiment, R⁸ and R⁹ are independently the residue of a naturally occurring amino acid.

In a specific embodiment, R⁸ and R⁹ are each:

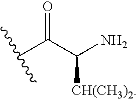

In another embodiment R⁸ and R⁹ join to form a —P(O)(OH)— group.

The present invention also provides compositions comprising an effective amount of a Purine Compound of Formula (168-Ib) and a physiologically acceptable carrier or vehicle.

The invention further provides Purine Compounds of Formula (168-Ib) that are in isolated and purified form.

The invention still further provides methods for treating or preventing a Condition, comprising administering an effective amount of a Purine Compound of Formula (168-Ib) to a subject in need thereof.

The invention further provides methods for reducing a subject's rate of metabolism, comprising administering an effective amount of a Purine Compound of Formula (168-Ib) to a subject in need thereof.

The invention further provides methods protecting a subject's heart against myocardial damage during cardioplegia, comprising administering an effective amount of a Purine Compound of Formula (168-Ib) to a subject in need thereof.

In still another embodiment, the invention provides compounds having the Formula (168-Ic): 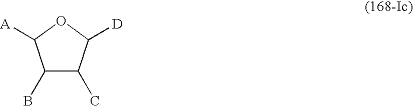
and pharmaceutically acceptable salts thereof, wherein

A is —CH₂NHR⁵;

B is —OR⁶;

C is —OR⁷;

wherein R⁶ and R⁷ are independently the residue of a naturally occurring amino acid that is attached via its C-terminus, or R⁶ and R⁷ join to form a —P(O)(OH)— group;

D is:

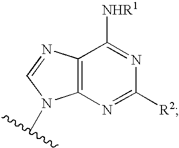

A and B are trans with respect to each other;

B and C are cis with respect to each other;

C and D are cis or trans with respect to each other;

R¹ is —H, —C₁-C₁₀ alkyl, -aryl, -3- to 7-membered monocyclic heterocycle, -8- to 12-membered bicyclic heterocycle, —C₃-C₈ monocyclic cycloalkyl, —C₃-C₈ monocyclic cycloalkenyl, —(C₃-C₈ monocyclic cycloalkylene)-OH, —C₈-C₁₂ bicyclic cycloalkyl, —C₈-C₁₂ bicyclic cycloalkenyl, —(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkyl), —(CH₂)ₙ—(C₈-C₁₂ bicyclic cycloalkenyl), or —(CH₂)ₙ-aryl;

R² is —NHR⁴, —OR⁴, —SR⁴, —NHC(O)R⁴, —NHC(O)OR⁴, —NHC(O)NHR⁴, —NHNHC(O)R⁴, —NHNHC(O)NHR⁴, or —NHNHC(O)OR⁴;

R⁴ is —C₁-C₁₅ alkyl, -aryl, —(CH₂)ₙ-aryl, —(CH₂)ₙ-(3- to 7-membered monocyclic heterocycle), —(CH₂)ₙ-(8- to 12-membered bicyclic heterocycle), —(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkyl), —(CH₂)ₙ—(C₈-C₁₂ bicyclic cycloalkenyl), —C≡C—(C₁-C₁₀ alkyl) or —C≡C-aryl;

R⁵ is —C(O)O(C₁-C₁₀ alkyl), —C(O)NH(C₁-C₁₀ alkyl), —C(O)N(C₁-C₁₀ alkyl)₂, —C(O)NH-aryl, —CH(NH₂)NH₂ or —CH(NH₂)NH(C₁-C₁₀ alkyl); and

each n is independently an integer ranging from 1 to 5.

In one embodiment, R¹ is —H.

In another embodiment, R¹ is —C₁-C₁₀ alkyl.

In one embodiment, R¹ is -aryl or —(CH₂)ₙ-aryl.

In another embodiment, R¹ is —C₃-C₈ monocyclic cycloalkyl.

In a specific embodiment, R¹ is cyclopentyl.

In another embodiment, R¹ is —C₃-C₈ monocyclic cycloalkenyl.

In another embodiment, R¹ is —C₈-C₁₂ bicyclic cycloalkyl or —C₈-C₁₂ bicyclic cycloalkenyl.

In still another embodiment, R¹ is —(CH₂)ₙ—(C₃-C₈ monocyclic cycloalkyl) or —(CH₂)ₙ—(C₃-C₈ monocyclic cycloalkenyl).

In another embodiment, R¹ is -3- to 7-membered monocyclic heterocycle or -8- to 12-membered bicyclic heterocycle.

In another embodiment, R² is —NHR⁴, —OR⁴ or —SR⁴.

In a further embodiment, R² is —NHC(O)R⁴, —NHC(O)OR⁴ or —NHC(O)NHR⁴.

In another embodiment, R² is —NHNHC(O)R⁴, —NHNHC(O)OR⁴ or —NHNHC(O)NHR⁴.

In one embodiment, R⁵ is —C(O)O(C₁-C₁₀ alkyl).

In another embodiment, R⁵ is —C(O)NH(C₁-C₁₀ alkyl), —C(O)N(C₁-C₁₀ alkyl)₂ or —C(O)NH-aryl.

In another embodiment, R⁵ is —CH(NH₂)NH₂ or —CH(NH₂)NH(C₁-C₁₀ alkyl).

In one embodiment, C and D are cis with respect to each other.

In another embodiment, C and D are trans with respect to each other.

In one embodiment, R⁶ and R⁷ are independently the residue of a naturally occurring amino acid.

In a specific embodiment, R⁶ and R⁷ are each:

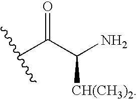

In another embodiment R⁶ and R⁷ join to form a —P(O)(OH)— group.

The present invention also provides compositions comprising an effective amount of a Purine Compound of Formula (168-Ic) and a physiologically acceptable carrier or vehicle.

The invention further provides Purine Compounds of Formula (168-Ic) that are in isolated and purified form.

The invention still further provides methods for treating or preventing a Condition, comprising administering an effective amount of a Purine Compound of Formula (168-Ic) to a subject in need thereof.

The invention further provides methods for reducing a subject's rate of metabolism, comprising administering an effective amount of a Purine Compound of Formula (168-Ic) to a subject in need thereof.

The invention further provides methods protecting a subject's heart against myocardial damage during cardioplegia, comprising administering an effective amount of a Purine Compound of Formula (168-Ic) to a subject in need thereof.

In a further embodiment, the invention provides compounds having the Formula (168-Id): 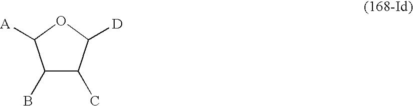
and pharmaceutically acceptable salts thereof, wherein

A is —R³;

B is —OR⁸;

C is —OR⁹;

wherein R⁸ and R⁹ are independently the residue of a naturally occurring amino acid that is attached via its C-terminus, or R⁸ and R⁹ join to form a —P(O)(OH)— group;

D is:

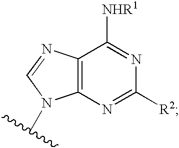

A and B are trans with respect to each other;

B and C are cis with respect to each other;

C and D are cis or trans with respect to each other;

R¹ is —H, —C₁-C₁₀ alkyl, -aryl, -3- to 7-membered monocyclic heterocycle, -8- to 12-membered bicyclic heterocycle, —C₃-C₈ monocyclic cycloalkyl, —C₃-C₈ monocyclic cycloalkenyl, —(C₃-C₈ monocyclic cycloalkylene)-OH, —(C₃-C₈ monocyclic cycloalkylene)-OH, —C₈-C₁₂ bicyclic cycloalkyl, —C₈-C₁₂ bicyclic cycloalkenyl, —(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkyl), —(CH₂)ₙ—(C₈-C₁₂ bicyclic cycloalkenyl), or —(CH₂)ₙ-aryl;

R² is —H, -halo, —CN, —NHR⁴, —OR⁴, —SR⁴, —NHC(O)R⁴, —NHC(O)OR⁴, —NHC(O)NHR⁴, —NHNHC(O)R⁴, —NHNHC(O)NHR⁴, —NHNHC(O)OR⁴or —NH—N═C(R⁶)R⁷;

R³ is —CH₂ONO or —CH₂OSO₃H;

R⁴ is —C₁-C₁₅ alkyl, -aryl, —(CH₂)ₙ-aryl, —(CH₂)ₙ-(3- to 7-membered monocyclic heterocycle), —(CH₂)ₙ-(8- to 12-membered bicyclic heterocycle), —(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkyl), —(CH₂)ₙ—(C₈-C₁₂ bicyclic cycloalkenyl), —C≡C—(C₁-C₁₀ alkyl) or —C≡C-aryl;

R⁶ is —C₁-C₁₀ alkyl, -aryl, —(CH₂)ₙ-aryl, —(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkyl), —(CH₂)ₙ—(C₈-C₁₂ bicyclic cycloalkenyl), —(CH₂)ₙ-(3- to 7-membered monocyclic heterocycle), —(CH₂)ₙ-(8- to 12-membered bicyclic heterocycle), -phenylene-(CH₂)ₙCOOH, or -phenylene-(CH₂)ₙCOO—(C₁-C₁₀ alkyl);

R⁷ is —H, —C₁-C₁₀ alkyl, -aryl, —(CH₂)ₙ-aryl, —(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkyl), —(CH₂)ₙ—(C₈-C₁₂ bicyclic cycloalkenyl), —(CH₂)ₙ-(3- to 7-membered monocyclic heterocycle), or —(CH₂)ₙ-(8- to 12-membered bicyclic heterocycle), or R⁶ and R⁷ together with the carbon atom to which they are attached form a —C₃-C₈ monocyclic cycloalkyl, a —C₈-C₁₂ bicyclic cycloalkyl, a —C₃-C₈ monocyclic cycloalkenyl or a —C₈-C₁₂ bicyclic cycloalkenyl; and

each n is independently an integer ranging from 1 to 5.

In one embodiment, R¹ is —H.

In another embodiment, R¹ is —C₁-C₁₀ alkyl.

In one embodiment, R¹ is -aryl or —(CH₂)ₙ-aryl.

In another embodiment, R¹ is —C₃-C₈ monocyclic cycloalkyl.

In a specific embodiment, R¹ is cyclopentyl.

In another embodiment, R¹ is —C₃-C₈ monocyclic cycloalkenyl.

In another embodiment, R¹ is —C₈-C₁₂ bicyclic cycloalkyl or —C₈-C₁₂ bicyclic cycloalkenyl.

In still another embodiment, R¹ is —(CH₂)ₙ—(C₃-C₈ monocyclic cycloalkyl) or —(CH₂)ₙ—(C₃-C₈ monocyclic cycloalkenyl).

In another embodiment, R¹ is -3- to 7-membered monocyclic heterocycle or -8- to 12-membered bicyclic heterocycle.

In one embodiment, R² is —H.

In one embodiment, R² is -halo.

In a specific embodiment, R² is —Cl.

In another embodiment, R² is —CN.

In another embodiment, R² is —NHR⁴, —OR⁴ or —SR⁴.

In a further embodiment, R² is —NHC(O)R⁴, —NHC(O)OR⁴ or —NHC(O)NHR⁴.

In another embodiment, R² is —NHNHC(O)R⁴, —NHNHC(O)OR⁴ or —NHNHC(O)NHR⁴.

In yet another embodiment, R² is —NH—N═C(R⁶)R⁷.

In another embodiment, R² is —NH—N═C(R⁶)R⁷ and R⁶ and R⁷ together with the carbon atom to which they are attached form a —C₃-C₈ monocyclic cycloalkyl, a —C₈-C₁₂ bicyclic cycloalkyl, a —C₃-C₈ monocyclic cycloalkenyl or a —C₈-C₁₂ bicyclic cycloalkenyl.

In one embodiment, R³ is —CH₂ONO.

In another embodiment, R³ is —CH₂OSO₃H.

In one embodiment, C and D are cis with respect to each other.

In another embodiment, C and D are trans with respect to each other.

In one embodiment, R⁸ and R⁹ are independently the residue of a naturally occurring amino acid.

In a specific embodiment, R⁸ and R⁹ are each:

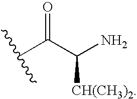

In another embodiment R⁸ and R⁹ join to form a —P(O)(OH)— group.

The present invention also provides compositions comprising an effective amount of a Purine Compound of Formula (168-Id) and a physiologically acceptable carrier or vehicle.

The invention further provides Purine Compounds of Formula (168-Id) that are in isolated and purified form.

The invention still further provides methods for treating or preventing a Condition, comprising administering an effective amount of a Purine Compound of Formula (168-Id) to a subject in need thereof.

The invention further provides methods for reducing a subject's rate of metabolism, comprising administering an effective amount of a Purine Compound of Formula (168-Id) to a subject in need thereof.

The invention further provides methods protecting a subject's heart against myocardial damage during cardioplegia, comprising administering an effective amount of a Purine Compound of Formula (168-Id) to a subject in need thereof.

In a further embodiment, the invention provides compounds having the Formula (168-Ie): 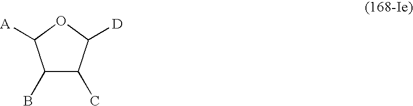
and pharmaceutically acceptable salts thereof, wherein

A is —R³;

B is —OR⁸;

C is —OR⁹;

wherein R⁸ and R⁹ are independently the residue of a naturally occurring amino acid that is attached via its C-terminus, or R⁸ and R⁹ join to form a —P(O)(OH)— group;

D is:

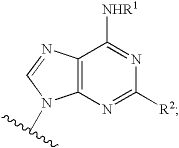

A and B are trans with respect to each other;

B and C are cis with respect to each other;

C and D are cis or trans with respect to each other;

R¹ is -3- to 7-membered monocyclic heterocycle, -8- to 12-membered bicyclic heterocycle, —C₃-C₈ monocyclic cycloalkyl, —(C₃-C₈ monocyclic cycloalkylene)-OH, —C₃-C₈ monocyclic cycloalkenyl, —C₈-C₁₂ bicyclic cycloalkyl, —C₈-C₁₂ bicyclic cycloalkenyl, —(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkyl), —(CH₂)ₙ—(C₈-C₁₂ bicyclic cycloalkenyl), or —(CH₂)ₙ-aryl;

R² is -halo, —CN, —NHR⁴, —OR⁴, —SR⁴, —NHC(O)R⁴, —NHC(O)OR⁴, —NHC(O)NHR⁴, —NHNHC(O)R⁴, —NHNHC(O)OR⁴, —NHNHC(O)NHR⁴, or —NH—N═C(R⁶)R⁷;

R³ is —CH₂OSO₂NH(C₁-C₁₀ alkyl), —CH₂OSO₂N(C₁-C₁₀ alkyl)₂, or —CH₂OSO₂NH-aryl, where each C₁-C₁₀ alkyl is independent;

R⁴ is —C₁-C₁₅ alkyl, -aryl, —(CH₂)ₙ-aryl, —(CH₂)ₙ-(3- to 7-membered monocyclic heterocycle), —(CH₂)ₙ-(8- to 12-membered bicyclic heterocycle), —(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkyl), —(CH₂)ₙ—(C₈-C₁₂ bicyclic cycloalkenyl), —C≡C—(C₁-C₁₀ alkyl) or —C≡C-aryl;

R⁶ is —C₁-C₁₀ alkyl, -aryl, —(CH₂)ₙ-aryl, —(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkyl), —(CH₂)ₙ—(C₈-C₁₂ bicyclic cycloalkenyl), —(CH₂)ₙ-(3- to 7-membered monocyclic heterocycle), —(CH₂)ₙ-(8- to 12-membered bicyclic heterocycle), -phenylene-(CH₂)ₙCOOH, or -phenylene-(CH₂)ₙCOO—(C₁-C₁₀ alkyl);

R⁷ is —H, —C₁-C₁₀ alkyl, -aryl, —(CH₂)ₙ-aryl, —(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkyl), —(CH₂)ₙ—(C₈-C₁₂ bicyclic cycloalkenyl), —(CH₂)ₙ-(3- to 7-membered monocyclic heterocycle), or —(CH₂)ₙ-(8- to 12-membered bicyclic heterocycle), or R⁶ and R⁷ together with the carbon atom to which they are attached form a —C₃-C₈ monocyclic cycloalkyl, a —C₈-C₁₂ bicyclic cycloalkyl, a —C₃-C₈ monocyclic cycloalkenyl or a —C₈-C₁₂ bicyclic cycloalkenyl; and

each n is independently an integer ranging from 1 to 5.

In one embodiment, R¹ is —(CH₂)ₙ-aryl.

In another embodiment, R¹ is —C₃-C₈ monocyclic cycloalkyl.

In a specific embodiment, R¹ is cyclopentyl.

In another embodiment, R¹ is —C₃-C₈ monocyclic cycloalkenyl.

In another embodiment, R¹ is —C₈-C₁₂ bicyclic cycloalkyl or —C₈-C₁₂ bicyclic cycloalkenyl.

In still another embodiment, R¹ is —(CH₂)ₙ—(C₃-C₈ monocyclic cycloalkyl) or —(CH₂)ₙ—(C₃-C₈ monocyclic cycloalkenyl).

In another embodiment, R¹ is -3- to 7-membered monocyclic heterocycle or -8- to 12-membered bicyclic heterocycle.

In one embodiment, R² is -halo.

In a specific embodiment, R² is —Cl.

In another embodiment, R² is —CN.

In another embodiment, R² is —NHR⁴, —OR⁴ or —SR⁴.

In a further embodiment, R² is —NHC(O)R⁴, —NHC(O)OR⁴ or —NHC(O)NHR⁴.

In another embodiment, R² is —NHNHC(O)R⁴, —NHNHC(O)OR⁴ or —NHNHC(O)NHR⁴.

In yet another embodiment, R² is —NH—N═C(R⁶)R⁷.

In another embodiment, R² is —NH—N═C(R⁶)R⁷ and R⁶ and R⁷ together with the carbon atom to which they are attached form a —C₃-C₈ monocyclic cycloalkyl, a —C₈-C₁₂ bicyclic cycloalkyl, a —C₃-C₈ monocyclic cycloalkenyl or a —C₈-C₁₂ bicyclic cycloalkenyl.

In one embodiment, R⁷ is —(CH₂)ₙ—(C₈-C₁₂ bicyclic cycloalkenyl).

In one embodiment, C and D are cis with respect to each other.

In another embodiment, C and D are trans with respect to each other.

In one embodiment, R⁸ and R⁹ are independently the residue of a naturally occurring amino acid.

In a specific embodiment, R⁸ and R⁹ are each:

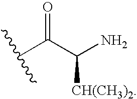

In another embodiment R⁸ and R⁹ join to form a —P(O)(OH)— group.

The present invention also provides compositions comprising an effective amount of a Purine Compound of Formula (168-Ie) and a physiologically acceptable carrier or vehicle.

The invention further provides Purine Compounds of Formula (168-Ie) that are in isolated and purified form.

The invention still further provides methods for treating or preventing a Condition, comprising administering an effective amount of a Purine Compound of Formula (168-Ie) to a subject in need thereof.

The invention further provides methods for reducing a subject's rate of metabolism, comprising administering an effective amount of a Purine Compound of Formula (168-Ie) to a subject in need thereof.

The invention further provides methods protecting a subject's heart against myocardial damage during cardioplegia, comprising administering an effective amount of a Purine Compound of Formula (168-Ie) to a subject in need thereof.

In another embodiment, the invention provides compounds having the Formula (168-If): 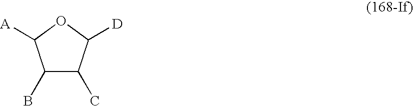
and pharmaceutically acceptable salts thereof, wherein

A is —CH₂ONO₂;

B is —OR³;

C is —OR⁴;

wherein R³ and R⁴ are independently the residue of a naturally occurring amino acid that is attached via its C-terminus, or R³ and R⁴ join to form a —P(O)(OH)— group;

D is:

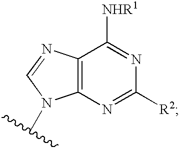

A and B are trans with respect to each other;

B and C are cis with respect to each other;

C and D are cis or trans with respect to each other;

R¹ is —C₃-C₈ monocyclic cycloalkyl or —(C₃-C₈ monocyclic cycloalkylene)-OH; and

R² is —H or -halo.

In one embodiment, R¹ is —C₅-C₆ monocyclic cycloalkyl.

In another embodiment, R¹ is cyclopentyl.

In one embodiment, R² is —H

In another embodiment R² is -halo.

In another embodiment, R² is —Cl.

In one embodiment, C and D are cis with respect to each other.

In another embodiment, C and D are trans with respect to each other.

In one embodiment, R³ and R⁴ are independently the residue of a naturally occurring amino acid.

In a specific embodiment, R³ and R⁴ are each:

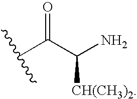

In another embodiment R³ and R⁴ join to form a —P(O)(OH)— group.

The present invention also provides compositions comprising an effective amount of a Purine Compound of Formula (168-If) and a physiologically acceptable carrier or vehicle.

The invention further provides Purine Compounds of Formula (168-If) that are in isolated and purified form.

The invention still further provides methods for treating or preventing a Condition, comprising administering an effective amount of a Purine Compound of Formula (168-If) to a subject in need thereof.

The invention further provides methods for reducing a subject's rate of metabolism, comprising administering an effective amount of a Purine Compound of Formula (168-If) to a subject in need thereof.

The invention further provides methods protecting a subject's heart against myocardial damage during cardioplegia, comprising administering an effective amount of a Purine Compound of Formula (168-If) to a subject in need thereof.

In another embodiment, the invention provides compounds having the Formula (168-Ig): 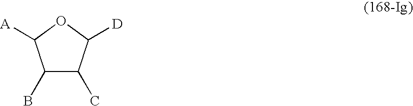
and pharmaceutically acceptable salts thereof, wherein

A is —CH₂ONO₂;

B is —OR³;

C is —OR⁴;

wherein R³ and R⁴ are independently the residue of a naturally occurring amino acid that is attached via its C-terminus, or R³ and R⁴ join to form a —P(O)(OH)— group;

D is:

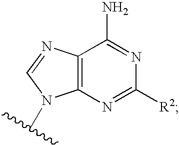

A and B are trans with respect to each other;

B and C are cis with respect to each other;

C and D are cis or trans with respect to each other; and

R² is —H or -halo.

In one embodiment, R² is —H.

In another embodiment R² is -halo.

In a specific embodiment, R² is —Cl.

In one embodiment, C and D are cis with respect to each other.

In another embodiment, C and D are trans with respect to each other.

In one embodiment, R³ and R⁴ are independently the residue of a naturally occurring amino acid.

In a specific embodiment, R³ and R⁴ are each:

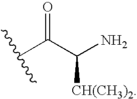

In another embodiment R³ and R⁴ join to form a —P(O)(OH)— group.

The present invention also provides compositions comprising an effective amount of a Purine Compound of Formula (168-Ig) and a physiologically acceptable carrier or vehicle.

The invention further provides Purine Compounds of Formula (168-Ig) that are in isolated and purified form.

The invention still further provides methods for treating or preventing a Condition, comprising administering an effective amount of a Purine Compound of Formula (168-Ig) to a subject in need thereof.

The invention further provides methods for reducing a subject's rate of metabolism, comprising administering an effective amount of a Purine Compound of Formula (168-Ig) to a subject in need thereof.

The invention further provides methods protecting a subject's heart against myocardial damage during cardioplegia, comprising administering an effective amount of a Purine Compound of Formula (168-Ig) to a subject in need thereof.

In another embodiment, the invention provides compounds having the Formula (168-Ih): 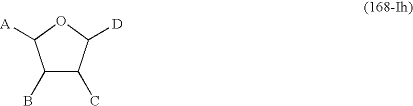
and pharmaceutically acceptable salts thereof, wherein

A is —CH₂ONO₂;

B is —OR²;

C is —OR³;

wherein R² and R³ are independently the residue of a naturally occurring amino acid that is attached via its C-terminus, or R² and R³ join to form a —P(O)(OH)— group;

D is:

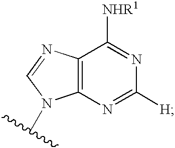

A and B are trans with respect to each other;

B and C are cis with respect to each other; and

C and D are cis or trans with respect to each other; and

R¹ is:

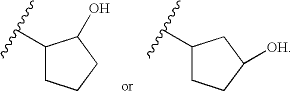

In one embodiment, R¹ is

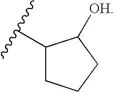

In another embodiment R¹ is

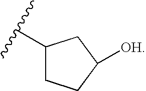

In one embodiment, C and D are cis with respect to each other.

In another embodiment, C and D are trans with respect to each other.

In one embodiment, R² and R³ are independently the residue of a naturally occurring amino acid.

In a specific embodiment, R² and R³ are each:

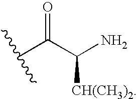

In another embodiment R² and R³ join to form a —P(O)(OH)— group.

The present invention also provides compositions comprising an effective amount of a Purine Compound of Formula (168-Ih) and a physiologically acceptable carrier or vehicle.

The invention further provides Purine Compounds of Formula (168-Ih) that are in isolated and purified form.

The invention still further provides methods for treating or preventing a Condition, comprising administering an effective amount of a Purine Compound of Formula (168-Ih) to a subject in need thereof.

The invention further provides methods for reducing a subject's rate of metabolism, comprising administering an effective amount of a Purine Compound of Formula (168-Ih) to a subject in need thereof.

The invention further provides methods protecting a subject's heart against myocardial damage during cardioplegia, comprising administering an effective amount of a Purine Compound of Formula (168-Ih) to a subject in need thereof.

In another embodiment, the invention provides compounds having the Formula (168-II): 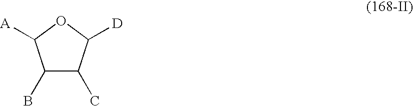
and pharmaceutically acceptable'salts thereof, wherein

A is —CH₂OH;

B is —OR⁸;

C is —OR⁹;

wherein R⁸ and R⁹ are independently the residue of a naturally occurring amino acid that is attached via its C-terminus, or R⁸ and R⁹ join to form a —P(O)(OH)— group;

D is:

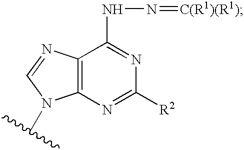

A and B are trans with respect to each other;

B and C are cis with respect to each other;

C and D are cis or trans with respect to each other;

each R¹ is independently —H, —C₁-C₁₀ alkyl, —(CH₂)ₘ-(3- to 7-membered monocyclic heterocycle), —(CH₂)ₘ-(8- to 12-membered bicyclic heterocycle), —(CH₂)ₘ—(C₈-C₁₂ bicyclic cycloalkyl), —(CH₂)ₘ—(C₈-C₁₂ bicyclic cycloalkenyl), or —(CH₂)ₘ-aryl, or both R¹ groups together with the carbon atom to which they are attached form a —C₃-C₈ monocyclic cycloalkyl, a —C₃-C₈ monocyclic cycloalkenyl, a —C₈-C₁₂ bicyclic cycloalkyl, or a —C₈-C₁₂ bicyclic cycloalkenyl;

R² is —OR⁴, —SR⁴, —NHNHC(O)R³, —NHNHC(O)NHR³, —NHNHC(O)OR⁷, or —NH—N═C(R⁵)R⁶;

R³ is —H, —C₁-C₁₀ alkyl, —(CH₂)ₙ-(3- to 7-membered monocyclic heterocycle), —(CH₂)ₙ-(8- to 12-membered bicyclic heterocycle), —(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkyl), —(CH₂)ₙ—(C₈-C₁₂ bicyclic cycloalkenyl), —(CH₂)ₙ-aryl, —O—(CH₂)ₙ—(C₈-C₁₂ bicyclic cycloalkyl), —O—(CH₂)ₙ—(C₃-C₈ monocyclic cycloalkyl), O—(CH₂)ₙ—(C₃-C₈ monocyclic cycloalkenyl), —C≡C—(C₁-C₁₀ alkyl) or —C≡C-aryl;

R⁴ is —C₁-C₁₀ alkyl, —(CH₂)ₙ-(3- to 7-membered monocyclic heterocycle), —(CH₂)ₙ-(8- to 12-membered bicyclic heterocycle), —(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkyl), —(CH₂)ₙ—(C₈-C₁₂ bicyclic cycloalkenyl), —(CH₂)ₙ-aryl, or —C≡C-aryl;

R⁵ and R⁶ are each independently —H, —C₁-C₁₀ alkyl, —(CH₂)ₙ-(3- to 7-membered monocyclic heterocycle), —(CH₂)ₙ-(8- to 12-membered bicyclic heterocycle), —(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkyl), —(CH₂)ₙ—(C₈-C₁₂ bicyclic cycloalkenyl), —(CH₂)ₙ-aryl, -phenylene-(CH₂)ₙCOOH, or -phenylene-(CH₂)ₙCOO—(C₁-C₁₀ alkyl), or R⁵ and R⁶ together with the carbon atom to which they are attached form a C₃-C₈ monocyclic cycloalkyl or a C₈-C₁₂ bicyclic cycloalkyl;

R⁷ is —H, —C₁-C₁₀ alkyl, —(CH₂)ₙ-(3- to 7-membered monocyclic heterocycle), —(CH₂)ₙ-(8- to 12-membered bicyclic heterocycle), —(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkyl), —(CH₂)ₙ—(C₈-C₁₂ bicyclic cycloalkenyl), —(CH₂)ₙ-aryl, —C≡C—(C₁-C₁₀ alkyl) or —C≡C-aryl;

m is an integer ranging from 0 to 3; and

each n is independently an integer ranging from 0 to 5.

In one embodiment, R¹ is —H.

In another embodiment, R¹ is —C₁-C₁₀ alkyl.

In still another embodiment, R¹ is —(CH₂)ₘ—(C₈-C₁₂ bicyclic cycloalkyl) or —(CH₂)ₘ—(C₈-C₁₂ bicyclic cycloalkenyl).

In another embodiment, R² is —OR⁴ or —SR⁴.

In another embodiment, R² is —NHNHC(O)R³, —NHNHC(O)OR⁷ or —NHNHC(O)NHR³.

In yet another embodiment, R² is —NH—N═C(R⁵)R⁶.

In a specific embodiment, R² is —NH—N═CH-cyclopentyl.

In one embodiment, one occurrence of R¹ is —H.

In another embodiment, both R¹ groups together with the carbon atom to which they are attached, join to form a —C₃-C₈ monocyclic cycloalkyl.

In one embodiment, R⁴ is —(CH₂)ₙ—(C₈-C₁₂ bicyclic cycloalkenyl).

In one embodiment, C and D are cis with respect to each other.

In another embodiment, C and D are trans with respect to each other.

In one embodiment, R⁸ and R⁹ are independently the residue of a naturally occurring amino acid.

In a specific embodiment, R⁸ and R⁹ are each:

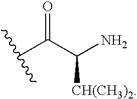

In another embodiment R⁸ and R⁹ join to form a —P(O)(OH)— group.

The present invention also provides compositions comprising an effective amount of a Purine Compound of Formula (II) and a physiologically acceptable carrier or vehicle.

The invention further provides Purine Compounds of Formula (II) that are in isolated and purified form.

The invention still further provides methods for treating or preventing a Condition, comprising administering an effective amount of a Purine Compound of Formula (168-II) to a subject in need thereof.

The invention further provides methods for reducing a subject's rate of metabolism, comprising administering an effective amount of a Purine Compound of Formula (168-II) to a subject in need thereof.

The invention further provides methods protecting a subject's heart against myocardial damage during cardioplegia, comprising administering an effective amount of a Purine Compound of Formula (168-II) to a subject in need thereof.

In still another embodiment, the invention provides compounds having the Formula (168-III): 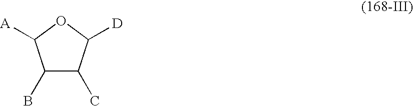
and pharmaceutically acceptable salts thereof, wherein

A is —R³;

B is —OR⁸;

C is —OR⁹;

wherein R⁸ and R⁹ are independently the residue of a naturally occurring amino acid that is attached via its C-terminus, or R⁸ and R⁹ join to form a —P(O)(OH)— group;

D is:

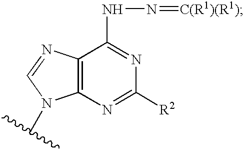

A and B are trans with respect to each other;

B and C are cis with respect to each other;

C and D are cis or trans with respect to each other;

each R¹ is independently —H, —C₁-C₁₀ alkyl, —(CH₂)ₘ-(3- to 7-membered monocyclic heterocycle), —(CH₂)ₘ-(8- to 12-membered bicyclic heterocycle), —(CH₂)ₘ—(C₃-C₈ monocyclic cycloalkyl), —(CH₂)ₘ—(C₃-C₈ monocyclic cycloalkylene)-OH, —(CH₂)ₘ—(C₃-C₈ monocyclic cycloalkenyl), —(CH₂)ₘ—(C₈-C₁₂ bicyclic cycloalkyl), —(CH₂)ₙ—(C₈-C₁₂ bicyclic cycloalkenyl), or —(CH₂)ₘ-aryl, or two R¹ groups, together with the carbon atom to which they are attached, form a —C₃-C₈ monocyclic cycloalkyl, a —C₃-C₈ monocyclic cycloalkenyl, a —C₈-C₁₂ bicyclic cycloalkyl, or a —C₈-C₁₂ bicyclic cycloalkenyl;

R² is —H, —CN, -halo, —N(R⁴)₂, —OR⁴, —SR⁴, —NHC(O)R⁴, —NHC(O)OR⁴, —NHC(O)NHR⁴, —NHNHC(O)R⁴, —NHNHC(O)NHR⁴, —NHNHC(O)OR⁴, or —NH—N═C(R⁶)R⁷;

R³ is —CH₂ONO₂, —CH₂ONO, —CH₂OSO₃H, —CH₂OSO₂NH₂, —CH₂OSO₂NH(C₁-C₁₀ alkyl), —CH₂OSO₂N(C₁-C₁₀ alkyl)₂, —CH₂OSO₂NH-aryl or —CH₂N(R⁵)₂;

each R⁴ is independently —H, —C₁-C₁₀ alkyl, —(CH₂)ₙ-(3- to 7-membered monocyclic heterocycle), —(CH₂)ₙ-(8- to 12-membered bicyclic heterocycle), —(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkyl), —(CH₂)ₙ—(C₈-C₁₂ bicyclic cycloalkenyl), —(CH₂)ₙ-aryl, —C(O)O(C₁-C₁₀ alkyl), —C(O)NH(C₁-C₁₀ alkyl), —C(O)N(C₁-C₁₀ alkyl)₂, —C(O)NH-aryl, —C(O)N(aryl)₂, —CH(NH₂)NH₂ or —CH(NH₂)NH(C₁-C₁₀ alkyl);

each R⁵ is independently —H, —C₁-C₁₀ alkyl, —(CH₂)ₙ-(3- to 7-membered monocyclic heterocycle), —(CH₂)ₙ-(8- to 12-membered bicyclic heterocycle), —(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkyl), —(CH₂)ₙ—(C₈-C₁₂ bicyclic cycloalkenyl) or —(CH₂)ₙ-aryl;

R⁶ and R⁷ are each independently —H, —C₁-C₁₀ alkyl, —(CH₂)ₙ-(3- to 7-membered monocyclic heterocycle), —(CH₂)ₙ-(8- to 12-membered bicyclic heterocycle), —(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkyl), —(CH₂)ₙ—(C₈-C₁₂ bicyclic cycloalkenyl), —(CH₂)ₙ-aryl, -phenylene-(CH₂)ₙCOOH, or -phenylene-(CH₂)ₙCOO—(C₁-C₁₀ alkyl), or R⁶ and R⁷ together with the carbon atom to which they are attached form a —C₃-C₈ monocyclic cycloalkyl, a —C₈-C₁₂ bicyclic cycloalkyl, a —C₃-C₈ monocyclic cycloalkenyl or a —C₈-C₁₂ bicyclic cycloalkenyl;

m is an integer ranging from 0 to 3; and

each n is independently an integer ranging from 0 to 5.

In one embodiment, R¹ is —H.

In another embodiment, R¹ is —C₁-C₁₀ alkyl.

In another embodiment, R¹ is —(CH₂)ₘ-(3- to 7-membered monocyclic heterocycle) or —(CH₂)ₘ-(8- to 12-membered bicyclic heterocycle).

In still another embodiment, R¹ is —(CH₂)ₘ—(C₃-C₈ monocyclic cycloalkyl) or —(CH₂)ₘ—(C₃-C₈ monocyclic cycloalkenyl).

In a further embodiment, R¹ is —(CH₂)ₘ—(C₈-C₁₂ bicyclic cycloalkyl) or —(CH₂)ₙ—(C₈-C₁₂ bicyclic cycloalkenyl).

In another embodiment, R¹ is —(CH₂)ₘ-aryl.

In still another embodiment, two R¹ groups, together with the carbon atom to which they are attached, form a —C₃-C₈ monocyclic cycloalkyl, a —C₃-C₈ monocyclic cycloalkenyl, a —C₈-C₁₂ bicyclic cycloalkyl, or a —C₈-C₁₂ bicyclic cycloalkenyl.

In a specific embodiment, R¹ is cyclopentyl.

In one embodiment, m is 0.

In another embodiment, m is 1.

In another embodiment, m is 2.

In still another embodiment, m is 3.

In one embodiment, R² is -halo.

In a specific embodiment, R² is —Cl.

In one embodiment, R² is —H.

In another embodiment, R² is —CN.

In another embodiment, R² is —N(R⁴)₂, —OR⁴ or —SR⁴.

In a further embodiment, R² is —NHC(O)R⁴, —NHC(O)OR⁴ or —NHC(O)NHR⁴.

In another embodiment, R² is —NHNHC(O)R⁴, —NHNHC(O)OR⁴ or —NHNHC(O)NHR⁴.

In yet another embodiment, R² is —NH—N═C(R⁶)R⁷.

In another embodiment, R² is —NH—N═C(R⁶)R⁷ and R⁶ and R⁷ together with the carbon atom to which they are attached form a —C₃-C₈ monocyclic cycloalkyl, a —C₈-C₁₂ bicyclic cycloalkyl, a —C₃-C₈ monocyclic cycloalkenyl or a —C₈-C₁₂ bicyclic cycloalkenyl.

In a specific embodiment, R² is —NH—N═CH-cyclopentyl.

In one embodiment, R³ is —CH₂ONO₂ or —CH₂ONO.

In another embodiment, R³ is —CH₂OSO₃H, —CH₂OSO₂NH₂, —CH₂OSO₂NH(C₁-C₁₀ alkyl), —CH₂OSO₂N(C₁-C₁₀ alkyl)₂ or —CH₂OSO₂NH-aryl.

In another embodiment, R³ is —CH₂N(R⁵)₂.

In one embodiment, one occurrence of R¹ is —H.

In another embodiment, one occurrence of R¹ is —H and the other occurrence of R¹ is —C₃-C₈ monocyclic cycloalkyl.

In yet another embodiment, R³is —CH₂ONO₂.

In one embodiment, C and D are cis with respect to each other.

In another embodiment, C and D are trans with respect to each other.

In one embodiment, R⁸ and R⁹ are independently the residue of a naturally occurring amino acid.

In a specific embodiment, R⁸ and R⁹ are each:

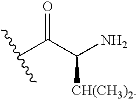

In another embodiment R⁸ and R⁹ join to form a —P(O)(OH)— group.

The present invention also provides compositions comprising an effective amount of a Purine Compound of Formula (168-III) and a physiologically acceptable carrier or vehicle.

The invention further provides Purine Compounds of Formula (168-III) that are in isolated and purified form.

The invention still further provides methods for treating or preventing a Condition, comprising administering an effective amount of a Purine Compound of Formula (168-III) to a subject in need thereof.

The invention further provides methods for reducing a subject's rate of metabolism, comprising administering an effective amount of a Purine Compound of Formula (168-III) to a subject in need thereof.

The invention further provides methods protecting a subject's heart against myocardial damage during cardioplegia, comprising administering an effective amount of a Purine Compound of Formula (168-III) to a subject in need thereof.

In a further embodiment, the invention provides compounds having the Formula (168-IV): 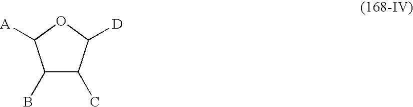
and pharmaceutically acceptable salts thereof, wherein

A is —CH₂OH;

B is —OR⁶;

C is —OR⁷;

wherein R⁶ and R⁷ are independently the residue of a naturally occurring amino acid that is attached via its C-terminus, or R⁶ and R⁷ join to form a —P(O)(OH)— group;

D is:

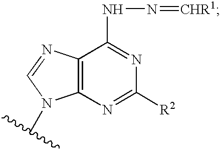

A and B are trans with respect to each other;

B and C are cis with respect to each other;

C and D are cis or trans with respect to each other;

R¹ is —C₃-C₈ monocyclic cycloalkyl, —(C₃-C₈ monocyclic cycloalkylene)-OH, or —C₃-C₈ monocyclic cycloalkenyl;

R² is —H, -halo, —CN, —OR₃, —SR³, —N(R³)₂, —NHNHC(O)R³, —NHNHC(O)NHR³, —NHNHC(O)OR³, or —NH—N═C(R⁴)R⁵;

each R³ is independently —H, —C₁-C₁₀ alkyl, —(CH₂)ₙ-(3- to 7-membered monocyclic heterocycle), —(CH₂)ₙ-(8- to 12-membered bicyclic heterocycle), —(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkyl), —(CH₂)ₙ—(C₈-C₁₂ bicyclic cycloalkenyl), —(CH₂)ₙ-aryl, —C≡C—(C₁-C₁₀ alkyl) or —C≡C-aryl;

R⁴ and R⁵ are each independently —H, —C₁-C₁₀ alkyl, —(CH₂)ₙ-(3- to 7-membered monocyclic heterocycle), —(CH₂)ₙ-(8- to 12-membered bicyclic heterocycle), —(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkyl), —(CH₂)ₙ—(C₈-C₁₂ bicyclic cycloalkenyl), —(CH₂)ₙ-aryl, -phenylene-(CH₂)ₙCOOH, or -phenylene-(CH₂)ₙCOO—(C₁-C₁₀ alkyl), or R⁴ and R⁵ together with the carbon atom to which they are attached form a C₃-C₈ monocyclic cycloalkyl, a C₃-C₈ monocyclic cycloalkenyl, a —C₈-C₁₂ bicyclic cycloalkyl, or a —C₈-C₁₂ bicyclic cycloalkenyl; and

each n is independently an integer ranging from 0 to 5. In one embodiment, R¹ is —C₃-C₈ monocyclic cycloalkyl.

In another embodiment, R¹ is —C₃-C₈ monocyclic cycloalkenyl.

In a specific embodiment, R¹ is cyclopentyl.

In one embodiment, R² is —H.

In another embodiment, R² is -halo.

In a specific embodiment, R² is —Cl.

In another embodiment, R² is —CN.

In another embodiment, R² is —N(R³)₂, —OR³ or —SR³.

In another embodiment, R² is —NHNHC(O)R³, —NHNHC(O)OR³ or —NHNHC(O)NHR³.

In yet another embodiment, R² is —NH—N═C(R⁴)R⁵.

In a specific embodiment, R² is —NH—N═CH-cyclopentyl.

In one embodiment, R³ is —(CH₂)ₙ—(C₈-C₁₂ bicyclic cycloalkenyl).

In one embodiment, C and D are cis with respect to each other.

In another embodiment, C and D are trans with respect to each other.

In one embodiment, R⁶ and R⁷ are independently the residue of a naturally occurring amino acid.

In a specific embodiment, R⁶ and R⁷ are each:

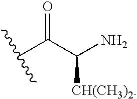

In another embodiment R⁶ and R⁷ join to form a —P(O)(OH)— group.

The present invention also provides compositions comprising an effective amount of a Purine Compound of Formula (168-IV) and a physiologically acceptable carrier or vehicle.

The invention further provides Purine Compounds of Formula (168-IV) that are in isolated and purified form.

The invention still further provides methods for treating or preventing a Condition, comprising administering an effective amount of a Purine Compound of Formula (168-IV) to a subject in need thereof.

The invention further provides methods for reducing a subject's rate of metabolism, comprising administering an effective amount of a Purine Compound of Formula (168-IV) to a subject in need thereof.

The invention further provides methods protecting a subject's heart against myocardial damage during cardioplegia, comprising administering an effective amount of a Purine Compound of Formula (168-IV) to a subject in need thereof.

In another embodiment, the invention provides compounds having the Formula (168-V): 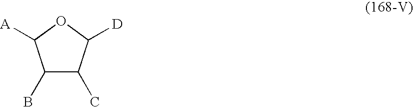
and pharmaceutically acceptable salts thereof, wherein

A is —CH₂OH;

B is —OR⁷;

C is —OR⁸;

wherein R⁷ and R⁸ are independently the residue of a naturally occurring amino acid that is attached via its C-terminus, or R⁷ and R⁸ join to form a —P(O)(OH)— group;

D is:

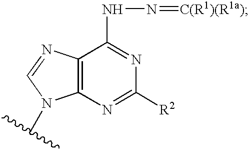

A and B are trans with respect to each other;

B and C are cis with respect to each other;

C and D are cis or trans with respect to each other;

R¹ is —C₁-C₁₀ alkyl, —(CH₂)ₘ-(3- to 7-membered monocyclic heterocycle), —(CH₂)ₘ-(8- to 12-membered bicyclic heterocycle), —(CH₂)ₘ—(C₈-C₁₂ bicyclic cycloalkyl), —(CH₂)ₙ—(C₈-C₁₂ bicyclic cycloalkenyl), —(CH₂)ₘ—(C₃-C₈ monocyclic cycloalkyl), —(CH₂)ₘ—(C₃-C₈ monocyclic cycloalkenyl), —(CH₂)ₘ—(C₃-C₈ monocyclic cycloalkylene)-OH or —(CH₂)ₘ-aryl, or R¹ and R^{1a} together with the carbon atom to which they are attached form a —C₃-C₈ monocyclic cycloalkyl, a —C₃-C₈ monocyclic cycloalkenyl, a —C₈-C₁₂ bicyclic cycloalkyl, or a —C₈-C₁₂ bicyclic cycloalkenyl;

R^{1a} is —C₃-C₈ monocyclic cycloalkyl or —C₃-C₈ monocyclic cycloalkenyl;

R² is —OR⁴, —SR⁴, —NHNHC(O)R³, —NHNHC(O)NHR³, —NHNHC(O)OR³, or —NH—N═C(R⁵)R⁶;

R³ is —H, —C₁-C₁₀ alkyl, —(CH₂)ₙ-(3- to 7-membered monocyclic heterocycle), —(CH₂)ₙ-(8- to 12-membered bicyclic heterocycle), —(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkyl), —(CH₂)ₙ—(C₈-C₁₂ bicyclic cycloalkenyl), —(CH₂)ₙ-aryl, —C≡C—(C₁-C₁₀ alkyl) or —C≡C-aryl;

R⁴ is —C₁-C₁₀ alkyl, —(CH₂)ₙ-(3- to 7-membered monocyclic heterocycle), —(CH₂)ₙ-(8- to 12-membered bicyclic heterocycle), —(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkyl), —(CH₂)ₙ—(C₈-C₁₂ bicyclic cycloalkenyl), —(CH₂)ₙ-aryl, —C≡C—(C₁-C₁₀ alkyl) or —C≡C-aryl;

R⁵ and R⁶ are each independently —H, —C₁-C₁₀ alkyl, —(CH₂)ₙ-(3- to 7-membered monocyclic heterocycle), —(CH₂)ₙ-(8- to 12-membered bicyclic heterocycle), —(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkyl), —(CH₂)ₙ—(C₈-C₁₂ bicyclic cycloalkenyl), —(CH₂)ₙ-aryl, -phenylene-(CH₂)ₙCOOH, or -phenylene-(CH₂)ₙCOO—(C₁-C₁₀ alkyl), or R⁵ and R⁶ together with the carbon atom to which they are attached form a C₃-C₈ monocyclic cycloalkyl, a C₃-C₈ monocyclic cycloalkenyl, a —C₈-C₁₂ bicyclic cycloalkyl, or a —C₈-C₁₂ bicyclic cycloalkenyl;

m is an integer ranging from 0 to 3; and

each n is independently an integer ranging from 0 to 5.

In one embodiment, R¹ is —C₁-C₁₀ alkyl.

In another embodiment, R¹ is —(CH₂)ₘ-(3- to 7-membered monocyclic heterocycle) or —(CH₂)ₘ-(8- to 12-membered bicyclic heterocycle).

In another embodiment, R¹ is —(CH₂)ₘ—(C₈-C₁₂ bicyclic cycloalkyl) or —(CH₂)ₙ—(C₈-C₁₂ bicyclic cycloalkenyl).

In still another embodiment, R¹ is —(CH₂)ₘ—(C₃-C₈ monocyclic cycloalkyl) or —(CH₂)ₘ—(C₃-C₈ monocyclic cycloalkenyl).

In a further embodiment, R¹ is —(CH₂)ₘ-aryl.

In one embodiment, R^{1a} is —C₃-C₈ monocyclic cycloalkyl.

In another embodiment, R^{1a} is —C₃-C₈ monocyclic cycloalkenyl.

In a specific embodiment, R^{1a} is cyclopentyl.

In another embodiment, R¹ and R^{1a} together with the carbon atom to which they are attached form a —C₃-C8 monocyclic cycloalkyl, a —C₃-C₈ monocyclic cycloalkenyl, a —C₈-C₁₂ bicyclic cycloalkyl, or a —C₈-C₁₂ bicyclic cycloalkenyl.

In one embodiment, R² is —OR⁴ or —SR⁴.

In another embodiment, R² is —NHNHC(O)R³, —NHNHC(O)OR³ or —NHNHC(O)NHR³.

In yet another embodiment, R² is —NH—N═C(R⁵)R⁶.

In a specific embodiment, R² is —NH—N═CH-cyclopentyl.

In one embodiment, R⁴ is —(CH₂)ₙ—(C₈-C₁₂ bicyclic cycloalkenyl).

In one embodiment, C and D are cis with respect to each other.

In another embodiment, C and D are trans with respect to each other.

In one embodiment, R⁷ and R⁸ are independently the residue of a naturally occurring amino acid.

In a specific embodiment, R⁷ and R⁸ are each:

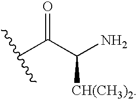

In another embodiment R⁷ and R⁸ join to form a —P(O)(OH)— group.

The present invention also provides compositions comprising an effective amount of a Purine Compound of Formula (168-V) and a physiologically acceptable carrier or vehicle.

The invention further provides Purine Compounds of Formula (168-V) that are in isolated and purified form.

The invention still further provides methods for treating or preventing a Condition, comprising administering an effective amount of a Purine Compound of Formula (168-V) to a subject in need thereof.

The invention further provides methods for reducing a subject's rate of metabolism, comprising administering an effective amount of a Purine Compound of Formula (168-V) to a subject in need thereof.

The invention further provides methods protecting a subject's heart against myocardial damage during cardioplegia, comprising administering an effective amount of a Purine Compound of Formula (168-V) to a subject in need thereof.

### 5.1.2 The Purine Compounds of Formula (II)

As stated above, the present invention encompasses Purine Compounds having the Formula (II): 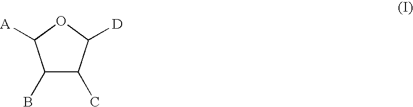
wherein A, B, C and D are defined above for the Purine Compounds of Formula (II), and A and B are trans with respect to each other; B and C are cis with respect to each other; and C and D are cis or trans with respect to each other.

It is to be understood that in group D of the Purine Compounds of Formula (II), depicted below: 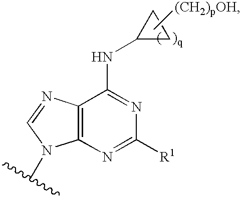
the —(CH₂)ₚOH group can be joined at any carbon atom of the 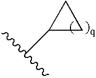
group to which it is attached.

In one embodiment, R¹ is —H.

In another embodiment, R¹ is -halo.

In a specific embodiment, R¹ is —Cl.

In another embodiment, R¹ is —CN.

In still another embodiment, R¹ is −N(R²)₂.

In yet another embodiment, R¹ is —OR².

In a further embodiment, R¹ is —SR².

In another embodiment, R¹ is —NHC(O)OR², —NHC(O)R² or —NHC(O)N(R²).

In another embodiment, R¹ is —C(O)OR², —C(O)R², —C(O)N(R²)₂, or —OC(O)N(R²)₂.

In still another embodiment, R¹ is CF₃.

In yet another embodiment, R¹ is —NO₂.

In one embodiment, p is 1.

In another embodiment, p is other than 1.

In one embodiment, q is 1.

In another embodiment, q is 2.

In still another embodiment, q is 3.

In yet another embodiment, q is 4.

In a further embodiment, q is 5.

In another embodiment, q is 6.

In one embodiment, R¹ is —H, p is 1 and q is 1.

In another embodiment, R¹ is -halo, p is 1 and q is 1.

In still another embodiment, R¹ is —Cl, p is 1 and q is 1.

In one embodiment, C and D are cis with respect to each other.

In another embodiment, C and D are trans with respect to each other.

In one embodiment, R³ and R⁴ are independently the residue of a naturally occurring amino acid.

In another embodiment, p is 1 and R³ and R⁴ are independently the residue of a naturally occurring amino acid.

In a specific embodiment, R³ and R⁴ are each:

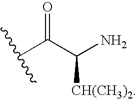

In one embodiment, p is 1 and R³ and R⁴ are each:

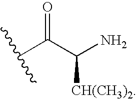

In another embodiment R³ and R⁴ join to form a —P(O)(OH)— group.

In yet another embodiment, p is 1 and R³ and R⁴ join to form a —P(O)(OH)— group.

### 5.1.3 The Purine Compounds of Formula (III)

As stated above, the present invention encompasses Purine Compounds having the Formula (III): 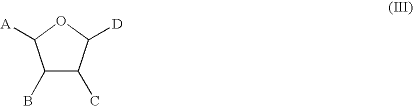
wherein A, B, C and D are defined above for the Purine Compounds of Formula (III), and A and B are trans with respect to each other; B and C are cis with respect to each other; and C and D are cis or trans with respect to each other.

In one embodiment, the invention provides compounds having the Formula (170-I): 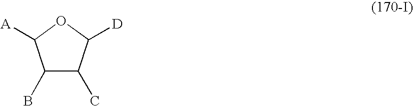
and pharmaceutically acceptable salts thereof, wherein

A is —C(O)NHR³;

B is —OR⁵;

C is —OR⁶;

wherein R⁵ and R⁶ join to form a —P(O)(OH)— group;

D is:

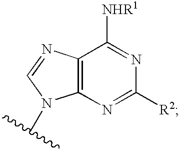

A and B are trans with respect to each other;

B and C are cis with respect to each other;

C and D are cis or trans with respect to each other;

R¹ is —H, —C₁-C₆ alkyl, —(C₁-C₆ alkylene)-aryl, or —(C₁-C₆ alkylene)-(arylene)-halo;

R² is —H, -halo, —OR⁴, —C(O)NH(CH₂)ₙR⁴, —C═C—R⁴, —CH═CHR⁴, —NH(C₁-C₆ alkyl), —NH((C₁-C₆ alkylene)-aryl), —NH((C₁-C₆ alkylene)-(arylene)-(CH₂)ₙ—COOH), or —NH((C₁-C₆ alkylene)-3- to 7-membered monocyclic heterocycle);

R³ is —C₁-C₆ alkyl;

R⁴ is —C₁-C₆ alkyl, -aryl, -3- to 7-membered monocyclic heterocycle, -8- to 12-membered bicyclic heterocycle, —C₃-C₈ monocyclic cycloalkyl, —C₈-C₁₂ bicyclic cycloalkyl, —(C₁-C₆ alkylene)-(C₃-C₈ monocyclic cycloalkylene)-CH₂OH; and

n is an integer ranging from 0 to 6.

In one embodiment, R¹ is —H.

In another embodiment, R¹ is —C₁-C₆ alkyl.

In a specific embodiment, R¹ is methyl.

In another embodiment, R¹ —(C₁-C₆ alkylene)-aryl.

In still another embodiment, R¹ is —(C₁-C₆ alkylene)-(arylene)-halo.

In a specific embodiment, R¹ is 3-iodobenzyl.

In one embodiment, R² is —H.

In another embodiment, R² is -halo.

In another embodiment, R² is —OR⁴.

In still another embodiment, R² is C(O)NH(CH₂)ₙR⁴.

In a further embodiment, R² is —C≡C—R⁴ or —CH═CHR⁴.

In a specific embodiment, R² is

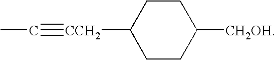

In another specific embodiment, R² is

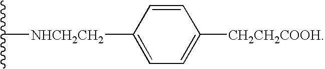

In another specific embodiment, R² is —C≡C—(CH₂)₅CH₃.

In a further specific embodiment, R² is —C≡C-phenyl.

In another embodiment, R² is —NH(C₁-C₆ alkyl), —NH((C₁-C₆ alkylene)-aryl), —NH((C₁-C₆ alkylene)-(arylene)-(CH₂)ₙ—COOH), or —NH((C₁-C₆ alkylene)-3- to 7-membered monocyclic heterocycle).

In one embodiment, R³ is methyl or ethyl.

In one embodiment, R⁴ is —C₁-C₆ alkyl.

In another embodiment, R⁴ is -aryl.

In another embodiment, R⁴ is -3- to 7-membered monocyclic heterocycle or -8- to 12-membered bicyclic heterocycle.

In still another embodiment, R⁴ is —C₃-C₈ monocyclic cycloalkyl or —C₈-C₁₂ bicyclic cycloalkyl.

In a further embodiment, R⁴ is —(C₁-C₆ alkylene)-(C₃-C₈ monocyclic cycloalkylene)-CH₂OH.

In one embodiment, C and D are cis with respect to each other.

In another embodiment, C and D are trans with respect to each other.

The present invention also provides compositions comprising an effective amount of a Purine Compound of Formula (170-I) and a physiologically acceptable carrier or vehicle.

The invention further provides Purine Compounds of Formula (170-I) that are in isolated and purified form.

The invention still further provides methods for treating or preventing a Condition, comprising administering an effective amount of a Purine Compound of Formula (170-I) to a subject in need thereof.

The invention further provides methods for reducing a subject's rate of metabolism, comprising administering an effective amount of a Purine Compound of Formula (170-I) to a subject in need thereof.

The invention further provides methods for protecting a subject's heart against myocardial damage during cardioplegia, comprising administering an effective amount of a Purine Compound of Formula (170-I) to a subject in need thereof.

In another embodiment, the invention provides compounds having the Formula (170-II): 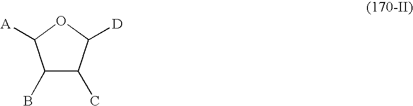
and pharmaceutically acceptable salts thereof, wherein

A is —CH₂OH;

B is —OR⁴;

C is —OR⁵;

wherein R⁴ and R⁵ join to form a —P(O)(OH)— group;

D is:

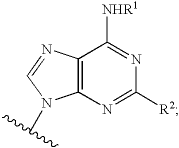

A and B are trans with respect to each other;

B and C are cis with respect to each other;

C and D are cis or trans with respect to each other;

R¹ is —H, —C₁-C₆ alkyl, -aryl, -(arylene)-C₁-C₆ alkyl, -3- to 7-membered monocyclic heterocycle, -8- to 12-membered bicyclic heterocycle, —C₃-C₈ monocyclic cycloalkyl, —(C₃-C₈ monocyclic cycloalkylene)-OH, —C₈-C₁₂ bicyclic cycloalkyl, -(3- to 7-membered monocyclic heterocyclene)-S-aryl, —(C₁-C₆ alkylene)-S-(8- to 12-membered bicyclic heterocycle) or —(C₁-C₆ alkylene)-aryl;

R² is -halo, —CN, —C≡C—R³, —C(O)NHR³, —CH═CHR³, —OH, —O—(C₁-C₆ alkyl), —NH—N═CHR³, —C₁-C₆ alkyl, -aryl, -3- to 7-membered monocyclic heterocycle, -8- to 12-membered bicyclic heterocycle, —NH(C₁-C₆ alkyl), —NH((C₁-C₆ alkylene)-aryl), —NH((C₁-C₆ alkylene)-C₃-C₈ monocyclic cycloalkyl), —NH—((C₁-C₆ alkylene)-C₈-C₁₂ bicyclic cycloalkyl), —CH₂—O—(C₁-C₆ alkyl), —CH₂—NH(C₁-C₆ alkyl) or —CH₂—NH-aryl; and

R³ is —C₁-C₆ alkyl, -aryl, -3- to 7-membered monocyclic heterocycle, -8- to 12-membered bicyclic heterocycle, —C₃-C₈ monocyclic cycloalkyl, —CH₂—(C₃-C₈ monocyclic cycloalkyl) or —C₈-C₁₂ bicyclic cycloalkyl.

In one embodiment, R¹ is -aryl.

In another embodiment, R¹ is —C₃-C₈ monocyclic cycloalkyl or —C₈-C₁₂ bicyclic cycloalkyl.

In another embodiment, R¹ is -3- to 7-membered monocyclic heterocycle or -8- to 12-membered bicyclic heterocycle.

In still another embodiment, R¹ is -(arylene)-(C₁-C₆ alkyl).

In yet another embodiment, R¹ is —(C₃-C₈ monocyclic cycloalkylene)-OH.

In a further embodiment, R¹ is -(3- to 7-membered monocyclic heterocycle)-S-aryl.

In another embodiment, R¹ is —(C₁-C₆ alkylene)-S-(8- to 12-membered bicyclic heterocycle.

In still another embodiment, R¹ is —(C₁-C₆ alkylene)-aryl.

In one embodiment, R² is —H.

In another embodiment, R² is —CN.

In a further embodiment, R² is -halo.

In another embodiment, R² is —C≡C—R³ or —CH═CHR³.

In yet another embodiment, R² is —OH.

In another embodiment, R² is —O—(C₁-C₆ alkyl).

In another embodiment, R² is —NH—N═CHR³.

In a further embodiment, R² is —C₁-C₆ alkyl.

In another embodiment, R² is -aryl.

In yet another embodiment, R² is -3- to 7-membered monocyclic heterocycle or -8- to 12-membered bicyclic heterocycle.

In a further embodiment, R² is —NH—(C₁-C₆ alkyl), —NH—(C₁-C₆ alkylene)-aryl or —NH—(C₁-C₆ alkylene)-cycloalkyl.

In another embodiment, R² is —CH₂—O—(C₁-C₆ alkyl).

In yet another embodiment, R² is —CH₂—NH—(C₁-C₆ alkyl) or —CH₂—NH-aryl.

In a further embodiment, R³ is —C₁-C₆ alkyl.

In another embodiment, R³ is -aryl.

In yet another embodiment, R³is -3- to 7-membered monocyclic heterocycle or -8- to 12-membered bicyclic heterocycle.

In a further embodiment, R³ is —C₃-C₈ monocyclic cycloalkyl or —C₈-C₁₂ bicyclic cycloalkyl.

In another embodiment, R³ is —CH₂—(C₃-C₈ monocyclic cycloalkyl).

In one embodiment, C and D are cis with respect to each other.

In another embodiment, C and D are trans with respect to each other.

The present invention also provides compositions comprising an effective amount of a Purine Compound of Formula (170-II) and a physiologically acceptable carrier or vehicle.

The invention further provides Purine Compounds of Formula (170-II) that are in isolated and purified form.

The invention still further provides methods for treating or preventing a Condition, comprising administering an effective amount of a Purine Compound of Formula (170-II) to a subject in need thereof.

The invention further provides methods for reducing a subject's rate of metabolism, comprising administering an effective amount of a Purine Compound of Formula (170-II) to a subject in need thereof.

The invention further provides methods protecting a subject's heart against myocardial damage during cardioplegia, comprising administering an effective amount of a Purine Compound of Formula (170-II) to a subject in need thereof.

In another embodiment, the invention provides compounds having the Formula (170-IIA): 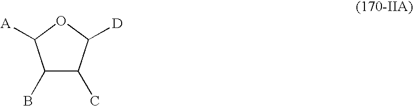
and pharmaceutically acceptable salts thereof, wherein

A is —CH₂OH;

B is —OR⁴;

C is —OR⁵;

wherein R⁴ and R⁵ join to form a —P(O)(OH)— group;

D is:

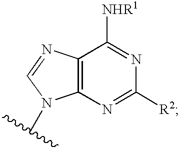

A and B are trans with respect to each other;

B and C are cis with respect to each other;

C and D are cis or trans with respect to each other;

R¹ is -aryl, -(arylene)-C₁-C₆ alkyl, -3- to 7-membered monocyclic heterocycle, -8- to 12-membered bicyclic heterocycle, —C₃-C₈ monocyclic cycloalkyl, —(C₃-C₈ monocyclic cycloalkylene)-OH, —C₈-C₁₂ bicyclic cycloalkyl, -(3- to 7-membered monocyclic heterocyclene)-S-aryl, —(C₁-C₆ alkylene)-S-(8- to 12-membered bicyclic heterocycle) or —(C₁-C₆ alkylene)-aryl;

R² is —H, -halo, —CN, —C≡C—R³, —C(O)NHR³, —CH═CHR³, —OH, —O—(C₁-C₆ alkyl), —NH—N═CHR³, —C₁-C₆ alkyl, -aryl, -3- to 7-membered monocyclic heterocycle, -8- to 12-membered bicyclic heterocycle, —NH(C₁-C₆ alkyl), —NH((C₁-C₆ alkylene)-aryl), —NH((C₁-C₆ alkylene)-C₃-C₈ monocyclic cycloalkyl), —NH((C₁-C₆ alkylene)-C₈-C₁₂ bicyclic cycloalkyl), —CH₂—O—(C₁-C₆ alkyl), —CH₂—NH(C₁-C₆ alkyl) or —CH₂—NH-aryl; and

R³ is —C₁-C₆ alkyl, -aryl, -3- to 7-membered monocyclic heterocycle, -8- to 12-membered bicyclic heterocycle, —C₃-C₈ monocyclic cycloalkyl, —CH₂—(C₃-C₈ monocyclic cycloalkyl) or —C₈-C₁₂ bicyclic cycloalkyl.

In one embodiment, R¹ is —H.

In another embodiment, R¹ is —C₁-C₆ alkyl.

In another embodiment, R¹ is -aryl.

In still another embodiment, R¹ is —C₃-C₈ monocyclic cycloalkyl or —C₈-C₁₂ bicyclic cycloalkyl.

In another embodiment, R¹ is -3- to 7-membered monocyclic heterocycle or -8- to 12-membered bicyclic heterocycle.

In still another embodiment, R¹ is -(arylene)-(C₁-C₆ alkyl).

In yet another embodiment, R¹ is —(C₃-C₈ monocyclic cycloalkylene)-OH.

In a further embodiment, R¹ is -(3- to 7-membered monocyclic heterocycle)-S-aryl.

In another embodiment, R¹ is —(C₁-C₆ alkylene)-S-(8- to 12-membered bicyclic heterocycle.

In still another embodiment, R¹ is —(C₁-C₆ alkylene)-aryl.

In another embodiment, R² is —CN.

In a further embodiment, R² is -halo.

In another embodiment, R² is —C≡C—R³ or —CH═CHR³.

In yet another embodiment, R² is —OH.

In another embodiment, R² is —O—(C₁-C₆ alkyl).

In another embodiment, R² is —NH—N═CHR³.

In a further embodiment, R² is —C₁-C₆ alkyl.

In another embodiment, R² is -aryl.

In yet another embodiment, R² is -3- to 7-membered monocyclic heterocycle or -8- to 12-membered bicyclic heterocycle.

In a further embodiment, R² is —NH—(C₁-C₆ alkyl), —NH—(C₁-C₆ alkylene)-aryl or —NH—(C₁-C₆ alkylene)-cycloalkyl.

In another embodiment, R² is —CH²—O—(C₁-C₆ alkyl).

In yet another embodiment, R² is —CH₂—NH—(C₁-C₆ alkyl) or —CH₂—NH-aryl.

In a further embodiment, R³ is —C₁-C₆ alkyl.

In another embodiment, R³ is -aryl.

In yet another embodiment, R³ is -3- to 7-membered monocyclic heterocycle or -8- to 12-membered bicyclic heterocycle.

In a further embodiment, R³ is —C₃-C₈ monocyclic cycloalkyl or —C₈-C₁₂ bicyclic cycloalkyl.

In another embodiment, R³ is —CH₂—(C₃-C₈ monocyclic cycloalkyl).

In one embodiment, C and D are cis with respect to each other.

In another embodiment, C and D are trans with respect to each other.

The present invention also provides compositions comprising an effective amount of a Purine Compound of Formula (170-IIA) and a physiologically acceptable carrier or vehicle.

The invention further provides Purine Compounds of Formula (170-IIA) that are in isolated and purified form.

The invention still further provides methods for treating or preventing a Condition, comprising administering an effective amount of a Purine Compound of Formula (170-IIA) to a subject in need thereof.

The invention further provides methods for reducing a subject's rate of metabolism, comprising administering an effective amount of a Purine Compound of Formula (170-IIA) to a subject in need thereof.

The invention further provides methods protecting a subject's heart against myocardial damage during cardioplegia, comprising administering an effective amount of a Purine Compound of Formula (170-IIA) to a subject in need thereof.

In still another embodiment, the invention provides compounds having the Formula (170-III): 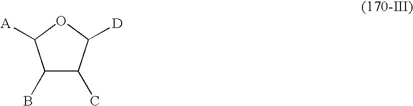
and pharmaceutically acceptable salts thereof, wherein

A is —CH₂OSO₃H;

B is —OR⁴;

C is —OR⁵;

wherein R⁴ and R⁵ join to form a —P(O)(OH)— group;

D is:

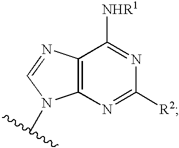

A and B are trans with respect to each other;

B and C are cis with respect to each other;

C and D are cis or trans with respect to each other;

R¹ is —H, —C₁-C₆ alkyl, -aryl, -3- to 7-membered monocyclic heterocycle, -8- to 12-membered bicyclic heterocycle, —C₃-C₈ monocyclic cycloalkyl or —C₈-C₁₂ bicyclic cycloalkyl;

R² is —C(O)NHR³, —C≡C—R³, —CH═CHR³, —CH₂—NH(C₁-C₆ alkyl), —CH₂—NH-aryl or —CH₂—O—(C₁-C₆ alkyl); and

R³ is —C₁-C₆ alkyl, -aryl, -3- to 7-membered monocyclic heterocycle, -8- to 12-membered bicyclic heterocycle, —C₃-C₈ monocyclic cycloalkyl or —C₈-C₁₂ bicyclic cycloalkyl.

In one embodiment, R¹ is —H.

In another embodiment, R¹ is —C₁-C₆ alkyl.

In another embodiment, R¹ is -aryl.

In still another embodiment, R¹ is —C₃-C₈ monocyclic cycloalkyl or —C₈-C₁₂ bicyclic cycloalkyl.

In another embodiment, R¹ is -3- to 7-membered monocyclic heterocycle or -8- to 12-membered bicyclic heterocycle.

In one embodiment, R² is —C≡C—R³ or —CH═CHR³.

In another embodiment, R² is —C(O)NHR³.

In another embodiment, R² is —CH₂—O—(C₁-C₆ alkyl).

In yet another embodiment, R²is —CH₂—NH—(C₁-C₆ alkyl) or —CH₂—NH-aryl.

In one embodiment, R³ is —C₁-C₆ alkyl.

In another embodiment, R³ is -aryl.

In yet another embodiment, R³ is -3- to 7-membered monocyclic heterocycle or -8- to 12-membered bicyclic heterocycle.

In a further embodiment, R³ is —C₃-C₈ monocyclic cycloalkyl or —C₈-C₁₂ bicyclic cycloalkyl.

In one embodiment, C and D are cis with respect to each other.

In another embodiment, C and D are trans with respect to each other.

The present invention also provides compositions comprising an effective amount of a Purine Compound of Formula (170-III) and a physiologically acceptable carrier or vehicle.

The invention further provides Purine Compounds of Formula (170-III) that are in isolated and purified form.

The invention still further provides methods for treating or preventing a Condition, comprising administering an effective amount of a Purine Compound of Formula (170-III) to a subject in need thereof.

The invention further provides methods for reducing a subject's rate of metabolism, comprising administering an effective amount of a Purine Compound of Formula (170-III) to a subject in need thereof.

The invention further provides methods protecting a subject's heart against myocardial damage during cardioplegia, comprising administering an effective amount of a Purine Compound of Formula (170-III) to a subject in need thereof.

In a further embodiment, the invention provides compounds having the Formula (170-IV): 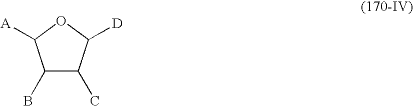
and pharmaceutically acceptable salts thereof, wherein

A is —CH₂ONO₂;

B is —OR⁴;

C is —OR⁵;

wherein R⁴ and R⁵ join to form a —P(O)(OH)— group;

D is:

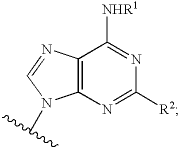

A and B are trans with respect to each other;

B and C are cis with respect to each other;

C and D are cis or trans with respect to each other;

R¹ is —C₁-C₆ alkyl, -aryl, -3- to 7-membered monocyclic heterocycle, -8- to 12-membered bicyclic heterocycle or —C₈-C₁₂ bicyclic cycloalkyl;

R² is —C(O)NHR³, —C≡C—R³, —CH═CHR³, -aryl, -3- to 7-membered monocyclic heterocycle, -8- to 12-membered bicyclic heterocycle, —CH₂—NH(C₁-C₆ alkyl), —CH₂—NH-aryl or —CH₂—O—(C₁-C₆ alkyl); and

R³ is —C₁-C₆ alkyl, -aryl, -3- to 7-membered monocyclic heterocycle, -8- to 12-membered bicyclic heterocycle, —C₃-C₈ monocyclic cycloalkyl or —C₈-C₁₂ bicyclic cycloalkyl.

In one embodiment, R¹ is —C₁-C₆ alkyl.

In another embodiment, R¹ is -aryl.

In still another embodiment, R¹ is —C₈-C₁₂ bicyclic cycloalkyl.

In another embodiment, R¹ is -3- to 7-membered monocyclic heterocycle or -8- to 12-membered bicyclic heterocycle.

In one embodiment, R² is —C≡C—R³ or —CH═CHR₃.

In another embodiment, R² is —C(O)NHR³.

In another embodiment, R² is -aryl.

In still another embodiment, R² is -3- to 7-membered monocyclic heterocycle or -8- to 12-membered bicyclic heterocycle.

In a further embodiment, R² is —CH₂—O—(C₁-C₆ alkyl).

In another embodiment, R² is —CH₂—NH—(C₁-C₆ alkyl) or —CH₂—NH-aryl.

In one embodiment, R³ is —C₁-C₆ alkyl.

In another embodiment, R³ is -aryl.

In yet another embodiment, R³ is -3- to 7-membered monocyclic heterocycle or -8- to 12-membered bicyclic heterocycle.

In a further embodiment, R³ is —C₃-C₈ monocyclic cycloalkyl or —C₈-C₁₂ bicyclic cycloalkyl.

In one embodiment, C and D are cis with respect to each other.

In another embodiment, C and D are trans with respect to each other.

The present invention also provides compositions comprising an effective amount of a Purine Compound of Formula (170-IV) and a physiologically acceptable carrier or vehicle.

The invention further provides Purine Compounds of Formula (170-IV) that are in isolated and purified form.

The invention still further provides methods for treating or preventing a Condition, comprising administering an effective amount of a Purine Compound of Formula (170-IV) to a subject in need thereof.

The invention further provides methods for reducing a subject's rate of metabolism, comprising administering an effective amount of a Purine Compound of Formula (170-IV) to a subject in need thereof.

The invention further provides methods protecting a subject's heart against myocardial damage during cardioplegia, comprising administering an effective amount of a Purine Compound of Formula (170-IV) to a subject in need thereof.

In a further embodiment, the invention provides compounds having the Formula (170-V): 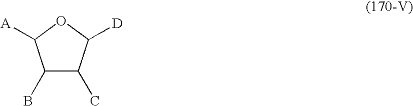
and pharmaceutically acceptable salts thereof, wherein

A is —R³;

B is —OR⁵;

C is —OR⁶;

wherein R⁵ and R⁶ join to form a —P(O)(OH)— group;

D is:

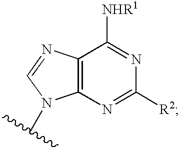

A and B are trans with respect to each other;

B and C are cis with respect to each other;

C and D are cis or trans with respect to each other;

R¹ is —H, —C₁-C₆ alkyl, -aryl, -3- to 7-membered monocyclic heterocycle, -8- to 12-membered bicyclic heterocycle, —C₃-C₈ monocyclic cycloalkyl, —C₈-C₁₂ bicyclic cycloalkyl or —CH((C₁-C₆ alkylene)-OH)((C₁-C₆ alkylene)-(arylene)-O—C₁-C₆ alkyl);

R² is —H, -halo, —CN, —C≡C—R⁴, —CH═CHR⁴, —OH, —O—(C₁-C₆ alkyl), —NH—N═CHR⁴, —C₁-C₆ alkyl, -aryl, -3- to 7-membered monocyclic heterocycle, -8- to 12-membered bicyclic heterocycle, —NH(C₁-C₆ alkyl), —NH-aryl, —NH(C₃-C₈ monocyclic cycloalkyl), —NH(C₈-C₁₂ bicyclic cycloalkyl), —CH₂—O—(C₁-C₆ alkyl), —CH₂—NH(C₁-C₆ alkyl) or —CH₂—NH-aryl;

R³ is —(C₁-C₆ alkylene)ₙ-(3- to 7-membered monocyclic heterocycle), —(C₁-C₆ alkylene)ₙ-(3- to 7-membered monocyclic heterocyclene)-C₁-C₆ alkyl or —(C₁-C₆ alkylene)ₙ-(8- to 12-membered bicyclic heterocycle);

R⁴ is —C₁-C₆ alkyl, -aryl, -3- to 7-membered monocyclic heterocycle, -8- to 12-membered bicyclic heterocycle, —C₃-C₈ monocyclic cycloalkyl, —(C₃-C₈ monocyclic cycloalkylene)-OH or —C₈-C₁₂ bicyclic cycloalkyl; and

n is 0 or 1.

In one embodiment, R¹ is —H.

In another embodiment, R¹ is —C₁-C₆ alkyl.

In another embodiment, R¹ is -aryl.

In still another embodiment, R¹ is —C₃-C₈ monocyclic cycloalkyl or —C₈-C₁₂ bicyclic cycloalkyl.

In another embodiment, R¹ is -3- to 7-membered monocyclic heterocycle or -8- to 12-membered bicyclic heterocycle.

In a further embodiment, R¹ is —H, —C₁-C₆ alkyl, -aryl, -3- to 7-membered monocyclic heterocycle, -8- to 12-membered bicyclic heterocycle, —C₃-C₈ monocyclic cycloalkyl or —C₈-C₁₂ bicyclic cycloalkyl;

In one embodiment, R² is —H.

In another embodiment, R² is —CN.

In a further embodiment, R² is -halo.

In another embodiment, R² is —C≡C—R⁴ or —CH═CHR⁴.

In yet another embodiment, R² is —OH.

In another embodiment, R² is —O—(C₁-C₆ alkyl).

In another embodiment, R² is —NH—N═CHR⁴.

In a further embodiment, R² is —C₁-C₆ alkyl.

In another embodiment, R² is -aryl.

In yet another embodiment, R² is -3- to 7-membered monocyclic heterocycle or -8- to 12-membered bicyclic heterocycle.

In a further embodiment, R² is —NH—(C₁-C₆ alkyl), —NH-aryl or —NH—(—C₃-C₈ monocyclic cycloalkyl).

In another embodiment, R² is —CH₂—O—(C₁-C₆ alkyl).

In yet another embodiment, R² is —CH₂—NH—(C₁-C₆ alkyl) or —CH₂—NH-aryl.

In a further embodiment, R³ is —(C₁-C₆ alkylene)ₙ-(3- to 7-membered monocyclic heterocycle).

In another embodiment, R³ is —(C₁-C₆ alkylene)ₙ-(8- to 12-membered bicyclic heterocycle).

In yet another embodiment, R³ is —(C₁-C₆ alkylene)ₙ-(3- to 7-membered monocyclic heterocycle) or —(C₁-C₆ alkylene)ₙ-(8- to 12-membered bicyclic heterocycle);

In one embodiment, n is 0.

In another embodiment, n is 1.

In one embodiment, R⁴ is —C₁-C₆ alkyl.

In another embodiment, R⁴ is -aryl.

In yet another embodiment, R⁴ is -3- to 7-membered monocyclic heterocycle or -8- to 12-membered bicyclic heterocycle.

In a further embodiment, R⁴ is —C₃-C₈ monocyclic cycloalkyl or —C₈-C₁₂ bicyclic cycloalkyl.

In another embodiment, R⁴ is —HO-substituted-C₃-C₈ monocyclic cycloalkyl.

In one embodiment, C and D are cis with respect to each other.

In another embodiment, C and D are trans with respect to each other.

The present invention also provides compositions comprising an effective amount of a Purine Compound of Formula (170-V) and a physiologically acceptable carrier or vehicle.

The invention further provides Purine Compounds of Formula (170-V) that are in isolated and purified form.

The invention still further provides methods for treating or preventing a Condition, comprising administering an effective amount of a Purine Compound of Formula (170-V) to a subject in need thereof.

The invention further provides methods for reducing a subject's rate of metabolism, comprising administering an effective amount of a Purine Compound of Formula (170-V) to a subject in need thereof.

The invention further provides methods protecting a subject's heart against myocardial damage during cardioplegia, comprising administering an effective amount of a Purine Compound of Formula (170-V) to a subject in need thereof.

In another embodiment, the invention provides compounds having the Formula (170-VI): 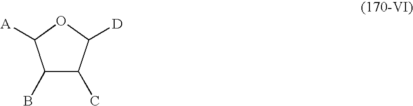
and pharmaceutically acceptable salts thereof, wherein

A is —C(O)NHR³;

B is —OR⁵;

C is —OR⁶;

wherein R⁵ and R⁶ are independently the residue of a naturally occurring amino acid that is attached via its C-terminus;

D is:

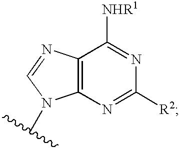

A and B are trans with respect to each other;

B and C are cis with respect to each other;

C and D are cis or trans with respect to each other;

R¹ is —H, —C₁-C₆ alkyl, —(C₁-C₆ alkylene)-aryl, or —(C₁-C₆ alkylene)-(arylene)-halo;

R² is —H, -halo, —OR⁴, —C(O)NH(CH₂)ₙR⁴, —C≡C—R⁴, —CH═CHR⁴, —NH(C₁-C₆ alkyl), —NH((C₁-C₆ alkylene)-aryl), —NH((C₁-C₆ alkylene)-(arylene)-(CH₂)ₙ—COOH), or —NH((C₁-C₆ alkylene)-3- to 7-membered monocyclic heterocycle);

R³ is —C₁-C₆ alkyl;

R⁴ is —C₁-C₆ alkyl, -aryl, -3- to 7-membered monocyclic heterocycle, -8- to 12-membered bicyclic heterocycle, —C₃-C₈ monocyclic cycloalkyl, —C₈-C₁₂ bicyclic cycloalkyl, —(C₁-C₆ alkylene)-(C₃-C₈ monocyclic cycloalkylene)-CH₂OH; and

n is an integer ranging from 0 to 6.

In one embodiment, R¹ is —H.

In another embodiment, R¹ is —C₁-C₆ alkyl.

In another embodiment, R¹ is —(C₁-C₆ alkyleneyaryl.

In another embodiment, R¹ is —(C₁-C₆ alkylene)-(arylene)-halo.

In one embodiment, R² is —H.

In another embodiment, R² is —OR⁴.

In a further embodiment, R² is -halo.

In another embodiment, R² is —C≡C—R⁴ or —CH═CHR⁴.

In another embodiment, R² is —C(O)NH(CH₂)ₙR⁴.

In a further embodiment, R² is —NH—(C₁-C₆ alkyl), —NH—(C₁-C₆ alkyl)-aryl, —NH((C₁-C₆ alkylene)-(arylene)-(CH₂)ₙ—COOH) or —NH((C₁-C₆ alkylene)-3- to 7-membered monocyclic heterocycle).

In one embodiment, R⁴ is —C₁-C₆ alkyl.

In another embodiment, R⁴ is -aryl.

In yet another embodiment, R⁴ is -3- to 7-membered monocyclic heterocycle or -8- to 12-membered bicyclic heterocycle.

In a further embodiment, R⁴ is —C₃-C₈ monocyclic cycloalkyl or —C₈-C₁₂ bicyclic cycloalkyl.

In another embodiment, R⁴ is —(C₁-C₆ alkylene)-(C₃-C₈ monocyclic cycloalkylene)-CH₂—OH.

In one embodiment, C and D are cis with respect to each other.

In another embodiment, C and D are trans with respect to each other.

In a specific embodiment, R⁵ and R⁶ are each:

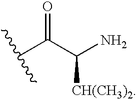

The present invention also provides compositions comprising an effective amount of a Purine Compound of Formula (170-VI) and a physiologically acceptable carrier or vehicle.

The invention further provides Purine Compounds of Formula (170-VI) that are in isolated and purified form.

The invention still further provides methods for treating or preventing a Condition, comprising administering an effective amount of a Purine Compound of Formula (170-VI) to a subject in need thereof.

The invention further provides methods for reducing a subject's rate of metabolism, comprising administering an effective amount of a Purine Compound of Formula (170-VI) to a subject in need thereof.

The invention further provides methods protecting a subject's heart against myocardial damage during cardioplegia, comprising administering an effective amount of a Purine Compound of Formula (170-VI) to a subject in need thereof.

In another embodiment, the invention provides compounds having the Formula (170-VII): 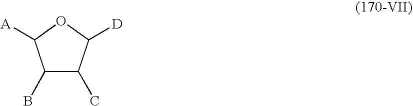
and pharmaceutically acceptable salts thereof, wherein

A is —CH₂OH;

B is —OR⁴;

C is —OR⁵;

wherein R⁴ and R⁵ are independently the residue of a naturally occurring amino acid that is attached via its C-terminus;

D is:

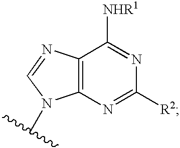

A and B are trans with respect to each other;

B and C are cis with respect to each other;

C and D are cis or trans with respect to each other;

R¹ is —C₁-C₆ alkyl, -aryl, -(arylene)-C₁-C₆ alkyl, -3- to 7-membered monocyclic heterocycle, -8- to 12-membered bicyclic heterocycle, —C₃-C₈ monocyclic cycloalkyl, —(C₃-C₈ monocyclic cycloalkylene)-OH, —C₈-C₁₂ bicyclic cycloalkyl, -(3- to 7-membered monocyclic heterocyclene)-S-aryl, —(C₁-C₆ alkylene)-S-(8- to 12-membered bicyclic heterocycle) or —(C₁-C₆ alkylene)-aryl;

R² is —H, -halo, —CN, —C≡C—R³, —C(O)NHR³, —CH═CHR³, —OH, —O—(C₁-C₆ alkyl), —NH—N═CHR³, —C₁-C₆ alkyl, -aryl, -3- to 7-membered monocyclic heterocycle, -8- to 12-membered bicyclic heterocycle, —NH(C₁-C₆ alkyl), —NH((C₁-C₆ alkylene)-aryl), —NH((C₁-C₆ alkylene)-C₃-C₈ monocyclic cycloalkyl), —NH((C₁-C₆ alkylene)-C₈-C₁₂ bicyclic cycloalkyl), —CH₂—O—(C₁-C₆ alkyl), —CH₂—NH—(C₁-C₆ alkyl) or —CH₂—NH-aryl; and

R³ is —C₁-C₆ alkyl, -aryl, -3- to 7-membered monocyclic heterocycle, -8- to 12-membered bicyclic heterocycle, —C₃-C₈ monocyclic cycloalkyl, —CH₂—(C₃-C₈ monocyclic cycloalkyl) or —C₈-C₁₂ bicyclic cycloalkyl.

In one embodiment, R¹ is —C₁-C₆ alkyl.

In another embodiment, R¹ is -aryl.

In another embodiment, R¹ is -(arylene)-C₁-C₆ alkyl.

In a further embodiment, R¹ is -3- to 7-membered monocyclic heterocycle or -8- to 12-membered bicyclic heterocycle.

In still another embodiment, R¹ is —C₃-C₈ monocyclic cycloalkyl or —C₈-C₁₂ bicyclic cycloalkyl.

In another embodiment, R¹ is —(C₃-C₈ monocyclic cycloalkylene)-OH.

In yet another embodiment, R¹ is -(3- to 7-membered monocyclic heterocycle)-S-aryl.

In another embodiment, R¹ is —(C₁-C₆ alkylene)-S-(8- to 12-membered bicyclic heterocycle).

In a further embodiment, R¹ is -(arylene)-C₁-C₆ alkyl.

In one embodiment, R² is —H.

In another embodiment, R² is —CN.

In another embodiment, R² is C₁-C₆ alkyl.

In another embodiment, R² is -aryl.

In a further embodiment, R² is -halo.

In another embodiment, R² is —C≡C—R³ or —CH═CHR³.

In yet another embodiment, R² is —OH.

In another embodiment, R² is —C(O)NHR³.

In a further embodiment, R² is —NH—(C₁-C₆ alkyl), —NH—(C₁-C₆ alkylene)-aryl or —NH((C₁-C₆ alkylene)-C₃-C₈ monocyclic cycloalkyl).

In another embodiment, R² is —CH₂—O—(C₁-C₆ alkyl).

In another embodiment, R² is —O—(C₁-C₆ alkyl).

In yet another embodiment, R² is —CH₂—NH—(C₁-C₆ alkyl) or —CH₂—NH-aryl.

In a further embodiment, R² is -3- to 7-membered monocyclic heterocycle or -8- to 12-membered bicyclic heterocycle.

In one embodiment, R³ is —C₁-C₆ alkyl.

In another embodiment, R³ is -aryl.

In another embodiment, R³ is —CH₂—(C₃-C₈ monocyclic cycloalkyl).

In a further embodiment, R³ is -3- to 7-membered monocyclic heterocycle or -8- to 12-membered bicyclic heterocycle.

In still another embodiment, R³ is —C₃-C₈ monocyclic cycloalkyl or —C₈-C₁₂ bicyclic cycloalkyl.

In one embodiment, C and D are cis with respect to each other.

In another embodiment, C and D are trans with respect to each other.

In a specific embodiment, R⁴ and R⁵ are each:

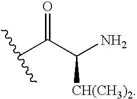

The present invention also provides compositions comprising an effective amount of a Purine Compound of Formula (170-VII) and a physiologically acceptable carrier or vehicle.

The invention further provides Purine Compounds of Formula (170-VII) that are in isolated and purified form.

The invention still further provides methods for treating or preventing a Condition, comprising administering an effective amount of a Purine Compound of Formula (170-VII) to a subject in need thereof.

The invention further provides methods for reducing a subject's rate of metabolism, comprising administering an effective amount of a Purine Compound of Formula (170-VII) to a subject in need thereof.

The invention further provides methods protecting a subject's heart against myocardial damage during cardioplegia, comprising administering an effective amount of a Purine Compound of Formula (170-VII) to a subject in need thereof.

In another embodiment, the invention provides compounds having the Formula (170-VIII): 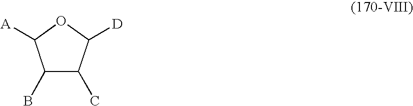
and pharmaceutically acceptable salts thereof, wherein

A is —CH₂OSO₃H;

B is —OR⁴;

C is —OR⁵;

wherein R⁴ and R⁵ are independently the residue of a naturally occurring amino acid that is attached via its C-terminus;

D is:

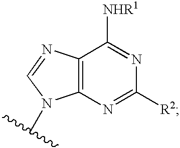

A and B are trans with respect to each other;

B and C are cis with respect to each other; and

C and D are cis or trans with respect to each other;

R¹ is —H, —C₁-C₆ alkyl, -aryl, -3- to 7-membered monocyclic heterocycle, -8- to 12-membered bicyclic heterocycle, —C₃-C₈ monocyclic cycloalkyl or —C₈-C₁₂ bicyclic cycloalkyl;

R² is —C(O)NHR³, —C≡C—R³, —CH═CHR³, —CH₂—NH(C₁-C₆ alkyl), —CH₂—NH-aryl or —CH₂—O—(C₁-C₆ alkyl); and

R³ is —C₁-C₆ alkyl, -aryl, -3- to 7-membered monocyclic heterocycle, -8- to 12-membered bicyclic heterocycle, —C₃-C₈ monocyclic cycloalkyl or —C₈-C₁₂ bicyclic cycloalkyl.

In one embodiment, R¹ is —C₁-C₆ alkyl.

In another embodiment, R¹ is -aryl.

In another embodiment, R¹ is -(arylene)-C₁-C₆.

In a further embodiment, R¹ is -3- to 7-membered monocyclic heterocycle or -8- to 12-membered bicyclic heterocycle.

In still another embodiment, R¹ is —C₃-C₈ monocyclic cycloalkyl or —C₈-C₁₂ bicyclic cycloalkyl.

In another embodiment, R² is —C≡C—R³ or —CH═CHR³.

In another embodiment, R² is —C(O)NHR³.

In yet another embodiment, R² is —CH₂—O—(C₁-C₆ alkyl).

In still another embodiment, R² is —CH₂—NH—(C₁-C₆ alkyl) or —CH₂—NH-aryl.

In one embodiment, R³ is —C₁-C₆ alkyl.

In another embodiment, R³ is -aryl.

In a further embodiment, R³ is -3- to 7-membered monocyclic heterocycle or -8- to 12-membered bicyclic heterocycle.

In still another embodiment, R³ is —C₃-C₈ monocyclic cycloalkyl or —C₈-C₁₂ bicyclic cycloalkyl.

In one embodiment, C and D are cis with respect to each other.

In another embodiment, C and D are trans with respect to each other.

In a specific embodiment, R⁴ and R⁵ are each:

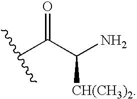

The present invention also provides compositions comprising an effective amount of a Purine Compound of Formula (170-VIII) and a physiologically acceptable carrier or vehicle.

The invention further provides Purine Compounds of Formula (170-VIII) that are in isolated and purified form.

The invention still further provides methods for treating or preventing a Condition, comprising administering an effective amount of a Purine Compound of Formula (170-VIII) to a subject in need thereof.

The invention further provides methods for reducing a subject's rate of metabolism, comprising administering an effective amount of a Purine Compound of Formula (170-VIII) to a subject in need thereof.

The invention further provides methods protecting a subject's heart against myocardial damage during cardioplegia, comprising administering an effective amount of a Purine Compound of Formula (170-VIII) to a subject in need thereof.

In another embodiment, the invention provides compounds having the Formula (170-IX): 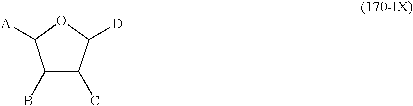
and pharmaceutically acceptable salts thereof, wherein

A is —CH₂ONO₂;

B is —OR⁴;

C is —OR⁵;

wherein R⁴ and R⁵ are independently the residue of a naturally occurring amino acid that is attached via its C-terminus;

D is:

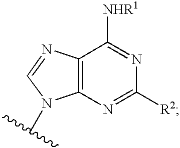

A and B are trans with respect to each other;

B and C are cis with respect to each other;

C and D are cis or trans with respect to each other;

R¹ is —C₁-C₆ alkyl, -aryl, -3- to 7-membered monocyclic heterocycle, -8- to 12-membered bicyclic heterocycle or —C₈-C₁₂ bicyclic cycloalkyl;

R² is —H, -halo, —C(O)NHR³, —C≡C—R³, —CH═CHR³, —C₁-C₆ alkyl, -aryl, -3- to 7-membered monocyclic heterocycle, -8- to 12-membered bicyclic heterocycle, —CH₂—NH(C₁-C₆ alkyl), —CH₂—NH-aryl or —CH₂—O—(C₁-C₆ alkyl); and

R³ is —C₁-C₆ alkyl, -aryl, -3- to 7-membered monocyclic heterocycle, -8- to 12-membered bicyclic heterocycle, —C₃-C8 monocyclic cycloalkyl or —C₈-C₁₂ bicyclic cycloalkyl.

In one embodiment, R¹ is —C₁-C₆ alkyl.

In another embodiment, R¹ is -aryl.

In a further embodiment, R¹ is -3- to 7-membered monocyclic heterocycle or -8- to 12-membered bicyclic heterocycle.

In still another embodiment, R¹ is —C₈-C₁₂ bicyclic cycloalkyl.

In one embodiment, R² is —H.

In another embodiment, R² is —C≡C—R³ or —CH═CHR³.

In another embodiment, R² is —C(O)NHR³.

In yet another embodiment, R² is —CH₂—O—(C₁-C₆ alkyl).

In still another embodiment, R² is —CH₂—NH—(C₁-C₆ alkyl) or —CH₂—NH-aryl.

In one embodiment, R³ is —C₁-C₆ alkyl.

In another embodiment, R³ is -aryl.

In a further embodiment, R³ is -3- to 7-membered monocyclic heterocycle or -8- to 12-membered bicyclic heterocycle.

In one embodiment, C and D are cis with respect to each other.

In another embodiment, C and D are trans with respect to each other.

In a specific embodiment, R⁴ and R⁵ are each:

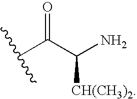

The present invention also provides compositions comprising an effective amount of a Purine Compound of Formula (170-IX) and a physiologically acceptable carrier or vehicle.

The invention further provides Purine Compounds of Formula (170-IX) that are in isolated and purified form.

The invention still further provides methods for treating or preventing a Condition, comprising administering an effective amount of a Purine Compound of Formula (170-IX) to a subject in need thereof.

The invention further provides methods for reducing a subject's rate of metabolism, comprising administering an effective amount of a Purine Compound of Formula (170-IX) to a subject in need thereof.

The invention further provides methods protecting a subject's heart against myocardial damage during cardioplegia, comprising administering an effective amount of a Purine Compound of Formula (170-IX) to a subject in need thereof.

In still another embodiment, the invention provides compounds having the Formula (170-X): 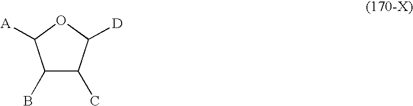
and pharmaceutically acceptable salts thereof, wherein

A is —R³;

B is —OR⁵;

C is —OR⁶;

wherein R⁵ and R⁶ are independently the residue of a naturally occurring amino acid that is attached via its C-terminus;

D is:

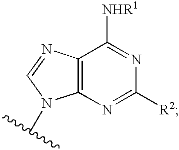

A and B are trans with respect to each other;

B and C are cis with respect to each other;

C and D are cis or trans with respect to each other;

R¹ is —H, —C₁-C₆ alkyl, -aryl, -3- to 7-membered monocyclic heterocycle, -8- to 12-membered bicyclic heterocycle, —C₃-C₈ monocyclic cycloalkyl, —C₈-C₁₂ bicyclic cycloalkyl or —CH((C₁-C₆ alkylene)-OH)((C₁-C₆ alkylene)-(arylene)-O—C₁-C₆ alkyl);

R² is —H, -halo, —CN, —C≡C—R⁴, —CH═CHR⁴, —OH, —O—(C₁-C₆ alkyl), —NH—N═CHR⁴, —C₁-C₆ alkyl, -aryl, -3- to 7-membered monocyclic heterocycle, -8- to 12-membered bicyclic heterocycle, —NH(C₁-C₆ alkyl), —NH-aryl, —NH(C₃-C₈ monocyclic cycloalkyl), —NH(C₈-C₁₂ bicyclic cycloalkyl), —CH₂—O—(C₁-C₆ alkyl), —CH₂—NH(C₁-C₆ alkyl) or —CH₂—NH-aryl;

R³ is —(C₁-C₆ alkylene)ₙ-(3- to 7-membered monocyclic heterocycle), —(C₁-C₆ alkylene)ₙ-(3 - to 7-membered monocyclic heterocyclene)-C₁-C₆ alkyl or —(C₁-C₆ alkylene)ₙ-(8- to 12-membered bicyclic heterocycle);

R⁴ is —C₁-C₆ alkyl, -aryl, -3- to 7-membered monocyclic heterocycle, -8- to 12-membered bicyclic heterocycle, —C₃-C₈ monocyclic cycloalkyl, —(C₃-C₈ monocyclic cycloalkylene)-OH or —C₈-C₁₂ bicyclic cycloalkyl; and

n is 0 or 1.

In one embodiment, R¹ is —H.

In another embodiment, R¹ is —C₁-C₆ alkyl.

In another embodiment, R¹ is -aryl.

In still another embodiment, R¹ is —C₃-C₈ monocyclic cycloalkyl or —C₈-C₁₂ bicyclic cycloalkyl.

In another embodiment, R¹ is -3- to 7-membered monocyclic heterocycle or -8- to 12-membered bicyclic heterocycle.

In a further embodiment, R¹ is —H, —C₁-C₆ alkyl, -aryl, -3- to 7-membered monocyclic heterocycle, -8- to 12-membered bicyclic heterocycle, —C₃-C₈ monocyclic cycloalkyl or —C₈-C₁₂ bicyclic cycloalkyl;

In one embodiment, R² is —H.

In another embodiment, R² is —CN.

In a further embodiment, R² is -halo.

In another embodiment, R² is —C≡C—R⁴ or —CH═CHR⁴.

In yet another embodiment, R² is —OH.

In another embodiment, R² is —O—(C₁-C₆ alkyl).

In another embodiment, R² is —NH—N═CHR⁴.

In a further embodiment, R² is —C₁-C₆ alkyl.

In another embodiment, R² is -aryl.

In yet another embodiment, R² is -3- to 7-membered monocyclic heterocycle or -8- to 12-membered bicyclic heterocycle.

In a further embodiment, R² is —NH—(C₁-C₆ alkyl), —NH-aryl or —NH—(C₃-C₈ monocyclic cycloalkyl).

In another embodiment, R² is —CH₂—O—(C₁-C₆ alkyl).

In yet another embodiment, R² is —CH₂—NH—(C₁-C₆ alkyl) or —CH₂—NH-aryl.

In a further embodiment, R³ is —(C₁-C₆ alkylene)ₙ-(3- to 7-membered monocyclic heterocycle).

In another embodiment, R³ is —(C₁-C₆ alkylene)ₙ-(8- to 12-membered bicyclic heterocycle).

In yet another embodiment, R³ is —(C₁-C₆ alkylene)ₙ-(3- to 7-membered monocyclic heterocycle) or —(C₁-C₆ alkylene)ₙ-(8- to 12-membered bicyclic heterocycle);

In one embodiment, R⁴ is —C₁-C₆ alkyl.

In another embodiment, R⁴ is -aryl.

In yet another embodiment, R⁴ is -3- to 7-membered monocyclic heterocycle or -8- to 12-membered bicyclic heterocycle.

In a further embodiment, R⁴ is —C₃-C₈ monocyclic cycloalkyl or —C₈-C₁₂ bicyclic cycloalkyl.

In another embodiment, R⁴ is —HO-substituted-C₃-C₈ monocyclic cycloalkyl.

In one embodiment, n is 0.

In another embodiment, n is 1.

In one embodiment, C and D are cis with respect to each other.

In another embodiment, C and D are trans with respect to each other.

In a specific embodiment, R⁵ and R⁶ are each:

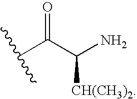

The present invention also provides compositions comprising an effective amount of a Purine Compound of Formula (170-X) and a physiologically acceptable carrier or vehicle.

The invention further provides Purine Compounds of Formula (170-X) that are in isolated and purified form.

The invention still further provides methods for treating or preventing a Condition, comprising administering an effective amount of a Purine Compound of Formula (170-X) to a subject in need thereof.

The invention further provides methods for reducing a subject's rate of metabolism, comprising administering an effective amount of a Purine Compound of Formula (170-X) to a subject in need thereof.

The invention further provides methods protecting a subject's heart against myocardial damage during cardioplegia, comprising administering an effective amount of a Purine Compound of Formula (170-X) to a subject in need thereof.

The Purine Compounds of Formula (I), (II) or (III) can exist in the form of a single enantiomer, for example, that depicted by either the Formula (a′) or Formula (a″): 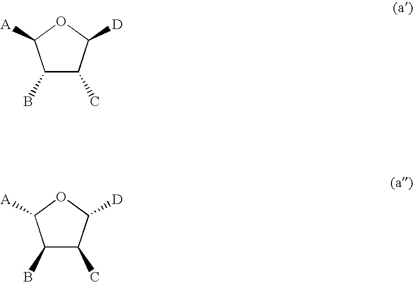
wherein A, B, C and D are defined above for the Purine Compounds of Formula (I), (II) or (III).

A Purine Compound of Formula (a′) is the corresponding opposite enantiomer of a Purine Compound of Formula (a″) when group A of the Purine Compound of Formula (a′) is the same as group A of the Purine Compound of Formula (a″) and when group D of the Purine Compound of Formula (a′) is the same as group D of the Purine Compound of Formula (a″).

A Purine Compound of Formula (a″) is the corresponding opposite enantiomer of a Purine Compound of Formula (a′) when group A of the Purine Compound of Formula (a″) is the same as group A of the Purine Compound of Formula (a′) and when group D of the Purine Compound of Formula (a″) is the same as group D of the Purine Compound of Formula (a′).

In one embodiment, the Purine Compounds of Formula (I), (II) or (III) have the formula (a′), depicted above, wherein A, B, C and D are defined above for the Purine Compounds of Formula (I), (II) or (III), and wherein the Purine Compounds of Formula (a′) are substantially free of their corresponding opposite enantiomer.

In another embodiment, the Purine Compounds of Formula (I), (II) or (III) have the formula (a″), depicted above, wherein A, B, C and D are defined above for the Purine Compounds of Formula (I), (II) or (III), and wherein the Purine Compounds of Formula (a″) are substantially free of their corresponding opposite enantiomer.

In another embodiment, the Purine Compounds of Formula (I), (II) or (III) exist as a mixture of a Purine Compound of Formula (a′) and a Purine Compound of Formula (a″) wherein the amount of the Purine Compound of Formula (a′) exceeds the amount of the Purine Compound of Formula (a″).

In a further embodiment, the Purine Compounds of Formula (I), (II) or (III) exist as a mixture of a Purine Compound of Formula (a′) and a Purine Compound of Formula (a″) wherein the amount of the Purine Compound of Formula (a″) exceeds the amount of the Purine Compound of Formula (a′).

In another embodiment, the Purine Compounds of Formula (I), (II) or (III) exist as a racemic mixture of a Purine Compound of Formula (a′) and a Purine Compound of Formula (a″).

In another embodiment, the Purine Compounds of Formula (I), (II) or (III) can exist in the form of a single enantiomer, for example, that depicted by either formula (aa′) or (aa″): 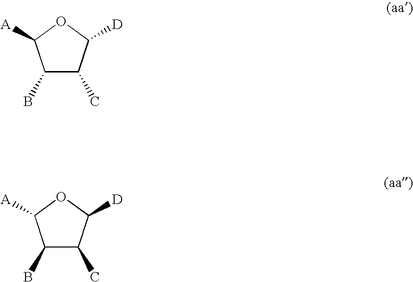
wherein A, B, C and D are defined above for the Purine Compounds of Formula (I), (II) or (III).

A Purine Compound of Formula (aa′) is the corresponding opposite enantiomer of a Purine Compound of Formula (aa″) when group A of the Purine Compound of Formula (aa′) is the same as group A of the Purine Compound of Formula (aa″) and when group D of the Purine Compound of Formula (aa′) is the same as group D of the Purine Compound of Formula (aa″).

A Purine Compound of Formula (aa″) is the corresponding opposite enantiomer of a Purine Compound of Formula (aa′) when group A of the Purine Compound of Formula (aa″) is the same as group A of the Purine Compound of Formula (aa′) and when group D of the Purine Compound of Formula (aa″) is the same as group D of the Purine Compound of Formula (aa′).

In one embodiment, the Purine Compounds of Formula (I), (II) or (III) have the formula (aa′), depicted above, wherein A, B, C and D are defined above for the Purine Compounds of Formula (I), (II) or (III), and wherein the Purine Compounds of Formula (aa′) are substantially free of their corresponding opposite enantiomer.

In another embodiment, the Purine Compounds of Formula (I), (II) or (III) have the formula (aa″), depicted above, wherein A, B, C and D are defined above for the Purine Compounds of Formula (I), (II) or (III), and wherein the Purine Compounds of Formula (aa″) are substantially free of their corresponding opposite enantiomer.

In another embodiment, the Purine Compounds of Formula (I), (II) or (III) exist as a mixture of a Purine Compound of Formula (aa′) and a Purine Compound of Formula (aa″) wherein the amount of the Purine Compound of Formula (aa′) exceeds the amount of the Purine Compound of Formula (aa″).

In a further embodiment, the Purine Compounds of Formula (I), (II) or (III) exist as a mixture of a Purine Compound of Formula (aa′) and a Purine Compound of Formula (aa″) wherein the amount of the Purine Compound of Formula (aa″) exceeds the amount of the Purine Compound of Formula (aa′).

In another embodiment, the Purine Compounds of Formula (Ia), (II) or (III) exist as a racemic mixture of a Purine Compound of Formula (aa′) and a Purine Compound of Formula (aa″).

A Purine Compound of Formula (aa′) is the corresponding other anomer of a Purine Compound of Formula (I), (II) or (III) when group A of the Purine Compound of Formula (aa′) is the same as group A of the Purine Compound of Formula (a′).and when group D of the Purine Compound of Formula (aa′) is the same as group D of the Purine Compound of Formula (a′).

A Purine Compound of Formula (a′) is the corresponding other anomer of a Purine Compound of Formula (aa′) when group A of the Purine Compound of Formula (a′) is the same as group A of the Purine Compound of Formula (aa′) and when group D of the Purine Compound of Formula (a′) is the same as group D of the Purine Compound of Formula (aa′).

A Purine Compound of Formula (aa″) is the corresponding other anomer of a Purine Compound of Formula (a″) when group A of the Purine Compound of Formula (aa″) is the same as group A of the Purine Compound of Formula (a″) and when group D of the Purine Compound of Formula (aa″) is the same as group D of the Purine Compound of Formula (a″).

A Purine Compound of Formula (a″) is the corresponding other anomer of a Purine Compound of Formula (aa″) when group A of the Purine Compound of Formula (a″) is the same as group A of the Purine Compound of Formula (aa″) and when group D of the Purine Compound of Formula (a″) is the same as group D of the Purine Compound of Formula (aa″).

In one embodiment, the Purine Compounds of Formula (I), (II) or (III) have the formula (aa′), depicted above, wherein A, B, C and D are defined above for the Purine Compounds of Formula (I), (II) or (III), and wherein the Purine Compounds of Formula (aa′) are substantially free of their corresponding other anomer.

In another embodiment, the Purine Compounds of Formula (I), (II) or (III) have the formula (aa″), depicted above, wherein A, B, C and D are defined above for the Purine Compounds of Formula (I), (II) or (III), and wherein the Purine Compounds of Formula (aa″) are substantially free of their corresponding other anomer.

In one embodiment, the Purine Compounds of Formula (I), (II) or (III) have the formula (a′), depicted above, wherein A, B, C and D are defined above for the Purine Compounds of Formula (I), (II) or (III), and wherein the Purine Compounds of Formula (a′) are substantially free of their corresponding other anomer.

In another embodiment, the Purine Compounds of Formula (I), (II) or (III) have the formula (a″), depicted above, wherein A, B, C and D are defined above for the Purine Compounds of Formula (I), (II) or (III), and wherein the Purine Compounds of Formula (a″) are substantially free of their corresponding other anomer.

In one embodiment, the Purine Compounds of Formula (I), (II) or (III) exist as a mixture of a Purine Compound of Formula (a′) and a Purine Compound of Formula (aa′) wherein the amount of the Purine Compound of Formula (a′) exceeds the amount of the Purine Compound of Formula (aa′).

In another embodiment, the Purine Compounds of Formula (I), (II) or (III) exist as a mixture of a Purine Compound of Formula (a′) and a Purine Compound of Formula (aa′) wherein the amount of the Purine Compound of Formula (aa′) exceeds the amount of the Purine Compound of Formula (a′).

In a further embodiment, the Purine Compounds of Formula (I), (II) or (III) exist as an equimolar mixture of a Purine Compound of Formula (a′) and a Purine Compound of Formula (aa′).

In one embodiment, the Purine Compounds of Formula (I), (II) or (III) exist as-a mixture of a Purine Compound of Formula (a″) and a Purine Compound of Formula (aa″) wherein the amount of the Purine Compound of Formula (a″) exceeds the amount of the Purine Compound of Formula (aa″).

In another embodiment, the Purine Compounds of Formula (I), (II) or (III) exist as a mixture of a Purine Compound of Formula (a″) and a Purine Compound of Formula (aa″) wherein the amount of the Purine Compound of Formula (aa″) exceeds the amount of the Purine Compound of Formula (a″).

In a further embodiment, the Purine Compounds of Formula (I), (II) or (III) exist as an equimolar mixture of a Purine Compound of Formula (a″) and a Purine Compound of Formula (aa″).

### 5.2.1 ILLUSTRATIVE EXAMPLES OF THE COMPOUNDS OF FORMULA (I)

Illustrative examples of the compounds of Formula (I) include the compounds listed below:

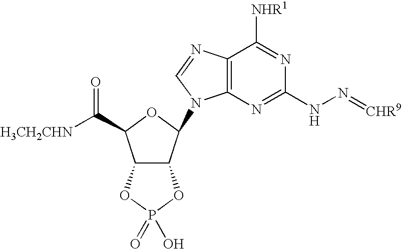

| Com- | | |
|---|---|---|
| pound | R¹ | R⁹ |
| 24 | —H | 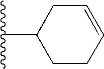 |
| 25 | —H | 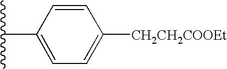 |
| 26 | —H | 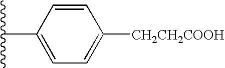 |
| 27 | —H | 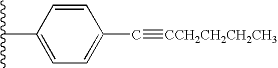 |
| 28 | —H | 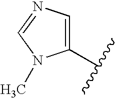 |
| 29 | —H | 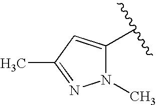 |
| 30 | —H | 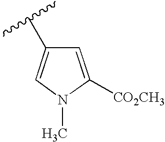 |
| 31 | —H | 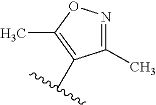 |
| 32 | —H | 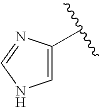 |
| 33 | —H | 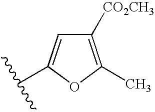 |
| 34 | —H | 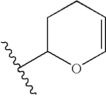 |
| 35 | —H | 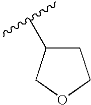 |
| 36 | —H | 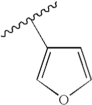 |
| 37 | —H | 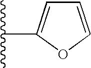 |
| 38 | —H | 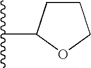 |
| 39 | —H | 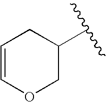 |
| 40 | —H | 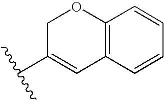 |
| 41 | —H | —C(O)-phenyl |
| 42 | —CH₂CH₃ | -isobutyl |
| 43 | —H | 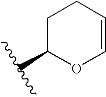 |
| 44 | —H | 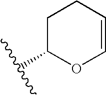 |
| 45 | —H | 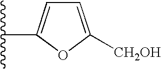 |
| 46 | —H | 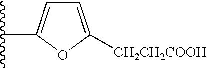 |
| 47 | —H | 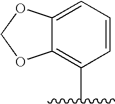 |
| 48 | —H | 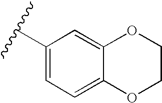 |
| 49 | —H | —CH₂CH₂CH(CH₃)₂ |

and pharmaceutically acceptable salts thereof.

Further illustrative examples of the compounds of Formula (I) include the compounds listed below:

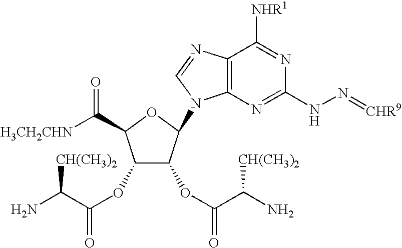

| Com- | | |
|---|---|---|
| pound | R¹ | R⁹ |
| 24a | —H | 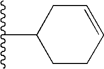 |
| 25a | —H | 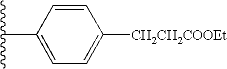 |
| 26a | —H | 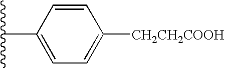 |
| 27a | —H | 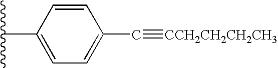 |
| 28a | —H | 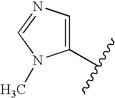 |
| 29a | —H | 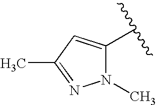 |
| 30a | —H | 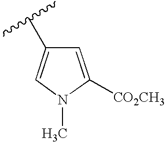 |
| 31a | —H | 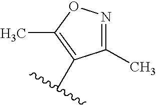 |
| 32a | —H | 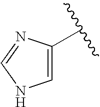 |
| 33a | —H | 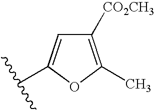 |
| 34a | —H | 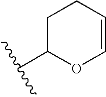 |
| 35a | —H | 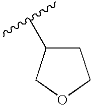 |
| 36a | —H | 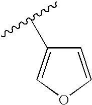 |
| 37a | —H | 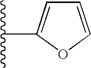 |
| 38a | —H | 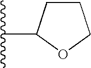 |
| 39a | —H | 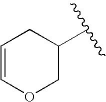 |
| 40a | —H | 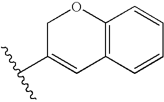 |
| 41a | —H | —C(O)-phenyl |
| 42a | —CH₂CH₃ | -isobutyl |
| 43a | —H | 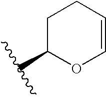 |
| 44a | —H | 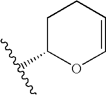 |
| 45a | —H | 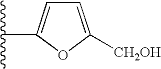 |
| 46a | —H | 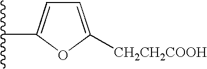 |
| 47a | —H | 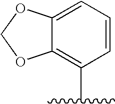 |
| 48a | —H | 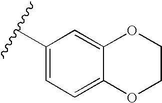 |
| 49a | —H | —CH₂CH₂CH(CH₃)₂ |

and pharmaceutically acceptable salts thereof.

Further illustrative examples of the compounds of Formula (I) include the compounds listed below:

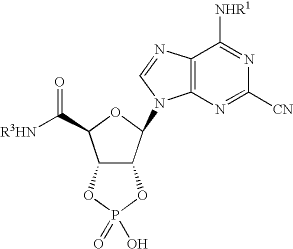

| Compound | R¹ | R³ |
|---|---|---|
| 50 | —H | —CH₂CH₃ |
| 51 | —H | —CH₃ |
| 52 | —CH₂CH₃ | —CH₂CH₃ |
| 53 | —CH₂CH₃ | —CH₃ |
| 54 | —CH₃ | —CH₃ |
| 55 | —CH₃ | —CH₂CH₃ |

and pharmaceutically acceptable salts thereof.

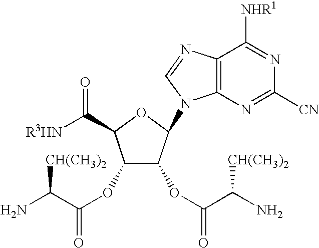

| Compound | R¹ | R³ |
|---|---|---|
| 50a | —H | —CH₂CH₃ |
| 51a | —H | —CH₃ |
| 52a | —CH₂CH₃ | —CH₂CH₃ |
| 53a | —CH₂CH₃ | —CH₃ |
| 54a | —CH₃ | —CH₃ |
| 55a | —CH₃ | —CH₂CH₃ |

and pharmaceutically acceptable salts thereof.

Other illustrative compounds of formula (I) are the following compounds: 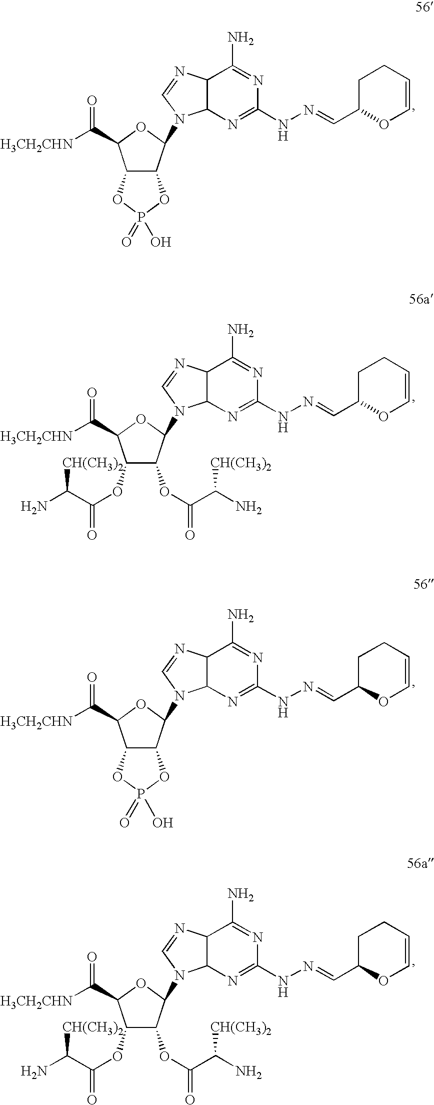
and pharmaceutically acceptable salts thereof.

Other illustrative compounds of Formula (I) are the following compounds: 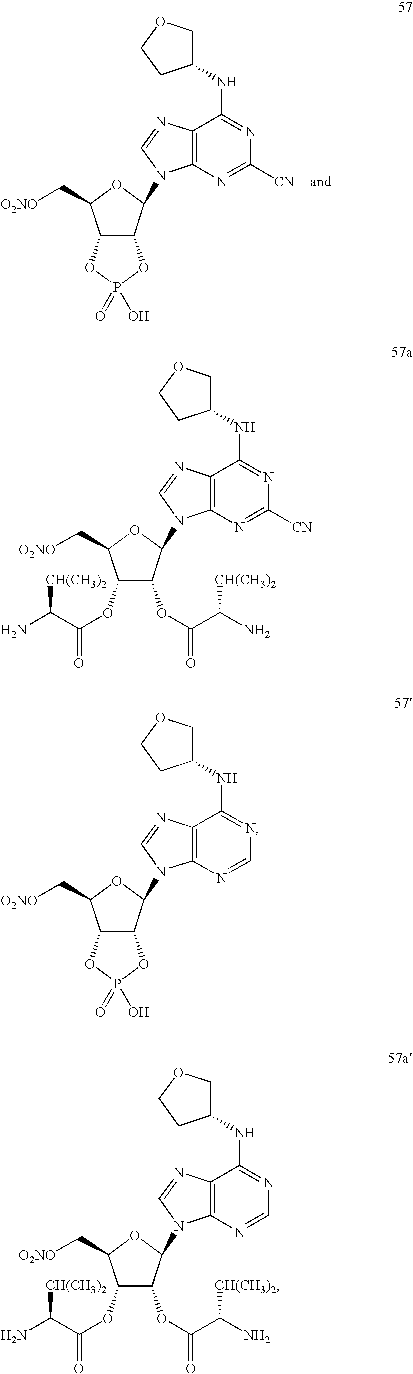
and pharmaceutically acceptable salts thereof.

Other illustrative compounds of Formula (I) are the following compounds:

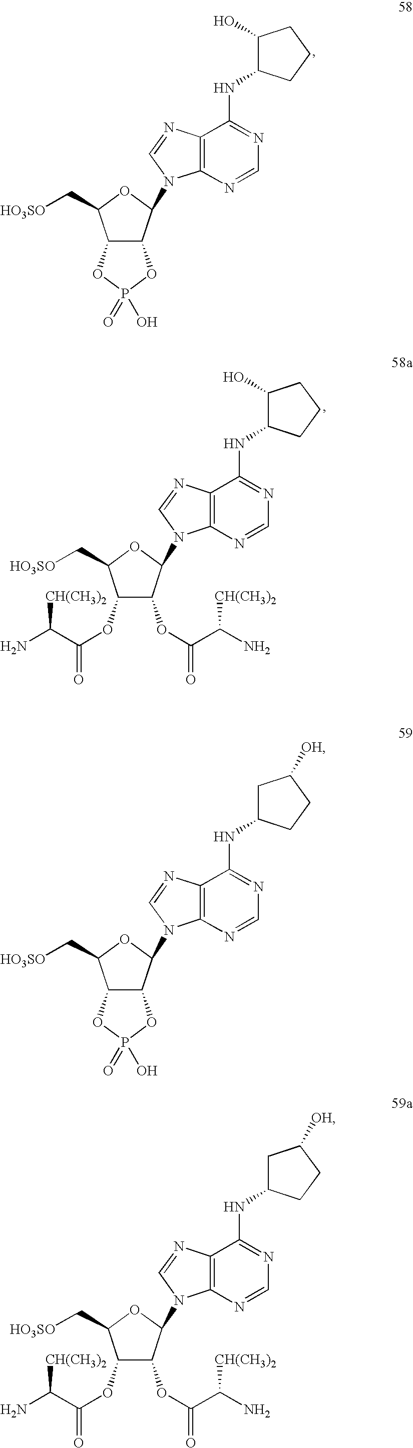

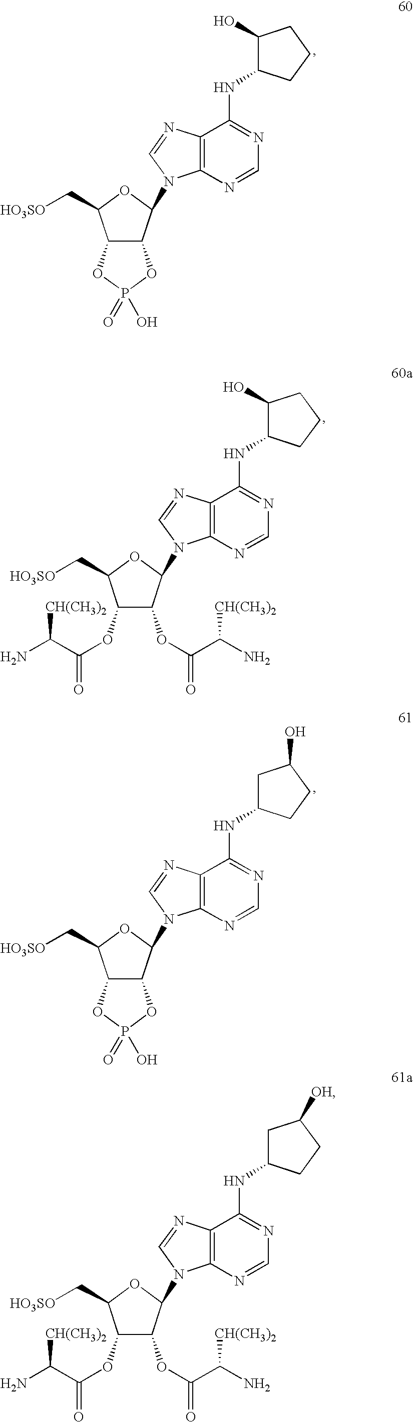

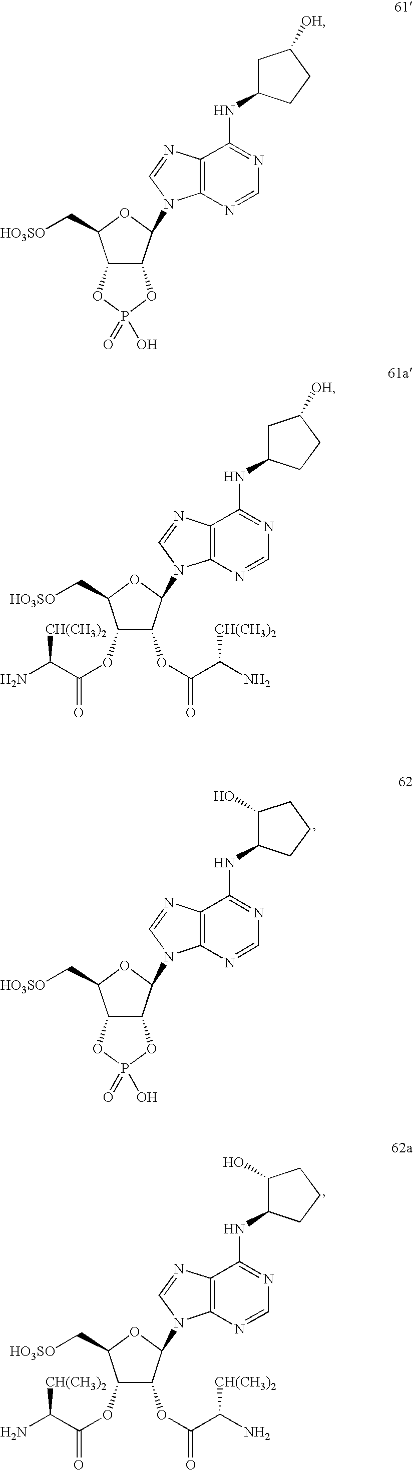

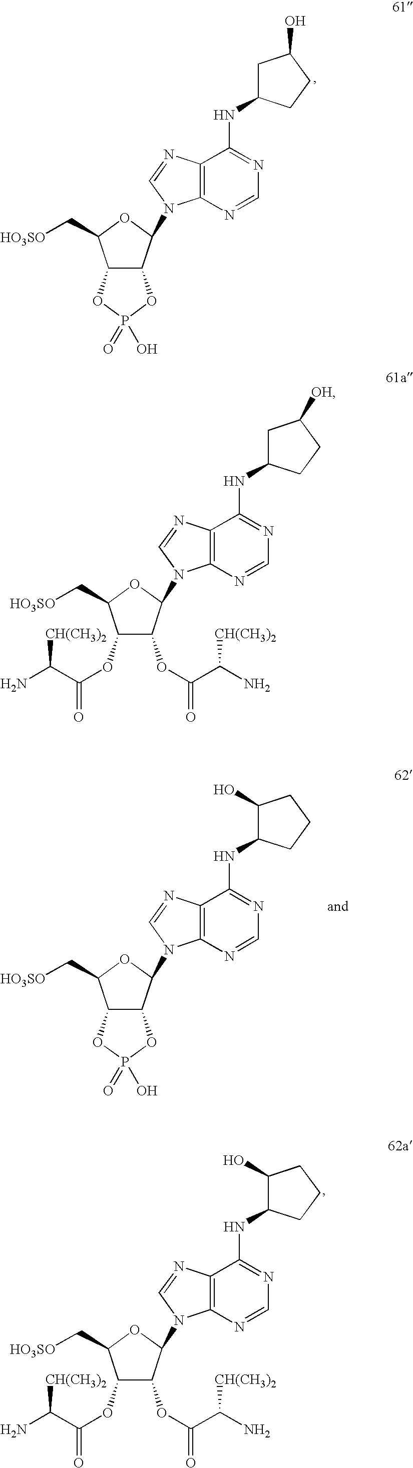

Other Illustrative Purine Compounds of Formula (I) include the compounds listed below: 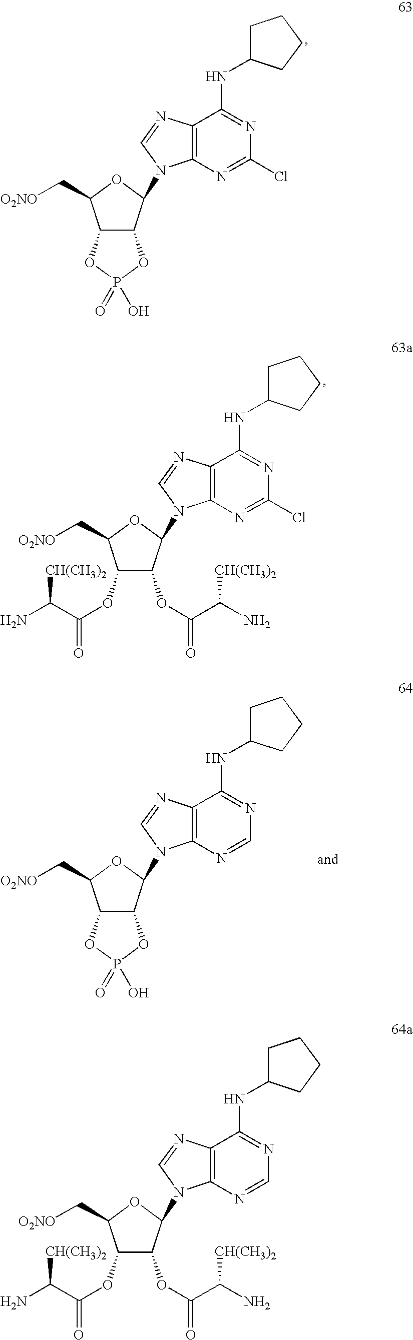
and pharmaceutically acceptable salts thereof.

Other Illustrative Purine Compounds of Formula (I) include the compounds listed below: 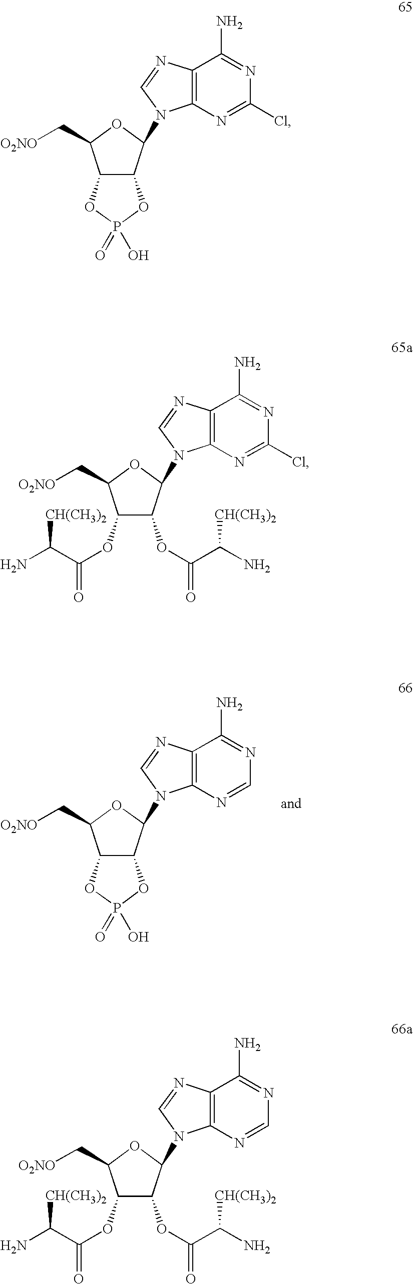
and pharmaceutically acceptable salts thereof.
Other illustrative compounds of Formula I include the compounds listed below: 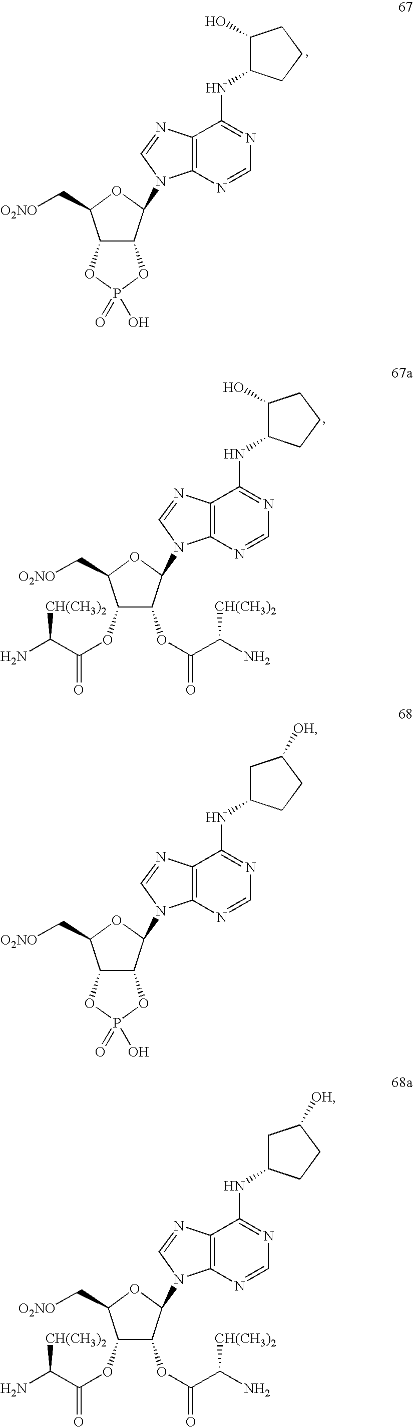 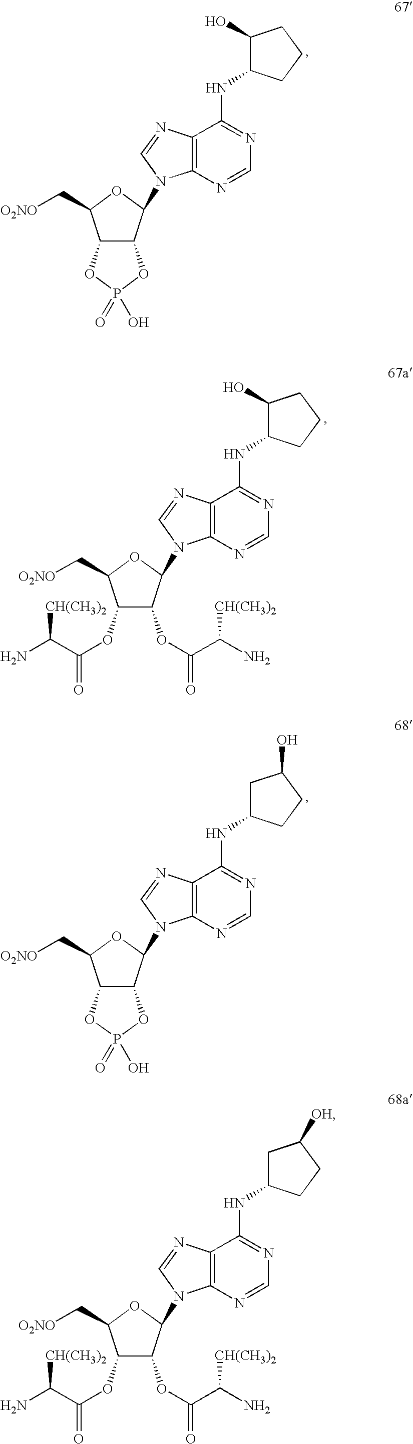 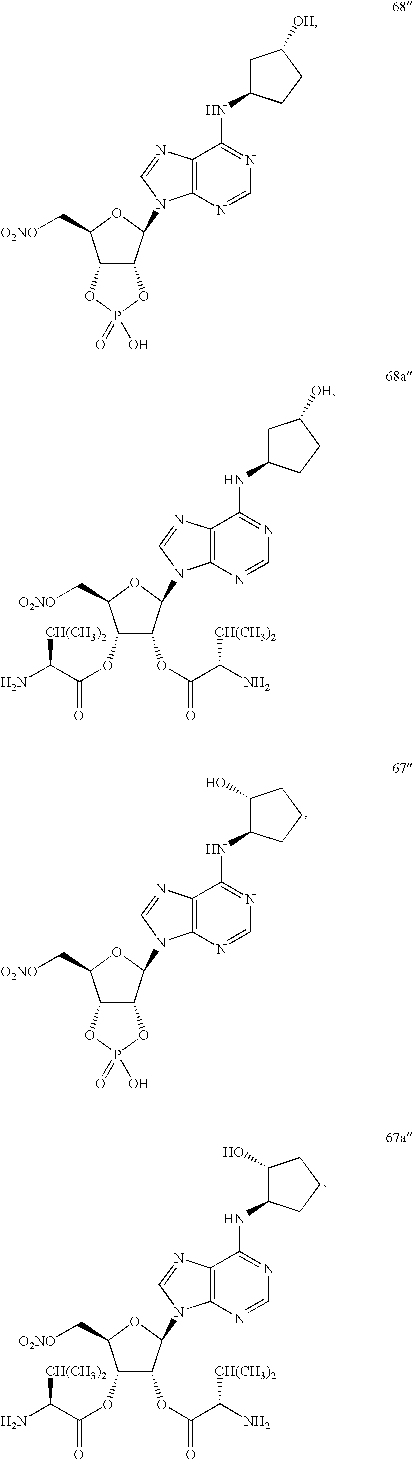 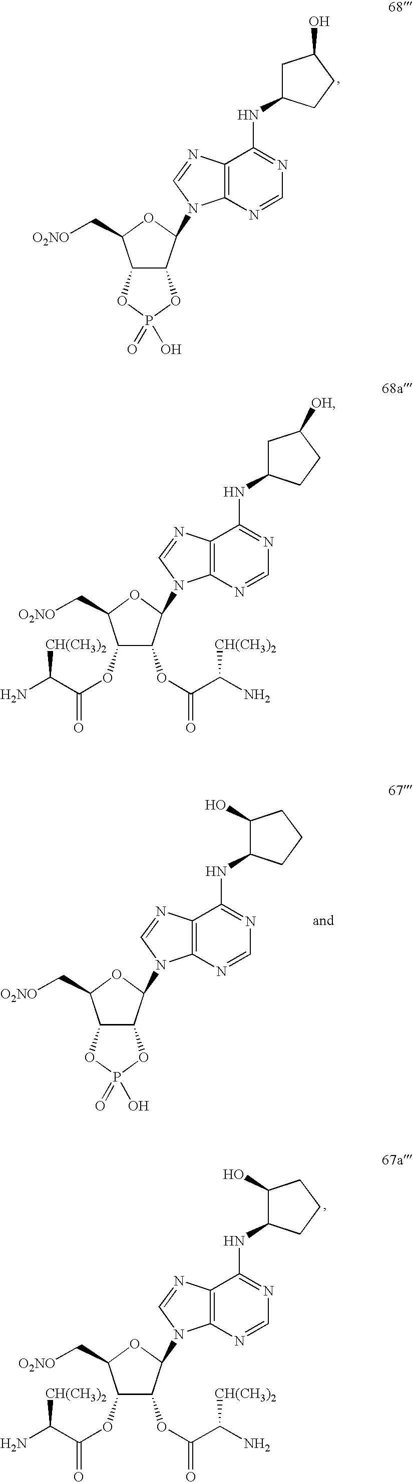
Other illustrative compounds of Formula (I) are the following compounds: 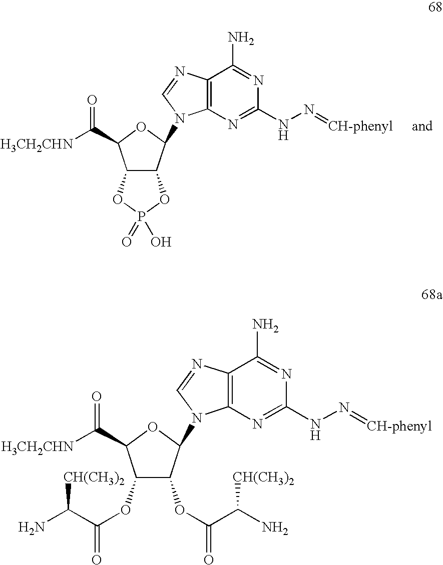
and pharmaceutically acceptable salts thereof.

Other illustrative Purine Compounds of Formula (I) include the following compounds: 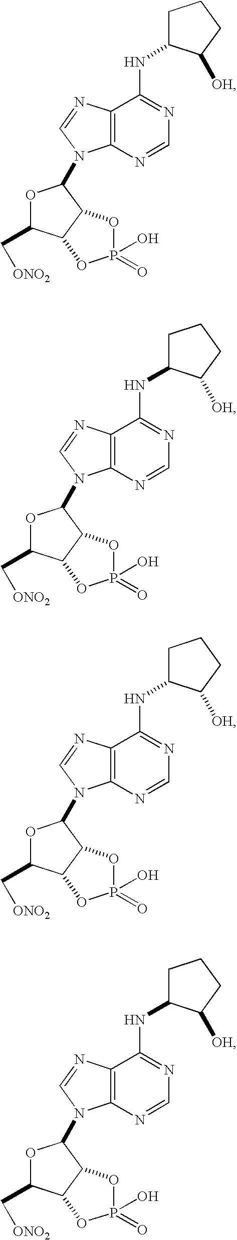 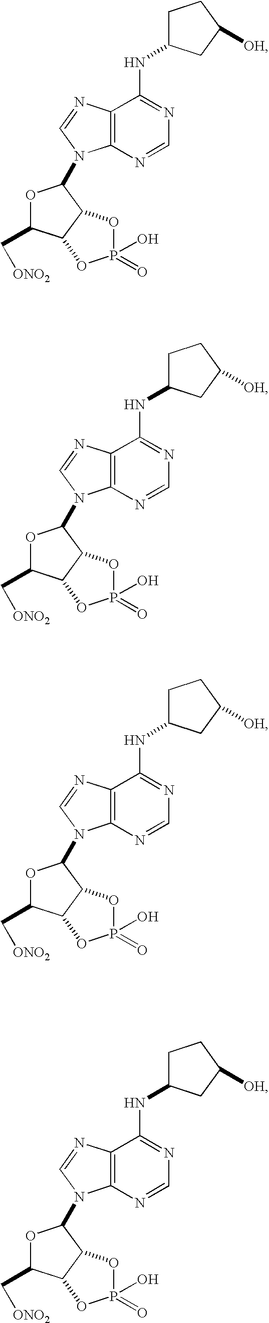 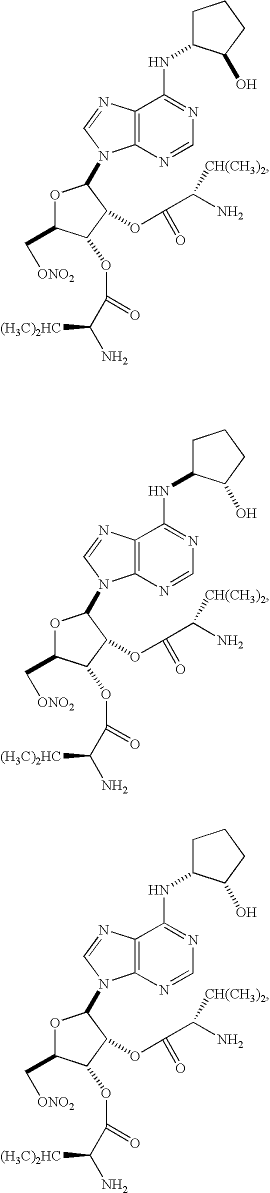 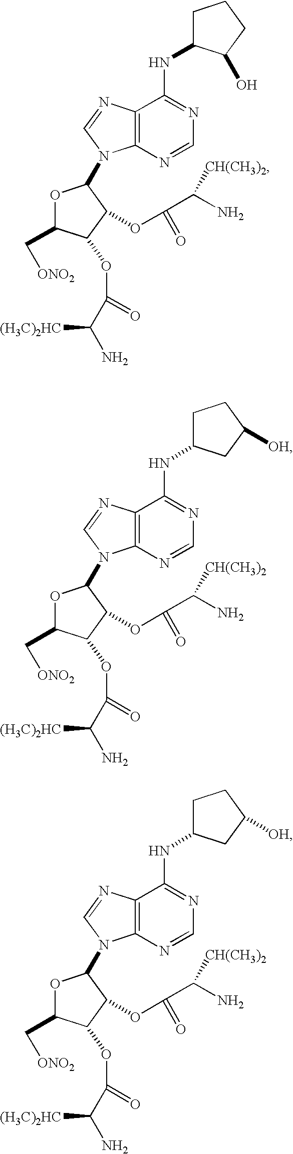 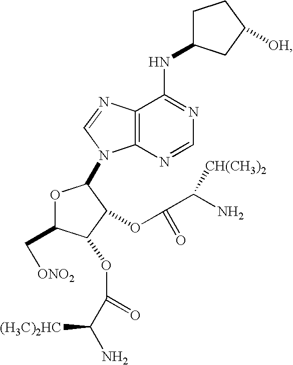 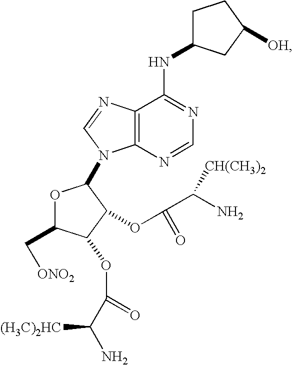
and pharmaceutically acceptable salts thereof.

Still other illustrative Purine Compounds of Formula (I) include the following compounds: 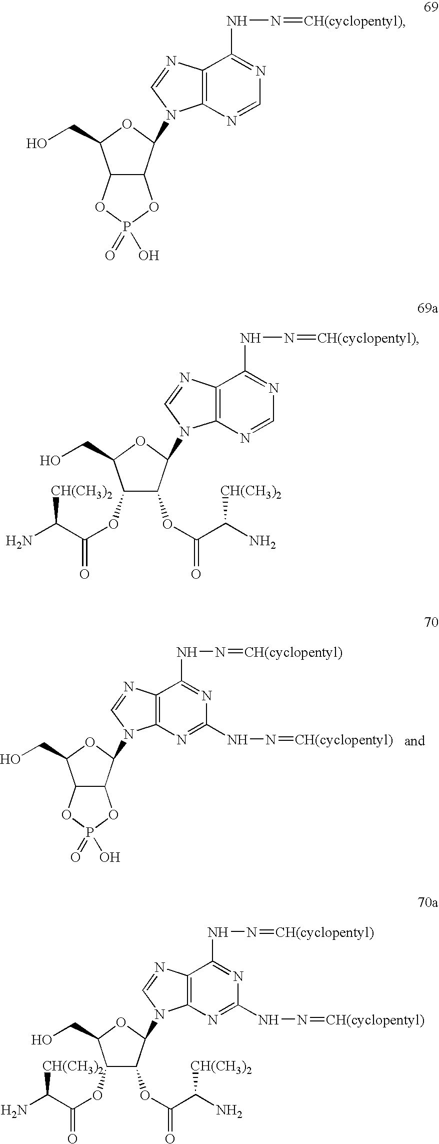
and pharmaceutically acceptable salts thereof.

### 5.2.2 ILLUSTRATIVE EXAMPLES OF THE COMPOUNDS OF FORMULA (II)

Illustrative Purine Compounds of Formula (II) include the compounds of Formula (II′) as set forth below:

| | |
|---|---|
| | (II′) |
| 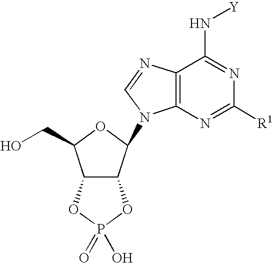 | |

| Compound | R¹ | Y |
|---|---|---|
| II′-1 | —H | 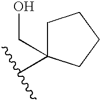 |
| II′-2 | —H | 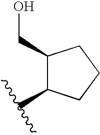 |
| II′-3 | —H | 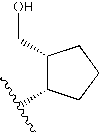 |
| II′-4 | —H | 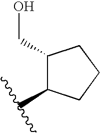 |
| II′-5 | —H | 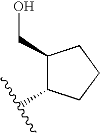 |
| II′-6 | —H | 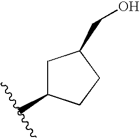 |
| II′-7 | —H | 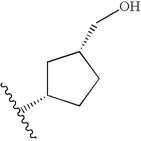 |
| II′-8 | —H | 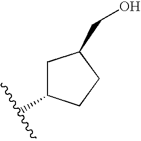 |
| II′-9 | —H | 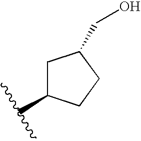 |
| II′-10 | —H | 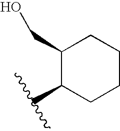 |
| II′-11 | —H | 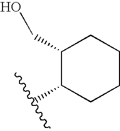 |
| II′-12 | —H | 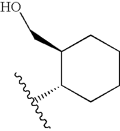 |
| II′-13 | —H | 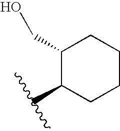 |
| II′-14 | —H | 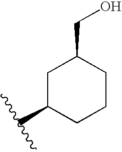 |
| II′-15 | —H | 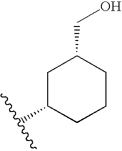 |
| II′-16 | —H | 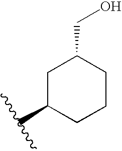 |
| II′-17 | —H | 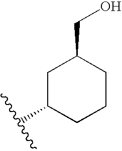 |
| II′-18 | —H | 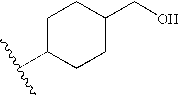 |
| II′-19 | —Cl | 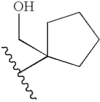 |
| II′-20 | —Cl | 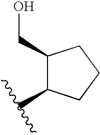 |
| II′-21 | —Cl | 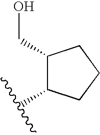 |
| II′-22 | —Cl | 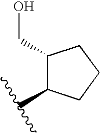 |
| II′-23 | —Cl | 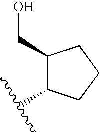 |
| II′-24 | —Cl | 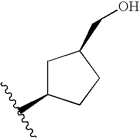 |
| II′-25 | —Cl | 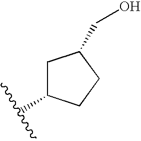 |
| II′-26 | —Cl | 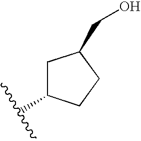 |
| II′-27 | —Cl | 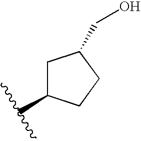 |
| II′-28 | —Cl | 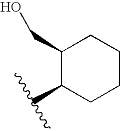 |
| II′-29 | —Cl | 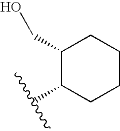 |
| II′-30 | —Cl | 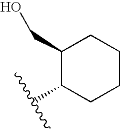 |
| II′-31 | —Cl | 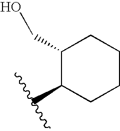 |
| II′-32 | —Cl | 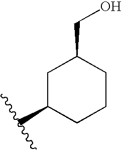 |
| II′-33 | —Cl | 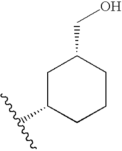 |
| II′-34 | —Cl | 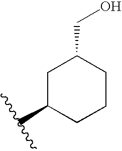 |
| II′-35 | —Cl | 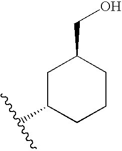 |
| II′-36 | —Cl | 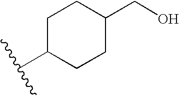 |
| II′-37 | —CN | 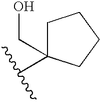 |
| II′-38 | —CN | 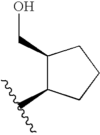 |
| II′-39 | —CN | 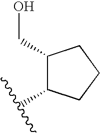 |
| II′-40 | —CN | 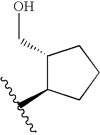 |
| II′-41 | —CN | 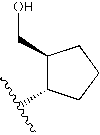 |
| II′-42 | —CN | 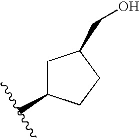 |
| II′-43 | —CN | 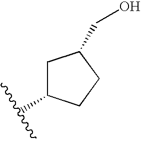 |
| II′-44 | —CN | 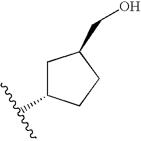 |
| II′-45 | —CN | 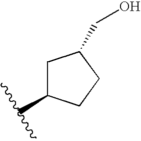 |
| II′-46 | —CN | 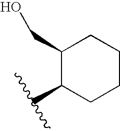 |
| II′-47 | —CN | 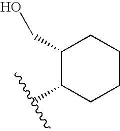 |
| II′-48 | —CN | 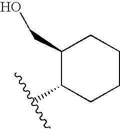 |
| II′-49 | —CN | 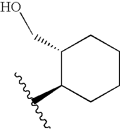 |
| II′-50 | —CN | 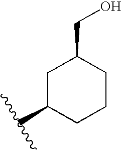 |
| II′-51 | —CN | 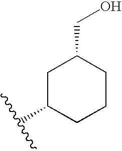 |
| II′-52 | —CN | 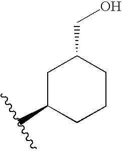 |
| II′-53 | —CN | 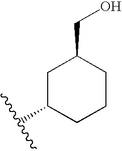 |
| II′-54 | —CN | 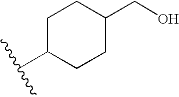 |
| II′-55 | —NH₂ | 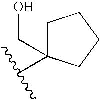 |
| II′-56 | —NH₂ | 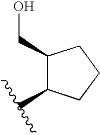 |
| II′-57 | —NH₂ | 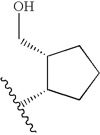 |
| II′-58 | —NH₂ | 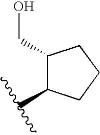 |
| II′-59 | —NH₂ | 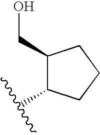 |
| II′-60 | —NH₂ | 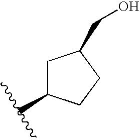 |
| II′-61 | —NH₂ | 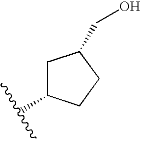 |
| II′-62 | —NH₂ | 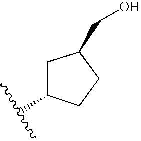 |
| II′-63 | —NH₂ | 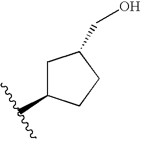 |
| II′-64 | —NH₂ | 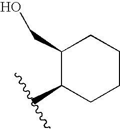 |
| II′-65 | —NH₂ | 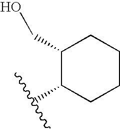 |
| II′-66 | —NH₂ | 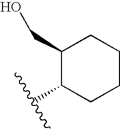 |
| II′-67 | —NH₂ | 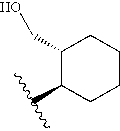 |
| II′-68 | —NH₂ | 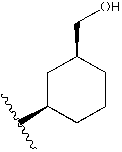 |
| II′-69 | —NH₂ | 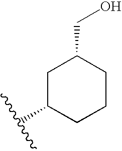 |
| II′-70 | —NH₂ | 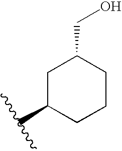 |
| II′-71 | —NH₂ | 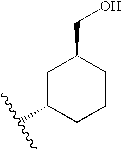 |
| II′-72 | —NH₂ | 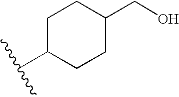 |
| II′-73 | —OCH₃ | 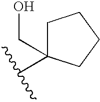 |
| II′-74 | —OCH₃ | 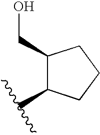 |
| II′-75 | —OCH₃ | 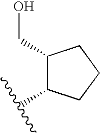 |
| II′-76 | —OCH₃ | 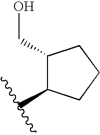 |
| II′-77 | —OCH₃ | 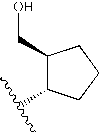 |
| II′-78 | —OCH₃ | 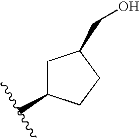 |
| II′-79 | —OCH₃ | 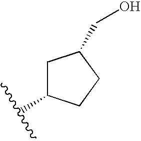 |
| II′-80 | —OCH₃ | 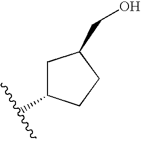 |
| II′-81 | —OCH₃ | 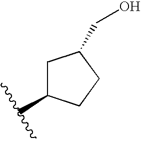 |
| II′-82 | —OCH₃ | 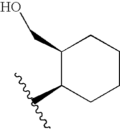 |
| II′-83 | —OCH₃ | 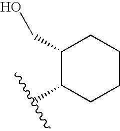 |
| II′-84 | —OCH₃ | 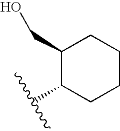 |
| II′-85 | —OCH₃ | 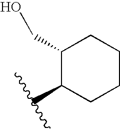 |
| II′-86 | —OCH₃ | 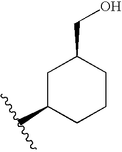 |
| II′-87 | —OCH₃ | 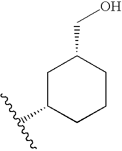 |
| II′-88 | —OCH₃ | 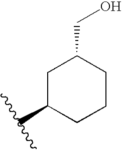 |
| II′-89 | —OCH₃ | 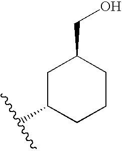 |
| II′-90 | —OCH₃ | 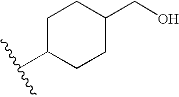 |

and pharmaceutically acceptable salts thereof.

Illustrative Purine Compounds of Formula (II) include the compounds of Formula (II″) as set forth below:

| | |
|---|---|
| | (II″) |
| 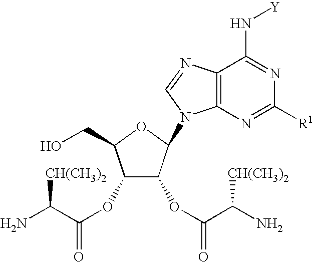 | |

| Compound | R¹ | Y |
|---|---|---|
| II″-1a | —H | 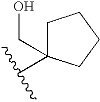 |
| II″-2a | —H | 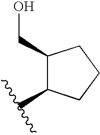 |
| II″-3a | —H | 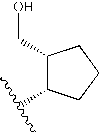 |
| II″-4a | —H | 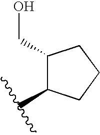 |
| II″-5a | —H | 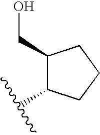 |
| II″-6a | —H | 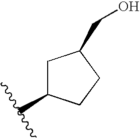 |
| II″-7a | —H | 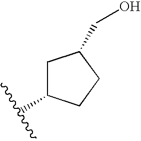 |
| II″-8a | —H | 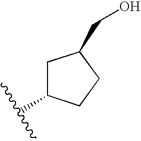 |
| II″-9a | —H | 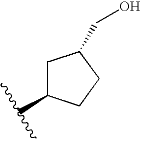 |
| II″-10a | —H | 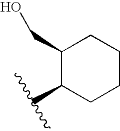 |
| II″-11a | —H | 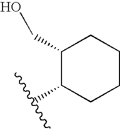 |
| II″-12a | —H | 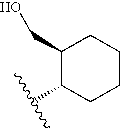 |
| II″-13a | —H | 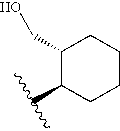 |
| II″-14a | —H | 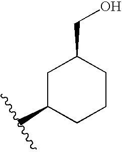 |
| II″-15a | —H | 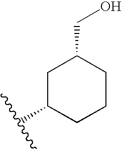 |
| II″-16a | —H | 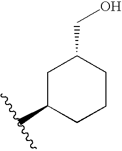 |
| II″-17a | —H | 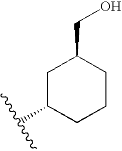 |
| II″-18a | —H | 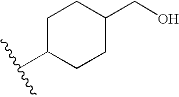 |
| II″-19a | —Cl | 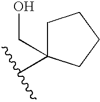 |
| II″-20a | —Cl | 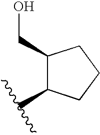 |
| II″-21a | —Cl | 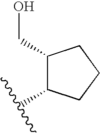 |
| II″-22a | —Cl | 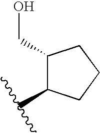 |
| II″-23a | —Cl | 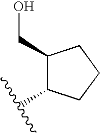 |
| II″-24a | —Cl | 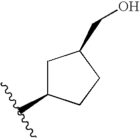 |
| II″-25a | —Cl | 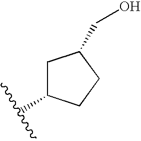 |
| II″-26a | —Cl | 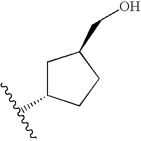 |
| II″-27a | —Cl | 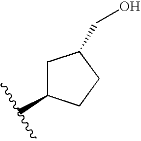 |
| II″-28a | —Cl | 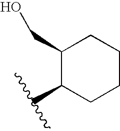 |
| II″-29a | —Cl | 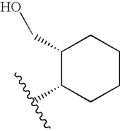 |
| II″-30a | —Cl | 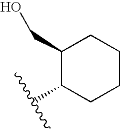 |
| II″-31a | —Cl | 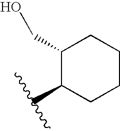 |
| II″-32a | —Cl | 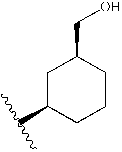 |
| II″-33a | —Cl | 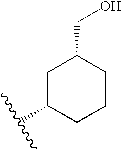 |
| II″-34a | —Cl | 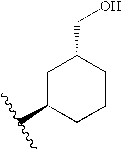 |
| II″-35a | —Cl | 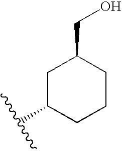 |
| II″-36a | —Cl | 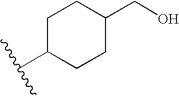 |
| II″-37a | —CN | 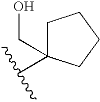 |
| II″-38a | —CN | 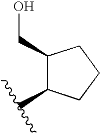 |
| II″-39a | —CN | 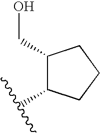 |
| II″-40a | —CN | 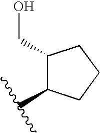 |
| II″-41a | —CN | 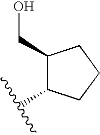 |
| II″-42a | —CN | 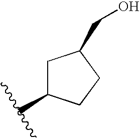 |
| II″-43a | —CN | 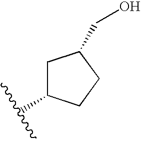 |
| II″-44a | —CN | 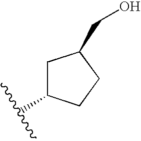 |
| II″-45a | —CN | 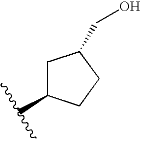 |
| II″-46a | —CN | 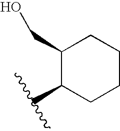 |
| II″-47a | —CN | 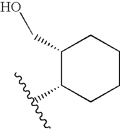 |
| II″-48a | —CN | 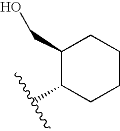 |
| II″-49a | —CN | 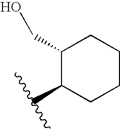 |
| II″-50a | —CN | 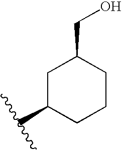 |
| II″-51a | —CN | 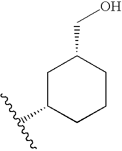 |
| II″-52a | —CN | 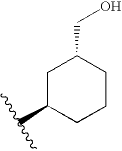 |
| II″-53a | —CN | 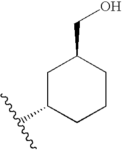 |
| II″-54a | —CN | 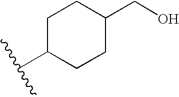 |
| II″-55a | —NH₂ | 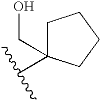 |
| II″-56a | —NH₂ | 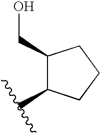 |
| II″-57a | —NH₂ | 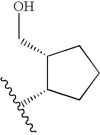 |
| II″-58a | —NH₂ | 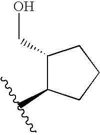 |
| II″-59a | —NH₂ | 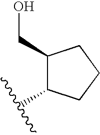 |
| II″-60a | —NH₂ | 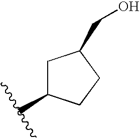 |
| II″-61a | —NH₂ | 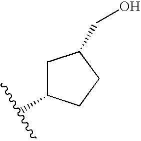 |
| II″-62a | —NH₂ | 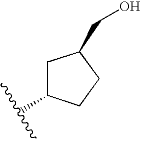 |
| II″-63a | —NH₂ | 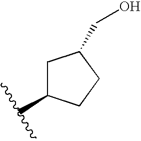 |
| II″-64a | —NH₂ | 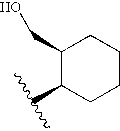 |
| II″-65a | —NH₂ | 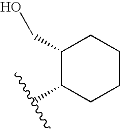 |
| II″-66a | —NH₂ | 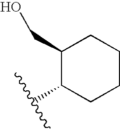 |
| II″-67a | —NH₂ | 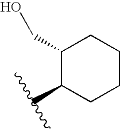 |
| II″-68a | —NH₂ | 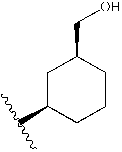 |
| II″-69a | —NH₂ | 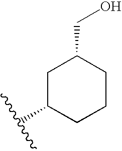 |
| II″-70a | —NH₂ | 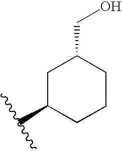 |
| II″-71a | —NH₂ | 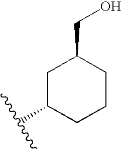 |
| II″-72a | —NH₂ | 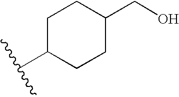 |
| II″-73a | —OCH₃ | 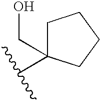 |
| II″-74a | —OCH₃ | 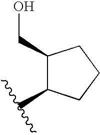 |
| II″-75a | —OCH₃ | 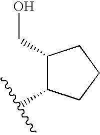 |
| II″-76a | —OCH₃ | 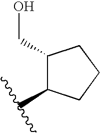 |
| II″-77a | —OCH₃ | 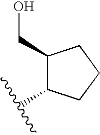 |
| II″-78a | —OCH₃ | 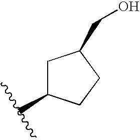 |
| II″-79a | —OCH₃ | 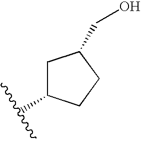 |
| II″-80a | —OCH₃ | 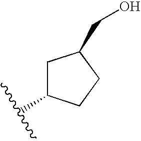 |
| II″-81a | —OCH₃ | 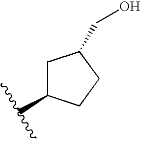 |
| II″-82a | —OCH₃ | 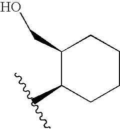 |
| II″-83a | —OCH₃ | 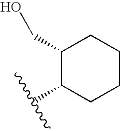 |
| II″-84a | —OCH₃ | 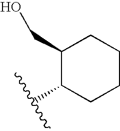 |
| II″-85a | —OCH₃ | 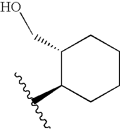 |
| II″-86a | —OCH₃ | 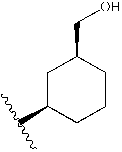 |
| II″-87a | —OCH₃ | 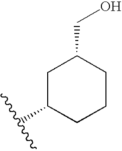 |
| II″-88a | —OCH₃ | 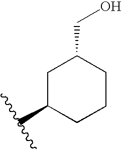 |
| II″-89a | —OCH₃ | 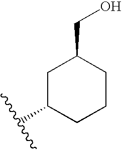 |
| II″-90a | —OCH₃ | 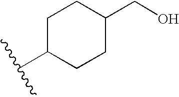 |

and pharmaceutically acceptable salts thereof.
Illustrative examples of Purine Compounds of Formula (II) include the following compounds: 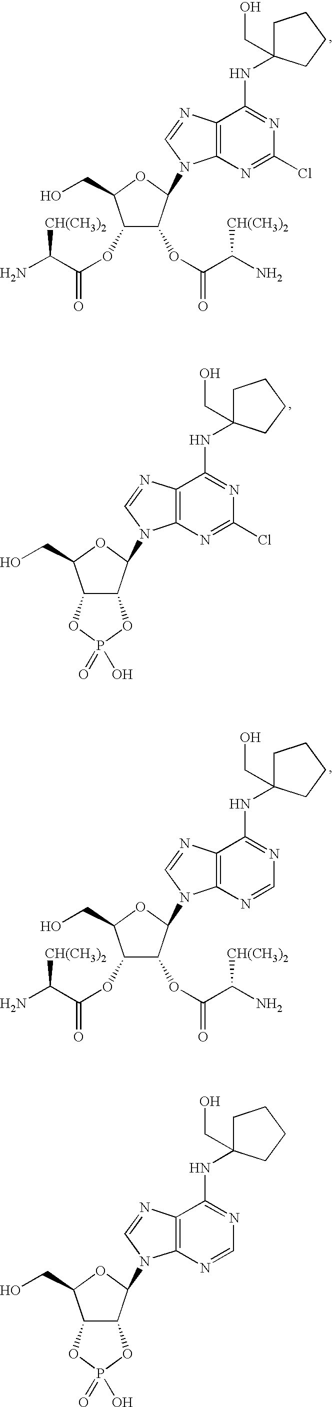
and pharmaceutically acceptable salts thereof.

### 5.2.3 ILLUSTRATIVE EXAMPLES OF THE COMPOUNDS OF FORMULA (III)

Illustrative examples of Purine Compounds of Formula (III) include the following compounds:

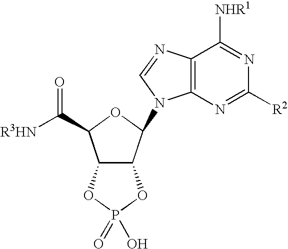

| Compound | R¹ | R² | R³ |
|---|---|---|---|
| III-1 | 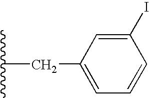 | —H | —CH₃ |
| III-2 | 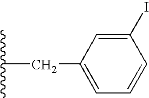 | —Cl | —CH₃ |
| III-3 | 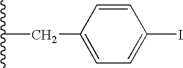 | —H | —CH₃ |
| III-4 | 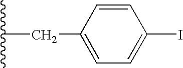 | —Cl | —CH₃ |
| III-5 | 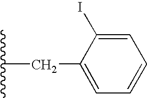 | —H | —CH₃ |
| III-6 | 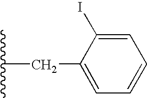 | —Cl | —CH₃ |
| III-7 | —H | —H | —CH₃ |
| III-8 | —H | —H | —CH₂CH₃ |
| III-9 | —CH₃ | —H | —CH₃ |
| III-10 | —H | 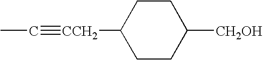 | —CH₂CH₃ |
| III-11 | —H | 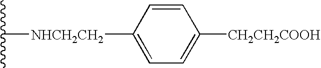 | —CH₂CH₃ |
| III-12 | —H | —C≡C(CH₂)₅CH₃ | —CH₂CH₃ |
| III-13 | —H | —C≡C-phenyl | —CH₂CH₃ |

and pharmaceutically acceptable salts thereof.

Further illustrative examples of Purine Compounds of Formula (III) include the following compounds:

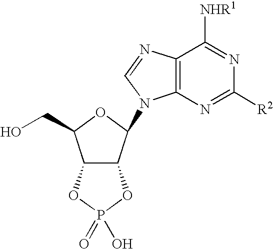

| Compound | R¹ | R² |
|---|---|---|
| III-14 | 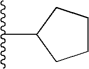 | —Cl |
| III-15 | 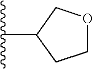 | —Cl |
| III-16 | 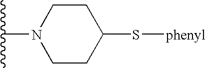 | —Cl |
| III-17 | 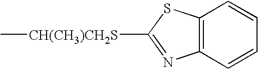 | —Cl |
| III-18 | 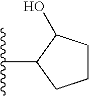 | —Cl |
| III-19 | 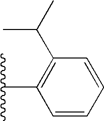 | —Cl |

and pharmaceutically acceptable salts thereof.

Further illustrative examples of Purine Compounds of Formula (III) include the following compounds:

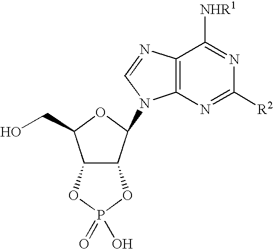

| Compound | R¹ | R² |
|---|---|---|
| III-20 | 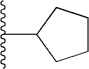 | —H |
| III-21 | 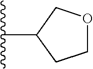 | —H |
| III-22 | 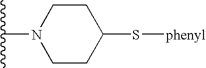 | —H |
| III-23 | 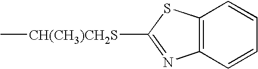 | —H |
| III-24 | 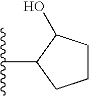 | —H |
| III-25 | 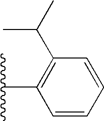 | —H |
| III-26 | 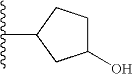 | —H |
| III-27 | 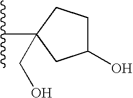 | —H |

and pharmaceutically acceptable salts thereof.
Other illustrative examples of the Purine Compounds of Formula (III) have the following structures:

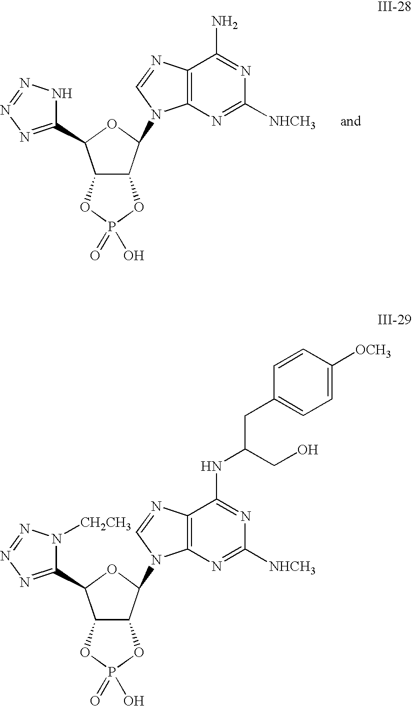

Other illustrative examples of Purine Compounds of Formula (III) include the following compounds:

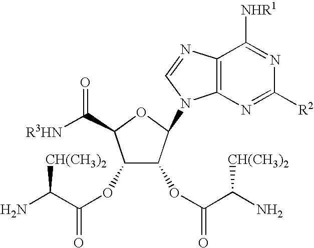

| Compound | R¹ | R² | R³ |
|---|---|---|---|
| III-1a | 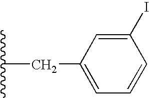 | —H | —CH₃ |
| III-2a | 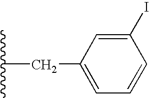 | —Cl | —CH₃ |
| III-3a | 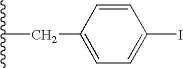 | —H | —CH₃ |
| III-4a | 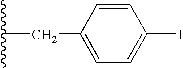 | —Cl | —CH₃ |
| III-5a | 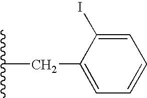 | —H | —CH₃ |
| III-6a | 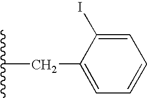 | —Cl | —CH₃ |
| III-7a | —H | —H | —CH₃ |
| III-8a | —H | —H | —CH₂CH₃ |
| III-9a | —CH₃ | —H | —CH₃ |
| III-10a | —H | 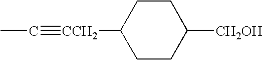 | —CH₂CH₃ |
| III-11a | —H | 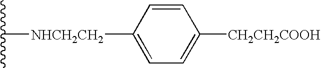 | —CH₂CH₃ |
| III-12a | —H | —C≡C(CH₂)₅CH₃ | —CH₂CH₃ |
| III-13a | —H | —C≡C-phenyl | —CH₂CH₃ |

and pharmaceutically acceptable salts thereof.

Other illustrative examples of Purine Compounds of Formula (III) include the following compounds:

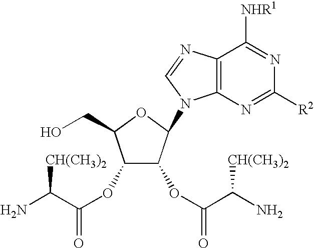

| Compound | R¹ | R² |
|---|---|---|
| III-20a | 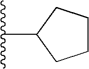 | —H |
| III-14a | 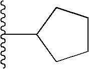 | —Cl |
| III-15a | 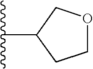 | —Cl |
| III-22a | 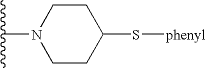 | —H |
| III-17a | 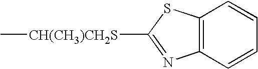 | —Cl |
| III-24a | 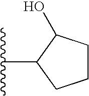 | —H |
| III-25a | 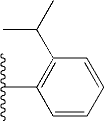 | —H |
| III-26a | 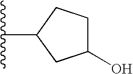 | —H |
| III-27a | 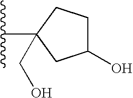 | —H |

and pharmaceutically acceptable salts thereof.

Other illustrative compounds of Formula (III) are:

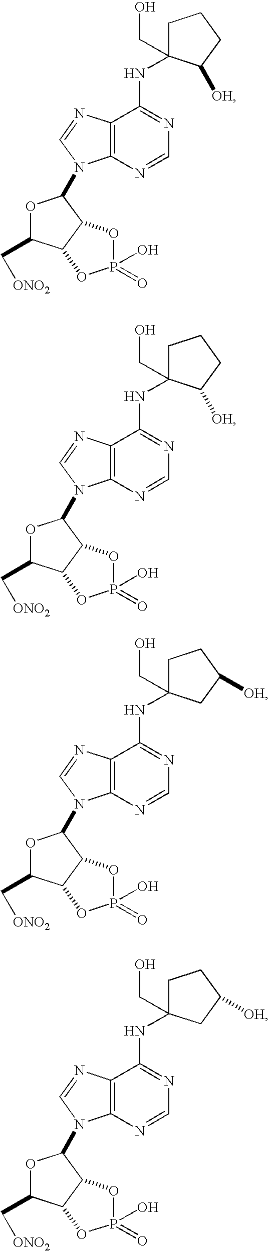

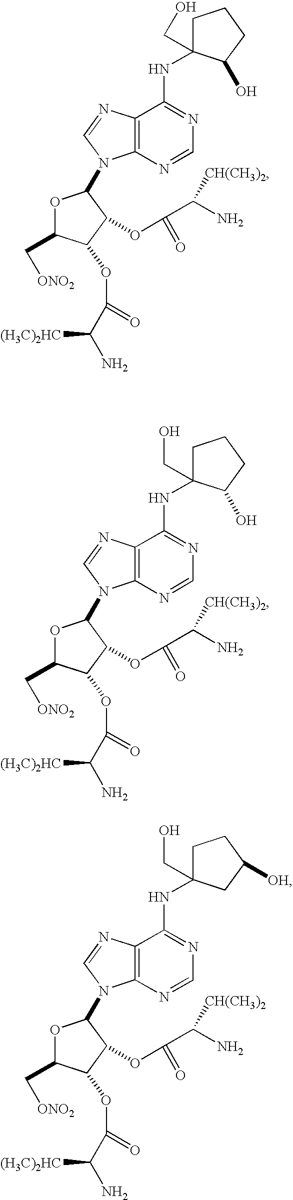

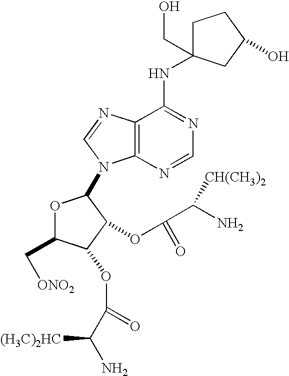

and pharmaceutically acceptable salts thereof.

Other illustrative examples of the Purine Compounds of Formula (III) have the structures: 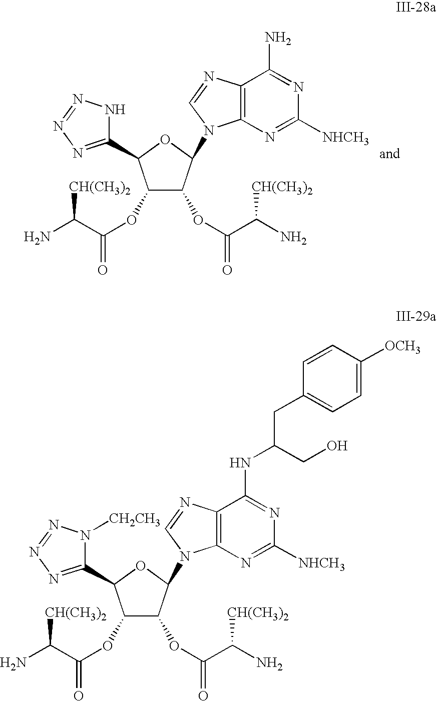
and pharmaceutically acceptable salts thereof.

The Purine Compounds of Formula (I), (II) or (III) may contain one or more chiral centers. Where no stereochemistry is indicated in a chemical structure or name, the structure or name encompasses both enantiomers, its racemate and all mixtures thereof.

Additionally, the Purine Compounds may contain one or more double bonds. Where no particular geometric isomer of a double bond is indicated in a chemical structure or name, the structure or name encompasses encompass the double bond's cis isomer, the trans isomer and all mixtures thereof.

### 5.3 METHODS FOR MAKING THE PURINE COMPOUNDS

The Purine Compounds can be made according to methods well-known to one skilled in the art of organic chemistry or by using the synthetic procedures outlined below in Schemes 1-34.

Scheme I shows methods for making adenosine intermediates that are useful for making the Purine Compounds of Formula (I). 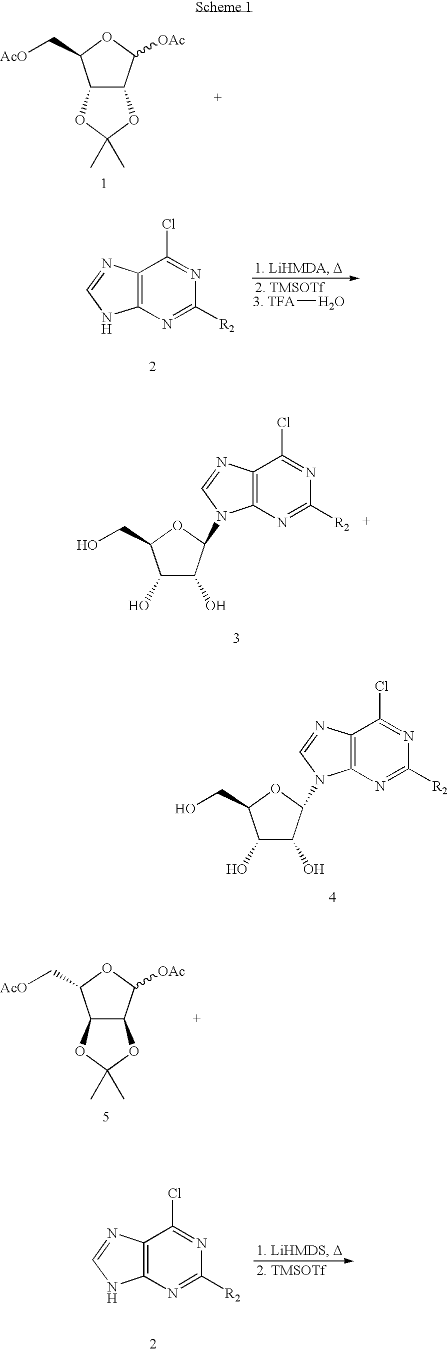 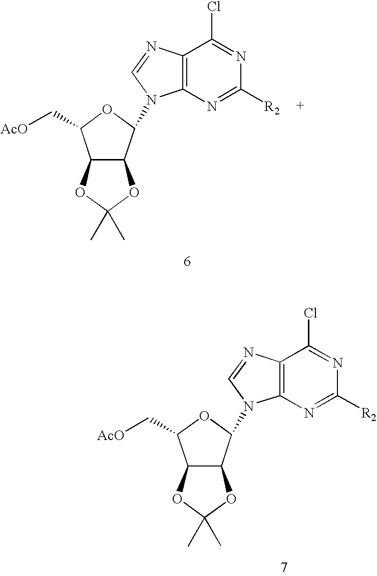
wherein R₂ is as defined above for the Purine Compounds of Formula (I).

The protected ribose compound of Formula 1 can be coupled with a purine compound of Formula 2 using lithium hexamethyldisilazide and TMS triflate, followed by acetonide removal using trifluoroacetic acid (TFA) to provide nucleoside intermediates of Formula 3 and their corresponding other anomers of Formula 4. Similarly, the protected ribose tetraacetate of Formula 5 can be coupled with a compound of Formula 2 to provide protected acetyl nucleoside intermediates of Formula 6 and their corresponding other anomers of Formula 7.

Scheme 2 shows a method useful for making the adenosine intermediates of Formula 8 which are useful for making the Purine Compounds of Formula (I). 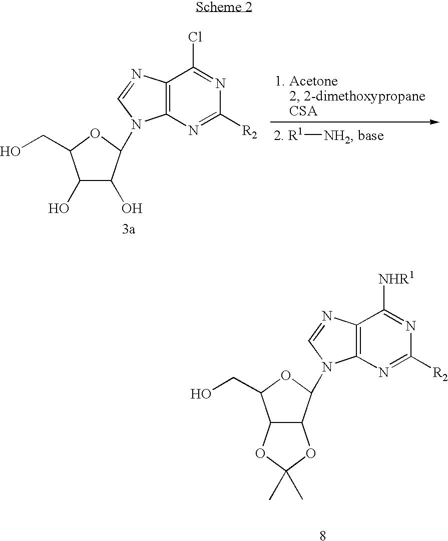
where R¹ and R² are defined herein for the Purine Compounds of Formula (I).

The 6-chloroadenosine derivative of formula 3a is converted to its 2′,3′-acetonide using acetone and 2,2-dimethoxypropane in the presence of camphorsulfonic acid (CSA). The acetonide can then be further derviatized using an amine of formula R¹—NH₂ in the presence of base to provide compounds of formula 8.

Alternatively, a purine compound of Formula 2 can be coupled with a tetraacetate protected ribose compound of formula Z using lithium hexamethyldisilazide and trimethylsilyl trifluormethanesulfonamide (TMS triflate). The resulting adduct can be protected as its acetonide derivative using using acetone and 2,2-dimethoxypropane in the presence of camphorsulfonic acid to provide compounds of formula 8 as shown in Scheme 3.

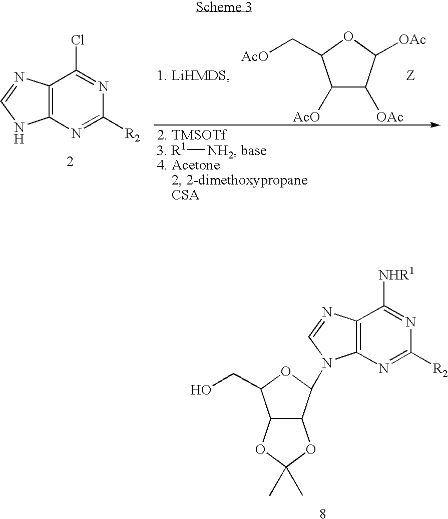

Scheme 4 illustrates a general method for making the Purine Compounds having a 2′,3′-cyclic phosphate. 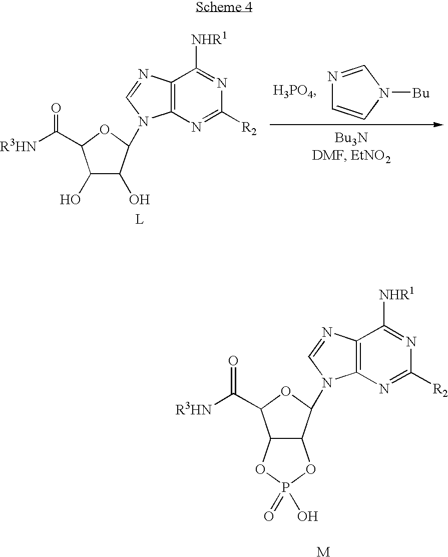
wherein R¹, R² and R³ are as defined above herein for the Purine Compounds of Formula (I).

A Compound of formula L can be reacted with phosphoric acid in the presence of 1-butylimidazole and n-butylamine in a mixture of N,N-dimethylformamide (DMF) and nitroethane as described in Sakakura et al., Org. Letters 7:1999-2002 (2005) to provide Purine Compounds of formula M, having a 2′,3′-cyclic phosphate group.

Scheme 5 illustrates a general method for making the Purine Compounds having a 2′,3′-diester, wherein the esters are derived from a naturally occurring amino acid. 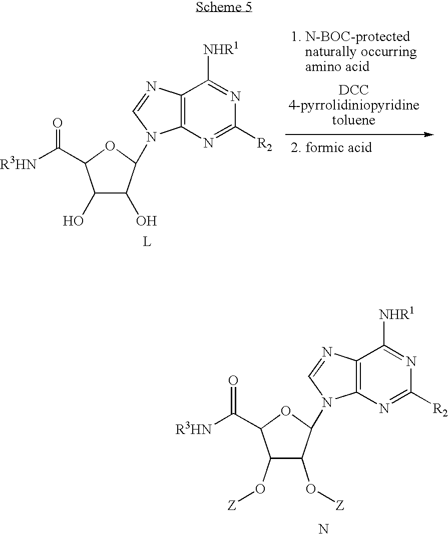
wherein R¹, R² and R³ are as defined above herein for the Purine Compounds of Formula (I); Z is R⁹ or R¹⁰ as defined for the Purine Compounds of Formula (I).

A Purine Compound of formula L can be coupled with the carboxy terminus of a BOC-protected naturally occurring amino acid using dicyclohexylcarbodiimide (DCC) and 4-pyrrolidinopyridine in toluene. The resulting ester is then treated with formic acid to remove the BOC protecting group and provide a Purine Compound of formula N, having a 2′,3′-diester.

Scheme 6 illustrates a method useful for making the Purine Compounds where R² is —NH—N═C(R⁵)R⁶. 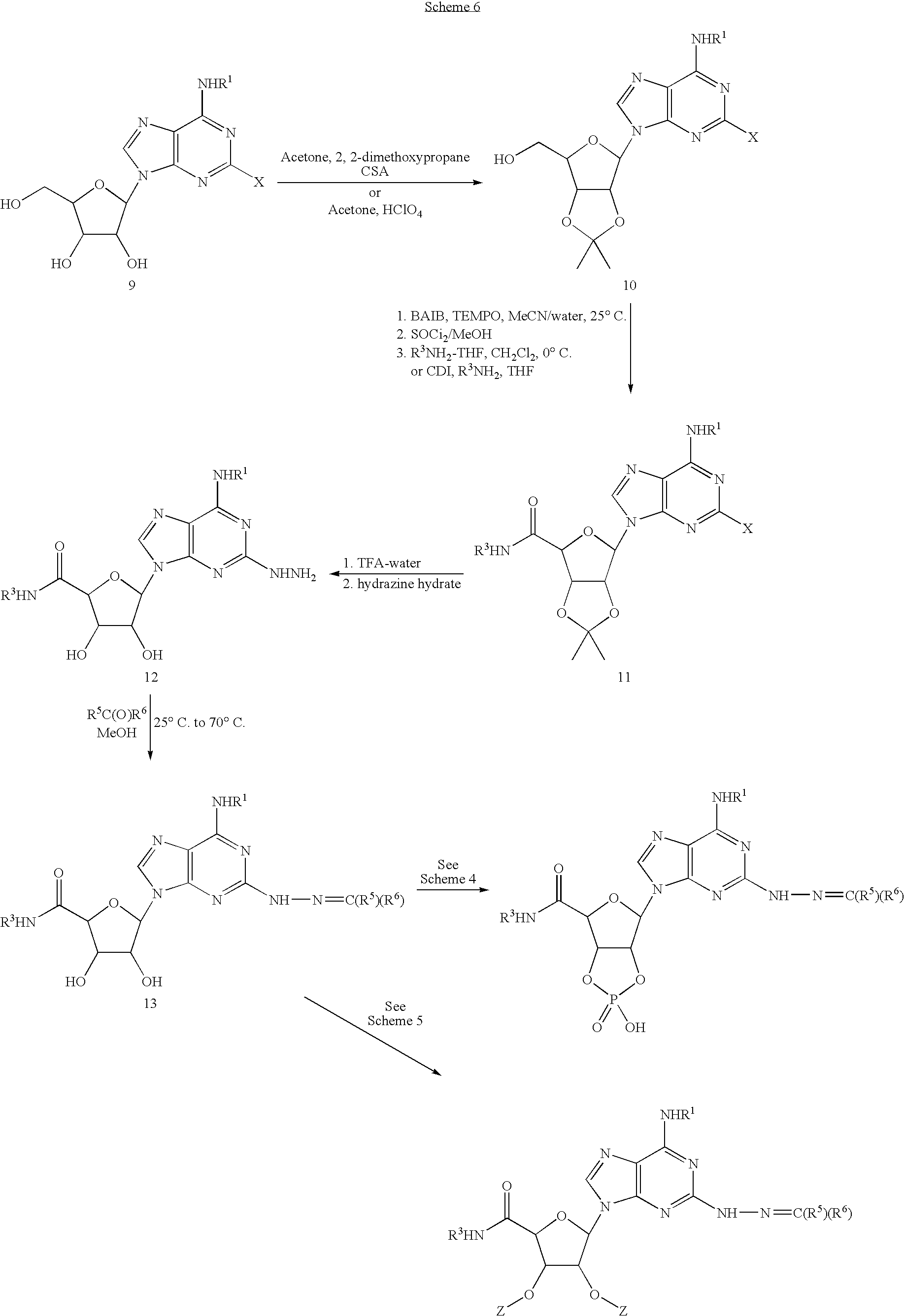
wherein X is —Cl or —I; Z is R⁹ or R¹⁰ as defined for the Purine Compounds of Formula (I); and R¹, R³, R⁵ and R⁶ are as defined above herein for the Purine Compounds.

The 2-chloroadenosine or 2-iodoadenosine derivatives of formula 9 are converted to their acetonide derivatives of formula 10 upon treatment with 2,2-dimethoxypropane in the presence of camphorsulfonic acid, or alternatively by treating with acetone in the presence of perchloric acid. The hydroxymethyl group of the compounds of formula 10 are then converted to the amides of formula 11 using a three-step procedure. The hydroxyl group of 10 is first oxidized using TEMPO (2,2,6,6-tetramethyl-1-piperidinyloxy, free radical) to provide the corresponding carboxylic acid intermediates, which are then converted to the corresponding acid chloride or ester intermediates using thionyl chloride in methanol. The acid chloride intermediates are then coupled with an amine of formula R³NH₂ to provide the amide compounds of formula 11. The acetonide protecting group of the compounds of formula 11 is then removed using TFA and, as specifically illustrated in Scheme 1, the deprotected compounds can then be treated with hydrazine hydrate to provide the hydrazines of formula 12 which can subseqently be coupled with a ketone or aldehyde having the formula R⁵C(O)R⁶ to provide compounds of formula 13. The 2′,3′-dihydroxy group of the compounds of formula 13 can then be converted to a cyclic phosphate using the methodology set forth in Scheme 4, or alternatively can be converted to a 2′,3′-diester using the methodology set forth in Scheme 5, to make the Purine Compounds wherein R² is —NH—N═C(R⁵)R⁶.

Scheme 7 illustrates a method for making the Purine Compounds wherein R² is —NHNHC(O)R⁴, —NHNHC(O)OR⁴ or —NHNHC(O)NHR⁴. 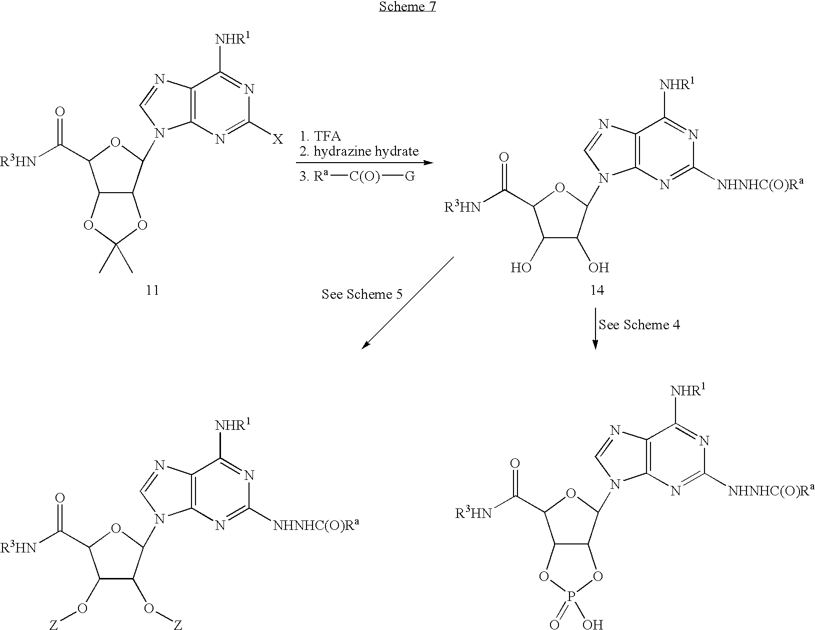
wherein X is —Cl or —I; Z is R⁹ or R¹⁰ as defined for the Purine Compounds of formula (I); R¹ and R³ are as defined above herein for the Purine Compounds of Formula (I); and R^{a} is R⁴,
—OR⁴ or —NHR⁴.

The 2′,3′-isopropylidine group of a 2-chloroadenosine or 2-iodoadenosine derivative of formula 11 is removed using TFA and the resultant dihydroxy derivatives can be reacted with hydrazine hydrate to provide the corresponding 2-hydrazino derivatives. The 2-hydrazino derivatives can then be coupled with a compound of formula R^{a}—C(O)-G to provide the compounds of formula 14. The 2′,3′-dihydroxy group of the compounds of formula 14 can then be converted to a cyclic phosphate using the methodology set forth in Scheme 4, or alternatively can be converted to a 2′,3′-diester using the methodology set forth in Scheme 5, to make the Purine Compounds wherein R² is —NHNHC(O)R⁴, —NHNHC(O)OR⁴ or —NHNHC(O)NHR⁴.

Scheme 8 shows a method useful for making the Purine Compounds where R² is —CN. 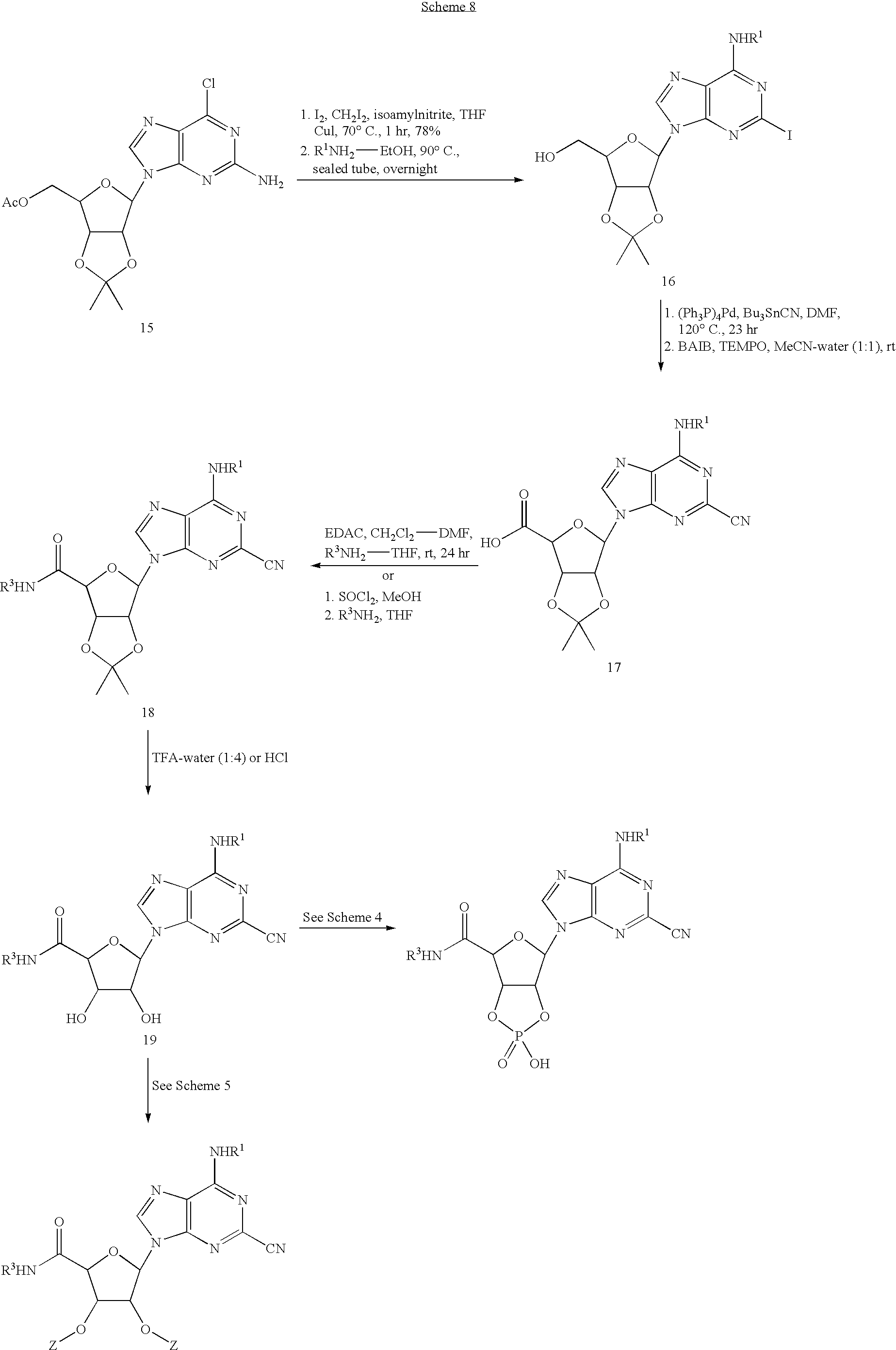
wherein Z is R⁹ or R¹⁰ as defined for the Purine Compounds of Formula (I); and R¹ and R³ are as defined above herein for the Purine Compounds.

The 2-amino purinyl acetate of formula 15 is converted to its 2-iodo analog, which is then reacted with an amine of formula R¹NH₂ to provide the 2-iodo adenosine derviatives of formula 16. The compounds of formula 16 are then converted to their corresponding 2-cyano derivatives upon Pd catalyzed cyanation of the aromatic iodide moiety and the, hydroxymethyl group is subsequently oxidized to the corresponding carboxylic acids of formula 17 using TEMPO. The carboxylic acids of formula 17 can then be coupled with an amine having the formula R³NH₂ in the presence of EDAC (1-(3-dimethylaminopropyl)-3-ethylcarbodiimide hydrochloride) to provide the corresponding amides of formula 18. Alternatively, the compounds of formula 17 may be converted to the compounds of formula 18 by first reacting a compound of formula 17 with thionyl chloride in methanol, then coupling the resultant methyl ester with an amine of formula R³NH₂. The compounds of formula 18 are then treated with acid (TFA or HCl) to remove the acetonide group and provide the compounds of formula 19. The 2′,3′-dihydroxy group of the compounds of formula 19 can then be converted to a cyclic phosphate using the methodology set forth in Scheme 3, or alternatively can be converted to a 2′,3′-diester using the methodology set forth in Scheme 4, to make the Purine Compounds wherein R is —CN.

Scheme 9 shows a method for making the Purine Compounds where R² is —NHC(O)OR⁴or —NHC(O)NHR⁴. 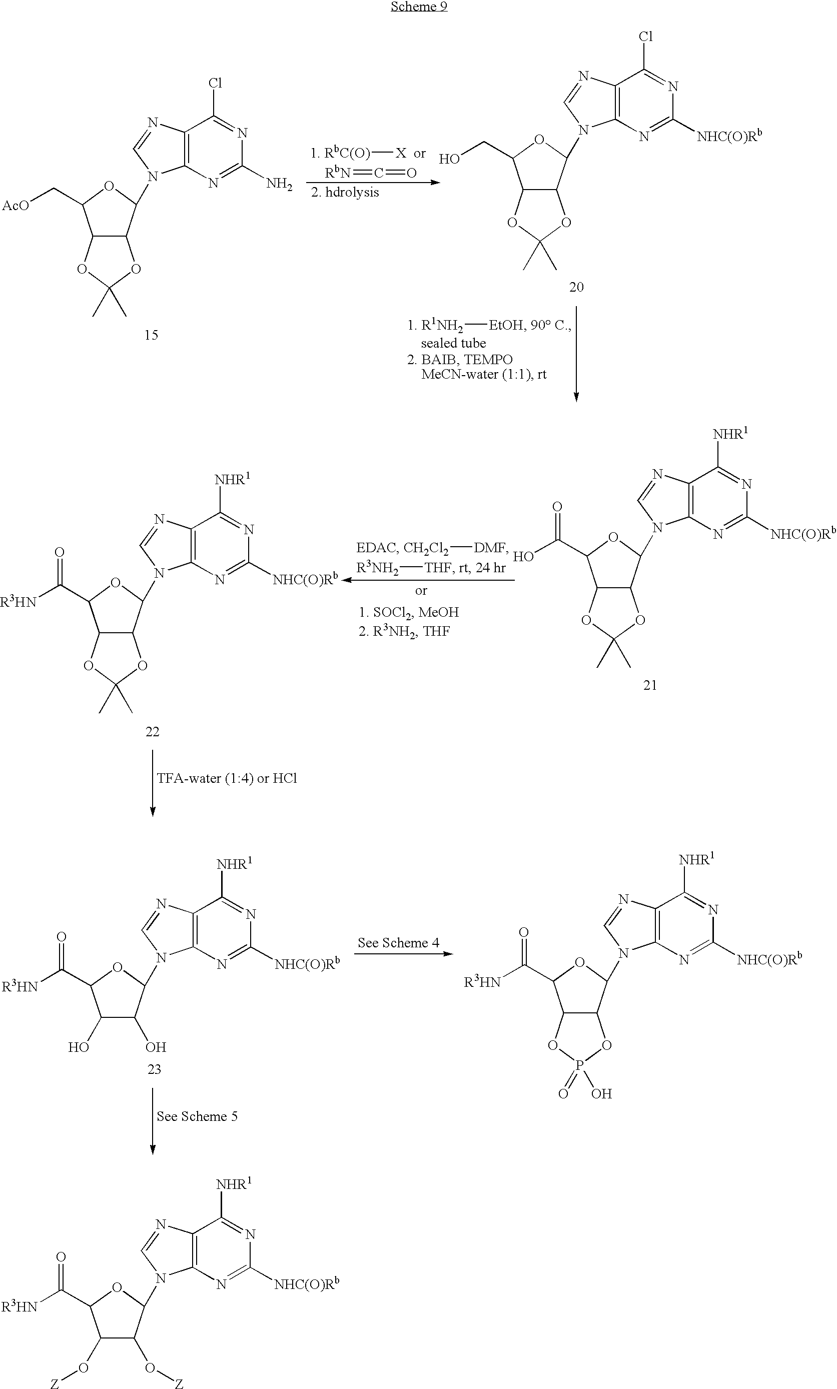
wherein R¹ and R³ are as defined above herein for the Purine Compounds; Z is R⁹ or R¹⁰ as defined for the Purine Compounds of Formula (I); R^{b} is —R⁴, —OR⁴, or —NHR⁴, R⁴ is defined as above for the Purine Compounds of Formula (I); and X is —Cl or —Br.

The 2-amino group of the purinyl acetate of formula 15 is coupled with an acyl halide, haloformate, or halocarbamyl of formula R^{b}C(O)—X or an isocyanate of formula R^{b}N═C═O, then treated with potassium carbonate in methanol to provide the hydroxymethyl compounds of formula 20. The chloro group of the compounds 20 is then reacted with an amine of formula R¹—NH₂ to provide the corresponding 6-amino compounds, which are then oxidized using TEMPO to provide the carboxylic acid intermediates of formula 21. The carboxylic acid compounds of formula 21 can then be coupled with an amine of formula R³NH₂ to provide the corresponding carboxamido compounds of formula 22. Alternatively, the compounds of formula 21 may be converted to the compounds of formula 22 by first reacting a compound of formula 21 with thionyl chloride in methanol, then coupling the resultant methyl ester with an amine of formula R³NH₂. The compounds of formula 22 are then treated with acid (TFA or HCl) which can be treated with acid to remove the acetonide group and provide the compounds of formula 23. The 2′,3′-dihydroxy group of the compounds of formula 23 can then be converted to a cyclic phosphate using the methodology set forth in Scheme 3, or alternatively can be converted to a 2′,3′-diester using the methodology set forth in Scheme 4, to make the Purine Compounds wherein R is —NHC(O)OR⁴ or —NHC(O)NHR⁴.

Scheme 10 shows another method useful for making the Purine Compounds where R² is —CN. 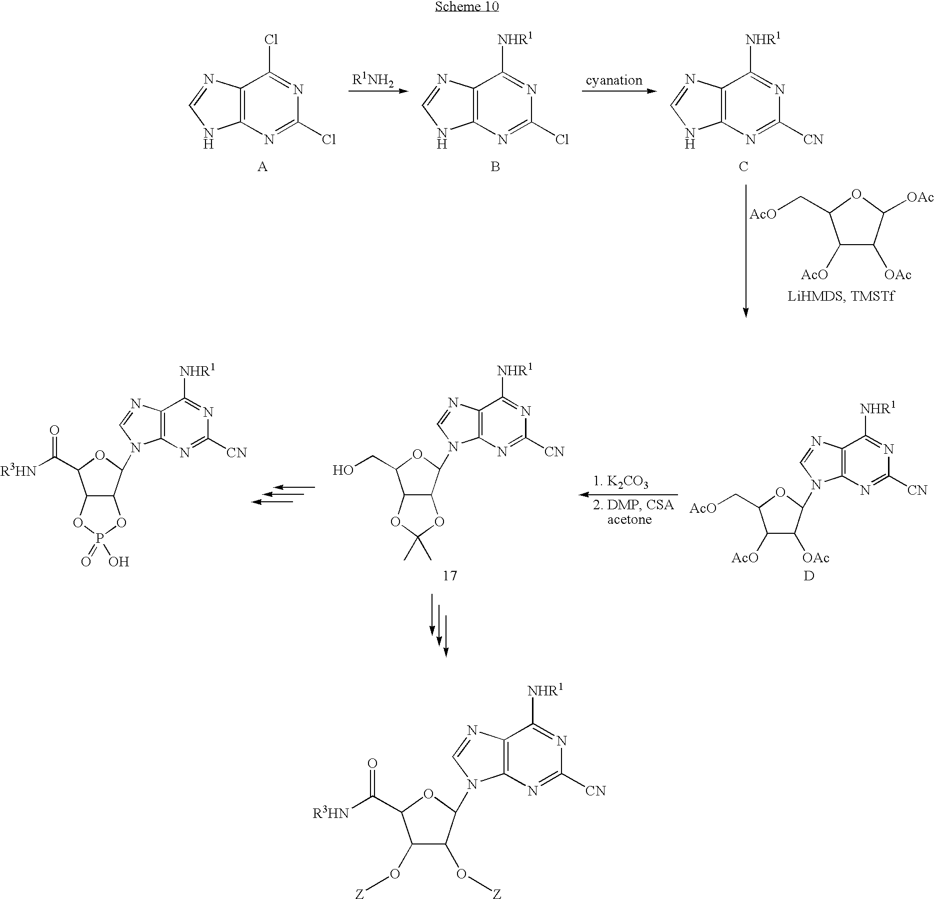
wherein Z is R⁹ or R¹⁰ as defined for the Purine Compounds of Formula (I); and R¹ and R³ are as defined above herein for the Purine Compounds.

2,6-dichloropurine (A) is reacted with an amine of formula R¹NH₂ to provide the corresponding amino compound of formula B. The 2-chloro group of B can then be converted to a nitrile using a palladium-catalyzed coupling reaction as described, for example, in Zapf, et al., Chemical Communications, 4:431-440 (2005), to provide a 2-cyano purinyl compound of formula C. The compound of formula C is then coupled with ribofuranose tetraacetate to provide a triacetate nucleoside compound of formula D. The acetate groups of D are subsequently hydrolyzed using, for example, potassium carbonate and the resultant compound of formula 17 can be further modified as described in Scheme 7 above to provide provide Purine Compounds wherein R² is —CN.

Scheme 11 shows a method useful for making the Purine Compounds where R² is —CN and wherein R¹ and R³ are the same. 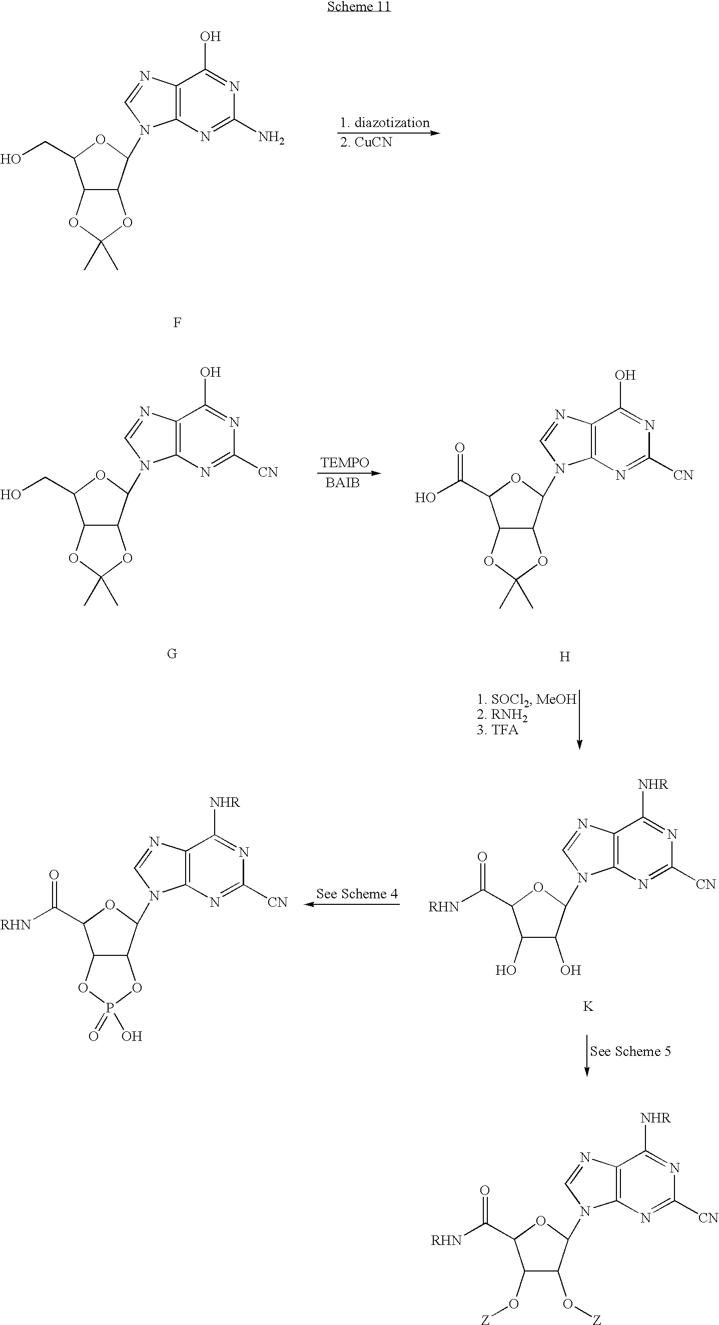
wherein Z is R⁹ or R¹⁰ as defined for the Purine Compounds of formula (Ia); and R¹ and R³ are as defined above herein for the Purine Compounds.

The 2-amino group of the purinyl compound of formula F is diazotized using, for example, nitrous acid or an alkyl nitrite, and the resultant diazonium salt can then be reacted with CuCN to provide a 2-cyano puriyl compound of formula G. The 5′-hydroxymethyl group of G is then oxidized to the corresponding carboxylic acid H using TEMPO. The compound of formula H is then reacted with thionyl chloride in methanol to provide an intermediate methyl ester, which is subsequently reacted with a amine of formula RNH₂, then treated with TFA to provide the 2′,3′-diol compounds of formula K. The 2′,3′-dihydroxy group of the compounds of formula K can then be converted to a cyclic phosphate using the methodology set forth in Scheme 4, or alternatively can be converted to a 2′,3′-diester using the methodology set forth in Scheme 5, to make the Purine Compounds wherein R² is —CN and wherein R¹ and R³ are the same.
Scheme 12 shows a method useful for making the Purine Compounds of Formula (Ia) 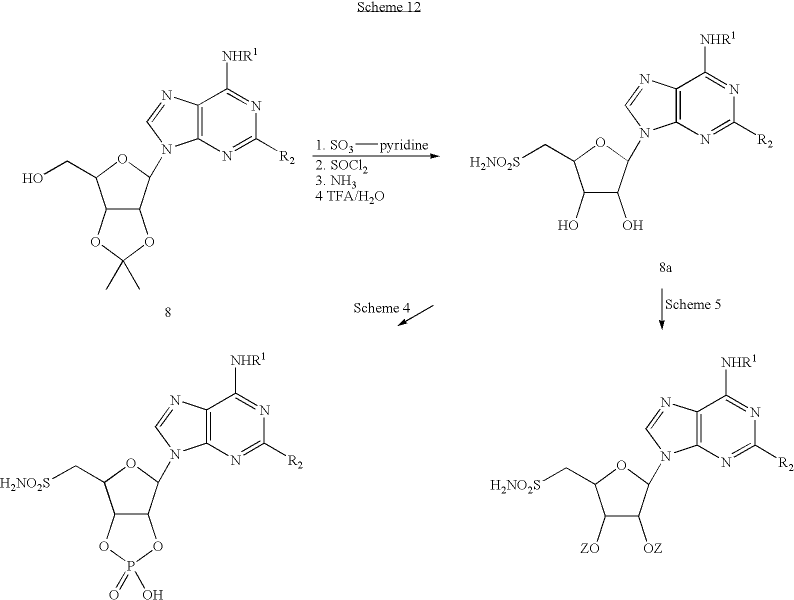
where R¹ and R² are defined above herein for the Purine Compounds of Formula (I) and Z is R⁹ or R¹⁰ as defined for the Purine Compounds of formula (I).

The adenosine intermediates of formula 8 can be converted to their 5′-sulfonic acid analogs, which can then be chlorinated using thionyl chloride to provide the corresponding 5′-chlorosulfonate intermediates. The chlorosulfonate intermediates can then be reacted with ammonia to provide the corresponding 5′-sulfonamide intermediates. Acetonide removal using TFA/water provides the Purine Compounds of Formula 8a. The 2′,3′-dihydroxy group of the compounds of formula 8a can then be converted to a cyclic phosphate using the methodology set forth in Scheme 4, or alternatively can be converted to a 2′,3′-diester using the methodology set forth in Scheme 5, to make the Purine Compounds of Formula (I).
Further methodology useful for making Purine Compounds of Formula (I) is described in Scheme 13. 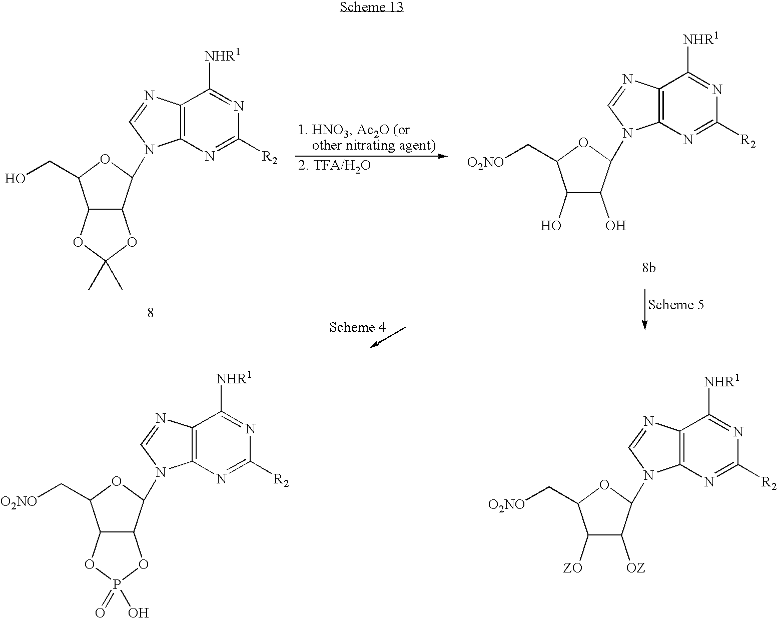
where R¹ and R² are defined above herein for the Purine Compounds of Formula (I) and Z is R⁹ or R¹⁰ as defined for the Purine Compounds of Formula (I).

The Adenosine intermediates of formula 8 can be converted to their 5′-nitrate analogs using nitric acid in the presence of acetic anhydride, or other nitrating agents, such as MsCl/ONO₃ or nitrosonium tetrafluoroborate. Acetonide removal using TFA/water provides Purine Compounds of formula 8b. The 2′,3′-dihydroxy group of the compounds of formula 8b can then be converted to a cyclic phosphate using the methodology set forth in Scheme 4, or alternatively can be converted to a 2′,3′-diester using the methodology set forth in Scheme 5, to make the Purine Compounds of (I).
Methodology useful for making the Purine Compounds of Formula (I) where A is —CH₂NHR⁵ is outlined below in Scheme 14. 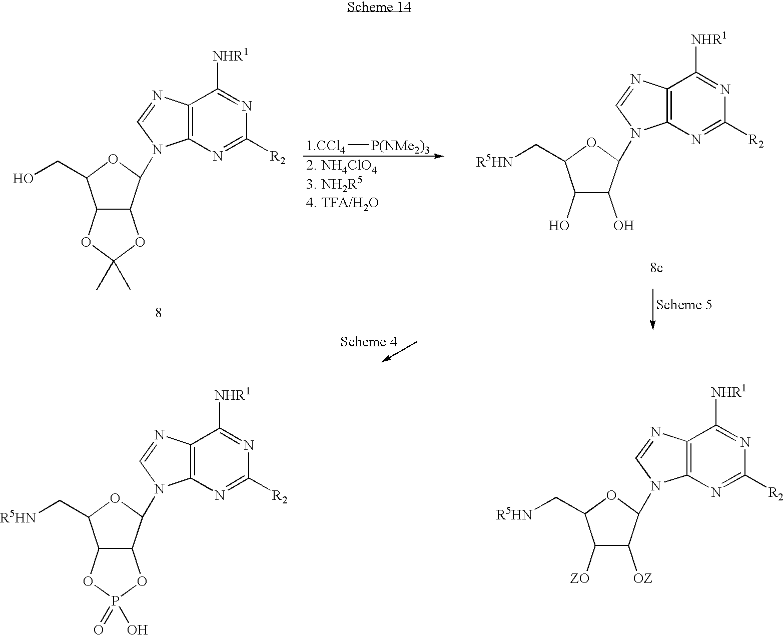
where R¹, R² and R⁵ are defined above herein for the Purine Compounds of Formula (I) and Z is R⁹ or R¹⁰ as defined for the Purine Compounds of Formula (I).

The adenosine intermediates of formula 8 can be converted to their 5′-alkoxyphosphonium perchlorate analogs using CCl₄—P(NMe₂)₃, then treating the product of this reaction with ammonium perchlorate. The intermediate 5′-alkoxyphosphonium perchlorates can subsequently be reacted with an amine of formula NH₂R⁵ to provide the 5′-amino analogs. Acetonide removal using TFA/water provides the Purine Compounds of formula 8c. The 2′,3′-dihydroxy group of the compounds of formula 8c can then be converted to a cyclic phosphate using the methodology set forth in Scheme 4, or alternatively can be converted to a 2′,3′-diester using the methodology set forth in Scheme 5, to make the Purine Compounds of Formula (I).
Methodology useful for making the Purine Compounds of Formula (I) wherein A is —CH₂OSO₃H is outlined in Scheme 15. 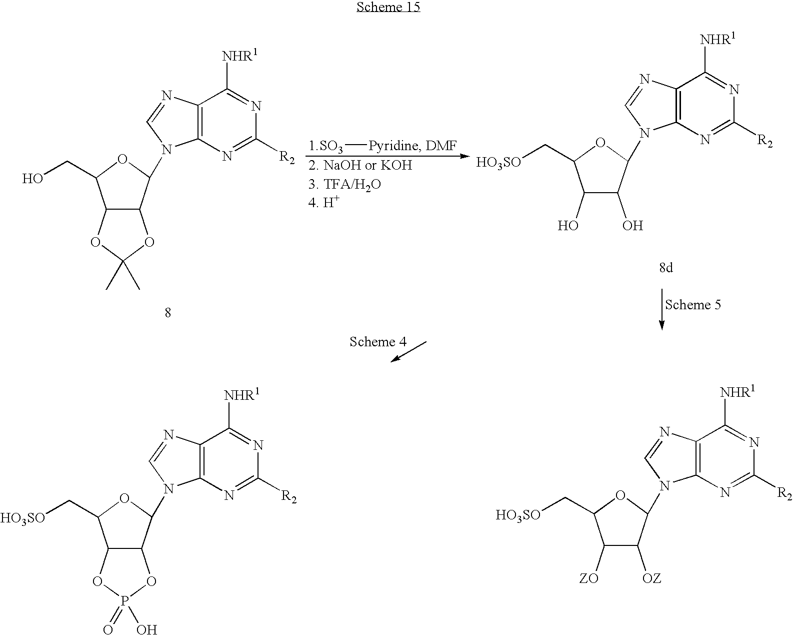
where R¹ and R² are defined above herein for the Purine Compounds of Formula (I) and Z is R⁹ or R¹⁰ as defined for the Purine Compounds of formula (I), wherein R³ is —CH₂OSO₃H.

The adenosine intermediates of formula 8 can be treated with sulfur trioxide-pyridine complex to provide the corresponding 5′-sulfonic acid pyridine salt intermediate. The pyridine salt intermediate can then be neutralized using NaOH or KOH, followed by acetonide removal using TFA/water to provide the corresponding sodium or potassium salt, respectively, of the Purine Compounds of Formula (Id) wherein R³ is —CH₂OSO₃H. Treatment of the sodium or potassium salt with strong aqueous acid, such as sulfuric or hydrochloric acid, provides the Purine Compounds of formula 8d. The 2′,3′-dihydroxy group of the compounds of formula 8d can then be converted to a cyclic phosphate using the methodology set forth in Scheme 4, or alternatively can be converted to a 2′,3′-diester using the methodology set forth in Scheme 5, to make the Purine Compounds of formula (I) wherein A is —CH₂OSO₃H.
Methodology useful for making the Purine Compounds of Formula (I) wherein R³ is —CH₂ONO is outlined in Scheme 16. 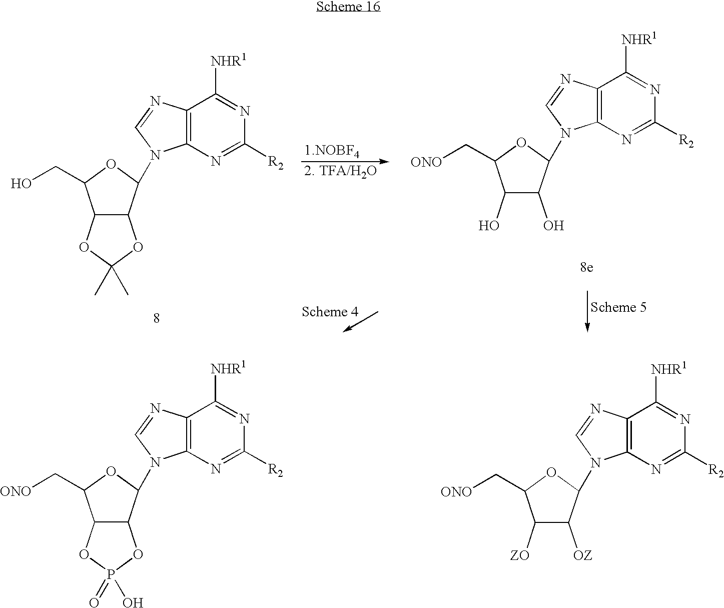
where R¹ and R² are defined above herein for the Purine Compounds of Formula (Id) and Z is R⁹ or R¹⁰ as defined for the Purine Compounds of Formula (I), wherein R³ is —CH₂ONO.

The adenosine intermediates of formula 8 can be treated with nitrosonium fluoroborate complex to provide the corresponding nitrosooxy intermediates. Acetonide removal using TFA/water provides the Purine Compounds of formula 8e. The 2′,3′-dihydroxy group of the compounds of formula 8e can then be converted to a cyclic phosphate using the methodology set forth in Scheme 2, or alternatively can be converted to a 2′,3′-diester using the methodology set forth in Scheme 3, to make the Purine Compounds of Formula (I) wherein R³ is —CH₂ONO.

Methodology useful for making the Purine Compounds of Formula (I) wherein R³ is —CH₂OSO₂NH(C₁-C₁₀ alkyl), —CH₂OSO₂N(C₁-C₁₀ alkyl)₂, or —CH₂OSO₂NH-aryl, is outlined in Scheme 17. 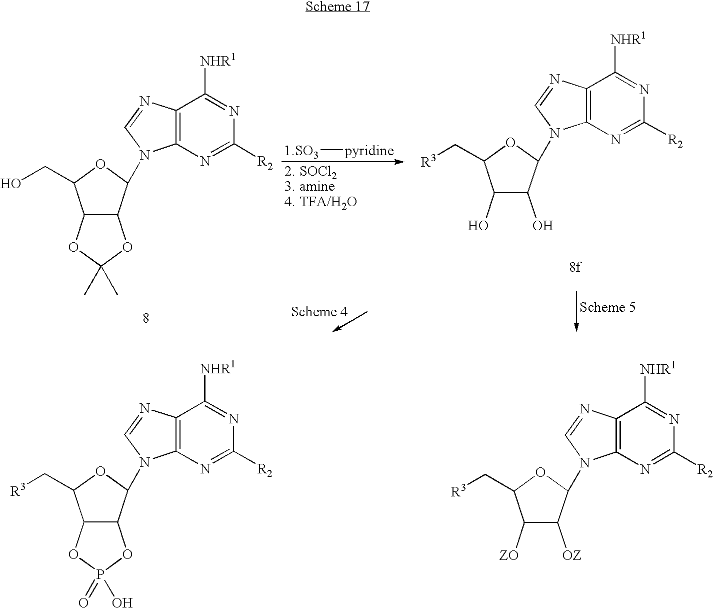
where R¹ and R² are defined above herein for the Purine Compounds of Formula (I) and Z is R⁹ or R¹⁰ as defined for the Purine Compounds of Formula (I), wherein R³ is —CH₂OSO₂NH(C₁-C₁₀ alkyl), —CH₂OSO₂N(C₁-C₁₀ alkyl)₂, or —CH₂OSO₂NH-aryl.

The adenosine intermediates of formula 8 can be reacted with sulfur trioxide-pyridine complex to provide the corresponding 5′-sulfonic acid intermediates, which can subsequently be treated with thionyl chloride to provide the intermediate 5′-chlorosulfonate intermediates. The chlorosulfonate intermediates can then be reacted with an amine of formula H₂N—(C₁-C₁₀ alkyl), HN(C₁-C₁₀ alkyl)₂ or H₂N-aryl to provide the corresponding 5′-sulfonamide intermediates. Acetonide removal using TFA/water provides the Purine Compounds of formula 8f. The 2′,3′-dihydroxy group of the compounds of formula 8f can then be converted to a cyclic phosphate using the methodology set forth in Scheme 4, or alternatively can be converted to a 2′,3′-diester using the methodology set forth in Scheme 5, to make the Purine Compounds of Formula (I) wherein wherein R³ is —CH₂OSO₂NH(C₁-C₁₀ alkyl), —CH₂OSO₂N(C₁-C₁₀ alkyl)₂, or —CH₂OSO₂NH-aryl.

Methodology useful for making the Purine Compounds of Formula (I) is outlined in Scheme 18. 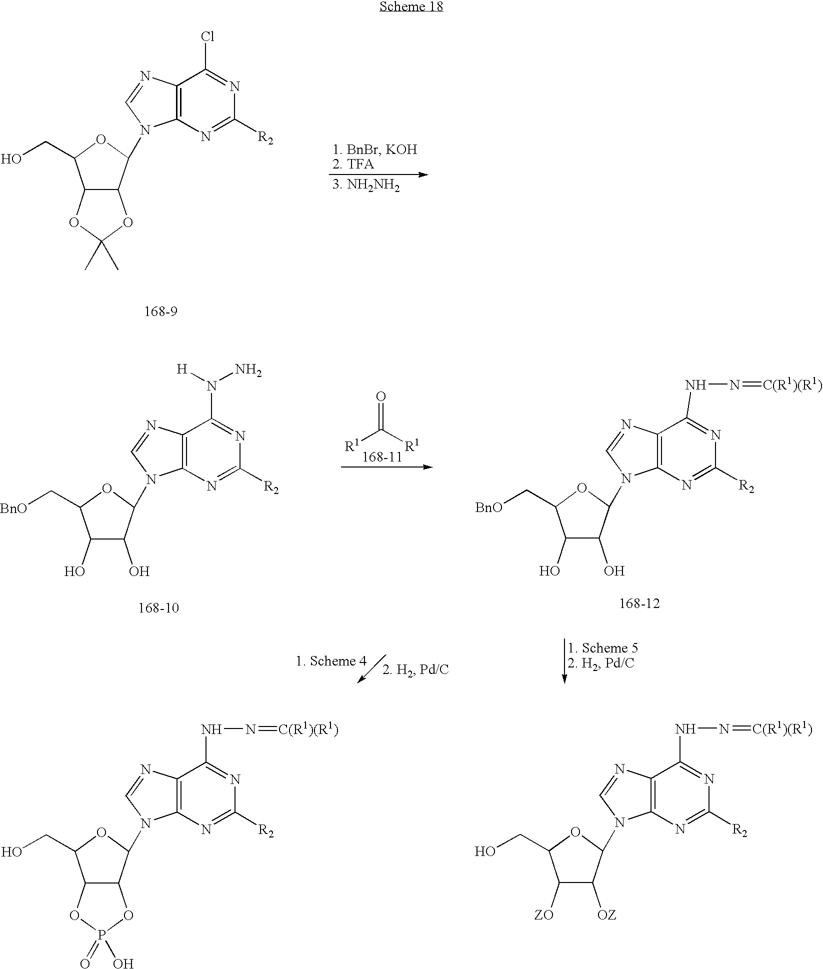
where R¹ and R² are defined above herein for the Purine Compounds of Formula (168-II) and Z is R⁸ or R⁹ as defined for the Purine Compounds of formula (168-II).

The 6-chloroadenosine derivatives of Formula 168-9 (prepared by protecting the 2′3′-dihydroxy group of the compounds of formula 3a as its isopropylidene derivative) can be reacted with benzyl bromide in the presence of KOH to provide the corresponding 5′-O-benzyl intermediates. Removal of the isopropylidene group using TFA, followed by reaction with hydrazine, provides the 6-hydrazino derivatives of Formula 168-10. The compounds of Formula 168-10 can then be treated with a carbonyl compound of formula 168-11 to provide the compounds of formula 168-12. The 2′,3′-dihydroxy group of the compounds of formula 168-12 can then be converted to a cyclic phosphate using the methodology set forth in Scheme 4, or alternatively can be converted to a 2′,3′-diester using the methodology set forth in Scheme 5. Removal of the benzyl group using catalytic hydrogenation provides the Purine Compounds of formula (168-II).

Methodology useful for making-the Purine Compounds of Formula (168-III) is outlined in Scheme 19. 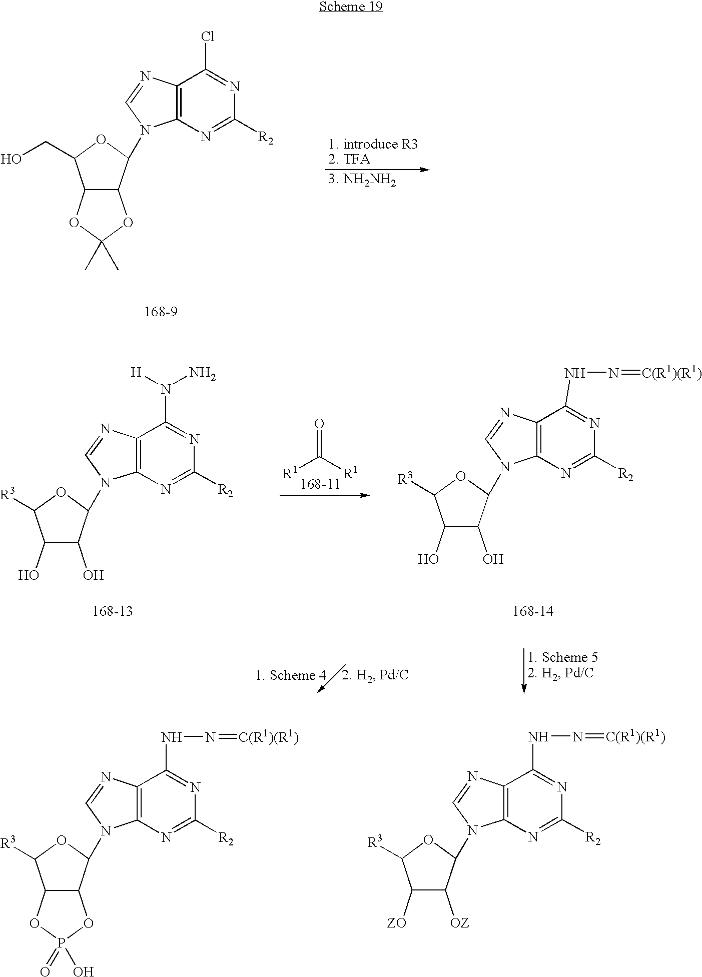
where R¹, R² and R³ are defined above herein for the Purine Compounds of Formula (168-III) and Z is R⁸ or R⁹ as defined for the Purine Compounds of Formula (168-III).

The 5′-OH group of the compounds of formula 168-9 can be converted to an R³ group by one skilled in the art of organic synthesis using methods set forth above in Schemes 5-10. Subsequent removal of the acetonide unit using TFA, followed by reaction with hydrazine, affords the 6-hydrazino compounds of formula 168-13 which can then be reacted with a carbonyl compound of formula 168-11 to provide the Purine Compounds of formula 168-14. The 2′,3′-dihydroxy group of the compounds of formula 168-14 can then be converted to a cyclic phosphate using the methodology set forth in Scheme 4, or alternatively can be converted to a 2′,3′-diester using the methodology set forth in Scheme 5, to make the Purine Compounds of Formula (168-III).

Methodology useful for making the Purine Compounds of Formula (168-IV) is outlined in Scheme 20. 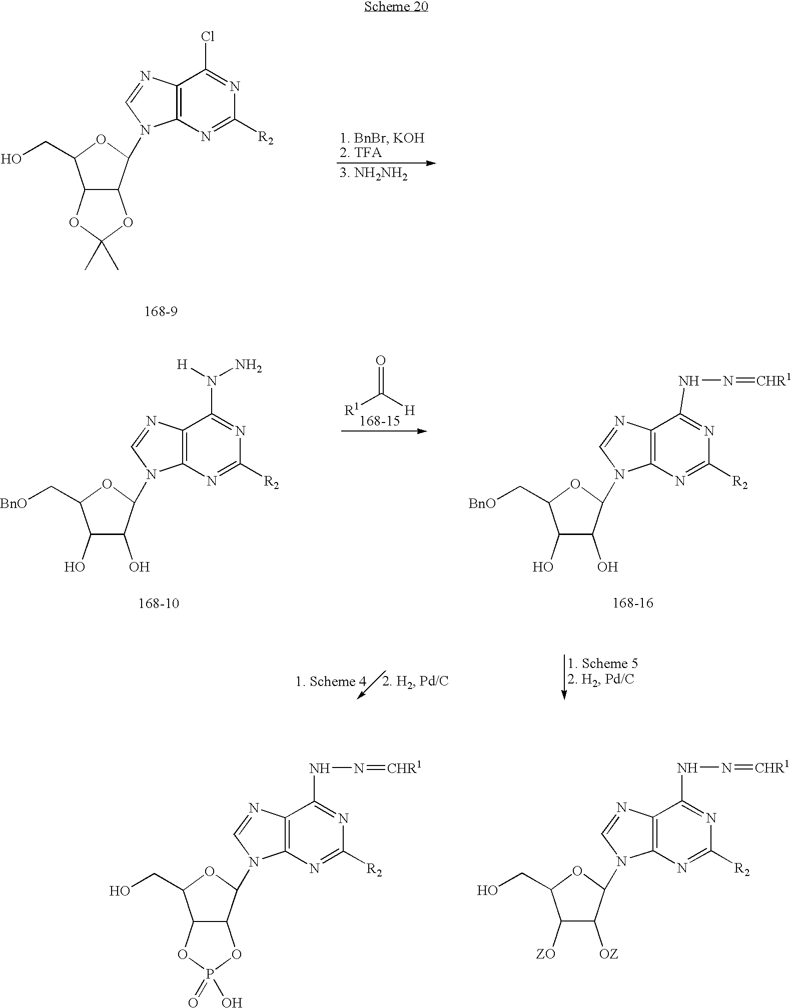
where R¹ and R² are defined above herein for the Purine Compounds of Formula (168-IV) and Z is R⁶ or R⁷ as defined for the Purine Compounds of formula (168-IV).

The 6-chloroadenosine derivatives of Formula 168-9 can be reacted with benzyl bromide in the presence of KOH to provide the corresponding 5′-O-benzyl intermediates. Removal of the isopropylidene group using TFA, followed by reaction with hydrazine, provides the 6-hydrazino derivatives of Formula 168-10. The compounds of Formula 168-10 can then be treated with a carbonyl compound of formula 168-15 to provide the compounds of formula 168-16. The 2′,3′-dihydroxy group of the compounds of formula 168-16 can then be converted to a cyclic phosphate using the methodology set forth in Scheme 4, or alternatively can be converted to a 2′,3′-diester using the methodology set forth in Scheme 5. Removal of the benzyl group using catalytic hydrogenation provides the Purine Compounds of formula (168-IV).

Methodology useful for making the Purine Compounds of Formula (168-V) is outlined in Scheme 21. 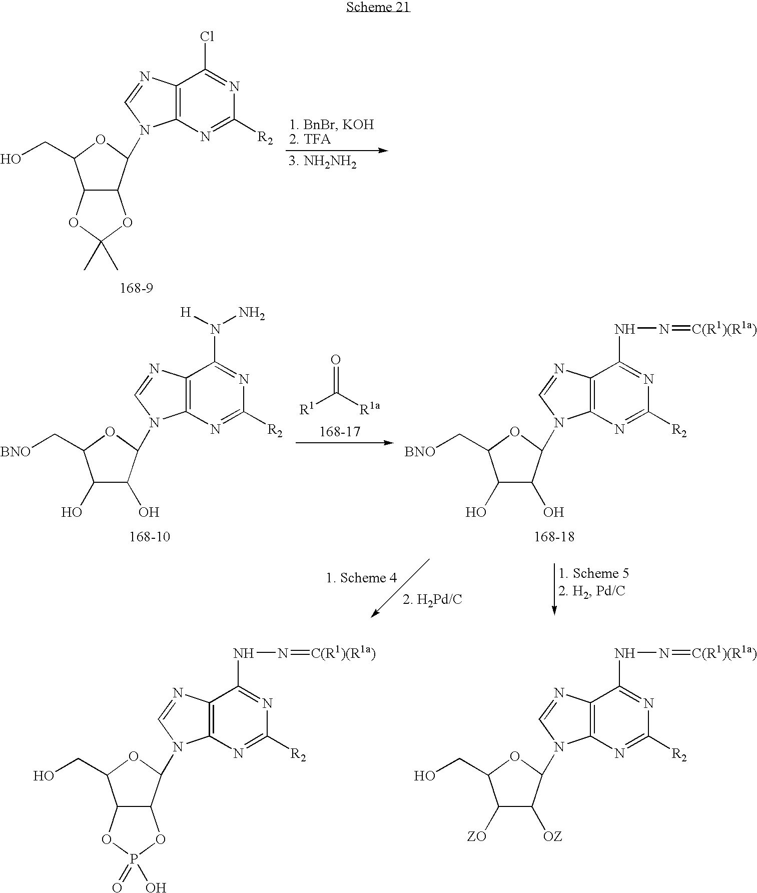
where R¹, R^{1a} and R² are defined above herein for the Purine Compounds of Formula (168-V) and Z is R⁷ or R⁸ as defined for the Purine Compounds of Formula (168-V).

The 6-chloroadenosine derivatives of Formula 168-9 can be reacted with benzyl bromide in the presence of KOH to provide the corresponding 5′-O-benzyl intermediates. Removal of the isopropylidene group using TFA, followed by reaction with hydrazine, provides the 6-hydrazino derivatives of Formula 168-10. The compounds of Formula 168-10 can then be treated with a carbonyl compound of formula 168-17 to provide the compounds of formula 168-18. The 2′,3′-dihydroxy group of the compounds of formula 168-18 can then be converted to a cyclic phosphate using the methodology set forth in Scheme 4, or alternatively can be converted to a 2′,3′-diester using the methodology set forth in Scheme 5. Removal of the benzyl group using catalytic hydrogenation provides the Purine Compounds of formula (168-V).
Methodology useful for making the Purine Compounds of Formula (I), wherein R¹ is cyclopent-1-ol-2-yl is outlined in Scheme 22.

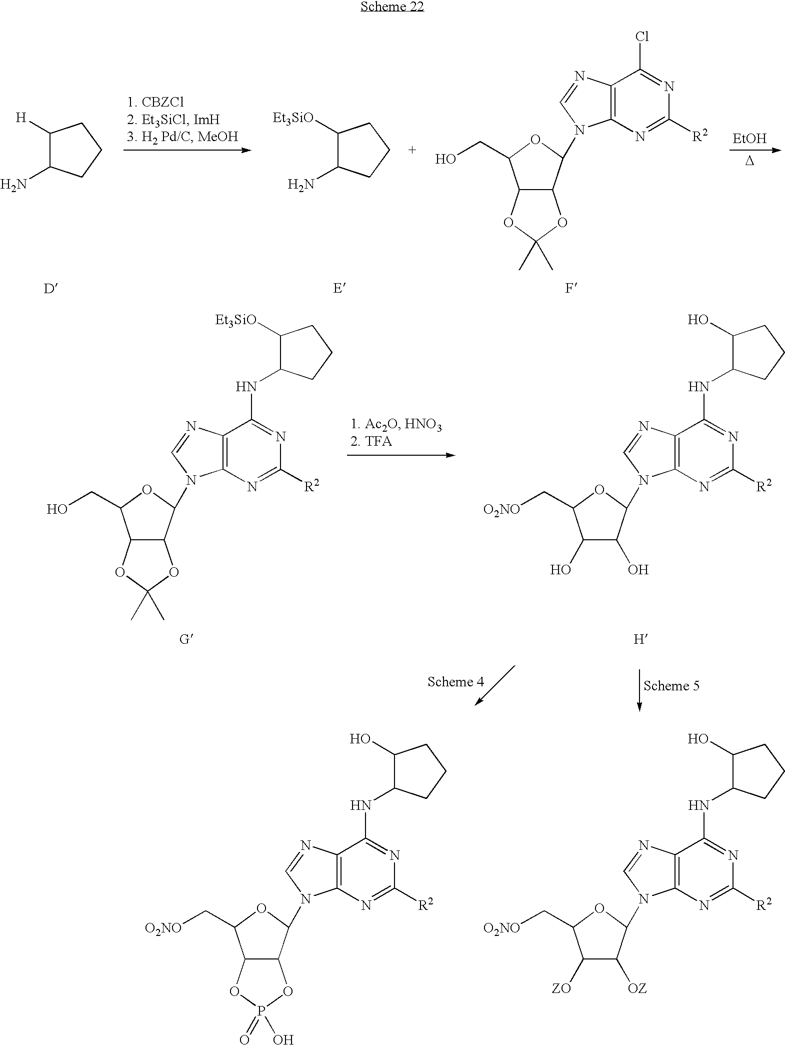

Wherein R² is defined above for the compounds of formula (I) and Z is R² or R³ as defined for the Purine Compounds of Formula (I).

2-Aminocyclopentanol (D′) is reacted with carbobenzyloxy chloride (CBZCl) to protect the amino functionality as its carbobenzyloxy derivative. The hydroxyl group of the carbobenzyloxy derivative is then converted to its corresponding triethylsilyl ether using triethylsilyl chloride in the presence of imidazole. The carbobenzyloxy protecting group is then removed via catalytic hydrogenation to provide amine compound E′. Compound E′ is coupled with compound F′ in refluxing ethanol to provide compound G′, which is subsequently nitrated using acetic anhydride/nitric acid and then reacted with trifluroacetic acid to remove the acetonide group and provide compound H′. The 2′,3′-dihydroxy group of the compound of formula H′ can then be converted to a cyclic phosphate using the methodology set forth in Scheme 4, or alternatively can be converted to a 2′,3′-diester using the methodology set forth in Scheme 5, to make the Purine Compounds of Formula (I), wherein R¹ is cyclopent-1-ol-2-yl. It is to be appreciated that Scheme 22 can give rise to 8 chiral isomers when R¹ is cyclopent-1-ol-2-yl. These isomers of —NHR¹ are depicted as follows: 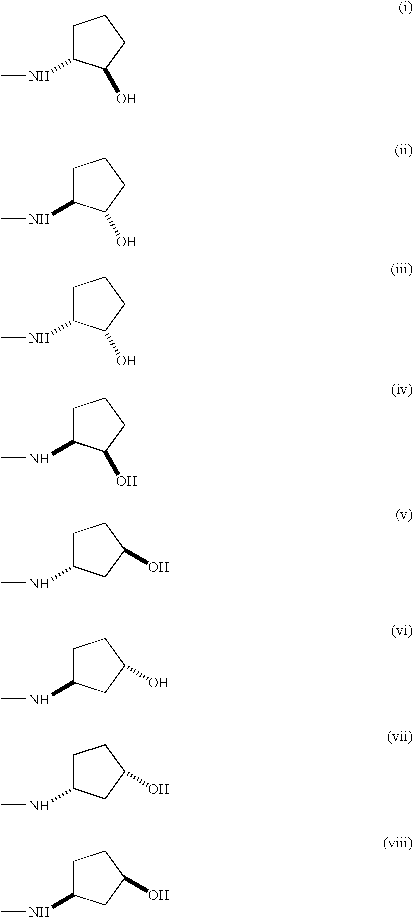
Methodology useful for making the Purine Compounds of Formula (I), wherein R¹ is cyclopent-1-ol-3-yl is outlined in Scheme 23.

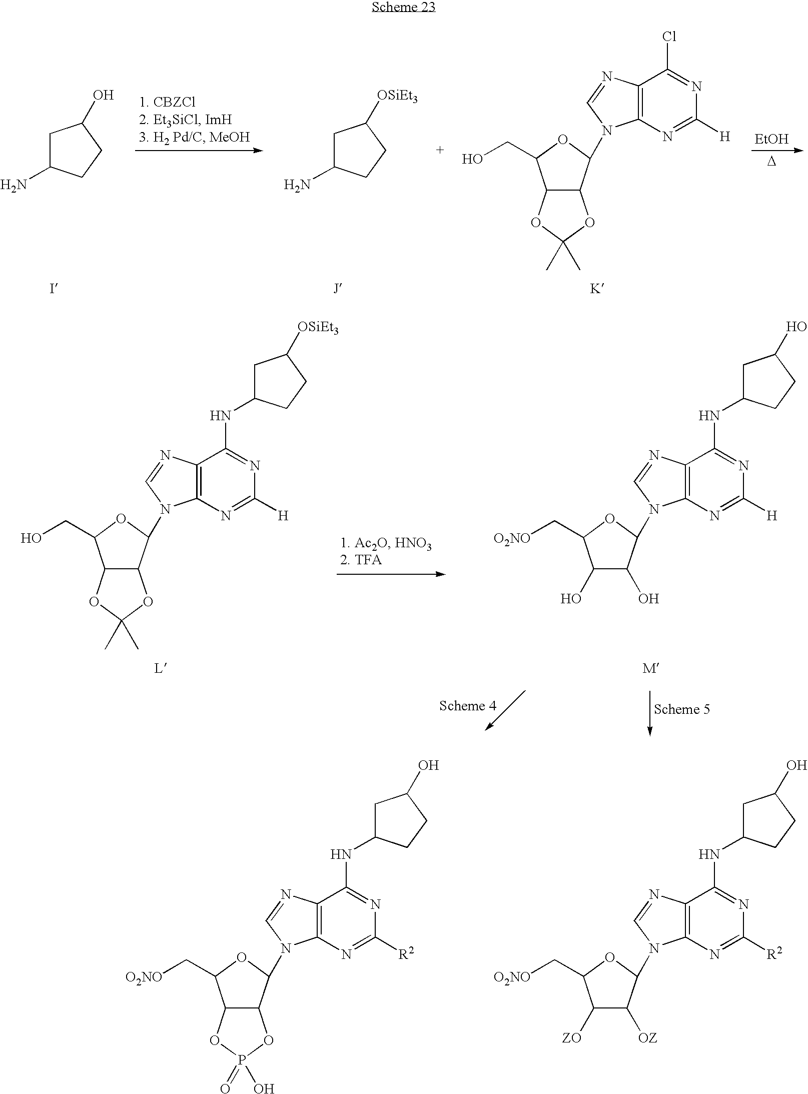

Wherein R² is defined above for the compounds of formula (I) and Z is R² or R³ as defined for the Purine Compounds of formula (I).

3-Aminocyclopentanol (I′) is reacted with CBZCl to protect the amino functionality as its carbobenzyloxy derivative. The hydroxyl group of the carbobenzyloxy derivative is then converted to its corresponding triethylsilyl ether using triethylsilyl chloride in the presence of imidazole. The carbobenzyloxy protecting group is then removed via catalytic hydrogenation to provide amine compound J′. Compound J′ is coupled with compound K′ in refluxing ethanol to provide compound L′, which is subsequently nitrated using acetic anhydride/nitric acid and then reacted with trifluroacetic acid to remove the acetonide group and provide compound M′. The 2′,3′-dihydroxy group of the compound of formula M′ can then be converted to a cyclic phosphate using the methodology set forth in Scheme 4, or alternatively can be converted to a 2′,3′-diester using the methodology set forth in Scheme 5, to make the Purine Compounds of Formula (I), wherein R¹ is cyclopent-1-ol-3-yl.

Scheme 24 shows methodology useful for making Compound P′, which is useful for making Purine Compounds of Formula (II): 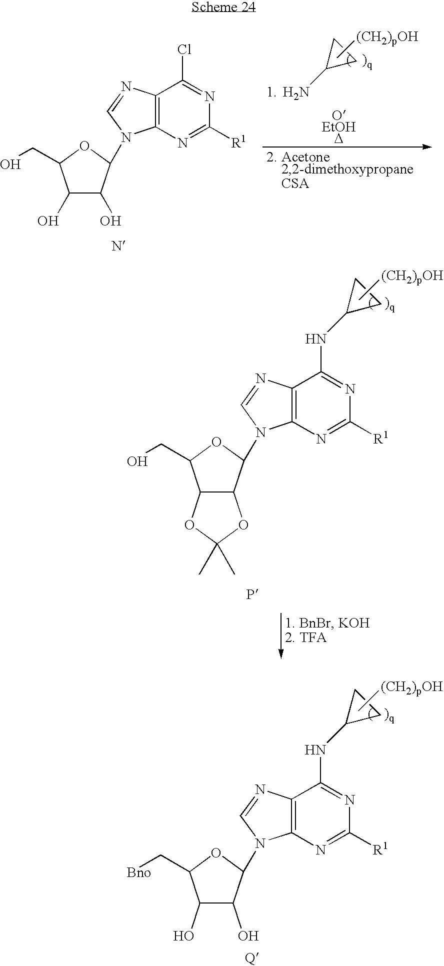
wherein R¹, p and q are as defined herein for the Purine Compounds of Formula (II). A compound of Formula N′ is reacted with a compound of Formula O′ in refluxing ethanol, followed by protection of the 2′,3′-dihydroxy group as its isopropylidene derviative, to provide compounds of Formula P′. The primary hydroxyl groups of the compounds of Formula P′ are then protected as their benzyl ethers, followed by acid-mediated removal of the isopropylidene group to provide the intermediate compounds of Formula Q′.

Scheme 25 illustrates a method for making the Purine Compounds having a 2′,3′-cyclic phosphate: 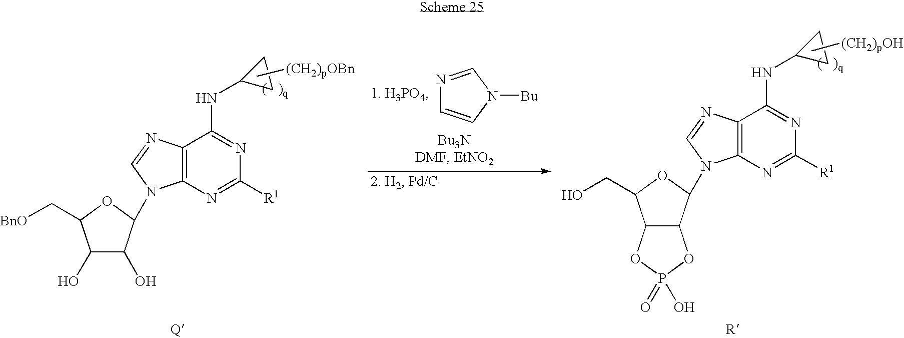
wherein R¹, p and q are as defined herein for the Purine Compounds of Formula (II).
A Purine Compound of Formula Q can be reacted with phosphoric acid in the presence of 1-butylimidazole and n-butylamine in a mixture of N,N-dimethylformamide (DMF) and nitroethane as described in Sakakura et al., Org. Letters 7:1999-2002 (2005) to provide the corresponding 2′,3′-cyclic phosphate derivative. The cyclic phosphate derviative is then subjected to catalytic hydrogenation to remove the benzyl protecting groups and provide a Purine Compound of Formula (II) having a 2′,3′-cyclic phosphate.

Scheme 26 illustrates a method-for making the Purine Compounds having a 2′,3′-diester, wherein the esters are derived from a naturally occurring amino acid: 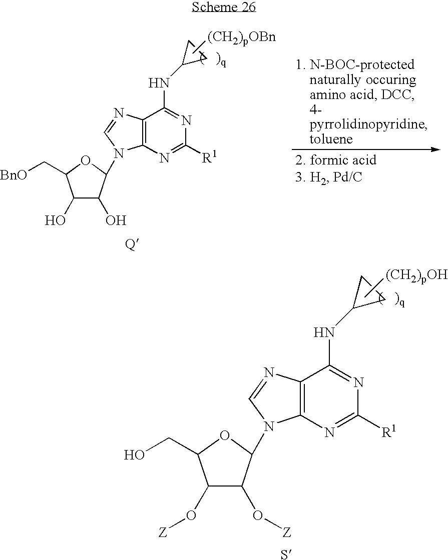
wherein R¹, p and q are as defined herein for the Purine Compounds of Formula (II); and Z is R³ or R⁴ as defined herein for the Purine Compounds of Formula (II). Scheme 27 sets forth methodology useful for making the compounds of Formula 169-9, wherein p is 1 and q is defined above for the Purine Derivatives of Formula (II): 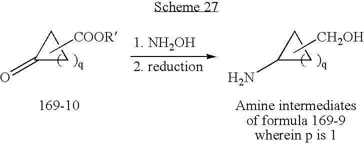
wherein R′ is —H or methyl, p is 1 and q is defined above for the Purine Derivatives of Formula (II).

A compound of Formula 169-10 is reacted with hydroxylamine in a solvent such as ethanol, and the resultant oxime is reduced, using for example, lithium aluminum hydride, to provide the compounds of Formula 169-9, wherein p is 1 and q is 1, 2, 3, 5 or 6.

The compounds of Formula 169-10 are commercially available, or alternatively, can be prepared from commercially available starting materials using methods known to one skilled in the art of organic synthesis. For example, 1,2-substituted keto-esters of Formula 169-10 can be synthesized by reacting a cycloalkanone enolate (prepared from a commercially available cycloalkanone) with an alkyl chloroformate; 1,3-substituted keto-esters of Formula 169-10 can be synthesized via 1,4 addition to a commercially available conjugated cycloalkenone; and 1,4-substituted keto-esters of Formula 169-10 can be synthesized via oxidation of commercially available 4-carboxylate substituted cycloalkanols.

Scheme 28 sets forth methodology useful for making the compounds of Formula 169-9, wherein p is an integer ranging from 3-6 and q is defined above for the Purine Deriviatives of Formula (II): 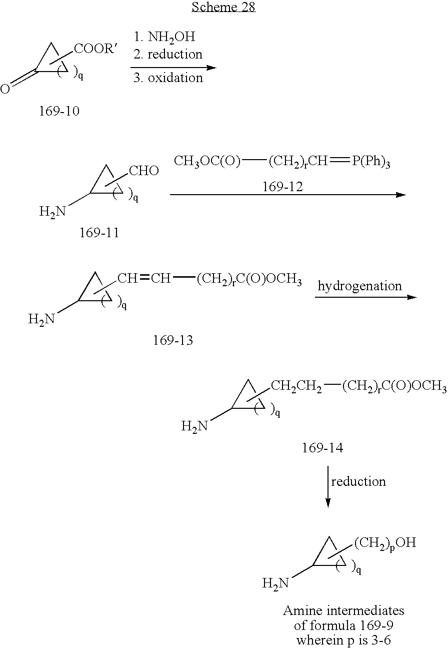
wherein R′ is —H or methyl, p is an integer ranging from 3 to 6, q is defined above for the Purine Derivatives of Formula (II), and r is an integer ranging from 0 to 3.

A compound of Formula 169-10 is reacted with hydroxylamine, and the resultant oxime is reduced, using for example, diisobutylaluminum hydride (DIBAL), followed by oxidation of the resultant —CH₂OH group, to provide a compound of Formula 169-11. The compound of Formula 169-11 can be reacted with a compound of Formula 169-12 via a Wittig reaction to provide a compound of Formula 169-13 (See March, Advanced Organic Chemistry: Reactions, Mechanisms, and Structure, 956-963 (4^{th} ed 1992)). Hydrogenation of the compound of Formula 169-13, using for example H₂ and Pd/C, provides the compound of Formula 169-14, which can then be reduced using, for example, lithium aluminum hydride to provide the compounds of Formula 169-9, wherein p is an integer ranging from 3-6 and q is defined above for the Purine Derivatives of Formula (II).

Scheme 29 sets forth methodology useful for making the amine intermediates of Formula 169-9, wherein p is 2 and q is defined above for the Purine Deriviatives of Formula (II): 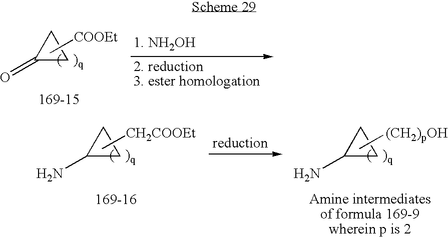
wherein R′ is —H or methyl, p is an integer ranging from 3 to 6, and q is defined above for the Purine Derivatives of Formula (I).

A compound of Formula 169-15 can be converted to the corresponding amine by reacting 169-15 with hydroxylamine followed by selective reduction of the resultant oxime using, for example, magnesium in the presence of ammonium formate (See Abiraj et al., Synth. Commun. 34:599-605 (2004)). A methylene group is then inserted between the the ethyl ester group and the carbocyclic ring of 169-15 using, for example, a Kowalski ester homologation reaction (Kowalski et al., J. Am. Chem. Soc. 57:7194 (1992)) to provide a compound of Formula 169-16. The compound of Formula 169-16 can then be reduced to the corresponding alcohol using, for example, lithium aluminum hydride to provide the compounds of Formula 169-9, wherein p is 2 and q is defined above for the Purine Derivatives of Formula (I).

The compounds of Formula 169-15 are commercially available, or alternatively, can be prepared from commercially available starting materials using methods known to one skilled in the art of organic synthesis.
Scheme 30 shows methodology useful for making Purine Compounds of Formula (III). 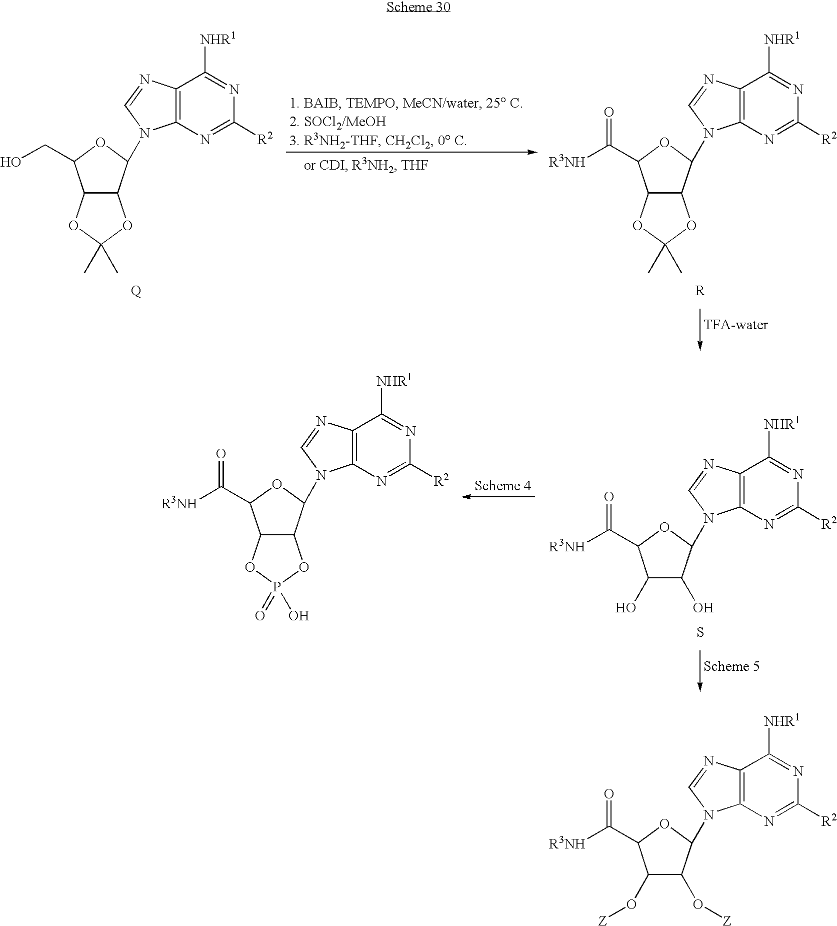
wherein R¹ and R² are as defined above herein for the Purine Compounds of Formula (III), and Z is R⁵ or R⁶ as defined above for the Purine Compounds of Formula (III).
The hydroxymethyl group of the compounds of formula Q are converted to the amides of formula R using a three-step procedure. The hydroxyl group of Q is first oxidized using TEMPO (2,2,6,6-tetramethyl-1-piperidinyloxy, free radical) to provide the corresponding carboxylic acid intermediates, which are then converted to the corresponding acid chloride or ester intermediates using thionyl chloride in methanol. The acid chloride intermediates are then coupled with an amine of formula R³NH₂ to provide the amide compounds of formula R. The acetonide protecting group of the compounds of formula R is then removed using TFA to provide compounds of formula S. The 2′,3′-dihydroxy group of the compounds of formula S can then be converted to a cyclic phosphate using the methodology set forth in Scheme 4, or alternatively can be converted to a 2′,3′-diester using the methodology set forth in Scheme 5, to provide the Purine Compounds of Formula (III).

Scheme 31 shows a method useful for making the Purine Compounds of Formula (III). 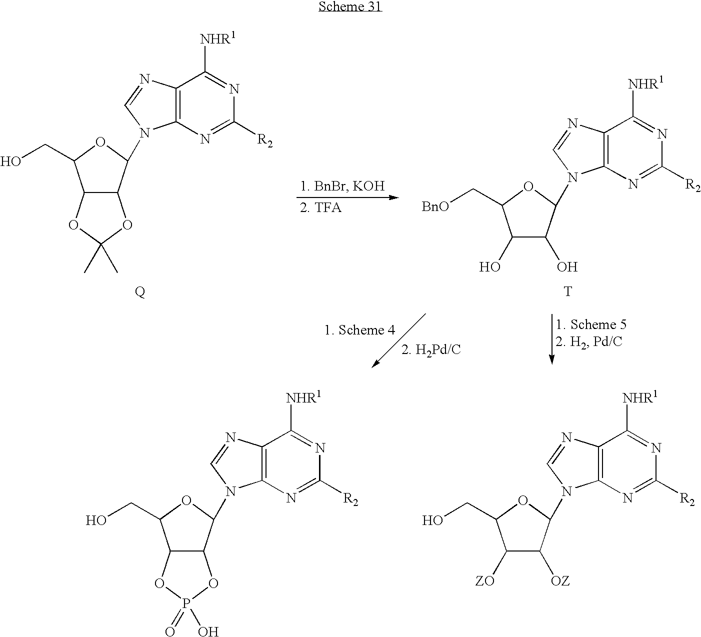
wherein R¹ and R² are as defined above herein for the Purine Compounds of Formula (III), and Z is R⁴ or R⁵ as defined above for the Purine Compounds of Formula (III).

The intermediate compounds of formula Q can be treated with benzyl bromide in the presence of a base, such as potassium hydroxide, followed by removal of the 2′,3′-isopropylidene group in acid to provide the 2′,3′-dihydroxy compounds of formula T. The 2′,3′-dihydroxy group of the compounds of formula T can then be converted to a cyclic phosphate using the methodology set forth in Scheme 4 to provide the Purine Compounds of Formula (III), or alternatively can be converted to a 2′,3′-diester using the methodology set forth in Scheme 5, to provide the Purine Compounds of Formula (III).
Methodology useful for making the Purine Compounds of Formulas (170-III) and (170-VIII) is outlined in Scheme 32. 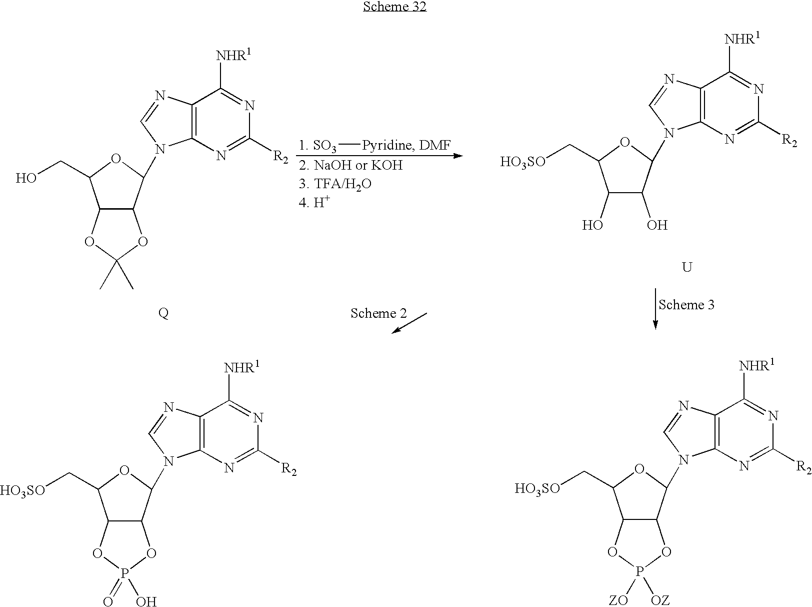
wherein R¹ and R² are as defined above herein for the Purine Compounds of Formulas (170-III) and (170-VIII), and Z is R⁴ or R⁵ as defined above-for the Purine Compounds of Formula (170-VIII).

The adenosine intermediates of formula Q can be treated with sulfur trioxide-pyridine complex to provide the corresponding 5′-sulfonic acid pyridine salt intermediate. The pyridine salt intermediate can then be neutralized using NaOH or KOH, followed by acetonide removal using TFA/water to provide the corresponding sulfonic acid sodium or potassium salt. Treatment of the sodium or potassium salt with strong aqueous acid, such as sulfuric or hydrochloric acid, provides the Purine Compounds of Formula U. The 2′,3′-dihydroxy group of the compounds of formula U can then be converted to a cyclic phosphate using the methodology set forth in Scheme 4 to provide the Purine Compounds of Formula (III), or alternatively can be converted to a 2′,3′-diester using the methodology set forth in Scheme 5, to provide the Purine Compounds of Formula (170-VIII).
Further methodology useful for making Purine Compounds of Formula (III) is described in Scheme 33. 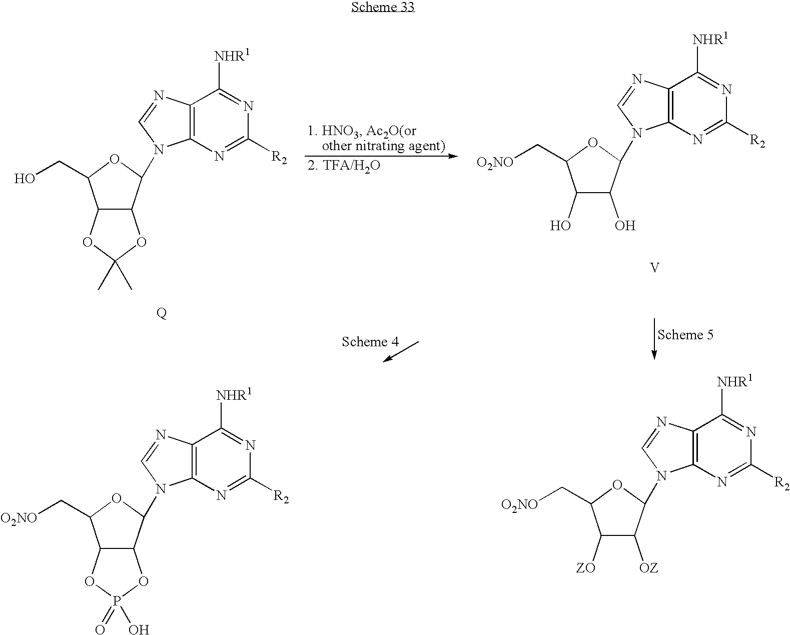
wherein R¹ and R² are as defined above herein for the Purine Compounds of Formulas (III), and Z is R⁴ or R⁵ as defined above for the Purine Compounds of Formula (III).

The adenosine intermediates of formula Q can be converted to their 5′-nitrate analogs using nitric acid in the presence of acetic anhydride, or other nitrating agents, such as MsCl/ONO₃ or nitrosonium tetrafluoroborate. Acetonide removal using TFA/water provides Purine Compounds of Formula V. The 2′,3′-dihydroxy group of the compounds of formula V can then be converted to a cyclic phosphate using the methodology set forth in Scheme 4, or alternatively can be converted to a 2′,3′-diester using the methodology set forth in Scheme 5, to provide the Purine Compounds of Formula (III).

Methodology useful for making Purine Compounds of Formulas (III), wherein n is 1 is described in Scheme 34. 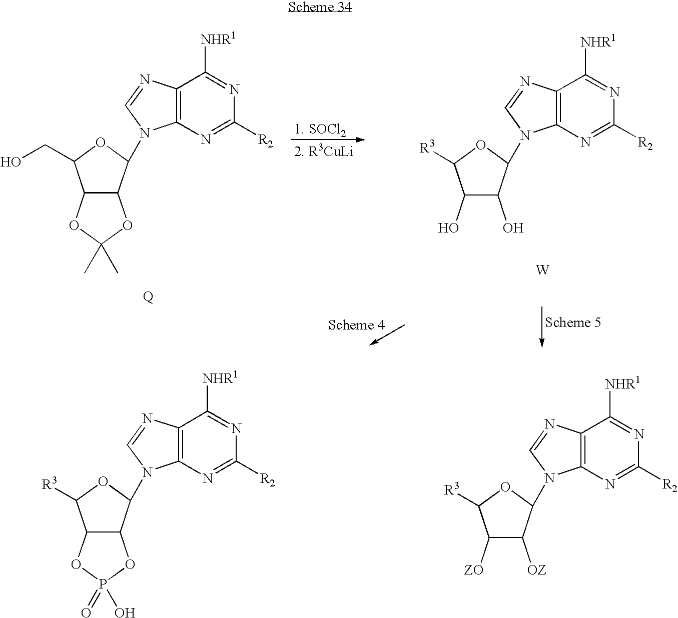
wherein R¹ and R² are as defined above herein for the Purine Compounds of Formulas (III), R³ is —(C₁-C₆ alkylene)ₙ-(3- to 7-membered monocyclic heterocycle) or —(C₁-C₆ alkylene)ₙ-(8- to 12-membered bicyclic heterocycle), wherein n is 1; and Z is R⁵ or R⁶ as defined above for the Purine Compounds of Formula (III).

The 5′-hydroxyl group of the adenosine intermediates of formula Q can reacted with thionyl chloride to provide the corresponding 5′-chloro derivative, which can then be coupled with a cuprate of formula R³CuLi to provide the compounds of formula W. The 2′,3′-dihydroxy group of the compounds of formula W can then be converted to a cyclic phosphate using the methodology set forth in Scheme 4 or alternatively can be converted to a 2′,3′-diester using the methodology set forth in Scheme 5, to provide the Purine Compounds of Formula (III), wherein R³ is —(C₁-C₆ alkylene)-(3- to 7-membered monocyclic heterocycle) or —(C₁-C₆ alkylene)-(8- to 12-membered bicyclic heterocycle).
Methodology useful for making Purine Compounds of Formulas (170-V) and ( 170-X), wherein n is 0 is described in Scheme 35. 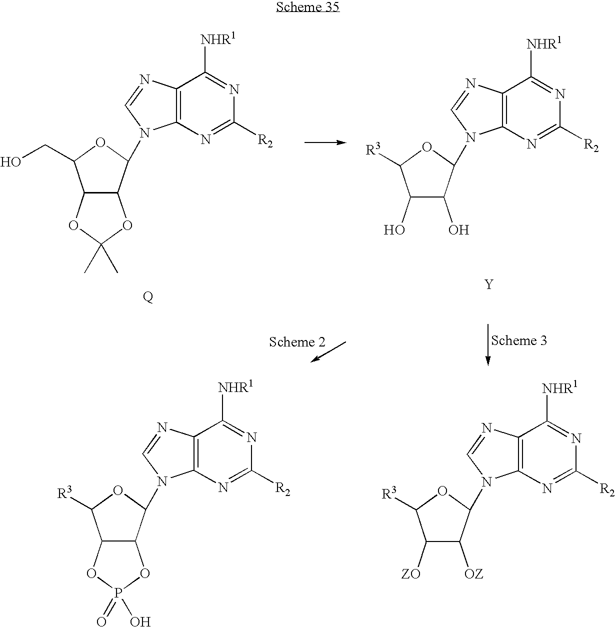
wherein R¹ and R² are as defined above herein for the Purine Compounds of Formulas (170-V) and (170-X); R³ is —(C₁-C₆ alkylene)ₙ-(3- to 7-membered monocyclic heterocycle) or —(C₁-C₆ alkylene)ₙ-(8- to 12-membered bicyclic heterocycle), wherein n is 0; and Z is R⁵ or R⁶ as defined above for the Purine Compounds of Formula (170-X).

The 5′-hydroxyl group of the adenosine intermediates of formula Q can converted to R³, wherein R³ is -3- to 7-membered monocyclic heterocycle or -8- to 12-membered bicyclic heterocycle, using chemistry well-known to one skilled in the art of organic synthesis, such as the methods set forth in March et al; Advanced Organic Chemistry, Appendix B, pages 1286-1287 (4th ed. 1992), to provide the compounds of formula Y. The 2′,3′-dihydroxy group of the compounds of formula Y can then be converted to a cyclic phosphate using the methodology set forth in Scheme 4 to provide the Purine Compounds of Formula (170-V), or alternatively can be converted to a 2′,3′-diester using the methodology set forth in Scheme 5, to provide the Purine Compounds of Formula (170-X), wherein R³ is -3- to 7-membered monocyclic heterocycle or -8- to 12-membered bicyclic heterocycle.

### 5.4 THERAPEUTIC/PROPHYLACTIC ADMINISTRATION AND COMPOSITIONS OF THE INVENTION

Due to their activity, the Purine Compounds are advantageously useful in veterinary and human medicine. As described above, the Purine Compounds are useful for: (i) treating or preventing a Condition in a subject in need thereof; (ii) reducing a subject's rate of metabolism; or (iii) protecting a subject's heart against myocardial damage during cardioplegia.

When administered to a subject, the Purine Compounds can be administered as a component of a composition that comprises a physiologically acceptable carrier or vehicle. The present compositions, which comprise a Purine Compound, can be administered orally. The Purine Compounds can also be administered by any other convenient route, for example, by infusion or bolus injection, by absorption through epithelial (e.g., skin) or mucocutaneous linings (e.g., oral, rectal, and intestinal mucosa, etc.), by intratracheal administration, or by inhalation, and can be administered together with another biologically active agent. Administration can be systemic or local. Various known delivery systems, including encapsulation in liposomes, microparticles, microcapsules, and capsules, can be used.

Methods of administration include, but are not limited to, intradermal, intratracheal, intramuscular, intraperitoneal, intravenous, subcutaneous, intranasal, epidural, oral, sublingual, intracerebral, intravaginal, transdermal, rectal, by inhalation, or topical, particularly to the ears, nose, eyes, or skin. In some instances, administration results in the release of the Purine Compounds into the bloodstream. The mode of administration can be left to the discretion of the practitioner.

In one embodiment, the Purine Compounds are administered orally.

In another embodiment, the Purine Compounds are administered intravenously.

In another embodiment, the Purine Compounds are administered topically.

In still another embodiment, the Purine Compounds are administered via inhalation.

In a further embodiment, the Purine Compounds are administered intratracheally.

In other embodiments, it can be desirable to administer the Purine Compounds locally. This can be achieved, for example, and not by way of limitation, by local infusion during surgery, topical application, (e.g., directly to a wound or in conjunction with a wound dressing), by injection, by intubation, by means of a catheter, by means of a suppository or enema, or by means of an implant, said implant being of a porous, non-porous, or gelatinous material, including membranes, such as sialastic membranes, or fibers.

In certain embodiments, it can be desirable to introduce the Purine Compounds into the central nervous system, circulatory system or gastrointestinal tract by any suitable route, including intraventricular, intrathecal injection, paraspinal injection, epidural injection, enema, and by injection adjacent to a peripheral nerve. Intraventricular injection can be facilitated by an intraventricular catheter, for example, attached to a reservoir, such as an Ommaya reservoir.

In yet other embodiments, it can be desirable to administer the Purine Compounds ocularly. Ocular administration of the Purine Compounds can be achieved using an eye-dropper or a contact lens coated or impregnated with the Purine Compound.

Pulmonary administration can also be employed, e.g., by use of an inhaler of nebulizer, by intubation, and formulation with an aerosolizing agent, or via perfusion in a fluorocarbon or synthetic pulmonary surfactant. In certain embodiments, the Purine Compounds can be formulated as a suppository, with traditional binders and excipients such as triglycerides.

In another embodiment the Purine Compounds can be delivered in a vesicle, in particular a liposome (see Langer, Science 249:1527-1533 (1990) and Lopez-Berestein et al., Liposomes in the Therapy of Infectious Disease and Cancer 317-327 and 353-365 (1989)).

In yet another embodiment the Purine Compounds can be delivered in a controlled-release system or sustained-release system (see, e.g., Goodson, in Medical Applications of Controlled Release, szipra, vol. 2, pp. 115-138 (1984)). Other controlled or sustained-release systems discussed in the review by Langer, Science 249:1527-1533 (1990) can be used. In one embodiment a pump can be used (Langer, Science 249:1527-1533 (1990); Sefton, CRC Crit. Ref. Biomed. Eng. 14:201 (1987); Buchwald et al., Surgery 88:507 (1980); and Saudek et al., N. Engl. J Med. 321:574 (1989)). In another embodiment polymeric materials can be used (see Medical Applications of Controlled Release (Langer and Wise eds., 1974); Controlled Drug Bioavailability, Drug Product Design and Performance (Smolen and Ball eds., 1984); Ranger and Peppas, J. Macromol. Sci. Rev. Macromol. Chem. 2:61 (1983); Levy et al., Science 228:190 (1935); During et al., Ann. Neural. 25:351 (1989); and Howard et al., J. Neurosurg. 71:105 (1989)).

In yet another embodiment a controlled- or sustained-release system can be placed in proximity of a target of the Purine Compounds, e.g., the spinal column, brain, colon, skin, heart, lung, eye, trachea or gastrointestinal tract, thus requiring only a fraction of the systemic dose.

The present compositions can optionally comprise a suitable amount of a physiologically acceptable excipient.

Such physiologically acceptable excipients can be liquids, such as water and oils, including those of petroleum, animal, vegetable, or synthetic origin, such as peanut oil, soybean oil, mineral oil, sesame oil and the like. The physiologically acceptable excipients can be saline, gum acacia, gelatin, starch paste, talc, keratin, colloidal silica, urea and the like. In addition, auxiliary, stabilizing, thickening, lubricating, and coloring agents can be used. In one embodiment the physiologically acceptable excipients are sterile when administered to a subject. Water is a particularly useful excipient when the Purine Compound is administered intravenously. Saline solutions and aqueous dextrose and glycerol solutions can also be employed as liquid excipients, particularly for injectable solutions. Suitable physiologically acceptable excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

The present compositions can take the form of solutions, suspensions, tablets, pills, pellets, capsules, capsules containing liquids, powders, sustained-release formulations, suppositories, emulsions, aerosols, sprays or any other form suitable for use. In one embodiment the composition is in the form of a capsule. Other examples of suitable physiologically acceptable excipients are described in Remington's Pharmaceutical Sciences 1447-1676 (Alfonso R. Gennaro eds., 19th ed. 1995), incorporated herein by reference.

In one embodiment the Purine Compounds are formulated in accordance with routine procedures as a composition adapted for oral administration to human beings. Compositions for oral delivery can be in the form of tablets, lozenges, aqueous or oily suspensions, granules, powders, emulsions, capsules, syrups, or elixirs for example. Orally administered compositions can contain one or more agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions can be coated to delay disintegration and absorption in the gastrointestinal tract thereby providing a sustained action over an extended period of time. Selectively permeable membranes surrounding an osmotically active platform driving a Purine Compound are also suitable for orally administered compositions. In these latter platforms, fluid from the environment surrounding the capsule can be imbibed by the driving compound, which swells to displace the agent or agent composition through an aperture. These delivery platforms can provide an essentially zero order delivery profile as opposed to the spiked profiles of immediate release formulations. A time-delay material such as glycerol monostearate or glycerol stearate can also be used. Oral compositions can include standard excipients such as mannitol, lactose, starch, magnesium stearate, sodium saccharin, cellulose, and magnesium carbonate. In one embodiment the excipients are of pharmaceutical grade.

In another embodiment the Purine Compounds can be formulated for intravenous administration. Typically, compositions for intravenous administration comprise sterile isotonic aqueous buffer. Where necessary, the compositions can also include a solubilizing agent. Compositions for intravenous administration can optionally include a local anesthetic such as lignocaine to lessen pain at the site of the injection. Generally, the ingredients are supplied either separately or mixed together in unit dosage form, for example, as a dry lyophilized powder or water- free concentrate in a hermetically sealed container such as an ampule or sachette indicating the quantity of active agent. Where the Purine Compounds are to be administered by infusion, they can be dispensed, for example, with an infusion bottle containing sterile pharmaceutical grade water or saline. Where the Purine Compounds are administered by injection, an ampule of sterile water for injection or saline can be provided so that the ingredients can be mixed prior to administration.

The Purine Compounds can be administered by controlled-release or sustained-release means or by delivery devices that are well known to those of skill in the art. Such dosage forms can be used to provide controlled- or sustained-release of one or more active ingredients using, for example, hydroxypropylmethyl cellulose, other polymer matrices, gels, permeable membranes, osmotic systems, multilayer coatings, microparticles, liposomes, microspheres, or a combination thereof to provide the desired release profile in varying proportions. Suitable controlled- or sustained-release formulations known to those skilled in the art, including those described herein, can be readily selected for use with the active ingredients of the invention. The invention thus encompasses single unit dosage forms suitable for oral administration such as, but not limited to, tablets, capsules, gelcaps, and caplets that are adapted for controlled- or sustained-release.

In one embodiment a controlled- or sustained-release composition comprises a minimal amount of a Purine Compound to treat or prevent the Condition, reduce a subject's rate of metabolism or protect a subject's heart against myocardial damage during cardioplegia in a minimal amount of time. Advantages of controlled- or sustained-release compositions include extended activity of the drug, reduced dosage frequency, and increased patient compliance. In addition, controlled- or sustained-release compositions can favorably affect the time of onset of action or other characteristics, such as blood levels of the Purine Compound, and can thus reduce the occurrence of adverse side effects.

Controlled- or sustained-release compositions can initially release an amount of a Purine Compound that promptly produces the desired therapeutic or prophylactic effect, and gradually and continually release other amounts of the Purine Compound to maintain this level of therapeutic or prophylactic effect over an extended period of time. To maintain a constant level of the Purine Compound in the body, the Purine Compound can be released from the dosage form at a rate that will replace the amount of Purine Compound being metabolized and excreted from the body. Controlled- or sustained-release of an active ingredient can be stimulated by various conditions, including but not limited to, changes in pH, changes in temperature, concentration or availability of enzymes, concentration or availability of water, or other physiological conditions or compounds.

The amount of the Purine Compound that is effective for treating or preventing a Condition, reducing a subject's rate of metabolism, or protecting a subject's heart against myocardial damage during cardioplegia, can be determined by standard clinical techniques. In addition, in vitro or in vivo assays can optionally be employed to help identify optimal dosage ranges. The precise dose to be employed can also depend on the route of administration, and the seriousness of the condition being treated and can be decided according to the judgment of a health-care practitioner. Suitable effective dosage amounts, however, range from about 10 micrograms to about 5 grams about every 4 h, although they are typically about 500 mg or less per every 4 hours. In one embodiment the effective dosage is about 0.01 mg, 0.5 mg, about 1 mg, about 50 mg, about 100 mg, about 200 mg, about 300 mg, about 400 mg, about 500 mg, about 600 mg, about 700 mg, about 800 mg, about 900 mg, about 1 g, about 1.2 g, about 1.4 g, about 1.6 g, about 1.8 g, about 2.0 g, about 2.2 g, about 2.4 g, about 2.6 g, about 2.8 g, about 3.0 g, about 3.2 g, about 3.4 g, about 3.6 g, about 3.8 g, about 4.0 g, about 4.2 g, about 4.4 g, about 4.6 g, about 4.8 g, and about 5.0 g, every 4 hours. Equivalent dosages may be administered over various time periods including, but not limited to, about every 2 hours, about every 6 hours, about every 8 hours, about every 12 hours, about every 24 hours, about every 36 hours, about every 48 hours, about every 72 hours, about every week, about every two weeks, about every three weeks, about every month, and about every two months. The number and frequency of dosages corresponding to a completed course of therapy can be determined according to the judgment of a health-care practitioner. The effective dosage amounts described herein refer to total amounts administered; that is, if more than one Purine Compound is administered, the effective dosage amounts correspond to the total amount administered.

The amount of a Purine Compound that is effective for treating or preventing a Condition, reducing a subject's rate of metabolism, or protecting a subject's heart against myocardial damage during cardioplegia, typically ranges from about 0.01 mg/kg to about 100 mg/kg of body weight per day, in one embodiment, from about 0.1 mg/kg to about 50 mg/kg body weight per day, and in another embodiment, from about 1 mg/kg to about 20 mg/kg of body weight per day.

The amount of a Purine Compound that is effective for reducing a subject's rate of metabolism or a subject's core body temperature typically range from about about 1 μg/kg to about 10 mg/kg, in one embodiment, from about 0.1 mg/kg to about 5 mg/kg body weight per day, and in another embodiment, from about 1 mg/kg to about 2.5 mg/kg of body weight per day.

When a Purine Derviative is a component of a solution that is useful for maintaining the viability of an organ ex vivo, the concentration of the Purine Compound in the solution that is effective for maintaining the viability of the organ is between about 1 nM to about 1 mM.

The Purine Compounds can be assayed in vitro or in vivo for the desired therapeutic or prophylactic activity prior to use in humans. Animal model systems can be used for safety and efficacy.

The present methods for treating or preventing a Condition, reducing a subject's rate of metabolism, or protecting a subject's heart against myocardial damage during cardioplegia, can further comprise administering another therapeutic agent to the subject being administered a Purine Compound. In one embodiment the other therapeutic agent is administered in an effective amount.

Effective amounts of the other therapeutic agents are well known to those skilled in the art. However, it is well within the skilled artisan's purview to determine the other therapeutic agent's optimal effective amount range. In one embodiment of the invention, where, another therapeutic agent is administered to a subject, the effective amount of the Purine Compound is less than its effective amount would be where the other therapeutic agent is not administered. In this case, without being bound by theory, it is believed that the Purine Compounds and the other therapeutic agent act synergistically.

In one embodiment the other therapeutic agent is an anti-inflammatory agent. Examples of useful anti-inflammatory agents include, but are not limited to, adrenocorticosteroids, such as cortisol, cortisone, fluorocortisone, prednisone, prednisolone, 6α-methylprednisolone, triamcinolone, betamethasone, and dexamethasone; and non-steroidal anti-inflammatory agents (NSAIDs), such as aspirin, acetaminophen, indomethacin, sulindac, tolmetin, diclofenac, ketorolac, ibuprofen, naproxen, flurbiprofen, ketoprofen, fenoprofen, oxaprozin, mefenamic acid, meclofenamic acid, piroxicam, meloxicam, nabumetone, rofecoxib, celecoxib, etodolac, and nimesulide.

In another embodiment the other therapeutic agent is an anti-diabetic agent. Examples of useful anti-diabetic agents include, but are not limited to, glucagons; somatostatin; diazoxide; sulfonylureas, such as tolbutamide, acetohexamide, tolazamide, chloropropamide, glybenclamide, glipizide, gliclazide, and glimepiride; insulin secretagogues, such as repaglinide, and nateglinide; biguanides, such as metformin and phenformin; thiazolidinediones, such as pioglitazone, rosiglitazone, and troglitazone; and α-glucosidase inhibitors, such as acarbose and miglitol.

In another embodiment, the other therapeutic agent is an anti-glaucoma agent. Examples of anti-glaucoma agents include, but are not limited to, apraclonidine HCl, brimonidine tartrate, dipivefrin HCl, epinephrine HCl, betaxolol HCl, carteolol HCl, levobunolol HCl, metipranolol HCl, timolol, timolol maleate, pilocarpine HCl, pilocarpine, dorzolamide HCl, brinzolamide and latanoprost.

In a further embodiment the other therapeutic agent is an anti-cardiovascular-disease agent. Examples of useful anti-cardiovascular-disease agents include, but are not limited to, carnitine; thiamine; and muscarinic receptor antagonists, such as atropine, scopolamine, homatropine, tropicamide, pirenzipine, ipratropium, tiotropium, and tolterodine. In another embodiment the other therapeutic agent is an analgesic agent. Examples of useful analgesic agents include, but are not limited to, buprenorphine, meperidine, morphine, codeine, propoyxphene, fentanyl, sufentanil, etorphine hydrochloride, hydrocodone, hydromorphone, nalbuphine, butorphanol, oxycodone, aspirin, ibuprofen, naproxen sodium, acetaminophen, xylazine, metedomidine, carprofen, naprosin, and pentazocine.

In a specific embodiment, the other therapeutic agent is buprenorphine.

In another embodiment the other therapeutic agent is an anti-emetic agent. Suitable anti-emetic agents include, but are not limited to, metoclopromide, domperidone, prochlorperazine, promethazine, chlorpromazine, trimethobenzamide, ondansetron, granisetron, hydroxyzine, acethylleucine monoethanolamine, alizapride, azasetron, benzquinamide, bietanautine, bromopride, buclizine, clebopride, cyclizine, dimenhydrinate, diphenidol, dolasetron, meclizine, methallatal, metopimazine, nabilone, oxyperndyl, pipamazine, scopolamine, sulpiride, tetrahydrocannabinols, thiethylperazine, thioproperazine and tropisetron.

A Purine Compound and the other therapeutic agent can act additively or, in one embodiment, synergistically. In one embodiment, a Purine Compound is adminsitered concurrently with another therapeutic agent. In another embodiment, the present compositions can further comprise another therapeutic agent. In a further embodiment, a composition comprising an effective amount of a Purine Compound and an effective amount of another therapeutic agent can be administered. Alternatively, a composition comprising an effective amount of a Purine Compound and a different composition comprising an effective amount of another therapeutic agent can be concurrently administered. In another embodiment, an effective amount of a Purine Compound is administered prior or subsequent to administration of an effective amount of another therapeutic agent. In this embodiment, the Purine Compound is administered while the other therapeutic agent exerts its therapeutic effect, or the other therapeutic agent is administered while the Purine Compound exerts its preventative or therapeutic effect for treating or preventing a Condition, reducing a subject's rate of metabolism or protecting a subject's heart against myocardial damage during cardioplegia.

In another embodiment, the other therapeutic agent is an hematopoietic colony stimulating factor. Suitable hematopoietic colony stimulating factors include, but are not limited to, filgrastim, sargramostim, molgramostim and epoietin alfa.

In still another embodiment, the other therapeutic agent is an analgesic agent. In one embodiment, the analgesic agent is an opioid analgesic. In another embodiment, the analgesic ia a non-opioid analgesic agent. Suitable opioid analgesic agents include, but are not limited to, morphine, heroin, codeine, nalbuphine, butorphanol, xylazine, metedomidine,hydromorphone, hydrocodone, oxymorphone, oxycodone, metopon, apomorphine, normorphine, etorphine, buprenorphine, meperidine, lopermide, anileridine, ethoheptazine, piminidine, betaprodine, diphenoxylate, fentanyl, sufentanil, alfentanil, remifentanil, levorphanol, dextromethorphan, phenazocine, pentazocine, cyclazocine, methadone, isomethadone and propoxyphene. Suitable non-opioid analgesic agents include, but are not limited to, acetaminophen, aspirin, celecoxib, rofecoxib, diclofinac, diflusinal, etodolac, fenoprofen, flurbiprofen, ibuprofen, ketoprofen, indomethacin, ketorolac, meclofenamate, mefanamic acid, nabumetone, naprosin, naproxen, piroxicam and sulindac.

In still another embodiment, the other therapeutic agent is an anxiolytic agent. Suitable anxiolytic agents include, but are not limited to, buspirone, and benzodiazepines such as diazepam, lorazepam, oxazapam, chlorazepate, clonazepam, chlordiazepoxide and alprazolam.

In another embodiment, the other therapeutic agent is an antibacterial agent. Suitable antibacterial agents include, but are not limited to, beta-lactams, such as the penicillins, the cephalosporins, moxalactam, imipenem/cilastatin, and aztreonam; aminoglycosides, such as amikasin, gentamycin, netilmycin and tobramycin; macrolides, such as erythromycin, azithromycin and clarithromycin; fluoroquinolines; metronidazole; sulfonamides; tetracyclines; trimethroprim; and vancomycin.

In still another embodiment, the other therapeutic agent is an antiviral agent. Suitable antiviral agents include, but are not limited to, acyclovir, amantadine, didanosine, famicyclovir, foscarnet, ganciclovir, rimatandine, stavudine, zalcitavine and zitovudine.

In yet another embodiment, the other therapeutic agent is an anti-fungal agent. Suitable anti-fungal agents include, but are not limited to, polyene anti-fungals, such as nystatin, amphotericin, candicidin; azole derivatives, such as itraconazole, clotrimazole, miconazole, ketoconazole and fluconazole; echinocandins; 5-fluorocytosine; griseofulvin; amphotericin B; flucytosine; triazoles, and terbinafine.

In a further embodiment, the other therapeutic agent is an anti-parasitic agent. Suitable anti-parasitic agents include, but are not limited to, ivermectin, mebendazole, mefloquine, pentamidine, praziquantel, pyrimethamine and quinine.

In another embodiment, the other therapeutic agent is an anti-pruritic agent. Suitable anti-pruritic agents include, but are not limited to, allantoin, lignocaine, meleleuca oil, pine tar and crotamiton.

A Purine Compound and the other therapeutic agent can act additively or, in one embodiment, synergistically. In one embodiment, a Purine Compound is adminsitered concurrently with another therapeutic agent. In another embodiment, the present compositions can further comprise another therapeutic agent. In a further embodiment, a composition comprising an effective amount of a Purine Compound and an effective amount of another therapeutic agent can be administered. Alternatively, a composition comprising an effective amount of a Purine Compound and a different composition comprising an effective amount of another therapeutic agent can be concurrently administered. In another embodiment, an effective amount of a Purine Compound is administered prior or subsequent to administration of an effective amount of another therapeutic agent. In this embodiment, the Purine Compound is administered while the other therapeutic agent exerts its therapeutic effect, or the other therapeutic agent is administered while the Purine Compound exerts its preventative or therapeutic effect for treating or preventing a Condition.

A composition of the invention can be prepared using a method comprising admixing a Purine Compound and a physiologically acceptable carrier or excipient. Admixing can be accomplished using methods well known for admixing a compound (or salt) and a physiologically acceptable carrier or exipient.

### 5.5 THERAPEUTIC OR PROPHYLACTIC USES OF THE PURINE COMPOUNDS

### 5.5.1 TREATMENT OR PREVENTION OF A CARDIOVASCULAR DISEASE

A cardiovascular disease can be treated or prevented by administration of an effective amount of a Purine Compound.

Cardiovascular diseases that can be treated or prevented by administering an effective amount of a Purine Compound include, but are not limited to, atherosclerosis, hypertension, congestive heart failure, circulatory shock, cardiomyopathy, cardiac transplant, cardiac ischemia, cardioplegia, myocardial infarction, and a cardiac arrhythmia, such as atrial fibrillation, supraventricular tachycardia, atrial flutter, and paroxysmal atrial tachycardia.

In one embodiment, the cardiovascular disease is a cardiac ischemia, hypertension or atherosclerosis.

In one embodiment, the cardiovascular disease is a cardiac arrhythmia, congestive heart failure, circulatory shock or cardiomyopathy.

In one embodiment, the cardiac arrhythmia is a tachycardia or an an idiotopic arrhythmia.

In another embodiment, the methods for treating a cardiovascular disease are useful for converting a cardiac arrhythmia to a normal sinus rhythm.

In still another embodiment, the tachycardia is atrial fibrillation, supraventricular tachycardia, atrial flutter, paroxysmal supraventricular tachycardia, paroxysmal atrial tachycardia, sinus tachycardia, atrioventricular nodal reentry tachycardia, or tachycardia caused by Wolff-Parkinson-White Syndrome.

In a further embodiment, the methods for treating a tachycardia are useful for lowering the subject's ventricular rate to a rate of not less than about 40 beats per minute. In a specific embodiment, the methods are useful for lowering a subject's ventricular rate to a rate of from about 60 beats per minute to about 100 beats per minute.

### 5.5.1.1 PROTECTING A SUBJECT'S HEART AGAINST MYOCARDIAL DAMAGE DURING CARDIOPLEGIA

In one embodiment, the invention provides methods for inducing cardioplegia comprising administering to a subject in need thereof an effective amount of a cardioplegia-inducing agent and a Purine Compound. Cardioplegia-inducing agents useful in the present invention include, but are not limited to, potassium chloride, procaine, lidocaine, novocaine, bupivocaine, nicorandil, pinacidil, halothane, St. Thomas solution, Fremes solution, 2,3-butanedione monoxime, and esmolol.

In one embodiment, the cardioplegia-inducing agent is lidocaine.

In one embodiment, a cardioplegia-inducing agent and a Purine Compound are present within the same composition. The present methods for inducing cardioplegia are useful for preventing or minimizing myocardial damage from occurring during cardioplegia.

In still another embodiment, the invention provides methods for protecting an subject's heart against myocardial damage during cardioplegia, the method comprising administering to a subject in need thereof an effective amount of:

(a) a cardioplegia-inducing agent; and

(b) a Purine Compound.

In one embodiment, the cardioplegia-inducing agent is administered prior to the administration of the Purine Compound.

In another embodiment, Purine Compound is administered prior to the administration of the cardioplegia-inducing agent.

In a further embodiment, the cardioplegia-inducing agent and the Purine Compound are administered concurrently.

In another embodiment, the cardioplegia-inducing agent and the Purine Compound are administered such that the Purine Compound exerts its prophylactic effect of protection against myocardial damage while the cardioplegia-inducing agent exerts its cardioplegic effect.

### 5.5.2 TREATMENT OR PREVENTION OF AN INFLAMMATORY DISEASE

An inflammatory disease can be treated or prevented by administration of an effective amount of a Purine Compound.

Inflammatory diseases that can be treated or prevented by administering an effective amount of a Purine Compound include, but are not limited to, organ transplant rejection; reoxygenation injury resulting from organ transplantation including, but not limited to, transplantation of the following organs: heart, lung, liver and kidney; systemic inflammatory response syndrome; chronic inflammatory diseases of the joints, including arthritis, rheumatoid arthritis, osteoarthritis and bone diseases associated with increased bone resorption; inflammatory bowel diseases such as ileitis, ulcerative colitis, Barrett's syndrome, and Crohn's disease; inflammatory lung diseases such as asthma, adult respiratory distress syndrome, and chronic obstructive airway disease; inflammatory diseases of the eye including corneal dystrophy, trachoma, onchocerciasis, uveitis, sympathetic ophthalmitis and endophthalmitis; chronic inflammatory diseases of the gum, including gingivitis and periodontitis; inflammatory diseases of the joints including arthritis and osteoarthritis; inflammatory diseases of the kidney including uremic complications, glomerulonephritis and nephrosis; inflammatory diseases of the skin including sclerodermatitis, psoriasis and eczema; inflammatory diseases of the central nervous system, including chronic demyelinating diseases of the nervous system, multiple sclerosis, AIDS-related neurodegeneration and Alzheimer's disease, infectious meningitis, encephalomyelitis, Parkinson's disease, Huntington's disease, amyotrophic lateral sclerosis and viral or autoimmune encephalitis; autoimmune diseases including Type I and Type II diabetes mellitus; diabetic complications, including, but not limited to, diabetic cataract, glaucoma, retinopathy, nephropathy, such as microaluminuria and progressive diabetic nephropathy, polyneuropathy, gangrene of the feet, atherosclerotic coronary arterial disease, peripheral arterial disease, nonketotic hyperglycemic-hyperosmolar coma, mononeuropathies, autonomic neuropathy, foot ulcers, joint problems, and a skin or mucous membrane complication, such as an infection, a shin spot, a candidal infection or necrobiosis lipoidica diabeticorum; immune-complex vasculitis, systemic lupus erythematosus (SLE); inflammatory diseases of the heart such as cardiomyopathy, ischemic heart disease hypercholesterolemia, and atherosclerosis; as well as various other diseases that can have significant inflammatory components, including preeclampsia; chronic liver failure, brain and spinal cord trauma, and cancer. The inflammatory disease can also be a systemic inflammation of the body, exemplified by gram-positive or gram negative shock, hemorrhagic or anaphylactic shock, or shock induced by cancer chemotherapy in response to pro-inflammatory cytokines, e.g., shock associated with pro-inflammatory cytokines. Such shock can be induced, e.g., by a chemotherapeutic agent that is administered as a treatment for cancer.

In one embodiment, the inflammatory disease is an inflammatory lung disease, an autoimmune inflammatory disease, an inflammatory disease of the eye, an inflammatory disease of the gum, an inflammatory disease of the central nervous system, an inflammatory disease of the skin, an inflammatory disease of the bowel or an inflammatory disease of a joint.

In one embodiment, the inflammatory disease of the skin is psoriasis.

In another embodiment, the inflammatory lung disease is asthma.

### 5.5.3 TREATMENT OR PREVENTION OF A NEUROLOGICAL DISORDER

A neurological disorder can be treated or prevented by administration of an effective amount of a Purine Compound.

Neurological disorders that can be treated or prevented by administering an effective amount of a Purine Compound include, but are not limited to, a seizure disorder, such as epilepsy;

pain, including acute postoperative pain, cancer pain, neuropathic pain, pain resulting from surgery, labor pain during childbirth, a psychogenic pain syndrome, and headache, including migraine headache and cluster headache; delirium and dementia, such as Lewy body dementia, Alzheimer's disease, Pick's disease, or a Creutzfeldt-Jakob disease; a sleep disorder, such as insomnia, hypersomnia, a sleep apnea syndrome, restless-leg syndrome, or a parasomnia; a cranial nerve disorder, such as Bell's palsy; a disorder of movement, such as tremor, dystonia, Tourette's Syndrome, myoclonus, Huntington's disease, cortico basal degeneration, chorea, a drug-induced movement disorder, progressive supranuclear palsy, Parkinson's disease, or a Parkinsonian Syndrome, such as multiple system atrophy, Wilson's disease or mult-infarct state; a demyelinating disease, such as multiple sclerosis or amyotrophic lateral sclerosis; a neuro-muscular disease, such as muscular dystrophy; a cerebrovascular disease, such as stroke; a neuroopthalmic disorder; and a psychiatric disorder, including but not limited to, somatoform disorders, such as hypochondriasis or body dysmorphic disorder; dissociation disorders, such as panic disorder, phobic disorders, or obsessive-compulsive disorders; mood disorders, such as depression or bipolar disorders; personality disorders; psychosexual disorders; suicidal behavior; schizophrenia; brief psychotic disorder; and delusional disorder.

In one embodiment, the neurological disorder treated or prevented is epilepsy, pain, or stroke.

In one embodiment, the present methods for treating pain further comprise the administration of an additional analgesic agent. In a specific embodiment, the additional analgesic agent is buprenorphine.

### 5.5.4 TREATMENT OR PREVENTION OF AN OPHTHALMIC CONDITION

An ophthalmic condition can be treated or prevented by administration of an effective amount of a Purine Compound.

Ophthalmic conditions that can be treated or prevented by administering an effective amount of a Purine Compound include, but are not limited to, glaucoma with normal intraocular pressure, glaucoma with intraocular hypertension, pseudoexfoliation syndrome, ischemic retinopathy, diabetic retinopathy, and acute macular degeneration.

In one embodiment, the neurological disorder treated or prevented is glaucoma with intraocular hypertension or glaucoma with normal intraocular pressure.

### 5.5.5 TREATMENT OR PREVENTION OF AN ISCHEMIC CONDITION

An ischemic condition can be treated or prevented by administration of an effective amount of a Purine Compound.

Ischemic conditions that can be treated or prevented by administering an effective amount of a Purine Compound include, but are not limited to, stable angina, unstable angina, myocardial ischemia, hepatic ischemia, mesenteric artery ischemia, intestinal ischemia, critical limb ischemia, chronic critical limb ischemia, cerebral ischemia, acute cardiac ischemia, and an ischemic disease of the central nervous system, such as stroke or cerebral ischemia.

In one embodiment, the ischemic condition is myocardial ischemia, stable angina, unstable angina, stroke, ischemic heart disease or cerebral ischemia.

### 5.5.6 TREATMENT OR PREVENTION OF A REPERFUSION INJURY

A reperfusion injury can be treated or prevented by administration of an effective amount of a Purine Compound. Reperfusion injury can result following a naturally occurring episode, such as a myocardial infarction, stroke, or during a surgical procedure where blood flow in vessels is intentionally or unintentionally blocked.

Reperfusion injuries that can be treated or prevented by administering an effective amount of a Purine Compound include, but are not limited to, intestinal reperfusion injury, myocardial reperfusion injury; and reperfusion injury resulting from cardiopulmonary bypass surgery, thoracoabrominal aneurysm repair surgery, carotid endaretectomy surgery, or hemorrhagic shock.

In one embodiment, the reperfusion injury results from cardiopulmonary bypass surgery, thoracoabrominal aneurysm repair surgery, carotid endarerectomy surgery or hemorrhagic shock.

### 5.5.7 TREATMENT OR PREVENTION OF A SKIN DISORDER

A skin disorder can be treated or prevented by administration of an effective amount of a Purine Compound.

Skin disorders that can be treated or prevented by administering an effective amount of a Purine Compound include, but are not limited to, pruritis; acne; skin rashes, such as psoriasis, dermatitis, rosacea, lichen planus, keratosis, drug rashes and granuloma annulare; sunburn and skin photosensitivity reactions; warts, such as plantar warts, common warts, filiform warts, flat worts, genital warts, and keratoses; and skin pigment disorders such as albinism, melasma and vitiligo.

In one embodiment, the skin disorder is psoriasis.

### 5.5.8 TREATMENT OR PREVENTION OF DIABETES

Diabetes can be treated or prevented by administration of an effective amount of a Purine Compound.

Types of diabetes that can be treated or prevented by administering an effective amount of a Purine Compound include, but are not limited to, Type I diabetes (Insulin Dependent Diabetes Mellitus), Type II diabetes (Non-Insulin Dependent Diabetes Mellitus), gestational diabetes, insulinopathy, diabetes due to pancreatic disease, diabetes associated with another endocrine disease (such as Cushing's Syndrome, acromegaly, pheochromocytoma, glucagonoma, primary aldosteronism or somatostatinoma), Type A insulin resistance, syndrome, Type B, insulin resistance syndrome, lipatrophic diabetes, and diabetes induced by β-cell toxins.

In one embodiment, the diabetes is Type I diabetes mellitus.

In another embodiment, the diabetes is Type II diabetes mellitus.

### 5.5.9 METHODS FOR REDUCING A SUBJECT'S RATE OF METABOLISM

In one embodiment, the invention provides methods for reducing a subject's rate of metabolism comprising administering to a subject in need thereof an amount of a Purine Compound that is effective to slow the subject's rate of metabolism.

Reducing a subject's rate of metabolism is useful for slowing a subject's heart rate during heart surgery; protecting a subject's tissue from damage during surgery, particular heart or brain surgery; reducing intracranial hypertension caused by brain injury in a subject; or inducing hibernation in a subject.

Accordingly, the present invention encompasses methods for slowing a subject's heart rate during heart surgery; protecting a subject's tissue from damage during surgery, particular heart or brain surgery; reducing intracranial hypertension caused by brain injury in a subject; or inducing hibernation in a subject, the methods comprising administering an effective amount of a Purine Compound to a subject in need thereof.

Reducing a subject's rate of metabolism is also useful for reducing a subject's rate of oxygen consumption. Accordingly, the present invention provides methods for reducing the rate of a subject's oxygen consumption, the method comprising administering to a subject in need thereof an amount of a Purine Compound that is effective to reduce the subject's rate of oxygen consumption. A subject's oxygen supply might be compromised due to: (i) a medical procedure, such as heart surgery, brain surgery, organ transplantation, mechanical occlusion of the vascular supply, or vascular stenosis; (ii) a disorder or medical condition such as ischemia, a respiratory disorder, respiratory failure, a pulmonary disorder, anemia, anaphylactic shock, hemmorhagic shock, dehydration, compartment syndrome, intravascular thrombus, septic shock, cystic fibrosis, lung cancer, stroke, a burn, or internal bleeding; (iii) an injury such as drowning, a crush injury to one or more limbs, choking, or suffocation; (iv) a compromised airway due to asthma, a tumor, a lung injury or a tracheal injury; (v) an external compression of one or more blood vessels; or (vi) an intrinsic obstruction of one or more blood vessels. Reducing a subject's rate of oxygen consumption is useful for treating or preventing tissue damage or stroke, resulting from an inadequate supply of oxygen to a cell, a tissue, an organ or an organ system.

In one embodiment, a subject's rate of oxygen consumption is reduced to increase emergency recussitation in an injured subject.

In another embodiment, a subject's rate of oxygen consumption is reduced prior to and during heart surgery. In a specific embodiment, the subject is a human child undergoing pediatric heart surgery.

In another embodiment, a subject's rate of oxygen consumption is reduced to treat respiratory failure in the subject.

In one embodiment, a subject's rate of oxygen consumption is reduced to aid tissue metabolism in the subject whose respiration and ventilation is facilitated by a ventilator. In a specific embodiment, the subject whose respiration and ventilation is facilitated by a ventilator is a geriatric human. In another specific. embodiment, the subject whose respiration and ventilation is facilitated by a ventilator is a premature human infant.

In one embodiment, an organ can be stored ex vivo in a composition comprising an effective amount of a Purine Compound. The composition is useful for preserving an organ's viability after being removed from a donor and before the organ is transplanted in a recipient. In one embodiment, the donor and recipient are the same.

In another embodiment, an effective amount of a Purine Compound can be administered to a subject awaiting organ transplantation to reduce the subject's rate of oxygen consumption prior to or during organ transplantation.

Reducing a subject's rate of metabolism is also useful for reducing a subject's core body temperature. Accordingly, the present invention provides methods for reducing a subject's core body temperature, the method comprising administering to a subject in need thereof an amount of a Purine Compound that is effective to reduce the subject's core body temperature.

In one embodiment, the subject's core body temperature is reduced to a temperature from about 4° C. to about 34° C. In certain embodiments, the subject's core body temperature is reduced to about 34° C., to about 30° C., to about 25° C., to about 20° C., to about 15° C., to about 10° C., or to about 4° C.

In a specific embodiment, a subject's core body temperature is reduced to induce therapeutic hypothermia.

### 5.5.10 TREATMENT OR PREVENTION OF OBESITY

Obesity can be treated or prevented by administration of an effective amount of a Purine Compound.

Types of obesity that can be treated or prevented by administering an effective amount of a Purine Compound include, but are not limited to, android obesity, gynoid obesity, abdominal obesity, age-related obesity, diet-induced obesity, fat-induced obesity, hypothalamic obesity, morbid obesity, multigenic obesity, and visceral obesity.

In one embodiment, the obesity is android obesity.

### 5.5.11 TREATMENT OR PREVENTION OF A WASTING DISEASE

In one embodiment, the invention provides methods for treating or preventing a wasting disease, comprising administering to a subject in need thereof an amount of a a Purine Compound that is effective to treat or prevent the wasting disease.

Types of wasting diseases that can be treated or prevented by administering an effective amount of a Purine Compound include, but are not limited to chronic wasting disease, cancer wasting syndrome, and AIDS wasting syndrome.

### 5.5.12 TREATMENT OR PREVENTION OF A CELLULAR PROLIFERATIVE DISORDER

A cellular proliferative disorder can be treated or prevented by administration of an effective amount of a Purine Compound.

Types of cellular proliferative disorders that can be treated or prevented by administering an effective amount of a Purine Compound include, but are not limited to, cancer, uterine fibroids, benign prostatic hyperplasia, familial adenomatosis polyposis, neuro-fibromatosis, atherosclerosis, pulmonary fibrosis, arthritis, psoriasis, glomerulonephritis, restenosis following angioplasty or vascular surgery, hypertrophic scar formation, inflammatory bowel disease, transplantation rejection, endotoxic shock, fungal infections, and defective apoptosis-associated conditions.

In one embodiment, the cellular proliferative disorder is cancer.

### 5.5.13 TREATMENT OR PREVENTION OF CANCER

In one embodiment, the Purine Compounds can also be administered to prevent progression to a neoplastic or malignant state, including but not limited to the cancers listed in Table 1. Such prophylactic use is indicated in conditions known or suspected of preceding progression to neoplasia or cancer, in particular, where non-neoplastic cell growth consisting of hyperplasia, metaplasia, or most particularly, dysplasia has occurred (for review of such abnormal growth conditions, see Robbins and Angell, 1976, Basic Pathology, 2d Ed., W.B. Saunders Co., Philadelphia, pp. 68-79). Hyperplasia is a form of controlled cell proliferation involving an increase in cell number in a tissue or organ, without significant alteration in structure or function. For example, endometrial hyperplasia often precedes endometrial cancer and precancerous colon polyps often transform into cancerous lesions. Metaplasia is a form of controlled cell growth in which one type of adult or fully differentiated cell substitutes for another type of adult cell. Metaplasia can occur in epithelial or connective tissue cells. A typical metaplasia involves a somewhat disorderly metaplastic epithelium. Dysplasia is frequently a forerunner of cancer, and is found mainly in the epithelia; it is the most disorderly form of non-neoplastic cell growth, involving a loss in individual cell uniformity and in the architectural orientation of cells. Dysplastic cells often have abnormally large, deeply stained nuclei, and exhibit pleomorphism. Dysplasia characteristically occurs where there exists chronic irritation or inflammation, and is often found in the cervix, respiratory passages, oral cavity, and gall bladder.

Alternatively or in addition to the presence of abnormal cell growth characterized as hyperplasia, metaplasia, or dysplasia, the presence of one or more characteristics of a transformed phenotype, or of a malignant phenotype, displayed in vivo or displayed in vitro by a cell sample from a subject, can indicate the desirability of prophylactic/therapeutic administration of the composition of the invention. Such characteristics of a transformed phenotype include morphology changes, looser substratum attachment, loss of contact inhibition, loss of anchorage dependence, protease release, increased sugar transport, decreased serum requirement, expression of fetal antigens, disappearance of the 250,000 dalton cell surface protein, etc. (see also id., at pp. 84-90 for characteristics associated with a transformed or malignant phenotype).

In a specific embodiment, leukoplakia, a benign-appearing hyperplastic or dysplastic lesion of the epithelium, or Bowen's disease, a carcinoma in situ, are pre-neoplastic lesions indicative of the desirability of prophylactic intervention.

In another embodiment, fibrocystic disease (cystic hyperplasia, mammary dysplasia, particularly adenosis (benign epithelial hyperplasia)) is indicative of the desirability of prophylactic intervention.

The prophylactic use of the compounds and methods of the present invention are also indicated in some viral infections that may lead to cancer. For example, human papilloma virus can lead to cervical cancer (see, e.g., Hernandez-Avila et al., Archives of Medical Research (1997) 28:265-271), Epstein-Barr virus (EBV) can lead to lymphoma (see, e.g., Herrmann et al., J Pathol (2003) 199(2):140-5), hepatitis B or C virus can lead to liver carcinoma (see, e.g., El-Serag, J Clin Gastroenterol (2002) 35(5 Suppl 2):S72-8), human T cell leukemia virus (HTLV)-I can lead to T-cell leukemia (see e.g., Mortreux et al., Leukemia (2003) 17(1):26-38), human herpesvirus-8 infection can lead to Kaposi's sarcoma (see, e.g., Kadow et al., Curr Opin Investig Drugs (2002) 3(11):1574-9), and Human Immune deficiency Virus (HIV) infection contribute to cancer development as a consequence of immunodeficiency (see, e.g., Dal Maso et al., Lancet Oncol (2003) 4(2): 110-9).

In other embodiments, a subject which exhibits one or more of the following predisposing factors for malignancy can treated by administration of the compounds or methods of the invention: a chromosomal translocation associated with a malignancy (e.g., the Philadelphia chromosome for chronic myelogenous leukemia, t(14;18) for follicular lymphoma, etc.), familial polyposis or Gardner's syndrome (possible forerunners of colon cancer), benign monoclonal gammopathy (a possible forerunner of multiple myeloma), a first degree kinship with persons having a cancer or precancerous disease showing a Mendelian (genetic) inheritance pattern (e.g., familial polyposis of the colon, Gardner's syndrome, hereditary exostosis, polyendocrine adenomatosis, medullary thyroid carcinoma with amyloid production and pheochromocytoma, Peutz-Jeghers syndrome, neurofibromatosis of Von Recklinghausen, retinoblastoma, carotid body tumor, cutaneous melanocarcinoma, intraocular melanocarcinoma, xeroderma pigmentosum, ataxia telangiectasia, Chediak-Higashi syndrome, albinism, Fanconi's aplastic anemia, and Bloom's syndrome; see Robbins and Angell, 1976, Basic Pathology, 2d Ed., W.B. Saunders Co., Philadelphia, pp. 112-113) etc.), and exposure to carcinogens (e.g., smoking, and inhalation of or contacting with certain chemicals).

In a preferred embodiment, the present invention provides methods for treating cancer, including but not limited to: killing a cancer cell or neoplastic cell; inhibiting the growth of a cancer cell or neoplastic cell; inhibiting the replication of a cancer cell or neoplastic cell; or ameliorating a symptom thereof, the methods comprising administering to a subject in need thereof an amount of the Purine Compounds effective to treat cancer.

In one embodiment, the invention provides a method for treating cancer, said method comprising administering to a subject in need thereof an amount of a Purine Compound or a pharmaceutically acceptable salt thereof, said amount sufficient to treat cancer.

In another embodiment, the invention provides a method for treating cancer, said method comprising administering to a subject in need thereof a pharmaceutical composition comprising an amount of a Purine Compound effective to treat cancer.

In a specific embodiment, the subject in need of treatment has previously undergone treatment for cancer. Such previous treatments include, but are not limited to, prior chemotherapy, radiotherapy, surgery, or immunotherapy, such as cancer vaccines.

Cancers that can be treated with the Compounds and methods of the Invention include, but are not limited to, cancers disclosed below in Table I and metastases thereof.

TABLE 1

Solid tumors, including but not limited to:
fibrosarcoma
myxosarcoma
liposarcoma
chondrosarcoma
osteogenic sarcoma
chordoma
angiosarcoma
endotheliosarcoma
lymphangiosarcoma
lymphangioendotheliosarcoma
synovioma
mesothelioma
Ewing's tumor
leiomyosarcoma
rhabdomyosarcoma
colon cancer
colorectal cancer
kidney cancer
pancreatic cancer
bone cancer
breast cancer
ovarian cancer
prostate cancer
esophageal cancer
stomach cancer
oral cancer
nasal cancer
throat cancer
squamous cell carcinoma
basal cell carcinoma
adenocarcinoma
sweat gland carcinoma
sebaceous gland carcinoma
papillary carcinoma
papillary adenocarcinomas
cystadenocarcinoma
medullary carcinoma
bronchogenic carcinoma
renal cell carcinoma
adrenal cancer
hepatoma
bile duct carcinoma
choriocarcinoma
seminoma
embryonal carcinoma
Wilms' tumor
cervical cancer
uterine cancer
testicular cancer
small cell lung carcinoma
bladder carcinoma
lung cancer
epithelial carcinoma
brain cancer
glioma
glioblastoma multiforme
astrocytoma
medulloblastoma
craniopharyngioma
ependymoma
pinealoma
hemangioblastoma
acoustic neuroma
oligodendroglioma
meningioma
skin cancer
melanoma
neuroblastoma
retinoblastoma
blood-borne cancers, including but not limited to:
acute lymphoblastic leukemia (“ALL”)
acute lymphoblastic B-cell leukemia
acute lymphoblastic T-cell leukemia
acute myeloblastic leukemia (“AML”)
acute promyelocytic leukemia (“APL”)
acute monoblastic leukemia
acute erythroleukemic leukemia
acute megakaryoblastic leukemia
acute myelomonocytic leukemia
acute nonlymphocyctic leukemia
acute undifferentiated leukemia
chronic myelocytic leukemia (“CML”)
chronic lymphocytic leukemia (“CLL”)
hairy cell leukemia
multiple myeloma
acute and chronic leukemias:
lymphoblastic
myelogenous
lymphocytic
myelocytic leukemias
Lymphomas:
Hodgkin's disease
non-Hodgkin's Lymphoma
Multiple myeloma
Waldenström's macroglobulinemia
Heavy chain disease
Polycythemia vera

In one embodiment, the cancer is lung cancer, breast cancer, colorectal cancer, prostate cancer, brain cancer, esophageal cancer, pancreatic cancer, stomach cancer, liver cancer, kidney cancer, adrenal cancer, testicular cancer, ovarian cancer, cervical cancer, leukemia, Hodgkin's disease, non-Hodgkin's lympoma, skin cancer, bone cancer, a cancer of the central nervous system, or a cancer of the blood or lymphatic system.

### 5.5.13.1 MULTI-MODALITY THERAPY FOR CANCER

The Purine Compounds can be administered to a subject that has undergone or is currently undergoing one or more additional anticancer treatment modalities including, but not limited to, chemotherapy, radiotherapy, surgery or immunotherapy, such as cancer vaccines.

In one embodiment, the invention provides methods for treating cancer comprising (a) administering to a subject in need thereof a therapeutically effective amount of a Purine Compound; and (b) administering to said subject one or more additional anticancer treatment modalities including, but not limited to, radiotherapy, chemotherapy, surgery or immunotherapy, such as a cancer vaccine. In one embodiment, the administering of step (a) occurs prior to the administering of step (b). In another embodiment, the administering of step (a) occurs subsequent to the administering of step (b). In still another embodiment, the administering of step (a) occurs concurrently with the administering of step (b).

In one embodiment, the additional anticancer treatment modality is chemotherapy.

In another embodiment, the additional anticancer treatment modality is surgery.

In yet another embodiment, the additional anticancer treatment modality is radiation therapy.

In still another embodiment, the additional anticancer treatment modality is immunotherapy, such as cancer vaccines.

The Purine Compound and the additional treament modalities of the combination therapies of the invention can act additively or synergistically. A synergistic combination allows the use of lower dosages of the Purine Compound and/or the additional treatment modality and/or less frequent administration of the Purine Compound and/or additional treatment modality to a subject with cancer. The ability to utilize lower dosages of a Purine Compound and/or an additional treatment modality and/or to administer a Purine Compound and said additional treament modality less frequently can reduce the toxicity associated with the administration of a Purine Compound and/or the additional treatement modality to a subject without reducing the efficacy of a Purine Compound and/or the additional treatement modality in the treatment of cancer. In addition, a synergistic effect can result in the improved efficacy of the treatment of cancer and/or the reduction of adverse or unwanted side effects associated with the administration of a Purine Compound and/or an additional anticancer treatment modality as monotherapy.

When the Purine Compound and additional anticancer treatment modality are administered to a subject concurrently, the term “concurrently” is not limited to the administration of a Purine Compound and an additional anticancer treatment modality at exactly the same time, but rather it is meant that they are administered to a subject in a sequence and within a time interval such that they can act synergistically to provide an increased benefit than if they were administered otherwise. For example, the Purine Compounds may be administered at the same time or sequentially in any order at different points in time as an additional anticancer treament modality; however, if not administered at the same time, they should be administered sufficiently close in time so as to provide the desired therapeutic effect, preferably in a synergistic fashion. The Purine Compound and the additional anticancer treatment modality can be administered separately, in any appropriate form and by any suitable route. When the Purine Compound and the additional anticancer treatment modality are not administered concurrently, it is understood that they can be administered in any order to a subject in need thereof. For example, a Purine Compound can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of an additional anticancer treatment modality (e.g., radiotherapy), to a subject in need thereof. In various embodiments the Purine, Compound and the additional anticancer treatment modality are administered 1 minute apart, 10 minutes apart, 30 minutes apart, less than 1 hour apart, 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, no more than 24 hours apart or no more than 48 hours apart. In one embodiment, the components of the combination therapies of the invention are administered within the same office or hospital visit. In another embodiment, the Purine Compound and the additional anticancer treatment modality are administered at 1 minute to 24 hours apart.

In one embodiment, a Purine Compound is administered prior or subsequent to an additional anticancer treatment modality, preferably at least an hour, five hours, 12 hours, a day, a week, a month, more preferably several months (e.g., up to three months), prior or subsequent to administration of an additional anticancer treatment modality.

When the combination theapy of the invention comprises administering a Purine Compound are with one or more additional anticancer agents, the Purine Compound and the additional anticancer agents can be administered concurrently or sequentially to a subject. The agents can also be cyclically administered. Cycling therapy involves the administration of one or more anticancer agents for a period of time, followed by the administration of one or more different anticancer agents for a period of time and repeating this sequential administration, i.e., the cycle, in order to reduce the development of resistance to one or more of the anticancer agents of being administered, to avoid or reduce the side effects of one or more of the anticancer agents being administered, and/or to improve the efficacy of the treatment.

An additional anticancer agent may be administered over a series of sessions; any one or a combination of the additional anticancer agents listed below may be administered.

The present invention includes methods for treating cancer, comprising administering to a subject in need thereof a Purine Compound, and one or more additional anticancer agents or pharmaceutically acceptable salts thereof. The Purine Compound and the additional anticancer agent(s) can act additively or synergistically.

In one embodiment, the additional anti-cancer agent can be, but is not limited to, a drug listed in Table 2.

TABLE 2

| | |
|---|---|
| Alkylating agents | |
| Nitrogen mustards: | Cyclophosphamide |
| | Ifosfamide |
| | Trofosfamide |
| | Chlorambucil |
| Nitrosoureas: | Carmustine (BCNU) |
| | Lomustine (CCNU) |
| Alkylsulphonates: | Busulfan |
| | Treosulfan |
| Triazenes: | Dacarbazine |
| Platinum complexes: | Cisplatin |
| | Carboplatin |
| | Oxaliplatin |
| Plant Alkaloids | |
| Vinca alkaloids: | Vincristine |
| | Vinblastine |
| | Vindesine |
| | Vinorelbine |
| Taxoids: | Paclitaxel |
| | Docetaxel |
| DNA Topoisomerase | |
| Inhibitors | |
| Epipodophyllins: | Etoposide |
| | Teniposide |
| | Topotecan |
| | 9-aminocamptothecin |
| | Camptothecin |
| | Crisnatol |
| Mitomycins: | Mitomycin C |
| | Anti-metabolites |
| Anti-folates: | |
| DHFR inhibitors: | Methotrexate |
| | Trimetrexate |
| IMP dehydrogenase | Mycophenolic acid |
| Inhibitors: | Tiazofurin |
| | Ribavirin |
| | EICAR |
| Ribonuclotide reductase | Hydroxyurea |
| Inhibitors: | Deferoxamine |
| Pyrimidine analogs: | |
| Uracil analogs: | 5-Fluorouracil |
| | Floxuridine |
| | Doxifluridine |
| | Ratitrexed |
| Cytosine analogs: | Cytarabine (ara C) |
| | Cytosine arabinoside |
| | Fludarabine |
| | Gemcitabine |
| | Capecitabine |
| Purine analogs: | Mercaptopurine |
| | Thioguanine |
| DNA Antimetabolites: | 3-HP |
| | 2′-deoxy-5-fluorouridine |
| | 5-HP |
| | alpha-TGDR |
| | aphidicolin glycinate |
| | ara-C |
| | 5-aza-2′-deoxycytidine |
| | beta-TGDR |
| | cyclocytidine |
| | guanazole |
| | inosine glycodialdehyde |
| | macebecin II |
| | Pyrazoloimidazole |
| Hormonal therapies: | |
| Receptor antagonists: | |
| Anti-estrogen: | Tamoxifen |
| | Raloxifene |
| | Megestrol |
| LHRH agonists: | Goserelin |
| | Leuprolide acetate |
| Anti-androgens: | Flutamide |
| | Bicalutamide |
| Retinoids/Deltoids | |
| | Cis-retinoic acid |
| Vitamin A derivative: | All-trans retinoic acid (ATRA-IV) |
| Vitamin D3 analogs: | EB 1089 |
| | CB 1093 |
| | KH 1060 |
| Photodynamic therapies: | Vertoporfin (BPD-MA) |
| | Phthalocyanine |
| | Photosensitizer Pc4 |
| | Demethoxy-hypocrellin A |
| | (2BA-2-DMHA) |
| Cytokines: | Interferon-α |
| | Interferon-β |
| | Interferon-γ |
| | Tumor necrosis factor |
| Angiogenesis Inhibitors: | Angiostatin (plasminogen fragment) |
| | antiangiogenic antithrombin III |
| | Angiozyme |
| | ABT-627 |
| | Bay 12-9566 |
| | Benefin |
| | Bevacizumab |
| | BMS-275291 |
| | cartilage-derived inhibitor (CDI) |
| | CAI |
| | CD59 complement fragment |
| | CEP-7055 |
| | Col 3 |
| | Combretastatin A-4 |
| | Endostatin (collagen XVIII fragment) |
| | Fibronectin fragment |
| | Gro-beta |
| | Halofuginone |
| | Heparinases |
| | Heparin hexasaccharide fragment |
| | HMV833 |
| | Human chorionic gonadotropin (hCG) |
| | IM-862 |
| | Interferon alpha/beta/gamma |
| | Interferon inducible protein (IP-10) |
| | Interleukin-12 |
| | Kringle 5 (plasminogen fragment) |
| | Marimastat |
| | Metalloproteinase inhibitors (TIMPs) |
| | 2-Methoxyestradiol |
| | MMI 270 (CGS 27023A) |
| | MoAb IMC-1C11 |
| | Neovastat |
| | NM-3 |
| | Panzem |
| | PI-88 |
| | Placental ribonuclease inhibitor |
| | Plasminogen activator inhibitor |
| | Platelet factor-4 (PF4) |
| | Prinomastat |
| | Prolactin 16kD fragment |
| | Proliferin-related protein (PRP) |
| | PTK 787/ZK 222594 |
| | Retinoids |
| | Solimastat |
| | Squalamine |
| | SS 3304 |
| | SU 5416 |
| | SU6668 |
| | SU11248 |
| | Tetrahydrocortisol-S |
| | Tetrathiomolybdate |
| | Thalidomide |
| | Thrombospondin-1 (TSP-1) |
| | TNP-470 |
| | Transforming growth factor-beta (TGF-β) |
| | Vasculostatin |
| | Vasostatin (calreticulin fragment) |
| | ZD6126 |
| | ZD 6474 |
| | farnesyl transferase inhibitors (FTI) |
| | Bisphosphonates |
| Antimitotic agents: | Allocolchicine |
| | Halichondrin B |
| | Colchicine |
| | colchicine derivative |
| | dolstatin 10 |
| | Maytansine |
| | Rhizoxin |
| | Thiocolchicine |
| | trityl cysteine |
| Others: | |
| Isoprenylation inhibitors: | |
| Dopaminergic neurotoxins: | 1-methyl-4-phenylpyridinium ion |
| Cell cycle inhibitors: | Staurosporine |
| Actinomycins: | Actinomycin D |
| | Dactinomycin |
| Bleomycins: | Bleomycin A2 |
| | Bleomycin B2 |
| | Peplomycin |
| Anthracyclines: | Daunorubicin |
| | Doxorubicin (adriamycin) |
| | Idarubicin |
| | Epirubicin |
| | Pirarubicin |
| | Zorubicin |
| | Mitoxantrone |
| MDR inhibitors: | Verapamil |
| Ca²⁺ | Thapsigargin |
| ATPase inhibitors: | |

In a further aspect of the invention the Purine Compounds can be administered in conjunction with chemical agents that are understood to mimic the effects of radiotherapy and/or that function by direct contact with DNA. Preferred agents for use in combination with the Purine Compounds for treating cancer include, but are not limited to cis-diamminedichloro platinum (II) (cisplatin), doxorubicin, 5-fluorouracil, taxol, and topoisomerase inhibitors such as etoposide, teniposide, irinotecan and topotecan.

Additionally, the invention provides methods of treatment of cancer using the Purine Compounds as an alternative to chemotherapy alone or radiotherapy alone where the chemotherapy or the radiotherapy has proven or can prove too toxic, e.g., results in unacceptable or unbearable side effects, for the subject being treated. The subject being treated can, optionally, be treated with another anticancer treatment modality such as chemotherapy, surgery, or immunotherapy, depending on which treatment is found to be acceptable or bearable.

The Purine Compounds can also be used in vitro or ex vivo, such as for the treatment of certain cancers, including, but not limited to leukemias and lymphomas, such treatment involving autologous stem cell transplants. This can involve a multi-step process in which the subject's autologous hematopoietic stem cells are harvested and purged of all cancer cells, the subject is then administered an amount of a Purine Compound effective to eradicate the subject's remaining bone-marrow cell population, then the stem cell graft is infused back into the subject. Supportive care can then be provided while bone marrow function is restored and the subject recovers.

### 5.5.14 TREATMENT OF WOUNDS

Also encompassed are method for treating a wound, comprising administering to a subject in need thereof an effective amount of a Purine Compound.

Wounds that can be treated by administering an effective amount of a Purine Compound include, but are not limited to, an avulsion, an incision, a bruise, a laceration, an amputation, a puncture wound, an abrasion, an ischemic ulcer, a decubitus ulcer, an ulcer due to an infectious processe, an ulcer due to an inflammatory processe, and a wound caused by a burn.

The wounds may be caused accidentally or may be inflicted intentionally, such as those which are inflicted during surgery or other medical procedures.

In one embodiment, the methods for treating a wound expedite would healing.

In another embodiment, the methods for treating a wound can further comprise administering an effective amount of another therapeutic agent. Other therapeutic agents useful in the methods for treating a wound include, but are not limited to, an antibacterial agent, an antiviral agent, an antifungal agent, an antiparasitic agent, an antiinflammatory agent, an analgesic agent, an antipruritic agent, or any combination thereof, for example, as disclosed herein.

In another embodiment, the present invention provides a method for stimulating the influx of fibroblasts, vascular endothelial cells or epithelial cells into a wound, comprising administering to a subject in need thereof an effective amount of a Purine Compound.

### 5.5.15 TREATMENT OR PREVENTION OF A RADIATION-INDUCED INJURY

A radiation-induced injury can be treated or prevented by administration of an effective amount of a Purine Compound to a subject.

Examples of a radiation-induced injury treatable or preventable using the present methods include, but are not limited to, an acute radiation syndrome, such as a cerebral syndrome; a gastrointestinal syndrome; a hematopoietic syndrome; acute radiation sickness; pulmonary fibrosis; radiation proctitis; neuropathy; nausea; vomiting; alopecia; pain; headache; esophageal stricture; gastric ulcer; radiation pneumonitis; cardiomyopathy; photodamaged skin, which is characterized by locally exaggerated pigmentation, looseness, fine lines, wrinkles, enlarged pores, and the development of darkened plugs in the sebacious glands; skin cancer; sunburn; solar dermatitis; photoallergic dermatitis; sun spots; age spots; and sun poisoning.

In one embodiment, treating a radiation-induced injury includes increasing a subject's survival time following exposure to radiation.

In another embodiment, death is an example of a radiation-induced injury that is preventable according to the present invention.

The Purine Compounds are also useful for protecting bystander healthy tissue from a radiation-induced injury during administration of therapeutic radiation.

A radiation-induced injury may result from exposure of a subject to ionizing radiation from numerous sources including, but not limited to, a nuclear weapon, such as an atomic bomb, a neutron bomb, or a “dirty bomb;” an industrial source, such as a nuclear power plant, a nuclear submarine, or a nuclear waste disposal site; sunlight; or a diagnostic or therapeutic medical or dental application, such as x-rays, CT scans, external radiation therapy, internal radiation therapy (e.g., radioactive “seed” implants used in cancer therapy). The injury might result from an accident, an act of war or terrorism, cumulative exposure at the home or workplace, purposeful exposure during medical diagnosis or treatment, or exposure to ultraviolet radiation, such as from sunlight.

Examples of a radiation-induced injury caused by exposure to sunlight include, but are not limited to photodamaged skin, which is characterized by locally exaggerated pigmentation, looseness, fine lines, wrinkles, enlarged pores, and the development of darkened plugs in the sebacious glands; skin cancer; sunburn; solar dermatitis; photoallergic dermatitis; sun spots; age spots; and sun poisoning. In one embodiment, a subject being treated for a radiation-induced injury caused by exposure to sunlight has been sensitized to sunlight by a disease or by medication (drug-induced sensitivity).

In one embodiment, the injury is induced by radiation from a nuclear weapon.

In another embodiment, the injury is induced by radiation from a nuclear power plant.

In still another embodiment, the injury is induced by radiation from radiation therapy that the subject is receiving for the treatment of a non-radiation related disorder.

In still another embodiment, the injury is induced by radiation from radiation therapy that the subject is receiving for the treatment of cancer.

In one embodiment, the injury is induced by radiation from a radioactive material that is ingested by a subject.

In another embodiment, the injury is caused by exposure to sunlight.

In one embodiment, the radiation-induced injury is in a cell or tissue that is exposed to a reactive species.

### 5.5.16 METHODS FOR REDUCING A SUBJECT'S CORE BODY TEMPERATURE

In one embodiment, the invention provides methods for reducing a subject's core body temperature, comprising administering to an animal in need thereof an effective amount of a Purine Compound.

Reducing a subject's core body temperature is useful for slowing metabolism or reducing oxygen consumption, particularly where oxygen delivery to a tissue is inadequate. Examples of conditions characterized by inadequate oxygen delivery to a tissue include, but are not limited to: (i) a medical procedure, such as heart surgery, brain surgery, organ transplantation, mechanical occlusion of the vascular supply, or vascular stenosis; (ii) a disorder or medical condition such as ischemia, a respiratory disorder, respiratory failure, a pulmonary disorder, anemia, anaphylactic shock, hemmorhagic shock, dehydration, compartment syndrome, intravascular thrombus, septic shock, cystic fibrosis, lung cancer, stroke, a burn, or internal bleeding; (iii) an injury such as drowning, a crush injury to one or more limbs, choking, or suffocation; (iv) a compromised airway due to asthma, a tumor, a lung injury or a tracheal injury; (v) an external compression of one or more blood vessels; or (vi) an intrinsic obstruction of one or more blood vessels.

Accordingly, the present invention encompasses methods for slowing a subject's heart rate during heart surgery; protecting a subject's tissue from damage during surgery, particular heart or brain surgery; reducing intracranial hypertension caused by brain injury in an animal; or inducing hibernation in a subject, each method comprising administering an effective amount of a Purine Compound to an animal in need thereof.

Reducing an animal's core body temperature is also useful for reducing a subject's rate of oxygen consumption. Accordingly, the present invention provides methods for reducing the rate of a subject's oxygen consumption, the method comprising administering to a subject in need thereof an effective amount of a Purine Compound.

Reducing a subject's core body temperature is useful for treating or preventing tissue damage or stroke, resulting from an inadequate supply of oxygen to a cell, a tissue, an organ or an organ system.

In one embodiment, a subject's core body temperature is reduced to increase emergency recussitation in an injured subject.

In another embodiment, a subject's core body temperature is reduced prior to and/or during heart surgery. In a specific embodiment, the subject is a human child undergoing pediatric heart surgery.

In another embodiment, a subject's core body temperature is reduced to treat respiratory failure in the subject.

In one embodiment, a subject's core body temperature is reduced to aid tissue metabolism in a subject whose respiration and ventilation is facilitated by a ventilator. In a specific embodiment, the subject whose respiration and ventilation is facilitated by a ventilator is a geriatric human. In another specific embodiment, the subject whose respiration and ventilation is facilitated by a ventilator is a premature human infant.

In one embodiment, an organ can be stored ex vivo in a composition comprising an effective amount of a Purine Compound. The composition is useful for preserving an organ's viability after being removed from a donor and before the organ is transplanted in a recipient. In one embodiment, the donor and recipient are the same.

In another embodiment, an effective amount of a Purine Compound can be administered to an animal awaiting organ transplantation to reduce the subject's core body temperature prior to or during organ transplantation.

In one embodiment, the subject's core body temperature is reduced to a temperature of from about 4° C. to about 34° C. In certain embodiments, the subject's core body temperature is reduced to about 34° C., to about 30° C., to about 25° C., to about 20° C., to about 15° C., to about 10° C., or to about 4° C.

In a specific embodiment, a subject's core body temperature is reduced to induce therapeutic hypothermia.

### 5.6 KITS

The invention encompasses kits that can simplify the administration of the Purine Compounds or composition of the invention to a subject.

A typical kit of the invention comprises a unit dosage of a Purine Compound. In one embodiment, the unit dosage form is in a container, which can be sterile, containing an effective amount of a Purine Compound and a pharmaceutically acceptable vehicle. In another embodiment, the unit dosage form is in a container containing an effective amount of a Purine Compound as a lyophilate or pharmaceutically acceptable salt. In this instance, the kit can further comprise another container that contains a solution useful for the reconstitution of the lyophilate or dissolution of the salt. The kit can also comprise a label or printed instructions for use of the Purine Compounds.

In a further embodiment, the kit comprises a unit dosage form of a composition of the invention.

Kits of the invention can further comprise one or more devices that are useful for administering the unit dosage forms of the Purine Compounds or a composition of the invention. Examples of such devices include, but are not limited to, a syringe, a drip bag, a patch or an enema, which optionally contain the unit dosage forms.

The present invention is not to be limited in scope by the specific embodiments disclosed in the examples which are intended as illustrations of a few aspects of the invention and any embodiments that are functionally equivalent are within the scope of this invention.

### 6. EXAMPLES

Materials: [³H]NECA was obtained from Du Pont NEN, Dreieich, Germany. All other unlabeled adenosine receptor agonists and antogonists can be obtained from RBI, Natick, Mass. The 96-well microplate filtration system (MultiScreen MAFC) was obtained from Millipore, Eschborn, Germany. Penicillin (100 U/mL), streptomycin (100 μg/mL), L-glutamine and G-418 were obtained from Gibco-Life Technologies, Eggenstein, Germany. Guanosine and 2′,3′-isopropylideneguanosine were purchased from Sigma Aldrich Chemical Co., USA. 2-Chloro-NECA was prepared using the methods set forth in Hutchison et al, J. Med. Chem. 33:1919-1924 (1990). 2-Iodo-NECA was prepared by following Cristalli et al., J. Med. Chem. 35:2363-2368 (1992), and Cristalli et al., J. Med. Chem. 38:1462-1472 (1995). All other materials can be obtained as described in Klotz et al., J. Biol. Chem., 260:14659-14664 (1985); Lohse et al., Naunyn-Schmiedeberg's Arch. Pharmacol., 336:204-210 (1987); and Klotz et al., Naunyn-Schmiedeberg's Arch. Pharmacol., 357:1-9 (1998).

General Methods: Proton nuclear magnetic resonance (NMR) spectra were obtained from Varian 300 MHz spectrophotometer and chemical shifts are reported in parts per million. Compounds were characterized on the basis of NMR and Mass spectral (MS) data.

### 6.1 EXAMPLE 1

### Synthesis of Compound 54

Step A—Synthesis of 2′,3′-Isopropylidene-2-cyano-N⁶-methyladenosine-5′-carboxylic acid: A mixture of 2′,3′-isopropylidene-2-cyano-N⁶-methyladenosine (670 mg, prepared using the procedure set forth in Nair et al., J. Am. Chem. Soc. 111:8502-8504 (1989)), iodobenzene diacetate (1.418 g) and 2,2,6,6-tetramethylpiperidinooxy nitroxide (64 mg) was diluted with a 1:1 mixture of acetonitrile:water (8 mL), and the resultant reaction was allowed to stir at about 25° C. for about 18 hours. The reaction mixture was extracted using ethyl acetate, and the organic layer was washed with water, dried over MgSO₄ and concentrated in vacuo. The resultant residue was suspended in methanol (10 mL) and the resultant solution was filtered. The collected solid was dried in vacuo to provide 2′,3′-isopropylidene-2-cyano-N⁶-methyladenosine-5′-carboxylic acid (340 mg). MS m/z 388.25 [M+H]⁺.

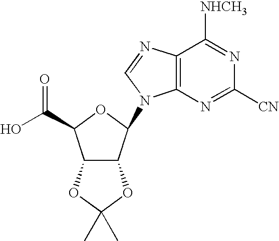

### 2′,3 ′-Isopropylidene-2-cyano-N⁶-methyladenosine-5 ′-carboxylic acid

Step B—Synthesis of N-methyl-2′,3′-isopropylidene-2-cyano-N⁶-methyladenosine-5′-N-carboxamide: A mixture of 2′,3′-isopropylidene-2-cyano-N⁶-methyladenosine-5′-carboxylic acid (150 mg), 1-ethyl-3-(3-dimethylaminopropyl)carbodiimide hydrochloride (1.5 eq.) in NAN-dimethylformamide (0.1 mL), and methylene chloride (5 mL) was stirred at room temperature and treated with the solution of methylamine (2M solution in tetrahydrofuran, 10 mL). The reaction mixture was allowed to stir at room temperature for about 15 hours and was then transferred to a separatory funnel. After aqueous workup, the organic layer was dried and concentrated in vacuo to provide a crude residue which was purified using column chromatography on silica gel (10% methanol/methylene chloride as eluent) to provide N-methyl-2′,3′-isopropylidene-2-cyano-N⁶-methyladenosine-5′-N-carboxamide (35 mg). MS m/z 388.25 [M+H]⁺.

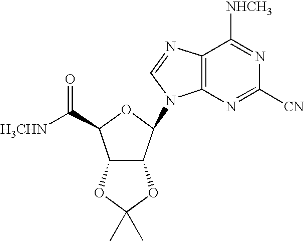

### N-methyl-2′,3′-isopropylidene-2-cyano-N⁶-methyladenosine-5′-N-carboxamide

Step C—Synthesis of N-methyl-2-cyano-N⁶-methyladenosine-5′-N-carboxamide: A solution of N-methyl-2′,3′-isopropylidene-2-cyanoadenosine-5′-carboxamide (34 mg) in trifluoroacetic acid (4 mL) and water (1 mL) was allowed to stir at room temperature for 1.5 hours. The resultant mixture was concentrated in vacuo to provide a crude residue which was recrystallized from ethyl acetate to provide N-methyl-2-cyano-N⁶-methyladenosine-5′-N-carboxamide (24 mg). MS m/z 347.95 [M+H]⁺.

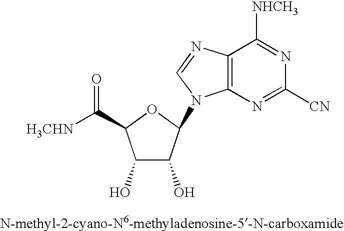

Step D—Synthesis of Compound 54: A mixture of N-methyl-2-cyano-N⁶-methyladenosine-5′-N-carboxamide (1.50 mmol, 1 eq) and phosphoric acid crystals (0.59 g, 6.00 mmol, 4 eq) were diluted with a 1:1 mixture of dimethylformamide:nitroethane (10 mL). To the resultant mixture was added tributylamine (1.43 mL, 6.00 mmol, 4 eq), followed by 1-butylimidazole (0.4 mL, 0.30 mmol, 0.2 eq). The reaction flask was then fitted with a pressure-equalizing addition funnel which was packed with molecular sieves. The reaction mixture was heated to reflux and allowed to stir at reflux for about 12 hours. The reaction was then allowed to cool to room temperature and then concentrated in vacuo to provide a crude residue. The crude residue was purified using reverse phase column chromatography (C18 resin, eluted with a gradient of 10% MeOH/H₂O to 25% MeOH/H₂O) to provide a white solid which was subsequently lyophilized to provide Compound 54 as a white fluffy solid. ¹H-NMR (300 MHz, d₆-DMSO) δ 3.0 (m, 3 H), 4.6 (m, 1 H), 5.1 (m, 1 H), 5.3 (m, 1 H), 6.4 (m, 1 H), 7.8 (m, 1 H), 8.5 (m, 1 H), 8.7 (s, 1 H).

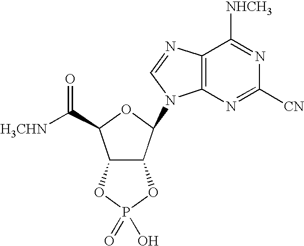

### 6.2 EXAMPLE 2

### Cell Culture and Membrane Preparation For Human Adenosine Receptor Binding Studies

CHO cells stably transfected with human adenosine A₁ receptor are grown and maintained in Dulbecco's Modified Eagles Medium with nutrient mixture F12 (DMEM/F12) without nucleosides, containing 10% fetal calf serum, penicillin (100 U/mL), streptomycin (100 μg/mL), L-glutamine (2 mM) and Geneticin (G-418, 0.2 mg/mL; A_{2B}, 0.5 mg/mL) at 37° C. in 5% CO₂/95% air. Cells are then split 2 or 3 times weekly at a ratio of between 1:5 and 1:20.

Membranes for radioligand binding experiments can be prepared from fresh or frozen cells as described in Klotz et al., Naunyn-Schmiedeberg's Arch. Pharmacol., 357:1-9 (1998). The cell suspension is then homogenized in ice-cold hypotonic buffer (5 mM Tris/HCl, 2 mM EDTA, pH 7.4) and the resultant homogenate is spun for 10 minutes (4° C.) at 1,000 g. The membranes are then sedimented from the supernatant for 30 minutes at 100,000 g and resuspended in 50 mM Tris/HCl buffer pH 7.4 (for A₃ adenosine receptors: 50 mM Tris/HCl, 10 mM MgCl₂, 1 mM EDTA, pH 8.25), frozen in liquid nitrogen at a protein concentration of 1-3 mg/mL and stored at −80° C.

### 6.3 EXAMPLE 3

### Adenosine Receptor Binding Studies

The affinities of the Purine Compounds for the adenosine Al receptor can be determined by measuring the displacement of specific [³H]2-chloro-N⁶-cyclopentyl adenosine (Perkin-Elmer Life Sciences) binding in CHO cells stably transfected with human recombinant Al adenosine receptor expressed as Ki (nM).

Dissociation constants of unlabeled compounds (Kᵢ-values) can be determined using competition experiments in 96-well microplates using the A₁ selective agonist 2-chloro-N⁶-[³H]cyclopentyladenosine ([³H]CCPA, 1 nM) for the characterization of A₁ receptor binding. Nonspecific binding is determined in the presence of 100 μM R-PIA and 1 mM theophylline, respectively. For details see Klotz et al., Naunyn-Schmiedeberg's Arch. Pharmacol., 357:1-9, 1998. Binding data can be calculated by non-linear curve fitting using the program SCTFIT (De Lean et al., Mol. Pharm. 1982, 21:5-16).

### 6.4 EXAMPLE 4

### Cell Culture and Membrane Preparation for Human Adenosine A_{2A} or A₃ Receptor-binding Studies

CHO cells stably transfected with either human adenosine A_{2A} receptor or human adenosine A₃ receptor are grown and maintained in Dulbecco's Modified Eagles Medium with nutrient mixture F12 (DMEM/F12) without nucleosides, containing 10% fetal calf serum, penicillin (100 U/mL), streptomycin (100 μg/mL), L-glutamine (2 mM) and Geneticin (G-418, 0.2 mg/mL; A_{2B}, 0.5 mg/mL) at 37° C. in 5% CO₂/95% air. Cells are then split 2 or 3 times weekly at a ratio of between 1:5 and 1:20.

Membranes for radioligand binding experiments are prepared from fresh or frozen cells as described in Klotz et al., Naunyn-Schmiedeberg's Arch. Pharmacol., 357:1-9 (1998). The cell suspension is then homogenized in ice-cold hypotonic buffer (5 mM Tris/HCl, 2 mM ethylenediamine-N,N-N′N′-tetraacetic acid, pH 7.4) and the homogenate is spun for 10 minutes (4° C.) at 1,000 g. The membranes are then sedimented from the supernatant for 30 minutes at 100,000 g and resuspended in 50 mM Tris/HCl buffer pH 7.4 (for A₃ adenosine receptors: 50 mM Tris/HCl, 10 mM MgCl₂, 1 mM EDTA, pH 8.25), frozen in liquid nitrogen at a protein concentration of 1-3 mg/mL and stored at −80° C.

### 6.5 EXAMPLE 5

### Anti-inflammatory Effects

Induction of Endotoxic Shock

For cytokine production, Male BALB/c mice (6-8 weeks of age) are treated with a Purine Compound (oral administration at 0.03 mg/kg) orally by gavage 30 minutes before being subjected to LPS (1 mg/kg i.p.) for 90 minutes. A blood sample is then taken and serum is obtained for analysis. Serum is diluted 1:5 prior to being assayed for cytokines using species-specific ELISA kits (R & D Systems) for the chemokine MIP-1α and the cytokine TNF-α levels.

### 6.6 EXAMPLE 6

### Function Recovery After Global Ischemia/Reperfusion

Heart Perfusion

Male Sprague-Dawley rats (each having a body weight of 250 to 300 g) are heparinized using sodium heparin (1,000 U/kg i.p.), followed 10 minutes later by introduction of anesthesia via intraperitoneal administration of sodium pentobarbital (40 mg/kg). Once the subject is anesthetized, the thorax is opened, and the heart is rapidly removed and perfused through the ascending aorta using Krebs-Ringer buffer consisting of NaCl (118 mmol/liter), KCl (4.75 mmol/liter), KH₂PO₄ (1.18 mmol/liter), MgSO₄ (1.18 mmol/liter), CaCl₂ (2.5 mmol/liter), NaHCO₃ (25 mmol/liter), and glucose (11 mmol/liter). A mixture of 95% O₂ and 5% CO₂ at 37° C. is then bubbled through the perfusate. The heart is initially perfused at a constant pressure of 70 mm Hg. About 10 minutes after the constant pressure perfusion, perfusion is switched to constant flow perfusion achieved using a microtube pump. The perfusion pressure is maintained at the same level of constant pressure perfusion by adjusting flow rate. Once the flow rate is determined, it is maintained throughout the experiment. The hearts are stimulated by rectangular pulses at a rate of 5 Hz and 2-millisecond duration and twice the diastolic threshold, delivered from a stimulus isolation unit (AD Instruments Ltd, Australia).

Function Recovery After Ischemia/Reperfusion

Rat hearts are initially perfused at a constant pressure of 70 mm Hg using the procedure described above under the heading “Heart Perfusion.” After a 20 minute stabilization period, the hearts are subjected to 30 minute no-flow ischemia followed by 40 minute reperfusion. The Purine Compounds are infused in hearts for 10 minutes prior to induction of ischemia. Bipolar epicardial electrocardiogram (ECG) is recorded by placing two electrodes on the surface of right appendage and apex. A stainless steel cannula is used as indifferent electrode. After a 20-minute equilibration period, regional ischemia is induced by ligation of the left anterior descending (LAD) coronary artery, and the ligature is released 30 minutes after occlusion. The hearts are then subject to 40 minutes of reperfusion A Purine Compound is applied interperfusate 10 minutes before LAD ligation and is present during LAD ligation. The Purine Compounds are typically tested in this model at 10, 30 and 100 pM concentrations.

To assess contractile function, a microtip catheter transducer (Millar Instruments Inc., Houston, Tex.) is inserted directly into the left ventricular cavity and data can be collected using a PowerLab data acquisition system (ADInstruments Ltd, Australia) in conjunction with a Macintosh computer, and analyzed using Chart.3 computer package. Coronary perfusion pressure (CPP), left ventricular systolic pressures (LVSP), left ventricular end diastolic pressures (LVEDP), maximal rates of development of left ventricular pressure (+dP/dtₘₐₓ, −dP/dtₘᵢₙ) can be measured using this method. Left ventricular developed pressure (LVDP) can be calculated as the difference between the systolic and diastolic pressure.

### 6.7 EXAMPLE 7

### Wound Healing

Endothelial Cell and Fibroblast Migration

In vitro wound assays can be performed as described by Shleef et al., Tissue Cell 14:629-636 (1982). Cells, for example, human umbilical or saphenous vein endothelial cells, dermal fibroblasts, etc., are cultured in Medium 199 containing 10% fetal bovine serum until they form confluent monolayers, for example, in 12 well culture plates. The confluent monolayers are treated with mitomycin C (10 μg/ml) and 60 minutes later are wounded using a razor blade. The wounded cells are rinsed several times with saline and a predetermined amount of a Purine Compound is then added to replicate wells. Cell migration into the wound is assessed at various times thereafter using phase contrast microscopy with an inverted microscope. Quantitation may be performed by aligning the original edge of the wound with the “0” line on a 10×10 grid-reticle and the counting the number of cells in each of the 10 rows defined by the reticle.

### 6.8 EXAMPLE 8

### Asthma-Associated Inflammation

Aerosol Exposure and Bronchoalveolar Lavage

Four-week old male, viral-antibody-free BALB/c mice (Jackson Laboratory, Bar Harbor, Me.) are intraperitoneally immunized with 10 μg ovalbumin (“OVA,” Grade III, Sigma Chemical Co., St. Louis, Mo.) and 1 mg alum (diluted from 2% Alhydrogel; Accurate Sci. Corp., Westbury, N.Y.) in 0.5 mL phosphate-buffered saline (“PBS”) on days 0 and 7. Control mice are intraperitoneally administered 1 mg alum in PBS solution on days 0 and 7.

On day 14, both immunized mice and control mice are administered a single aerosol exposure to 3% OVA (in PBS) for 30 minutes, followed by intraperitoneal administration of a Purine Compound (5 μg per mouse in 0.2 mL buffer solution). About 18 hours after treatment, the mice are sacrificed and bronchoalveolar lavage (“BAL”) is performed on their lungs. The fluid obtained from the mice via the BAL procedure is analyzed and the inflammatory cell counts and level of inflammatory mediators in the fluid samples are measured as described in Virag et al., Med. Sci. Monit. 10:BR77-83 (2004).

### 6.9 EXAMPLE 9

### TPA-Induced Dermatitis

Induction of Dermatitis

Dermatitis is induced in the right ear of unanesthetized mice via the topical application of 12-O-tetradecanoylphorbol-13-acetate (TPA) (10 μL, 1% in DMSO) on both the inner and outer surfaces of the right ear. The left ear of each mouse has only vehicle (DMSO, 10 μL) topically applied on both the inner and outer surfaces.

Administration to Dermatitis-Induced Ear with a Purine Compound

Immediately after application of TPA, the mice are topically treated on the inner and outer surfaces of their right ear only with either: (1) a Purine Compound (10 μL, 0.1% in normal saline), (2) a Purine Compound (10 μL, 0.3% in normal saline), or (3) normal saline (10 μL).

Six hours after the application of the Purine Compound or normal saline, the animals are euthanized using CO₂ asphyxiation and a ¼ inch biopsy of both the left and right ear is taken and weighed. The biopsy samples are then analyzed for myloperoxidase (MPO) activity as a marker of neutrophil infiltration using standard methods.

### 6.10 EXAMPLE 10

### Dextran Sodium Sulfate-Induced Colitis

Colitis is induced in Swiss Webster mice by administration of dextran sodium sulfate (DSS) (5%, dissolved in distilled water, molecular weight 30-40 kDa) ad libitum for a total period of seven days. During this seven-day period, and concomitant with the administration of DSS, the mice are separately administered a Purine Compound twice daily by gavage at a total daily dose of 0.1 mg/kg/day, 0.3 mg/kg/day or 1 mg/kg/day. At the end of the seventh day of administration of both DSS and the Purine Compound, the mice are euthanized and their colon is removed, measured, visually analyzed and colon biopsy samples are taken and analyzed for malondialdehyde (MDA) and myeloperoxidase (MPO) levels.

### 6.11 EXAMPLE 11

### LPS-Induced Chemokine and Cytokine Response

Male BALB/c mice are intraperitoneally administered a Purine Compound (at a dose of either 0.3 mg/kg or 1.0 mg/kg) over a 30-minute period. Lipopolysaccharide (LPS) is then administered intraperitoneally at a dose of 1 mg/kg. Ninety minutes after LPS administration, serum is collected and the levels of MIP-1α and TNF-α are analyzed using specific ELISA.

### 6.12 EXAMPLE 12

### Plasma and Lung Tissue Concentrations of Compound 54 after Intratracheal Administration

Plasma Concentration

A 10 mg/mL solution of Compound 54 in saline was prepared and 0.2 mL of this solution was administered into the trachea of two Sprague-Dawley rats via intubation. Blood samples were taken at 10 minutes, 30 minutes, 60 minutes and 120 minutes, post-intubation. The plasma concentration of Compound 54 was measured at each of these time points using HPLC and the peak area of the amount of Compound X in the plasma was measured at each of these time points using LC/MS.

Lung Tissue Concentration

After 120 minutes post-intubation, the rats are sacrificed and lung samples are collected, homogenized in PBS at 4° C. The concentration of Compound 54 in the homogenate can be measured using HPLC, and the concentration of Compound X in the homogenate can be measured using LC/MS.

Table 3 below shows the plasma concentration of Compound 54 at 10 minutes, 30 minutes, 60 minutes and 120 minutes in both rat test subjects.

TABLE 3
Plasma Concentration of Compound 54 in Rats

| | Plasma | Plasma | Mean |
|---|---|---|---|
| | Concentration | Concentration | Plasma |
| Time | Rat 1 | Rat 1 | Concentration |
| (min.) | (ng/mL) | (ng/mL) | (ng/mL) |
| 10 | 830 | 627 | 728.5 |
| 30 | 232 | 202 | 217 |
| 60 | 69.3 | 26 | 47.65 |
| 120 | 9.7 | 9.5 | 9.6 |

Table 4 below shows the mean peak area of Compound X at 10 minutes, 30 minutes, 60 minutes and 120 minutes in both rat test subjects.

TABLE 4
Peak Area of Compound X in Rats

| Time | Peak Area | Peak Area | Mean |
|---|---|---|---|
| (min.) | Rat 1 | Rat 2 | Peak Area |
| 10 | 140 | 64 | 102 |
| 30 | 9.6 | 12 | 10.8 |
| 60 | 4.4 | 1.8 | 3.1 |
| 120 | 0.4 | n/a | 0.4 |

n/a—not available

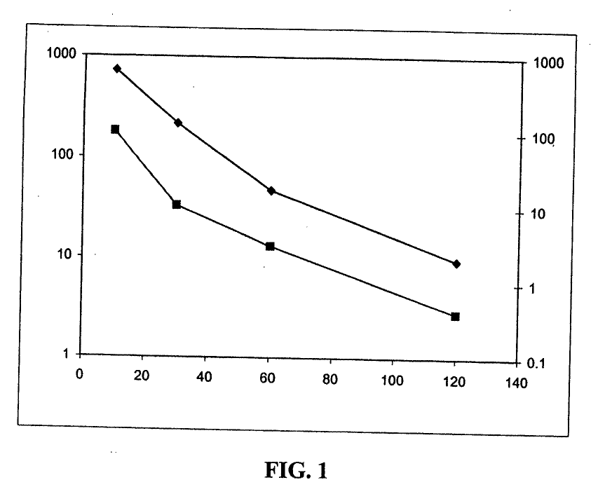

FIG. 1 shows a line graph illustrating the blood plasma concentration of Compound 54 and the mean peak area of Compound X at 10 minutes, 30 minutes, 60 minutes and 120 minutes. The peak area of Compound X correlates with the plasma concentration of this compound.

Compound X has the structure:

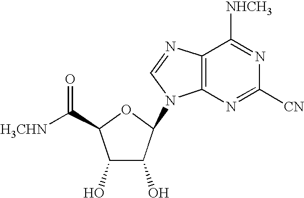

### Synthesis of Compound 64a

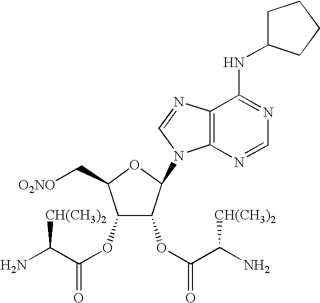

### 64a

N⁶-Cyclopentyladenosine: A solution of 6-chloroadenosine (43 g) and cyclopentylamine (5 eq.) in ethanol (50 eq.) was heated at reflux for 3 hours then cooled to room temperature. The resultant reaction mixture was concentrated in vacuo and the resultant residue was diluted with water (400 ml) and ethyl acetate (400 ml). The organic layer was separated and the aqueous layer was extracted into ethyl acetate (2×400 ml). The combined organic layers were washed with water (2×200 ml), dried over sodium sulfate, concentrated in vacuo and dried under vacuum to provide a solid which was suspended in MeOH (400 mL), filtered and dried to provide N⁶-cyclopentyladenosine (43.8 g).

2′,3′-isopropylidene-N⁶-cyclopentyladenosine: N⁶-cyclopentyladenosine (43 g) was diluted with acetone (75 eq.) and to the resultant solution was added 2,2-dimethoxypropane (5 eq.), followed by D-camphorsulphonic acid (1 eq) and the resultant reaction was allowed to stir at room temperature for 3 hours. The resultant reaction mixture was concentrated in vacuo and the resultant residue was diluted with ethyl acetate, then neutralized to pH 7.0 using concentrated aqueous NaHCO₃. The organic layer was separated, dried over sodium sulfate, concentrated in vacuo and dried under vacuum to provide a solid which was suspended in hexane (250 mL), filtered, washed with hexane and dried under vacuum to provide 2′,3′-isopropylidene-N⁶-cyclopentyl adenosine (43 g).

2′,3′-isopropylidene-N -cyclopentyladenosine-5′-O-nitrate: Acetic anhydride (22 eq) was slowly added to a stirred solution of nitric acid (5 eq., 63%) at −10° C. (acetonitrile-CO₂ bath used for cooling) over a period of 4 hours with the reaction temperature maintained at −5 to 5° C. during the addition. The resultant solution was cooled to −20° C. and a solution of 2′,3′-isopropylidene-N⁶-cyclopentyladenosine (18.250 gm, 0.048 mol) in acetic anhydride (37 mL, 8 eq.) was added slowly. The resultant reaction was allowed to stir at −15 to −5° C. for 1 hour and the resultant reaction mixture was slowly poured slowly into an ice-cold solution of aqueous NaHCO₃ (168 gm in 800 mL water) and ethyl acetate (350 mL) and the resultant solution was allowed to stir for 5 minutes. The organic layer was separated and the aqueous layer was extracted using ethyl acetate (350 mL). The combined organic layers were washed with water, and dried over sodium sulfate, concentrated in vacuo and purified using flash column chromatograpy on silica gel using 70% ethyl acetate-hexane as eluent to provide 2′,3′-isopropylidene-N⁶-cyclopentyladenosine-5′-O-nitrate (14.9 g).

N⁶-cyclopentyladenosine-5 ′-O-nitrate: 2′,3′-isopropylidene-N⁶-cyclopentyladenosine-5′-O-nitrate (4.8 g) was diluted with a mixture of TFA (20 mL) and water (5 mL) and the resultant reaction was allowed to stir for 30 minutes at room temperature. The resultant reaction mixture was concentrated in vacuo and the resultant residue was diluted with water (10 mL) and concentrated in vacuo. The resultant residue was diluted with ethyl acetate and washed with saturated aqueous sodium bicarbonate, and the organic layer was dried over sodium sulfate and concentrated in vacuo to provide a white solid residue which was dried under vacuum and then recrystalized from cold ethanol to provide N⁶-cyclopentyladenosine-5′-O-nitrate (3.1 gm). ¹H-NMR (DMSO-d₆): δ 1.49-1.58 (m, 4H), 1.66-1.72 (m, 2H), 1.89-1.94 (m, 2H), 4.12-4.17 (m, 1H), 4.28-4.33 (m, 1H), 4.48 (bs, 1H), 4.65-4.87 (m, 3H), 5.5 (d, J=5.1 Hz, 1H), 5.63 (d, J=5.7 Hz, 1H), 5.91 (d, J=5.1 Hz, 1H), 7,75 (d, J=7.5 Hz, 1H), 8.17 (bs, 1H), 8.30 (s, 1H); MS (ES+): m/z 381.35 (M+1); Anal. Calcd for C₁₅H₂₀N₆O₆: C, 47.37; H, 5.30; N, 22.10; Found: C, 47.49; H, 5.12, N, 21.96.

Compound 64a: A mixture of t-BOC-valine (651 mg) and DCC (620 mg) was diluted with toluene (20 ml) and to the resultant solution was added 4-pyrrolidinopyridine (15 mg), followed by N⁶-cyclopentyladenosine-5′-O-nitrate (190 mg) and the resultant reaction was allowed to stir at room temperature for about 1 hour. The reaction temperature was then raised to 45° C. and the reaction mixture was allowed to stir at this temperature for about 15 hours. The reaction mixture was then concentrated in vacuo and the resultant residue was purified using flash column chromatography on silica gel (60% ethyl acetate/hexane eluent) to provide a crude residue. The crude residue (270 mg) was diluted with formic acid (20 mL) and the resultant reaction was allowed to stir at room temperature for about 15 hours. The reaction mixture was then concentrated in vacuo and the crude residue obtained was recrystallized from methanol/ether to provide Compound 64a (135 mg). MS (ES⁺): m/z 579.5 (M+1).

### 6.13 EXAMPLE 13

### Septic Shock

Male BALB/c mice (6-8 weeks of age) are used in studies investigating lipopolysaccharide-induced cytokine production and survival. For cytokine production the mice are treated with an illustrative Purine Compound (oral administration of 0.03 mg/kg) orally by gavage 30 minutes before being subjected to lipopolysaccharide (1 mg/kg i.p.) for 90 minutes, after this period blood is taken and serum is obtained for analysis. Serum is diluted 1:5 prior to being assayed for cytokines using species-specific ELISA kits (R & D Systems) for the chemokine MIP-1α and the cytokine TNF-α levels, which are expressed as pg/mL. For survival studies mice are treated with an illustrative Purine Compound (oral administration of 0.03 mg/kg) starting 30 minutes prior to the mice being subjected to lipopolysaccharide (55 mg/kg i.p.). The survival of the mice is followed over 72 hours and expressed as a percentage of surviving mice at each time point.

### 6.14 EXAMPLE 14

### Anti-Arrhythmia Effects

Heart Perfusion

Male Sprague-Dawley rats (having a body weight of 250 to 300 g) are heparinized using sodium heparin (1,000 U/kg i.p.), followed 10 minutes later by introduction of anesthesia via intraperitoneal administration of sodium pentobarbital (40 mg/kg). Once the animal is anesthetized, the thorax is opened, and the heart is rapidly removed and perfused through the ascending aorta using Krebs-Ringer buffer consisting of NaCl (118 mmol/liter), KCl (4.75 mmol/liter), KH₂PO₄ (1.18 mmol/liter), MgSO₄ (1.18 mmol/liter), CaCl₂ (2.5 mmol/liter), NaHCO₃ (25 mmol/liter), and glucose (11 mmol/liter). A mixture of 95% O₂ and 5% CO₂ at 37° C. is then bubbled through the perfusate (the heart is initially perfused at a constant pressure of 70 mm Hg). About 10 minutes after the constant pressure perfusion, perfusion is switched to constant flow perfusion achieved using a microtube pump. The perfusion pressure is maintained at the same level of constant pressure perfusion by adjusting flow rate. Once the flow rate is determined, it is maintained throughout the experiment. The hearts are stimulated by rectangular pulses at a rate of 5 Hz and 2-millisecond duration and twice the diastolic threshold, delivered from a stimulus isolation unit (ADInstruments Ltd, Australia).

### Effect of Purine Compounds on Ischemia-induced Arrhythmias

Rat hearts are perfused at constant pressure of 70 mmHg without pacing as described above. Bipolar epicardial electrocardiogram (ECG) is recorded by placing two electrodes on the surface of right appendage and apex. A stainless steel cannula is used as an indifferent electrode. The ECG and heart rate are continuously monitored and data are recorded using a PowerLab data acquisition system (ADInstruments Ltd, Australia) in conjunction with a computer, and are analyzed using the Chart.3 computer.package. After a 20-minute equilibration period, regional ischemia is induced by ligation of the left anterior descending (LAD) coronary artery, and the ligature is released 30 minutes after occlusion. An illustrative Purine Compound is applied interperfusate 10 minutes before LAD ligation and is present during LAD ligation. An illustrative Purine Compound can be tested at 10, 30 and 100 pM concentrations.

### 6.15 EXAMPLE 15

### Determination of the Effect of the Purine Compounds on Pain

Male mice (body weight of 25-35 grams) are put into groups as, follows: a first group which is to be intreperitoneally administered buprenorphine (0.3 mg/kg), a second group which is to be intreperitoneally administered buprenorphine (1 mg/kg), a third group which is to be intreperitoneally administered an illustrative Purine Compound (3 mg/kg), a fourth group which is to be intreperitoneally co-administered an illustrative Purine Compound (3 mg/kg) and buprenorphine (1.0 mg/kg), and a fifth group which is to be intreperitoneally co-administered an illustrative Purine Compound (3 mg/kg) and buprenorphine (0.3 mg/kg). The analgesic effects in mice are measured using an IITC model 33 tail-flick analgesia meter (IITC Inc., Woodland Hills, Calif.) at 0 minutes (baseline control), 5 minutes, 15 minutes, 30 minutes and 60 minutes (in some cases also 90 and 120 minutes) post-treatment, compound or vehicle treatment. The average recording value of two readings can be used for each time point. A baseline of between 2-4 seconds of latency for each mouse and a 10-second cut-off time is set for the maximum possible effect of analgesia (% MPE). % MPE is calculated using the following Formula: %MPE=[(post-drug value−baseline)/(cut-off time−baseline)]×100.

### 6.16 EXAMPLE 16

### Determination of the Effect of the Purine Compounds on Pain

Male mice (each having a body weight of 20-30 g) are subcutaneously administered 20 μl of a 1% formalin solution in formaldehyde (prepared by diluting a commercial 4% [w/v] stock formalin solution) into the dorsal region of their left hind paw. The mice are then assigned to either a control group and administered vehicle, or to a treatment group. Each group is then intraperitoneally administered an illustrative Purine Compound (1.0 mg/kg). Both groups of animals are then monitored for a reaction for 30 minutes post-treatment to determine how much time each animal spends licking the treated paw. The licking time in control group (vehicle pretreated animals) is then compared to the licking time in the treatment group in order to calculate the analgesic effect. The 30 minute reaction period is divided into two phases: an early phase which lasts from 0-5 minutes post-treatment, and a late phase which lasts from 10-30 minutes post-treatment.

### 6.17 EXAMPLE 17

### Determination of the Effect of the Purine Compounds on Pain

BALB/C mice (6-8 weeks of age) are intraperitoneally administered streptozotocin (40 mg/kg, once per day for 5 consecutive days) to induce diabetes (blood glucose levels are greater than 200 mg/mL). Three weeks after the first streptozotocin injection, the animals are intraperitoneally administered an illustrative Purine Compound (1 mg/kg) into a rear paw and post-treatment allodynia is measured using an Electrovonfrey anesthesiometer (IITC Inc., Woodland Hills Calif. 91367). The analgesic activity of an illustrative Purine Compound is measured at 0 minutes (control), 15 minutes, 30 minutes and 60 minutes after administration of the illustrative Purine Compound.

### 6.18 EXAMPLE 18

### Determination of the Effect of the Purine Compounds on Pain

Male Wistar rats (each weighing between 200-250 g, kept under pathogen-free conditions at 24-25° C. and provided with standard rat chow and water ad libitum) are anaesthetized via intraperitoneal administration of pentobarbital (50 mg/kg) and placed in a stereotaxic frame. The atlanto-occipital membrane is exposed and a PE-10 catheter (7.5 cm) is inserted through an incision into the subarachnoidal space. The external end of the catheter is then fixed to the skull, the wound is closed, and the rats are allowed to recover for 7 days post-surgery. Animals without neurological deficits are placed in a plexiglass observation chamber on a metal mesh surface and mechanical thresholds of the plantar surface of the paw can be determined using a Dynamic Plantar Aesthesiometer (Ugo Basile, Italy) as follows: Following acclimation, the touch stimulator unit is placed under the animal's paw such that the filament is positioned under the target area of the paw. The filament is then lifted such that it contacted the pad of the animal's paw and continually exerted an increasing upward force on the paw until the animal withdrew the paw. The paw withdrawal threshold is measured 5 times in this manner in turns and the mean of the 5 values is calculated. After control threshold measurements are complete, carrageenan (3%, 100 μl) is administered subcutaneously into a hindpaw, resulting in marked swelling and redness of the treated paw. Three hours after the carrageenan administration, the threshold values are measured again. The animals are then divided into a control group (administered vehicle intrathecally) and a treatment group (adminstered an illustrative Purine Compound intrathecally at in a 10 μl injection volume). Threshold determinations are repeated as described above at 15 minutes, 30 minutes, 60 minutes, 90 minutes and 120 minutes after the administration of vehicle or an illustrative Purine Compound.

### 6.19 EXAMPLE 19

### Determination of the Effect of the Purine Compounds on Pain

Male CD rats (each weighing from 220 g to 250 g) are prepared according to the procedure set forth in Z. Seltzer et al., Pain, 43:205-218 (1990). The rats are then anesthetized via intraperitoneal administration of sodium pentobarbital (50 mg/kg). A skin incision is made at the upper ⅓ and ⅔ left thigh area of each rat and the left sciatic nerve is exposed and freed from the surrounding connective tissue. An 8-0 nylon suture is then used to tightly ligate the left sciatic nerve of each rat so that the dorsal ⅓ to ½ of the nerve thickness is trapped in the ligature. The incision is closed using 4-0 sterile suture. Seven days post-surgery, the animals are put into four groups: a first group that is administered vehicle (control group); a second group that is administered an illustrative Purine Compound at 0.1 mg/kg; a third group that is administered buprenorphine at 0.3 mg/kg; and a fourth group that is co-administered an illustrative Purine Compound at 0.1 mg/kg and buprenorphine at 0.3 mg/kg. Animals in all four groups are assessed for allodynia immediately prior to treatment and at 10, 20, 30 and 60 minutes post-treatment using the Von Frey Hair test (G. M. Pitcher et al., J Neurosci Methods, 87:185-93 (1999)).

### 6.20 EXAMPLE 20

### Determination of the Effect of the Purine Compounds on Heart Rate

Adult male Wistar rats (each weighing from about 350 g to about 400 g) are anesthetized as in Example 9, then prepared for monitoring of blood pressure and heart rate.

Each animal's heart rate is measured, then an illustrative Purine Compound is intravenously administered via the femoral vein at a dose of 1 ng/kg/minute, 10 ng/kg/minute, or 1000 ng/kg/minute (n=2 animals per dosage size) for a total administration period of 20 minutes. Each animal's heart rate is then remeasured. The post-treatment heart rate is then compared to the pre-treatment heart rate.

### 6.21 EXAMPLE 21

### Determination of the Effect of the Purine Compounds on Core Body Temperature

Two male Sprague-Dawley rats of about 400 g each are kept at 13° C. and slowly injected with 20 mg/mL an illustratire Purine Compound dissolved in saline through a jugular venus (JV) catheter for about 2 minutes to reach a dose of 15 mg/kg. After the rats fall asleep, 20 mg/mL of an illustrative Purine Compound is continuously injected through the jugular venus catheter via a syringe pump for 4 hours at a rate 1 mL/h. The rats are then returned to their cages at room temperature. The rectal temperature, respiratory rate, and behavior of the rats are recorded following 5 min, 10 min, 20 min, 30 min, 1 h, 1.5 h, 2 h, 2.5 h, 3 h, 3.5 h, and 4 h.

After the experiments, the animals are kept in the animal room and their behavior observed.

### 6.22 EXAMPLE 22

### Determination of the Effect of the Purine Compounds on Treatment or Prevention of Glaucoma with Intraocular Hypertension

The effect of an illustrative Purine Compound on intraocular pressure (IOP) is examined in New Zealand white rabbits. New Zealand white rabbits undergo a circadian change in intraocular pressure, such that lowest pressure values occur in the early morning and peak pressure values occur in the afternoon.

An illustrative Purine Compound is dissolved in saline, at concentrations 0.3, 1.0, 3.0, 10.0, and 30.0 mg/mL. One rabbit is administered with each each dose level. One drop (about 100 μL) of the saline solution of the Purine Compound is applied to the external surface of one eye of each rabbit. The illustrative Purine Compound is administered at t=0 hours, 3 hours after the animal house dark period ended (lights came on in the rabbit house at t=−3 hours). Thus, the illustrative Purine Compound is administered when the level of intraocular pressure is low relative to other timepoints during the day and night.

An illustrative Purine Derivative that reduces an animal's intraocular pressure would accordingly be useful for treating or preventing glaucoma with intraocular hypertension.

The present invention is not to be limited in scope by the specific embodiments disclosed in the examples which are intended as illustrations of a few aspects of the invention and any embodiments that are functionally equivalent are within the scope of this invention.

All references cited herein are incorporated by reference in their entirety.

What is claimed is:

1. A compound of Formula (I): 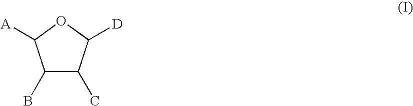
or a pharmaceutically acceptable salt thereof,
wherein
A is —C(O)NHR³, —CH₂NHR, —CH₂OSO₂NH₂, —CH₂ONO₂, —CH₂ONO, —CH₂OSO₃H, —CH₂OSO₂NH(C₁-C₁₀ alkyl), —CH₂OSO₂N(C₁-C₁₀ alkyl)₂, —CH₂OH or —CH₂OSO₂NH-aryl, where each C₁-C₁₀ alkyl is independent;
B is —OR⁹;
C is —OR¹⁰;
R⁹ and R¹⁰ are independently the residue of a naturally occurring amino acid that is attached via its C-terminus, or R⁹ and R¹⁰ join to form a —P(O)(OH)— group;
D is: 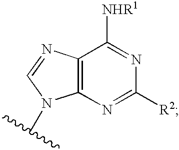
A and B are trans with respect to each other;
B and C are cis with respect to each other;
C and D are cis or trans with respect to each other;
when A is —C(O)NHR³, —CH₂OSO₂NH(C₁-C₁₀ alkyl), —CH₂OSO₂N(C₁-C₁₀ alkyl)₂, or —CH₂OSO₂NH-aryl, where each C₁-C₁₀ alkyl is independent, then R¹ is H, —C₁-C₁₀ alkyl, -aryl, —(C₁-C₆alkylene)-aryl, —(C₁-C₆alkylene)-(arylene)-halo, -3 to 7-membered monocyclic heterocycle, -8 to 12-membered bicyclic heterocycle, —(CH₂)ₙ—C₃-C₈ monocyclic cycloalkyl, —(CH₂)ₙ—C₃-C₈ monocyclic cycloalkenyl, —(C₃-C₈ monocyclic cycloalkene)-OH, —(CH₂)ₙ—C₈-C₁₂ bicyclic cycloalkyl, —(CH₂)ₙ—C₈-C₁₂ bicyclic cycloalkenyl, or —(CH₂)ₙ-aryl;
when A is —CH₂OSO₂NH₂, then R¹ is —C₃-C₈ monocyclic cycloalkyl, —(C₃-C₈ mionocyclic cycloalkylene)-OH, —C₃-C₈ monocyclic cycloalkenyl, —(CH₂)ₙ—(C₃-C₈ monocyclic cycloalkyl), —(CH₂)ₙ—(C₃-C₈ monocyclic cycloalkenyl), —C₈-C₁₂ bicyclic cycloalkyl, or —C₈-C₁₂ bicyclic cycloalkenyl;
when A is —CH₂NHR¹¹, —CH₂ONO₂, —CH₂ONO, —CH₂OH, or —CH₂OSO₃H, then R¹ is —H, —C₁-C₁₀ alkyl, -aryl, -3 to 7-membered monocyclic heterocycle, -8 to 12-membered bicyclic heterocycle, —C₃-C₈ monocyclic cycloalkyl, -C₃-C₈ monocyclic cycloalkenyl, —(C₃-C₈ monocyclic cycloalkyl)-OH, —(C₃-C₈ monocyclic cycloalkylene)-OH, —C₈-C₁₂ bicyclic cycloalkyl, —C₈-C₁₂ bicyclic cycloalkenyl, —(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkyl), —(CH₂)ₙ—(C₈-C₁₂ bicyclic cycloalkenyl), or —(CH₂)ₙ-aryl;
R² is —H, halo, —CN, —NHR⁴, —OR⁴, —SR⁴, —NHC(O)OR⁴, —NHC(O)R⁴ —NHC(O)NHR⁴, —NHNHC(O)R⁴, —NHNHC(O)NHR⁴, —NHNHC(O)OR⁴, —NH—N═C(R⁵)R⁶, —NR⁵—N═C(R⁵)R⁶ or —NR⁵—N(R⁷)R⁸;
R³ is —C₁-C₁₀ alkyl, -aryl, -3 to 7-membered monocyclic heterocycle, -8 to 12-membered bicyclic heterocycle, —C₃-C₈ monocyclic cycloalkyl, —(CH₂)—(C₃-C₈ monocyclic cycloalkyl), —C₃-C₈ monocyclic cycloalkenyl, —C₈-C₁₂ bicyclic cycloalkyl or —C₈-C₁₂ bicyclic cycloalkenyl;
R⁴ is —H, —C₁-C₁₅ alkyl, -aryl, —(CH₂)ₙ-aryl, —(CH₂)ₙ—(C₃-C₈ monocyclic cycloalkyl), —O—(CH₂)ₙ—(C₃-C₈ monocyclic cycloalkyl), —(CH₂)ₙ—(C₃-C₈ monocyclic cycloalkenyl), —O—(CH₂)ₙ—(C₃-C₈ monocyclic cycloalkenyl), —(CH₂)ₙ—(C₈-C₁₂ bicyclic cycloalkyl), —O—(CH₂)ₙ—(C₈-C₁₂ bicyclic cycloalkyl), —(CH₂)ₙ—(C₈-C₁₂ bicyclic cycloalkenyl), —(CH₂)ₙ-(3 to 7-membered monocyclic heterocycle) or —(CH₂)ₙ-(8 to 12-membered bicyclic heterocycle) —C≡C—(C₁-C₁₀ alkyl) or —C≡C-aryl;
each occurrence of R⁵ is independently —H, —C₁-C₁₀ alkyl, -aryl, —(CH₂)ₙ-aryl, —(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkyl), —(CH₂)ₙ—(C₈-C₁₂ bicyclic cycloalkenyl), —(CH₂)ₙ-(3 to 7-membered monocyclic heterocycle), —(CH₂)ₙ-(8 to 12-membered bicyclic heterocycle), —(CH₂)ₘ-phenylene-(C₂-C₁₀ alkynyl), —(CH₂)ₘ-phenylene-(CH₂)ₘCOOH, —(CH₂)ₘ-phenylene-(CH₂)ₘCOO—(C₁-C₁₀ alkyl), —(CH₂)ₘ-phenylene-(CH₂)ₘ—(C₃-C₇-membered monocyclic heterocycle), or —(CH₂)ₘ—C(O)—(C₁-C₁₀ alkyl);
or R⁵ and R⁶, together with the carbon atom to which they are attached, join to form a cyclopentyl, 2-cyclopentenyl, 3-cyclopentenyl, cyclohexyl, 2-cyclohexenyl, 3-cyclohexenyl ring or 1,2,3,4-tetrahydronaphthalene group;
or when A is —CH₂OSO₂NH₂, —CH₂ONO, —CH₂OH or —CH₂OSO₃H, then R⁵ and R⁶, together with the carbon atom to which they are attached, join to form —C₃-C₈ monocyclic cycloalkyl, a —C₈-C₁₂ bicyclic cycloalkyl, a —C₃-C₈ monocyclic cycloalkenyl or a —C₈-C₁₂ bicyclic cycloalkenyl;
R⁶ is —H, —C₁-C₁₀ alkyl, -aryl, —(CH₂)ₙ-aryl, —(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkyl), —(CH₂)ₙ—(C₈-C₁₂ bicyclic cycloalkenyl), —(CH₂)ₙ-(3 to 7-membered monocyclic heterocycle), —(CH₂)ₙ-(8 to 12-membered bicyclic heterocycle), —(CH₂)ₘ-phenylene-(C₂-C₁₀ alkynyl), —(CH₂)ₘ-phenylene-(CH₂)ₘ-(-3 to 7-membered monocyclic heterocycle), —(CH₂)ₘ-phenylene-(CH₂)ₘCOOH or —(CH₂)ₘ-phenylene-(CH₂)ₘCOO—(C₁-C₁₀ alkyl);
R⁷ is —C₁-C₁₀ alkyl, -aryl, —(CH₂)ₙ-aryl, —(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkyl), —(CH₂)ₙ—(C₈-C₁₂ bicyclic cycloalkenyl), —(CH₂)ₙ-(-3 to 7-membered monocyclic heterocycle), —(CH₂)ₙ-(-8 to 12-membered bicyclic heterocycle), —(CH₂)ₘ-phenylene-(C₂-C₁₀ alkynyl), —(CH₂)ₘ-phenylene-(CH₂)ₘ—(C₃-C₇membered monocyclic heterocycle), —(CH₂)ₘ-phenylene-(CH₂)ₘCOOH, —(CH₂)ₘ-phenylene-(CH₂)ₘCOO—(C₁-C₁₀ alkyl), —(CH₂)ₘ—C(O)—(C₁-C₁₀ alkyl), or R⁷ and R⁸, together with the nitrogen atom to which they are attached, join to form a -3- to 7-membered nitrogen-containing monocyclic heterocycle or a -8- to 12-membered nitrogen-containing bicyclic heterocycle;
R⁸ is —C₁-C₁₀ alkyl, -aryl, —(CH₂)ₙ-aryl, —(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkyl), —(CH₂)ₙ—(C₈-C₁₂ bicyclic cycloalkenyl), —(CH₂)ₙ-(-3- to 7-membered monocyclic heterocycle), —(CH₂)ₙ-(-8- to 12-membered bicyclic heterocycle), —(CH₂)ₘ-phenylene-(C₂-C₁₀ alkynyl), —(CH₂)ₘ-phenylene-(CH₂)ₘCOOH, —(CH₂)ₘ-phenylene-(CH₂)ₘCOO—(C₁-C₁₀ alkyl), or —(CH₂)ₘ—C(O)—(C₁-C₁₀ alkyl);
R¹¹ is —C(O)O(C₁-C₁₀ alkyl), —C(O)NH(C₁-C₁₀ alkyl), —C(O)N(C₁-C₁₀ alkyl)₂, —C(O)NH-aryl, —CH(NH₂)NH₂ or —CH(NH₂)NH(C₁-C₁₀ alkyl);
each m independently is an integer ranging from 0 to 6; and
each n is independently an integer ranging from 0 to 5.

2. A compound of Formula (II): 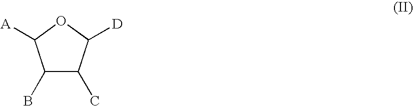
or a pharmaceutically acceptable salt thereof,
wherein
A is —CH₂OH,
B is —OR³;
C is —OR⁴;
wherein R³ and R⁴ are independently the residue of a naturally occurring amino acid that is attached via its C-terminus, or R³ and R⁴ join to form a —P(O)(OH)-group;
D is: 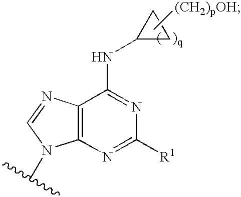
A and B are trans with respect to each other;
B and C are cis with respect to each other;
C and D are cis or trans with respect to each other;
R¹ is —H, -halo, —CN, —N(R²)₂, —OR², —SR², —NHC(O)R², —NHC(O)N(R²), —NHC(O)OR², —C(O)OR², —C(O)R², —C(O)N(R²)², —OC(O)N(R²)², —C(halo)₃, or —NO₂;
each R² is independently —H, —C₁-C₁₀ alkyl, —C₂-C₆ alkenyl, —C₂-C₆ alkynyl, —(CH₂)ₙ-aryl, —(CH₂)ₙ-(3- to 7-membered monocyclic heterocycle), —(CH₂)ₙ-(8- to 12-membered bicyclic heterocycle), —(CH₂)ₙ—(C₃-C₈ monocyclic cycloalkyl), —(CH₂)ₙ—(C₃-C₈ monocyclic cycloalkenyl), —(CH₂)ₙ—(C₈-C₁₂ bicyclic cycloalkyl), or —(CH₂)ₙ—(C₈-C₁₂ bicyclic cycloalkenyl);
each n is an integer ranging from 0 to 6;
each p is an integer ranging from 1 to 6; and
each q is an integer ranging from 1 to 6.

3. A compound of formula (III): 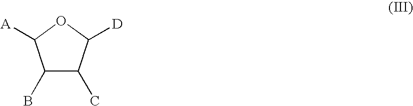
or a pharmaceutically acceptable salt thereof,
wherein
A is —C(O)NHR³; —CH₂OH, —CH₂ONO₂ or —CH₂OSO₃H;
B is —OR⁵;
C is —OR⁶;
wherein R⁵ and R⁶ are independently the residue of a naturally occurring amino acid that is attached via its C-terminus, or join to form a —P(O)(OH)-group;
D is: 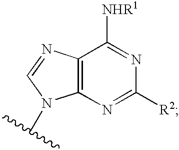
A and B are trans with respect to each other;
B and C are cis with respect to each other;
C and D are cis or trans with respect to each other;
when A is —C(O)NHR³, then R¹ is —H, —C₁-C₆ alkyl, —(C₁-C₆ alkylene)-aryl, or —(C₁-C₆ alkylene)-(arylene)-halo;
when A is —CH₂OH, —CH₂ONO₂ or —CH₂OSO₃H, then R¹ is —H, —C₁-C₆ alkyl, -aryl, -(arylene)-C₁-C₆ alkyl, -3- to 7-membered monocyclic heterocycle, -8- to 12-membered bicyclic heterocycle, —C₃-C₈ monocyclic cycloalkyl, —(C₃-C₈ monocyclic cycloalkylene)-OH, —(CH₂)ₙOH—(C₃-C₈ monocyclic cycloalkylene)-OH, —C₈-C₁₂ bicyclic cycloalkyl, -(3- to 7-membered monocyclic heterocyclene)-S-aryl, —(C₁-C₆ alkylene)-S-(8- to 12-membered bicyclic heterocycle) or —(C₁-C₆ alkylene)-aryl;
R² is —H, -halo, —C₁-C₆ alkyl, -aryl, —CN, —OR⁴, —C(O)NH(CH₂)ₙR⁴, —C≡C—R⁴, —CH═CHR⁴, —NH—N═CHR⁴, —NH(C₁-C₆ alkyl), 3- to 7-membered monocyclic heterocycle, -8- to 12-membered bicyclic heterocycle, —NH((C₁-C₆ alkylene)-C₃-C₈ monocyclic cycloalkyl), —NH—((C₁-C₆ alkylene)-C₈-C₁₂ bicyclic cycloalkyl), —NH((C₁-C₆ alkylene)-aryl), —NH((C₁-C₆ alkylene)-(arylene)—(CH₂)ₙ-COOH),—NH((C₁-C₆ alkylene)-3- to 7-membered monocyclic heterocycle), —CH₂—O—(C₁-C₆ alkyl), —CH₂—NH(C₁-C₆ alkyl) or —CH₂—NH-aryl;
R³ is —C₁-C₆ alkyl;
R⁴ is —H, —C₁-C₆ alkyl, -aryl, -3- to 7-membered monocyclic heterocycle, -8- to 12-membered bicyclic heterocycle, —C₃-C8 monocyclic cycloalkyl, —CH₂—(C₃-C₈ monocyclic cycloalkyl), —C₈-C₁₂ bicyclic cycloalkyl, —(C₁-C₆ alkylene)-(C₃-C₈ monocyclic cycloalkylene)—CH₂OH; and
n is an integer ranging from 0 to 6.

4. A composition comprising an effective amount of a compound or pharmaceutically acceptable salt of a compound of claim 1, and a physiologically acceptable carrier or vehicle.

5. A composition comprising a cardioplegia-inducing agent, an effective amount of a compound or pharmaceutically acceptable salt of a compound of claim 1 and a physiologically acceptable carrier or vehicle.

6. A method for treating a neurological disorder, the method comprising administering to a subject in need thereof a compound or pharmaceutically acceptable salt of the compound claim 1 in an amount effective to treat the neurological disorder.

7. A method for treating a cardiovascular disease, the method comprising administering to a subject in need thereof a compound or pharmaceutically acceptable salt of the compound of claim 1 in an amount effective to treat the cardiovascular disease.

8. A method for treating an ischemic condition, the method comprising administering to a subject in need thereof a compound or pharmaceutically acceptable salt of the compound of claim 1 in an amount effective to treat the ischemic condition.

9. A method for treating diabetes, the method comprising administering to a subject in need thereof a compound or pharmaceutically acceptable salt of the compound of claim 1 in an amount effective to treat the diabetes.

10. A method for protecting a subject's heart against myocardial damage during cardioplegia, the method comprising administering to a subject in need thereof a cardioplegia-inducing agent and an effective amount of a compound or pharmaceutically acceptable salt of the compound of claim 1.

11. A method for reducing a subject's rate of metabolism, the method comprising administering to a subject in need thereof a compound or pharmaceutically acceptable salt of the compound of claim 1 in an amount effective to reduce the subject's metabolism.

12. A method for reducing a subject's rate of oxygen consumption, the method comprising administering to a subject in need thereof a compound or pharmaceutically acceptable salt of the compound of claim 1 in an amount effective to reduce the subject's rate of oxygen consumption.

13. A method for treating obesity, the method comprising administering to a subject in need thereof a compound or pharmaceutically acceptable salt of the compound of claim 1 in an amount effective to treat the obesity.

14. A method for treating a wasting disease, the method comprising administering to a subject in need thereof a compound or pharmaceutically acceptable salt of the compound of claim 1 in an amount effective to treat the wasting disease.

15. A method for treating a reperfusion injury, the method comprising administering to a subject in need thereof a compound or pharmaceutically acceptable salt of the compound of claim 1 in an amount effective to treat the reperfusion injury.

16. A method for treating an ophthalmic condition, the method comprising administering to a subject in need thereof a compound or pharmaceutically acceptable salt of the compound of claim 1 in an amount effective to treat the ophthalmic condition.

17. A method for reducing an subject's core body temperature, the method comprising administering to a subject in need thereof a compound or pharmaceutically acceptable salt of the compound of claim 1 in an amount effective to reduce the subject's core body temperature.

18. A composition comprising an effective amount of a compound or pharmaceutically acceptable salt of a compound of claim 2, and a physiologically acceptable carrier or vehicle.

19. A composition comprising a cardioplegia-inducing agent, an effective amount of a compound or pharmaceutically acceptable salt of a compound of claim 2 and a physiologically acceptable carrier or vehicle.

20. A method for treating a neurological disorder, the method comprising administering to a subject in need thereof a compound or pharmaceutically acceptable salt of the compound claim 2 in an amount effective to treat the neurological disorder.

21. A method for treating a cardiovascular disease, the method comprising administering to a subject in need thereof a compound or pharmaceutically acceptable salt of the compound of claim 2 in an amount effective to treat the cardiovascular disease.

22. A method for treating an ischemic condition, the method comprising administering to a subject in need thereof a compound or pharmaceutically acceptable salt of the compound of claim 2 in an amount effective to treat the ischemic condition.

23. A method for treating diabetes, the method comprising administering to a subject in need thereof a compound or pharmaceutically acceptable salt of the compound of claim 2 in an amount effective to treat the diabetes.

24. A method for protecting a subject's heart against myocardial damage during cardioplegia, the method comprising administering to a subject in need thereof a cardioplegia-inducing agent and an effective amount of a compound or pharmaceutically acceptable salt of the compound of claim 2.

25. A method for reducing a subject's rate of metabolism, the method comprising administering to a subject in need thereof a compound or pharmaceutically acceptable salt of the compound of claim 2 in an amount effective to reduce the subject's metabolism.

26. A method for reducing a subject's rate of oxygen consumption, the method comprising administering to a subject in need thereof a compound or pharmaceutically acceptable salt of the compound of claim 2 in an amount effective to reduce the subject's rate of oxygen consumption.

27. A method for treating obesity, the method comprising administering to a subject in need thereof a compound or pharmaceutically acceptable salt of the compound of claim 2 in an amount effective to treat the obesity.

28. A method for treating a wasting disease, the method comprising administering to a subject in need thereof a compound or pharmaceutically acceptable salt of the compound of claim 2 in an amount effective to treat the wasting disease.

29. A method for treating a reperfusion injury, the method comprising administering to a subject in need thereof a compound or pharmaceutically acceptable salt of the compound of claim 2 in an amount effective to treat the reperfusion injury.

30. A method for treating an ophthalmic condition, the method comprising administering to a subject in need thereof a compound or pharmaceutically acceptable salt of the compound of claim 2 in an amount effective to treat the ophthalmic condition.

31. A method for reducing an subject's core body temperature, the method comprising administering to a subject in need thereof a compound or pharmaceutically acceptable salt of the compound of claim 2 in an amount effective to reduce the subject's core body temperature.

32. A composition comprising an effective amount of a compound or pharmaceutically acceptable salt of a compound of claim 3, and a physiologically acceptable carrier or vehicle.

33. A composition comprising a cardioplegia-inducing agent, an effective amount of a compound or pharmaceutically acceptable salt of a compound of claim 3 and a physiologically acceptable carrier or vehicle.

34. A method for treating a neurological disorder, the method comprising administering to a subject in need thereof a compound or pharmaceutically acceptable salt of the compound claim 3 in an amount effective to treat the neurological disorder.

35. A method for treating a cardiovascular disease, the method comprising administering to a subject in need thereof a compound or pharmaceutically acceptable salt of the compound of claim 3 in an amount effective to treat the cardiovascular disease.

36. A method for treating an ischemic condition, the method comprising administering to a subject in need thereof a compound or pharmaceutically acceptable salt of the compound of claim 3 in an amount effective to treat the ischemic condition.

37. A method for treating diabetes, the method comprising administering to a subject in need thereof a compound or pharmaceutically acceptable salt of the compound of claim 3 in an amount effective to treat the diabetes.

38. A method for protecting a subject's heart against myocardial damage during cardioplegia, the method comprising administering to a subject in need thereof a cardioplegia-inducing agent and an effective amount of a compound or pharmaceutically acceptable salt of the compound of claim 3.

39. A method for reducing a subject's rate of metabolism, the method comprising administering to a subject in need thereof a compound or pharmaceutically acceptable salt of the compound of claim 3 in an amount effective to reduce the subject's metabolism.

40. A method for reducing a subject's rate of oxygen consumption, the method comprising administering to a subject in need thereof a compound or pharmaceutically acceptable salt of the compound of claim 3 in an amount effective to reduce the subject's rate of oxygen consumption.

41. A method for treating obesity, the method comprising administering to a subject in need thereof a compound or pharmaceutically acceptable salt of the compound of claim 3 in an amount effective to treat the obesity.

42. A method for treating a wasting disease, the method comprising administering to a subject in need thereof a compound or pharmaceutically acceptable salt of the compound of claim 3 in an amount effective to treat the wasting disease.

43. A method for treating a reperftision injury, the method comprising administering to a subject in need thereof a compound or pharmaceutically acceptable salt of the compound of claim 3 in an amount effective to treat the reperfusion injury.

44. A method for treating an ophthalmic condition, the method comprising administering to a subject in need thereof a compound or pharmaceutically acceptable salt of the compound of claim 3 in an amount effective to treat the ophthalmic condition.

45. A method for reducing an subject's core body temperature, the method comprising administering to a subject in need thereof a compound or pharmaceutically acceptable salt of the compound of claim 3 in an amount effective to reduce the subject's core body temperature.
